SEQUENCE LISTING (95 sequences)

SEQ ID NO: 1 - DNA, 23 bp, Artificial sequence
  crispr RNA (crRNA) nucleic acid sequence
gggcaagtattggggagaaatgg

SEQ ID NO: 2 - DNA, 23 bp, Artificial sequence
  crispr RNA (crRNA) nucleic acid sequence
gtttgagtgagctgggtgtttgg

SEQ ID NO: 3 - DNA, 23 bp, Artificial sequence
  crispr RNA (crRNA) nucleic acid sequence
gctagttgcatcctcttaaaggg

SEQ ID NO: 4 - DNA, 23 bp, Artificial sequence
  crispr RNA (crRNA) nucleic acid sequence
ggttgccaccaaagttggcgagg

SEQ ID NO: 5 - DNA, 83 bp, Artificial sequence
  tracrRNR amino acid sequence
gttttagagctagaaatagcaagttaaaataaggctagtccgttatcaacttgaaaaagtggcaccgagtcggtgcttttttt

SEQ ID NO: 6 - DNA, 28 bp, Artificial sequence
  Single strand DNA oligonucleotide
taattacaaagcaaggagatgctctcat

SEQ ID NO: 7 - DNA, 26 bp, Artificial sequence
  Single strand DNA oligonucleotide
ttgccatgtagcagcatcccaataat

SEQ ID NO: 8 - DNA, 27 bp, Artificial sequence
  Single strand DNA oligonucleotide
cagtagagtatgcactccagaatcttg

SEQ ID NO: 9 - DNA, 28 bp, Artificial sequence
  Single strand DNA oligonucleotide
ctgactcagatatctgtcacacaaagag

SEQ ID NO: 10 - DNA, 27 bp, Artificial sequence
  Single strand DNA oligonucleotide
atatcaacatgtggatattccgtgcac

SEQ ID NO: 11 - DNA, 27 bp, Artificial sequence
  Single strand DNA oligonucleotide
tctaacaatcgtttgagcacaaacatc

SEQ ID NO: 12 - DNA, 26 bp, Artificial sequence
  Single strand DNA oligonucleotide
tacatagctgttttcttggtcccgac

SEQ ID NO: 13 - DNA, 28 bp, Artificial sequence
  Single strand DNA oligonucleotide
cttcattatgcaggcataagggtgtttg

SEQ ID NO: 14 - DNA, 25 bp, Artificial sequence
  Single strand DNA oligonucleotide
tatcagttagaggcattggagacgg

SEQ ID NO: 15 - DNA, 27 bp, Artificial sequence
  Single strand DNA oligonucleotide
tctaacaatcgtttgagcacaaacatc

SEQ ID NO: 16 - DNA, 27 bp, Artificial sequence
  Single strand DNA oligonucleotide
cagtagagtatgcactccagaatcttg

SEQ ID NO: 17 - DNA, 28 bp, Artificial sequence
  Single strand DNA oligonucleotide
ctgactcagatatctgtcacacaaagag

SEQ ID NO: 18 - DNA, 27 bp, Artificial sequence
  Single strand DNA oligonucleotide
cagtagagtatgcactccagaatcttg

SEQ ID NO: 19 - DNA, 26 bp, Artificial sequence
  Single strand DNA oligonucleotide
cgaaatgcttgatgcagaactgatgg

SEQ ID NO: 20 - DNA, 24 bp, Artificial sequence
  Single strand DNA oligonucleotide
gagacgagttccagaagtgtatag

SEQ ID NO: 21 - DNA, 21 bp, Artificial sequence
  Single strand DNA oligonucleotide
ttccttagccaaatccttcca

SEQ ID NO: 22 - DNA, 24 bp, Artificial sequence
  Single strand DNA oligonucleotide
gggagatgctgaggaaggagagga

SEQ ID NO: 23 - DNA, 20 bp, Artificial sequence
  Single strand DNA oligonucleotide
gctacatggcagtggaagaa

SEQ ID NO: 24 - DNA, 23 bp, Artificial sequence
  Single strand DNA oligonucleotide
cagcgaatgtcttcaaagtaacc

SEQ ID NO: 25 - DNA, 24 bp, Artificial sequence
  Single strand DNA oligonucleotide
tgctacttgggctacagctaggga

SEQ ID NO: 26 - DNA, 22 bp, Artificial sequence
  crispr RNA (crRNA) nucleic acid sequence
gcatgcattgaacacgccttgg

SEQ ID NO: 27 - DNA, 23 bp, Artificial sequence
  crispr RNA (crRNA) nucleic acid sequence
gttgttatgatccagtgaggtgg

SEQ ID NO: 28 - DNA, 23 bp, Artificial sequence
  crispr RNA (crRNA) nucleic acid sequence
gaaactctttggtatagctttgg

SEQ ID NO: 29 - DNA, 24 bp, Artificial sequence
  crispr RNA (crRNA) nucleic acid sequence
gcttcaaagttcagttctgaccgg

SEQ ID NO: 30 - DNA, 22 bp, Artificial sequence
  crispr RNA (crRNA) nucleic acid sequence
gaaatctgcaacgcacatgtgg

SEQ ID NO: 31 - DNA, 23 bp, Artificial sequence
  crispr RNA (crRNA) nucleic acid sequence
ggatgagaagtgcctaaaattgg

SEQ ID NO: 32 - protein, 5 aa, Artificial sequence
  C-terminal canonical catalytic domain amino acid sequence
DLDGD

SEQ ID NO: 33 - DNA, 34 bp, Artificial sequence
  sequencing products (nucleic acid sequence)
tggtgaaggctagttgcatcctcttaaaggggga

SEQ ID NO: 34 - DNA, 53 bp, Artificial sequence
  sequencing products (nucleic acid sequence)
catactcacttgaaagtgggcaagtattggggagaaatgggtgtcgattattg

SEQ ID NO: 35 - DNA, 54 bp, Artificial sequence
  sequencing products (nucleic acid sequence)
catactgacttgaaagtgggcaagtattggggagaaaatgggtgtcgattattg

SEQ ID NO: 36 - DNA, 32 bp, Artificial sequence
  sequencing products (nucleic acid sequence)
catactgacttgaaagtgggtgtcgattattg

SEQ ID NO: 37 - DNA, 51 bp, Artificial sequence
  sequencing products (nucleic acid sequence)
ttgcacccttgttttggtgaaggctagttgcatcctcttaaagggggatca

SEQ ID NO: 38 - DNA, 45 bp, Artificial sequence
  sequencing products (nucleic acid sequence)
ttgcacccttgttttggtgaaggctagttgcaaaaagggggatca

SEQ ID NO: 39 - DNA, 54 bp, Artificial sequence
  sequencing products (nucleic acid sequence)
ggaaggctagttgcatcctcttaaagggggatcactatgagcgccatcaaatgg

SEQ ID NO: 40 - DNA, 46 bp, Artificial sequence
  sequencing products (nucleic acid sequence)
ggaaggctagttgcatagggggatcactatgagcgccatcaaatgg

SEQ ID NO: 41 - DNA, 23 bp, Artificial sequence
  sequencing products (nucleic acid sequence)
ggaaggctagttgcatccaatgg

SEQ ID NO: 42 - DNA, 59 bp, Artificial sequence
  sequencing products (nucleic acid sequence)
gctctcattatgcatactgacttgaaagtgggcaagtattggggagaaatgggtgtcga

SEQ ID NO: 43 - DNA, 48 bp, Artificial sequence
  sequencing products (nucleic acid sequence)
gctctcattatgcatactgacttgaaagtgggcaaaaatgggtgtcga

SEQ ID NO: 44 - DNA, 17 bp, Artificial sequence
  sequencing products (nucleic acid sequence)
gctcaaatgggtgtcga

SEQ ID NO: 45 - DNA, 75 bp, Artificial sequence
  sequencing products (nucleic acid sequence)
agtgggcaagtattggggagaaatgggttgtttgagtgagctgggtgtttggctgactttcaaggtaataagttc

SEQ ID NO: 46 - DNA, 13 bp, Artificial sequence
  sequencing products (nucleic acid sequence)
agtgggcaagttc

SEQ ID NO: 47 - DNA, 34 bp, Artificial sequence
  sequencing products (nucleic acid sequence)
tgaaggctagttgcatcctcttaaagggggatca

SEQ ID NO: 48 - DNA, 35 bp, Artificial sequence
  sequencing products (nucleic acid sequence)
tgaaggctagttgcatcctcttaaaagggggatca

SEQ ID NO: 49 - DNA, 36 bp, Artificial sequence
  sequencing products (nucleic acid sequence)
tgaaggctagttgcatcctcttaaaaagggggatca

SEQ ID NO: 50 - DNA, 61 bp, Artificial sequence
  sequencing products (nucleic acid sequence)
tgaaggctagttgcatcctcttaaagggggatcaggttgccaccaaagttggcgaggaagg

SEQ ID NO: 51 - DNA, 30 bp, Artificial sequence
  sequencing products (nucleic acid sequence)
tgaaggctagttgcatcctcttaaggaagg

SEQ ID NO: 52 - DNA, 36 bp, Artificial sequence; identical to SEQ ID NO: 49
  sequencing products (nucleic acid sequence)

SEQ ID NO: 53 - DNA, 33 bp, Artificial sequence
  sequencing products (nucleic acid sequence)
tattttgtttgagtgagctgggtgtttggccgg

SEQ ID NO: 54 - DNA, 69 bp, Artificial sequence
  sequencing products (nucleic acid sequence)
acttgaaagtgggcaagtattggggagaaatggagtgtttgagtgagctgggtgtttggccggctttca

SEQ ID NO: 55 - DNA, 37 bp, Artificial sequence
  sequencing products (nucleic acid sequence)
acttgaaagtgggcaagtattggggaggtttggccgg

SEQ ID NO: 56 - DNA, 69 bp, Artificial sequence; identical to SEQ ID NO: 54
  sequencing products (nucleic acid sequence)

SEQ ID NO: 57 - DNA, 22 bp, Artificial sequence
  sequencing products (nucleic acid sequence)
acttgaaagtgggcaagtattg

SEQ ID NO: 58 - DNA, 40 bp, Artificial sequence
  sequencing products (nucleic acid sequence)
tggtgaaggctagttgcatcctcttaaagggggatcacta

SEQ ID NO: 59 - DNA, 27 bp, Artificial sequence
  sequencing products (nucleic acid sequence)
tggtgaaggctagttgcatcctctaaa

SEQ ID NO: 60 - DNA, 49 bp, Artificial sequence
  sequencing products (nucleic acid sequence)
tggtgaaggctagttgcatcctcttaaagggggatcactatgagcgcca

SEQ ID NO: 61 - DNA, 48 bp, Artificial sequence
  sequencing products (nucleic acid sequence)
tggtgaaggctagttgcatcctcttaagggggatcactatgagcgcca

SEQ ID NO: 62 - DNA, 46 bp, Artificial sequence
  sequencing products (nucleic acid sequence)
tggtgaaggctagttgcatcctaaagggggatcactatgagcgcca

SEQ ID NO: 63 - DNA, 84930 bp, Nicotiana tabacum
  misc_feature (12218)..(12218): n is a, c, g, or t
  misc_feature (22802)..(22802): n is a, c, g, or t
  misc_feature (28440)..(28440): n is a, c, g, or t
  misc_feature (29634)..(29634): n is a, c, g, or t
  misc_feature (33195)..(33195): n is a, c, g, or t
  misc_feature (33641)..(33641): n is a, c, g, or t
  misc_feature (34400)..(34400): n is a, c, g, or t
  misc_feature (36044)..(36044): n is a, c, g, or t
  misc_feature (37025)..(37025): n is a, c, g, or t
  misc_feature (42114)..(42114): n is a, c, g, or t
  misc_feature (71426)..(71463): n is a, c, g, or t
tatatatatagtaacttatagtactttgttatgtatattttgaaatattaactattgtaacttgtagtactttttatgtattttctaaatgtgtaaatgttatttcaaaatacctaaaaaaatctatgtctgaattgacaaaaaaaagtcatttggccctctaactctgaaaaggttcaaacaaattaaaacgaaaggagtgcaatttatcgactattattttttagagccgttgaaggtatacatttctcttgtttagttgtatgttaatattgtaatactcctatattttatttcattgttactttaaatactttatagttacaatttatagtattgtaatacaaagggatttgaaagaagttttcctacttttcaaagtacaaatggaaaagatataacgataagcgcgcagggacttcaagatattgttatataccgatagtgtaaaagattcttttaccctatcatgtcatctaaatgataattatagataataatctatatataataagtaaaattagtgacctgaaaaatacgataaattacctgttacaacatattaaaatcattgatagtgtgaaaagttctttacattattgttgtatatgaattaaatccaaattaaaacaagtattttccctttcttctaggtaatatcaattagagaccaaaaataaaatataaaatagtatagtagttatttgtagtggaataaattgagtcaaattttttccaagaaaatagtacaaaaaaagagcaaaataagaaattgttgtgaggtgatccaacttttaagggaaaaaaggaattcagacattcaaagatttattgaatggtatctccttttttgtagacattttcaaaggtaaccatagaccaaaattatatggttgtatataaatgaatggtagaggtttcaaataagcaaacattaataattctctctctttcaacacccgagccttataaatatttaaggggattgaaagagggagatagaaaccgacgtttttgtgtatttggagattccaagaaaagtgaaaaagtaaaataaacaacaaaaaaataaggagagagacagagcaattctgcaaataagtctctctccatttattttttttaacaagaaagcttttttagtctttcttgttttttctcatgtgttgaaaaaaatattaattttccttctcttggttcctcttattaaattatcatcatgacgtctaatttatcattagaagatataaaaaatgagcaagttgaccttgtaagttacatctctaattctccttatctcttttttccccttgtatatttttcattttcttgaatttttttactacatatacctcgcattaggggcacaagctaattcagtttctagagttaatgagtttaaaatgaatttgatcgtatatgtatatattctattttttttgttgcataaatatatacatagttcgagtcgaaagcaataagtttagttaaactggctctaataagatgcatgtcatggagaccacatattatacatgcacaagcacttacctttggaaagagtacatcatgcattaggggcatgtcacgtagaccatatattatacgtcgcggacaccatgcattaagaggcatgtcacgtagaccatatattatatgtcgcggacaccatgcatcgggggcatgtcacgtagaccatatattatatgtcgcggacaccatgcatcgggggcatgtcacgtagaccatatattattcgtagcgaacaccatgcattaggggcatgtcacgtagaccctatattatacgtcgcagacacttataatgacaatgcaaatattttttatattattagtgtaattttaatatgtgataacaggttagttatttttaattaagcagttgaaaatgatatattgaatgaatgtgcaggaaaacattccggtggaggaagtgtttcagcagctaaaatgtagcaaagaaggattatcatctgaagagggccaaaaaaggatccaaatatttggccccaacaaacttgaagagaaaaaggtagtctttttttgttcattaataatcttcatattctattttaattaaatcttgaatattagcattaataatttatgtttttttctaattgtaggagaacaagcttctcaaattcttggggtttatgtggaatcctctctcatgggttatggaatcagctgctattatggccattgttttggccaatggaggagtaattacaatattaacttatacagttattttttatattaataaggttaattagtgcataattagtaatggagaaatcttcaaaaaaatgcagggcaagccaccggattggccagatttcgttggtatcactgtattgctcataatcaactctactatcagttttattgaagaaaataatgcaggaaatgctgcagcttcccttatggctaatcttgctcccaaaactaaggtatcccataattcattatcatacatgctttagtgagaggtcagaagcggtaagattcagacttaatgagttcaatctttatgttttcatcgctgaactcagtggacttttgcaataatggcttcaagatttaatatttgttaaaattttaatgattttcacgtatatatctatgatccgtgtgaaatatgatggttcagttgaagccattgaacctatattgcttccgcctctagtttagtcctctacagagacggatacaacatattaacaccgtgttcatctgaatccaatactttcagtttggagtataaatttatgtgtaaaaattcactaaaattgctatatatagtaggtctgaacccataattttagaaatataatagattcaatgctaagaatcttaaagattgaacctatagagttagattctggctccgcctttggtcctctatcgatttcttaatgtgctatgatgatattgatcataatctttgaaaaaatttgtgacttaattcagattctgagagatggaaaatggtcagaggaggatgcctcaatcttggttccaggtgatcttattagtatcaagttgggagatattgtcccagccgatgctcgtcttcttgagggtgatcccctcaagattgatcaggctgcactcactggtgagtccttacctgtgacaaagttccctggggctgaggttttctctggttccactgtcaagcaaggagaaatcgaggcagtagtcatcgctactggtgttcacaccttctttggaaaggctgctcatcttgtagatagcacaaaccaagttggtcatttccaaaaggtaaaagaaaataactaatgatttgtggtctgaagttaaagattcacttaattcttgaaaaaacgattgtgatttgggattctgaactttggctggattgcgatgatcaggtattgactgcaatcggaaacttctgtatctgctctattgctgtgggaatggtgattgagatagtggttatgtacccaatccaaaggcgcaaatatagagatggaattgacaacttgcttgtgttgcttatcggaggtatcccaattgccatgccaacagtcttgtctgtgacaatggctattggatctcataggcttgcacaacaaggtgccattacaaagagaatgactgcaattgaggaaatggccggtatggatgtcctttgcagtgacaagaccggtactctcaccctcaacaagctcaccgttgacaaaaatttgattgaggttggtaatcaggacaacgttgtaccttttttcgtgattattatagttatatttgaggaaatatgaaggcgaaaacgttatcaatgtttggtggttgtaggttttccctaaagatgcggacaaggataccgttatgttacttgctgctagggcttccagggttgaaaaccaagatgctatcgatgcttgcatcgttaacatgttgggtgatcccaaggaggtaatatattttatcatgaggtgatattagtagttttttcactattccaagaaattgtaaaatggcattatatgcttcttggaactttgcaggcaagatcaggcatccaagaggttcatttcttacctttcaatccggttgaaaagcgcacagcaattacatatattgatgacaaaggaaactggcacagggctagcaaaggagctcctgagcaagtacgatagtgtttctatatttgccttcacttcatgtatttcctgtgttcggcatctcatctgccgttaccctttgccagattattgaactttgtgagctcaaaggagatatcaggaagaagtccttagatatcatcgacagctatgccaaccgtggtcttcgttccttggctgtggcaagacaggtaaactaacatactgttagataactctgtttggataagtgcatcatattattgattgtcgttaactaagcatgaatacatgatatttatgcagactgtaccagaaaagagcaaggaaagcgatggttcaccttgggaattcgttggccttctgcccctttttgatcctccaaggcatgacagtgctgaaacaatccgcaaagctcttgatcttggtgttaatgttaagatgatcactggtgaccagctcgccatcggtaaggaaactgcccgtaggcttggcatgggaaccaacatgtatccttcttcagctcttcttggagagcacaaagatgcagccattgcttcaattcctgtcgatgagctcattgaaaaggcagatggttttgccggagtcttcccaggtaaatattagtagtactatcattgaactacaatatagaaaagtttaggcaaaagaactaacataaaaaggtttgattttgtgtagagcataaatatgagattgtgaagaagctccaagaaagaaaccatatctgcggtatgacaggagatggagtgaacgatgcaccagcactaaagaaggcagacattggtattgctgtcgatgatgcaacagacgctgccaggagtgcatctgatatcgtcttgactgaaccaggtcttagtgttatcgtgagtgctgtgttgacaagcagagccatcttccagaggatgaagaactacaccatctatgcagtttccatcacaatccgtgtcgttatgggattcatgctcattgcccttatctggaagttcgacttctcgccattcatggtccttatcattgccatactcaatgatggaactatcatgaccatctctaaggacagggtgaaaccatctccattgcccgattcgtggaaactcaaagaaatctttgccactggtgttgtactcgggacctaccaagcgatcatgactgttgtgttcttctaccttgcagctgacactgatttcttctctgtatgttattctccccctaaacttaaacaaaaattgaaaacataacatgaaataaactaacaaattttatcatatatgtgcaggaaaacttccatgttagatcaatcagaaactctccaactgagcttacagctgcactataccttcaagtgagcattattagtcaagctctcatctttgtgaccagatcaagaagctggtcattcgtcgaacgccctggccttatgcttgttgctgccttctttgccgcccaattggtaaggatcttgaaaaatgaattacttgacttggtttttaatgtgttgttctatcggaaacagcctctctaccttctcgaaggtaggggaaggtctgcgtacaccccccgactccacattatgggattacactgggtttgttgttgttggttttcaatatgtggctacctgaaattcataattttctcctattttttccaggttgctaccgtgctcgctgtatatgcaaactgggaatttgccaggatcaagggagttggttggggatgggcagcagttatctggatctacactattattacctatatccctcttgatattcttaaattcatcatccgtttcgctttgagtggaagggcatgggattcaatgatccagaacaaggtaaaagaagcaacaaaaattgacataacatgaacatttccattgaattctattcttgactaattagtaatatcatttcttgaatatattagactgccttcacaaccaagaaggactatggaaggggtgaaagggaagcacaatgggctttagctcaacgtacacttcacggtctccagacaccagaaaacactggcctattcaatgacaagaactacagggaattgtccgagattgccgagcaggctaaacgtcgtgctgaagttgcaaggtatatgaacaatatcatagatgctaaattaaaaagggcagtcaggtgcacaaaacatcttgcattcatgcagggtccagggaagggccgcaccccaagagagtgtgatgtaggcagcctaatgcaagcatcagtggctgattccacggctcgaacccctgacctataggtcacacggagacaatttaactgttgctccaaggacagcccggtgcacaaagtatctcacatgcacacagggtccagggaagggtcgcaccccaagagggtgtgatgtaagcagcctaatgcaagcatcagtggctgatttcacgggtcgaactcctgagctataggtcacacggagacaacttgactgttgctccaaggacagccccgtgcacaaagtatcccacattcacgaagggtccaagtaagggccgcaccccaatagggtgtgatgtaggcagcctaatgcaagcaacaatgactgattccacggctcgaacccctgacctataggtcacacgaacacaacttgaccgctgctccaaggacctattgatgtgaaattactatgataaattgttttgagtgacacaaacctatacttacaagaactatgttattgcaggttgagagagctacacactctaaagggacatgttgaatcagtggtcaagctaaagggactagatattgaaacaatccaacaacactatactgtctaaagaagctgcaaatcaaatcaactttctttttttcccatataaggatggacatttttcagctggcattatgttggatgaggatattgttttgttgaacaacaaagggatagctgttcatttaaaaaaaaatcttgagtattaacagttatctatagaaaaaaatagaaaataaaagaaaaaaattgtaactagtagttattttgtatagtaatttatcctatctatatgttgtatgctatatcttatttggttagaacaaatctctttgtctccacaatcttttgaattactttgtgttgcaaatggagttcctctatagaaatgattattattatttttttttgcggcaaatcaataaatgagagttgatgcagataaagggagtagagtgacatgaaaagcaatggaaaaaatatgtagttatgtatatgtaataggttgaagatgttattgtaataatgtagtttgtaaggttattgggaatattaatagaaataacctacttatattaatactatttatagcttcaatgtgcatgttgtcatgcatggtttcgtcttgcttttttcaactaaaagaacaaggaaatacagcaaagagaaggggggaggaaagaatttcctttttttttaaagtagaataaaataaagatgaagaatattataaataaatcttgtaaagaaaattaaagaagcctgcaatttatggcctcacttgcacaaaaagaaaagattaattaaccaatcaactctatctggatcccaaattatttgaagcaagttgttaataaaagcaattaattttggttccaaggctttctaagaaaacaaagacatccattttttttttcaattcacaaacttatgtttaaattctattattcctggttattatcaaactttattaagggtgaagaactcatagttgacatgtaatatagatcaaaacattctctcttatatgactaactagtggcagttagcccttgcttagcaccggcccaatccaaaacttgaactaatttacattgttctgtgaaaatgtaaagtttggcacactaattaatacttcctcatgctccctttaattaattttttaatctttttttatagtccacaatatttgatttttcagatatcaagaaaggattaatttatttattttaagttggccttggaataaaaagcctaggagtatttgttatattttcaatgagtaaattaaggttaatatggtcatattgttaattaatgctaaaaggtgaattccataatatatgaaaacaaacaaaaattaaattaaagtagaccggaggagtatgtggttttcgtggcatataaacaacttcaattacatgtgtttccttattaattatctcaattaatatcgcccttttaagtaaccccaaataatagtgaatactacttccattttaatatgatcatattgagtgcatatgaagttgtaggcccaaattcaacataaatctttaatttaatttggatgaacttcattttagtaattttgtagattcttttttacttaccaattctacgtatttgctgagaaataaaaaaaaataagataaccaagaatttcaattgcactccacattgtggaattcttgttaaatcaagttatttaagatggaaaattgagattttaaattgatagaacaaaaatattttcacagtccagattatgtgattcttcttaccaatttcatattaagtttctcatttctgctacatatacaaagaatagtaaataacatatttcaacaacacaatagtaacaaccacaacttacatttaaatgtaaggtcatacctgaatttataagtacactaataaaaatattatttggtccttccgtagaaataatattatttttataatgcactaattaattttttttcacaagggcttcccactacagtcaaagctatgggtgctcccgaataactaatgtataaagatgtcgtgtattagagaaaaatatacacaatcataacttgtcaaagaagcgaattatttcatttaagataaagtttactaatcataacttgacaagagtgagttgctctagtggtaagcaccatccactttcaaccaagaggttatgagttcgagtcatcccaagagcagggtgaagagttcttggagggatggagccgagggtctatcggaaatagcctctctacctcagggtaggggtaaggtctgcgtaaactccactagtgggattatactgttgttgttgttgcattagagaaaaatatacacaatcataacttgtcaaagatgcgaattatttcatttaagataaagtttacaaatatgatacacatggcatttcttgctatttccaaaatacttgatactctatgaaagaaagaaaaaatagtagttcgttcttttattcacttcaacttaatcttctcaacaaccattcataagcgatgatatattattttgttcgtcattttacagcctgctcttcaaaaagtaattttgttaactcatgatttgtaagaaaggatactccactaacatacttaaaatatgagaaatacatcacataagcataaggtgagctacctattattggcaatacaatctaaaccattcctgaaatatttgtaaagttctttgcttctttttgtgttgtatgttagaaatttcttatgacattctaaagtaccaaataattagtgtagattttcaaataggatcaattctaatagcaaaagaaaataaagtcttagtgttattctccactttgcacaagaaattttcgaagagaataaaaattaagctcaaatattttattgatttagtcaaaccaagtaaactcaattaaccttaaaataattgtccaccttcttttctctaagaaataaattaaatcaaagttaaagcggagaaaatacaaattcaaaaaggctaaggatagaattcaaaaggctttcaattttagaaagctaggcacatatatcaaaaacccaaaagcttaatgaaggaactgtatattgctagttacataaataccctataggcccaggttacaaagtcagcatataattggcattttaactaagatcctgattttttctgtcattagccatgttgacccgaagctccttaaactgtgccttattaatagtaggaatgtactgctgatcttcaaaaataaaaataaaaataaaaataaaaaacttctgttttacttgctaaaaagccttctgacttaacacataaaaactactacactacatcattgttaaaagttctgaactttagttacatcttttgtgcttttgcacaatacaataatgactgcgcctcaattccaagcaaattagatgtgactaataagattacacaaccagaaacaaaaccagatatgcttttgcaccatacaaaacataaagtaattagttttctctattcttttagcaacccaacaacctaccattacattaatatacatttacattaaaaaatttgttgttacaataatagagcaagactatcatttcccactagaaaggaagtgaagttgttggagagaccgctagccacaacagcaactactacgcctcaattcaagtaagtttctggtcaattatatgatttctcactatccatatcgctccattaagaactactacaacgctcaatcccaagcaggtttggggtcggctatatgaatcatcattgtccatgtcgctccatttaagcccattctggtccaatattattataagaaaaataatgaggacatttgacgttctcttctcctactgcttcactctatgtttccatgaagttagttcagaagggatcaagtcttcattacatgaaagaacagcctttgccattagttttccaagccaagcatggtagaacaaacccctggaaccaaggcctgtaaataaccagtacttacacttgctccgacaaccaatgaattcatctatacaacccaagagtggaggtgatccttctgcagtgagtggtggcattgcccttagccctgcacatgctcctgttacggtccaatcttttattgctggatatacagcagatgcctttggcagtaactcttcaagagctttagaagcttcttcttctgggacatgtctggaataattcctcgatttccattcccacgttgaccccaagtacaacgttcgaggtccctgaatagcaagccatgcatctgaaagaatggaaggactgtgctctggatactcctctctgaagtttatcattcagaaaaacttctaataagttgcatgttggaagcaatgaatgttaaaataatttcaaccaataactttgtgccttagatactcgacatagaagaaaatagcagaggacacatcaaaaaaggttttttttttttgttgggggggggtgatgatgatggtaggttgaaaattgaactaggcaacaaatccctcatgctagaaatcagaaccaactaaactcaatcttctcaaataaatattactgtatacagatattctgttatccagaaagaagaataaagggacacaaggagctggaatagcttcatgaagcaacaacaacaagaagcccagtaatttcccacaggtccgaaatagcagtttttcacccattggcagtttttagctgtggattaccttttggggtaattgtctaaattgtgaccatgcttttgaaaaacccgtgtaaacagctcatttgacacgccttgctctatggaatgaaatgcaaggtatgaaaacgggaaagtaattacaattagcctatcacaatcatgagagacttattgtcatcaaagtctgatgtacaatcaagaaagactaatgtaaatacatcgacgtatttaaagataggatatttcagtattccccataaaaatgtcacttcattgctaaagaatatattcatgatatatgggaaaattacaaaagtcattcaatgtcatattttctttataatatttagcaaataaagtatggataaaaatgaggatatcaagaaaatggagatatctgatggacactgtagatatatccatgtaagagatgtataaactataaagatatcaagacaccccagttctcaaggtttgaactcatcttcttgagacaagagtgggttggcatagtggaaagaaccttccaccttcaatctatgagattagggggtttgaatcatctgggggggntgcttgatatgggaattacacacatgtacctgatgtgatcagggagctgcaagtgagcaataacacctctacatgtcctcaaaggaagccttccagagaactcaggaagaaaagctgctctagcacctagacatacgatgactgcatcatattcacctgaaaagaatatgcaaatcactaaaattgacaagatcatgaaacaataaaatcacctaaagtcattgatcagtctgctgtcatattcacctgaaaagaacatgctaatcactagaattagacaggaccgccaaaaaatagaaaatcgcctaaataattgatcggtttgctttaaacactagcacattctgcccaaagaaacctataataggtaaaaaaatccaactccgacctagtctagactcttcacaaaataaaggtattcaccgttagaagtctaagtcttaacagtacctttgaggagtacaaagagaagcagcaaagttaattcccttatttgtttaggaaaagcggatgataatgcaatcgtcccagttagcttattaaccccttcactttggacaaaagagaagaaattgaattaattttaccctcattctgttgtatgaagcaagtaaacagttcacctagagagtggaggttgcaagctcttgaatttattatcaacaagatttttctttttaagaaaagaaaaaacaaatgaaaggagaaatgtttgttttgctacttcacttgtatatgagaaaccacttcttgactacattttttctggtgatacaaagctacagttggaatccaaggaagctaaggtgattcaataaaatacctgctaattcttgaagattatgaactgatttcttgtgcagatataactctttacatgaaatgcctgaactgcacttggattccacaaaagttctgcatgctaagtagagagcctgagagataatcaacagtaaaaacaccttaacgaaataaaggaatagggactagttcagagttctatagttattaggacttctccctccagacctcaagataattctgaggatgaacattcacagcttcacgcatatggaacgccaagtccaggggcacacacaaaccaggtaccagcttctgtgcagcatccttgtgaattgactttattctgcagctggcaatgcagttttgagcattctactcaaggacaagaagacacattcccataatcatgaaattacatataagtttcttattccatgtaataatttgatgtactcacatcattcatcatgtccattttcttcacagagagtgctggtctcacgattccactgttaatcattcaaaccaatttgcttagacactaaaaagatggatacacctcttccgtataatacaaagaaaatagaagctcaatgaaggaataaaagatcttgagtctagggcattgggtcgactaaagtgtacatatcagttttccaactaatggtagtgaaaatatttaaagaaagaaaaggttcacttatacgctcagctaaactgcttcagttcaatcatgagtacatagtaaattggcagataatactaagtacaaactacaccaacgagggatttgttaatggaatatgcttaatgtgatgctaaagacaaattggattcacaaggaaagcaagttaaaaacaggatgcaacagtgaatatcttccccccttctttacttttcttgttggaagtaaaattatttggacttacttttctttcggtcagtgatttaaactaaaacgcttaaaatcaaagaaaaatgtatcaccttttttttttttacattttttaaaattcgaactcatttccattatacccaaaagcatatatcaagacatcttcttagcagcttatatataacatgctttacactgctaaccttctcctgactataaagccaccttcttttgctacagtttctggatttgatcgagcatcttcagcaatatttagaagctttaaactttctttccagcattcttctccccgccacagaagcttaactgcaaattttgagttaaatctcaaaaaggaataacagtaaaaataaattctaaatctttaagcaaatgttgagagacagtgtaaggaattaatttgaaggaagaactctgaacccaactagtaaaagcgttcacaacaacctttgggagagtagggatgaaggagtccaccagacattccagatgcacctccaccaacaccaacttcatcatatatatctatacgtaaattcaattcctcagaactttgctgcaaaacataaataaaagatgcagctgtaagcctatgacaattgaactaaaaaggagcaagtaacttagcattatgactcatagataaaataagttgtcaacggatttaagttatacaagctcaaagatttggtagacagctagggtactttaacttgttgttgcaatttacttgtattatttaacactatagactcagataaaatgaatttgctaaaagaagaaaacaattgtaaaaggatctaaatatactccatccattccaatttgtttggtatagtttgactcgacacaaagacttttaaaacttgtggtcttaaacttgtggctataaaagtttcttaaaatgggaagtttaaaaataattgttttcaaatacataaatgtatcaatctttttggaacggactgataagaaaatattgtcaagcacattggaacgaagggaatatataaatacactgtaaatattttttacatggtgcttaccttatttcccaggttagcaacacacttattatagacagtagcgtgtaattagctaacatataacctcaattataaataacttaaacccattgtaaaatctgactttatgaacaggattaactattgtgaactcagctaagaaagtgtaacacatactgcataatacctgcaacaaatgccaagcaacagacagcccggcaaatccagcaccaagaactgcatatctgaatataaaatccaaacaaaacccaattgagaagagaaaaaaaaaagaaaaaaaaattaaaaagacagtgtaatgtacactctccccattggcttactaactaaccggaggggcctttgtgaaacagcagaatagcaacggacagagaaaagggaagaagaagaagaagaagacacaattttgggagaagaatgtaaaaggccaaaacttgctgataatttcattgaaaatacagctgggttttggattataagcaccattgttaacctctagtagaggtattggtaatagtgtctggttgcaggtttctaaggtttaagaaaacccgttaacccgaatttgtgtttcagcatatcgggttataaaaacggataattcatttaagctccttgaagtttctaaagattttgatatatcccctcaacttgtatagatattctttttacacaccttttcttttgtcatacgccttaagttgaaaattgtttatatgacatactatatttactagtccactcaaaaaaaaaaatacatttctatcttttggaaataatttaatttcaaaccttttattttatctattataaaaggttttataataacacaaatgttattgatcccacaaagcttttattcattaaacttttaggatcacaaatttcaaaagttcttttttcttattaaattctatgacaattgctataaaaaaactcacaccttaacaccaaagattgtttgtccatctgataatctatggattttttaattgacctgaatcatttttggatatgctttccaaatccatctgaattaagaactctttttggattaaggatgaaattaaactttatgatttcattttgtactgtgaagatctctttgaatgtgaaatgataactttgctttgaatcatctgtatccatataagagtccatagcaagttaccaaatattcaaacagtaggtgaaatgtaaccataattctcttatcttttcacgttgtgttttacattgtgaactgttgagctccgacttccaaattacagaaaagaaggtaatcttgataccacgatacaaaatgttgaagcgaagagcaaatttggtactaaacatttgactcttaggtttagagtttacaagataaccaaattacataaatatccacttctaaattctgttgaaattgcagaaagtgcagaattcttaagcattctggagtcacacggataaattacattagatggctcaaagtaactcaaaccatttgacacgagtggacgtgcattcctaccttggacactgcagttatcagtagactaatgcagaggtgttatcacaacactaattccgaaaattaaatatttctgcttgatttgctgaaagcattaaaatcatcaaattacatgtgatttgatgaccagttgactggagatagtgtccttgtgatgtgaacttgctgaaagtgctttagaaggccaagtagagcacatcattaggcttttcggactatgattctccgttctcgtttgtactccacttcatccctttgatgcataattatcagcgctctccttacctgacaattttttagcacttgcatcaatacaaggtggcctaggaatattgttctggtataatttaggaggcttctactggtttactatcaaagaaagtcaatcagatagcaccagaaatacaaataaatggagaaactaggtgaatattgatgactaaatggtatataagtgaagcagtggaaggactcttacaattgattcattcatgcccatcctggtcagttcatcaattagccgtctctctgatatatggctaccaatacccattcttctcttaatttgggcttccgcttgctattcatcgagaaaaaagtaacttcatcaaaacagtgactagaaggacatataactggaatatgagtattgattgcccaagagtcaaacctttatctcatgtgccatctcaggagtaagatttatatgttgattgattccagatcgtgctgcatccattgtggaattttggaagagcctaattgcttccgtgacgtgattctcatttgcaatttgggcgctaaaatcatgataagaaagaggcagattattgatcaaagaactgtttatataggcgaatcttataaaaggacaaagaaaacagtgaaaagaaacacatggaaaaaaggaggtttcctaaacagtcttaaattgcatagttaatagcaggatgattcaagtccctgaagcttgatcctatgcaagagacaaagaaactacttataatcgtggttacttacagtctcattcttgcaagagcttcactcaacctcactatagcctctagttgccgcacagtaattggtattgctgctgcctccccattatcatttgattgccgcctcatttgctgcagcagataacagaagaagcaaatatcagtgagatgatgtacaacattttcacagtcaagaaaggttttggtaaaatgttgcagaaacgggaaatctaagttttgaacaactcccctagtgaatcccaagtttttcagacggccatttgcaagaggtatacttttctgttatcatcctaaaacaacttaaaccttttattagaggtatggagcatataaatgactagaaagttagcaaacttccccacttgagagcaatccaccagaactgcgtacttgaacaaaataaaccaaaaagtacccacttctaatttcagtttcagtatttgaaatttcttgatacatcccataagcactttttcaataaataggttggtgatccagcagactgagcatcaggagcccaatgatgtaattacctgcctaattttgacataactctcttgtaacattgaggctgcagcgggtgatagtcgtggatggcaattggtccgacaatattgtatatacctgcacgaggaactcaattgcttcaacatacagaccttgacaattacaacaatagtgtagtgctcaacttaaagccaggttagaaaccaacctcttcaaccaattatactccttagaagttctagtgtcatccctagatgcctcagcagatgcatggaccttgatgatatggctagcaatactctgtagtgaaaaaaatactcagtggaagcacaaacttaaggaaatatatatcatctttagatgacatagaaatttcattgctagcatcaagtagacgcaagaagatagtacaagggtactacaattgagctcacaaggatattttgaaaattaattacccatttatttgcgtattcatgcccagcatactaagcatactatctagatgcctagtagctagattaaaatgaagtctaccatacaaattttcatttcacccacagacttgggagagaatctttctcttaccttgtcctgactacccatttattcgtgtattcatgcccagcatactaagcatactacctagatgcctagtagctatattaaaatgaagtctgtacccacacaaattttcatttcacccagacttgggaaagagaattttttcttaccttgtcctgactgaacatcctaatgtctttcacaatgaatatcaaatcgaatcttgaaagaattgttgtctgcaagtcaatgttatcttgtgcagtctgcaatgcaaacagcacatcaactcctgttctattgccaacacaagctgcagaaaattgtaaaaacattaaaacaattgaaaggaattatattgaccttgagatcatcatagcgtccagatggaggattagccgctgcaagcacggaggttcttgaattaaggactgttgttattcctgccttagcaatggaaattgtttgctgctccatagcttcatgaatagcaaccctaggcaccagtaagaagctctttagaagagctcatgatagagtagacaagagtatgatgttctaactaaaatctacctatcttctgccctcattttgtcaaactcatctatacagacaactcctccatctgccaagaccatggctcctccttctaggtaaaattcacgctgaattgtttcataaagaaactcatcatttcaagatatattgaaaatgacacatttatctgaaatggcttctagactagggaaaatatggaaacttacagagctattatccctgatgacagaagctgtgagaccagcagctgaagatcctttgccagaagtgtatacggctactggggccgtcttttccacaaattttagaaactaacaggtaaaaataaataattcagactctcatttgcattggaaataaaaactaggatgagtaacaaaagaaaacttttgaaccaaatgccaacttaaatggctccctcatgaagatgaaatttcaatattaacagcaaactgccaccattgtattcagtgaagtaaaactgacctgtgatttagcagttgagggatcccccaagagtaatacattgatatcacctcttaatcttacaccatcgggcaagaactgcacaaatagagttagaaataaaccaataaataaacaaagagcatttaacattgaaggcaaaatgacataccttccttgaccctccaaataaaaggcatgctacagctttctttacatcaacatgcccaaatattgagggagcaatctttgagcatatcttttcataagcatccttatcggatgcaaatttctgaaattcttctttctgcaaaaaggaaggccatgagaactgatacatacaggaaaatccaaacagagtaaaacaatgaccaaattggaaactaatgctgtccaaacagtacaggaacgtcatcggcgtactattagcaaactgttatatcctctaactgcaaggcatattcattaagtacttgatctgctacaaagaggtaaaccctccccccagtccatagcttgaccgacattacttaatcttccccatcctttctccactctatgttcatgcttctttgcatgtttttctactgcgacttgtggagatatgtaggtggagacaattatctgtatgcagtccactcacttgaaagcatgttgctagctgtagttcaaatctctcaattagcacattctattgataatttagaaattacctcatctactgtgaagttggctgaccctcgagaattggcctcattcgtttcttccattccaacaactctaatataaggctgcctaacagcaacagctcctttgtgactgcatcccagttcagaacatagatcccggaagttaaaagaagataacaaagtgacaacactacaaaaacaattaaagttcatttatcacttggtagactcttacgatgttgatgaatttgaagcctgaaaaacactgtagatccccattatagtcagccttgtaccaggcactattgtttgaacaagatggcgatctatagacagaagcatatttcttggaagctctcccgtagggacatcctacaattaccccaacagaaaacacaaatgaaatgagagtatgattataaagaaagccagcagtcaagcttctattctggaactaaatgttccctcacctcagggttttcctgcaatttgagcgtctgctggtcaacatacttacttttatctggaaccactacccatggatcaataggacatgggtcttcccccacctggaaaaaatcctaccattagaatgtcatccaaattacaacctcaaataaagagtttcaaattattattttattccatcaaacctgaggaacatgatcacaagaacgtggtacaatagctcctccaagccctgggcgacatggaacgatcttcacatttttgcagttcttacatagcaaggttacataagttgcttttgccttggtccttgatgcagcaataacgatcccagataccttgaccagctttgatatatattgtgcctaaacaagacaaataggattataaaaatagggggataagtagatcaattagagcaaaacttaagataataaagaaaatgaaaaagttcaaacagaaaactaaccccaagggaacgcattgacattggatcctgctctgaccttaagagtatttgtacttcaccaatctctggctcctccatttctccactctcaccagctaccctcgatctcaaactagccagaacttcagctgctgcagtttcaaactagataaccccgagaccatccagtagaggacacaagaaaaccaaatgttattcagtagtaggttcttaaaagcttccacattaaagaaactacagtatttcatgcaattgaagacaatgatgcactatctaaaacacactgatcttaagtgtacttttcacttacaaaaaggcgtattagttcaacagtgaataaaggacacacccactaccttcggttgtaacaaaatacacacaaaaactatgcctaaatcccaaactagttgatgttgaacagtatatgcccgcttctacaactattccataacccaaaaatgcaactacagggacatcccgtaaaatacatatacatttacctttttgaaaattcaaatttgtttagaaactcaacccatttggtaagcatggagtaaatcaggctataacatacatcagtacaacaaccatcattcattcattcatttgcctcaccaaacataaccctaactatataaacctcaaatgcagaaagctatatacacaaacttcaataacatgagccataatttgcatcaaccgtattgttttacctaatcaacgcaatcttaatccatcgtaatcattcattccagcaaatccggatataatttcaagacaaaaaaacatgttacaactaagcagagccttgtgtttttagtaaacagttgctactccctatacaactacaaaaaatcaagtattctaattggattaaattaatatcaaatggatcctttatgcgtcaaccattgatgataaacaactacaagttcaattctctagtaagctctatagttcatcaatcacaaattcacatccacatatggctaacaatgccaagaaatgtaattagtgcatatttaatatttgtatatcttttgtagatttcttaacacatatctaggatctccaaagctactacgttcagcaaaagctttacctcacctttgtacatccacccttgtcaaacaatcatagtccaacataaccatacatttcaacaaatccaaatgtaattacaaaccaaaaaaagaagaaaaactaaatatcaagggatcctttatcagtcatttattgaatgataactaactaccaaaaaacgttaggtgagttatttctatttgcttaaccattttttagcagagtataaatacaagtgacgtgatatacggatttaaataacggaaagccctctattaagatctcaagtgcatcagccataaattcacatccacctacggccaaaaacacagagaaaggtaattaaatcagtttcataccagagggagataatcagcagggttctgacggaggagttcgggtagggtttggtctcgctcgtacgaaaggaggtctgaaaggttaataattaggtgagtttggttctcgagaagtgcttcngttcttctctctttctctctctgtttaactcactttcagtttcagatctctctctctttgtggcttcagcttctgactttgcggagggttttagagggagtatgattttggcgggaaccacttggcgggaaaatatgcacgtgagtagtgcgtgcgtgtaaagagggggctcctgttcaaatatataaaacttgcgagtcataaagaaaaatagtgaacaatacacattgaatgagtgaaaatcatctccgcctcccaacttttttttttttatacaatatacattcttccccctcctaacttacgtaatatttattttgcccgttgacatttccaattcgcattgtatgtaatactttttaaaaaaattatatataatatttattttaccctagatgatattacttattatttagattattttatattttattatgaattgatatgagttcaataaaactaaaaatacaatagttcttttataaattttgttactgtatctaccgctaatttttatgattattattttaatattatgcgcgctacgtatatatatttaagttctacattctcttactgatgaaatattttctaaatgtaacttaaatttctctgaaagtataaatagataatcactaaaaatgcattatcaaatctatctctatttattttttgtattacctgcattaagtgtatgtaagggttgacaaacgggcgggacggggcggatatgtgtgggtcgaaaataggtatgcaaaaacgaataaactatttgacccatctatatttaacatggatagaaaataggttaatcgatggataatatgaatacttgtgttattcatgacttcttgaatgtgatcacttttggggaaatttctagtcttccaaatttaagggactcccgatttgagtatttgcaaatgtaacacattggttatccgttgagtatttattttctaaatggataatatagattttatacatatttgattcatttttaaaaagttcattatccaacctatttttagtggataatatggatggataatttgcttttttatccatttgtatgtataaatgtgatgacccgatatgtcatttttaattttaacctttatttctgtattccgagaccttaaatagctccgtttagccttcctcgattttgcgtgcactccatatctcttttctgaaaaaatttaatgtgaaaaactaaagaaaatgtgaaattctgtcttacaaactcattgagttgacttcggtcaacgttttgtgtaaatatacccggatcagtattttgacggtcctgattagtccgtatcgttatttgggacttgggcatatgcccggaatcgaattcagaagtccctagcttgatttaacgccatttgtcgaaaactataaatttaaaggtttaaagaattcctaagtttgaccgtaaattgactttgttcatgtcgggttcggattatgattccgagagttagagtagctctattatatcatttggtacttacatgcaaaatttgagattattctgaattgatttgacatgttcccgcgcgagttatagaattgaaaattccataaattcattaagttcgaatcgaggtgtgatttgtagttttgacgttgtttgatgtgatctaagacctcaagtaagtccgtgttatattacgggacatgttggaatattcagacggggacccgaggggctcagatgagtttcggatcgtttggatcattcttggttaagttgggcaggtctattgtggtgtgtccgcatctacgagggctggtcccagatgcgagcccgcagatgcggcacattgatcgcagaaggggaaacatactaggcttggggaccatcgcatatgcggagaaatgaagtgcacctgcgtgcgcttcgaagcaagaaactggcgcagtagcggccttatatatgcgcgtttacttgtcgcacctgcaatgccgcaggagcggatagttttctgcaagtgcgagagccatttctccaatgaccttccgcagaaagcggaacttctctcgcacaagcgacgccgcatatgcggccaatatgtcgcaggggcgaacatgtctcgacagaaacacatatttcggggtttggtatttttatcatttttgagtttgaagcctcggctttgagcgatttcagagggatttttcacgagtatgaggtgggtaagtgttctttatccgaaattgattatatttcgtgatttcataactattttatatcattagtgtcacgacccaaactccctatgtataacatcgtgatgacacctagtctctacgactaggtaagcctaacaaaatgcgaaaaataacaaaacgaaagtaaaatttggcaactaacaacactggataattgaataactagtaacaatatcgctcgacatgtacaataaccaatactctataaatacacagtcttcccaaaatctggaacatcgtaagtcacaagctacagaagaaaaatagcatttctatacagcagagtctaataaaaggaatacaggaagtgtttgacataggggatgaggtaccgccttgtagtgtcaaaagtaaatgtgcagggagaactcatgaggaaccgcttcgtagtctcaaaagtaaatgtgcagggagaactcatgaggaaccgccacgtagtctcaaaagtaattttacagggagaactcccaaggaaccgtctcgtagtctcaaaagtaaacacacaactcaaactgataaatacaacagttaacaacaagatttctatagttaatactgataaagaacaaggaaaaacagaaaatcaactaggcatgcttcacagagttcaaataagcagttaaagattgtagacatgcgatattagactaaacaggataactacacatattggtataacgcaaataagaatgaaaagcaaattactacttagaaaatggtataacccaaattaaaaggaaaacaggttgctacttagtaaaatcaggttttacaacaaatagctcatatacgtactcgtcacctcgcgtacatggcgctcacatatcacaacagtaccaaatcctaaggggatttcccccgcacaaggttaggcaagccacttacctcgcaccaagctcaatcaatcagtaagaatgccctttcctcaattatccgactctgaatggcccaattctagccaaaacaatttcataccatgaatacaactataatagactaatttaatttatgaaatcaagactttaacaagaatttcgaaaatcgccctaaaaagcctccccgggcccacatttcgaaatcgggtaaaagtcaaaaaatatgaacacccattcactcacgagtccactcgtaccaaaattatccaaatatgatatcaaaatatcaatcaaaacccaaaatcttagtggaaaaacttcttcccattttccctaaattttccacccaaattcgaaattaaaagatgaaattagtgatagattagtgggatataaacaaaatcaagggaaaaatcgttacccaattgatgtctctgaaaatccctaaaaatctcgcccaaatccgagctctcaaactcaagttttgataaaaatatccaaccctcgattttggaaactttaacaggcatttccttagcttcttcgcgaacgcgaccacaccctcgcgttcgcgaagcacaaatttacttcggcccaaaatcttccattgcaaacgcgatgtccttgtcgcgaatgcgaagaccactcagctcgaccctacacgaatgcgggaccaacgtcgcgaacgcgtaggcaaaaagggcatggggacccagttggccaatacctccacgcgaacgcgggacctccgtcgcgaacgcgtagaccaagtacctcagtgcttcgcgaacgcgagaccttcatcgcgaacgcgaagcacaaaaattttgcctgcctccagttcctcttcgagaacgcgagactcccctcgcgaacgcgaagaaggaaacaagaactaaagaaaaccaaaaatcagcaatctctcctaagtccaaaatgatccgttaaccacccgaaatcaacacgaggcccccgagacctcaaccaaatatacctacaagttctaaaacactatacgaacttagtcgaaccttcaaatcacatcaaacaacgctaaaaacacgaatcaccctccaattcacacttaatgaactttgaaacttcaacttctacaactgatgccgaaacatatcaaatcacgttcgattaaccttaaattttgcacacaggtcataattgacattacggacctactccaacttccggaatcggaatccgaccccgatatcaaaaagtccactaccggtcaaaatctccaaaaattcgactttcgccaattcaacccaaaatcagctacatacctccaaaacacaatccagacacgctcctaagttcaaaatcacccaacggagctaacggaaccgacgaaacttcatttcagagtcgtcttcacatagtccgactacggtcaaaatgctaagacttaagcttccattttagggactaagtatcccaaaacactccgaaaccaaaaacggaacctcccaaatcacataagcataaaaagatacggggaatgcagtaaataggggatctgggctaatacactcaaaacgactggttgggtcgttacaattaaatggaagaaatgagaatttttgtaaaatctttcaagaatttaaaatgaggatttgaagggcgatccgatatcagaatttgttaattttggtatggttggactcgtatcggaacgagtgttcgggctttgtgaattttattaggtttcgagatgcgggcccgggggagggggaatggggttgactttcgttaaatttttcgaaaatgatctaaattggacctctttaaaatttgtgagtagtttctaaagcttaatttgaattgttggccgattaattgctaggtttggttggtttggaggcttgttcaaaaggcaaggccgtggttgagtgattgcttgaattgtggagcgaggtaagtgttgtggttaaccttgacttgagggattatgacttgtttgccgatttgctacataattaacgtgtgggtacaacgtatatacgaggtgacgagcatttatgtattgttgtgggataaagcatgcgggtgaaatttgtttccttgtgatttattgcctttttatttatgatatccatgcctaggttacattattacttaattgatcatttctcccattattattgcttaattagtaatggtggagtattttggaagttgaggtttggtatcgtggcatcatatttgacataaaacatattcactatgctattttatcttccgaatttatattatttttactaagggagagtgttaaagcacgaagggtgatgccgtgccatctttataccgtatgttgtccgtctttattggttgataatttatttggctatcttgtgatgtcatacctgttgcagttatcgtatctctttcccctttggcatgtcccttcccaattatacattgttaaatctctattttgttgctgtctcgtacatatatatatatatatatatatatatatatatatatgtttgtacaggttntgtcctatcatagcctcgtcactacctcgtcaaggttaggctcgacacttacggggtacattgggtcggttgtactcatattacactctccacttcttgtgcagattttggtactggacctaactatacgtgaggtgcgccaactcgaattatcgcatacggaggcttgaggtagatatattgacgtgcgcaaaccttgaagtccccttctaccttatcctagctgtttatttcatttcagacagttttactttcattcagaccattatttatattattctagtcgttcgtgcacttgtcacaccaattctaggatggtatttagacatcattgttaatttttggtttactcattctattctagatttatttcgattatattggtttctttattattattcaatttgaagtgttaaaaatggctaaaattattctaacgttggcttgcctaacaagtaaaatgttaggcgccatcacatcacgttattctaacgttggcttgcttagaattgtattttctactattataccttgaaagaaaaagaagtgcctatcattttcttgttaaattaacgttatatcataagaaacaattaagtaaaactgataatagaaaatgacagacttggtacaaattagcaagttcctacatactctgtaaggtaatcttttccctcatttacactcaacggcaatagcacaggaatttcaagtagcaggcatctgtgagactgtgactggtttaatcaagaagaaagcgaacaaaaaaatgaagaaaaaagagaattgaaataaaaacaccaagaatgggatactttttttcccaccaagaatgggatgacctccaaaaaatggcaggcctgatatgatttcaatgcctaaagcaatttgccaagtacaactaattacatcatttagcacctattctatttgcagcgacaccaggagtgagtgagaaagacccctttatcacctggttaatatacaaattatatacctgtatacatataggatagacatacagccgtattatcgttgcgctaaagatcatacatacattttttgttacttttaattaagtcagtattcagagggtaagcactctcagttattgaacatatatccttcctgcttcaagaaccaaagtgccccttccgctgcatcctcagctgcatctttctttttcgcccgagnaggtaggtgccataggctataaaatgtcaaggattttcagtgcacagaaataatcaagaaattgaggatgatgttagtggtttttaccttttctggttagatcagatgacaaacttaaagacatggattcccaaggacagcattaaaccacataaccagcaaggattttctttatgttaataggttccaagaattaaacttgagaactacaagtgcaaaataaaaatttaaacaatcaaatagtgcagaaattaaccagatacaaagtatgattgcatatatcctcccatctcaagctgaacttttcttctatatggtgcattgatttctacttggtagattccccacatcggttgtggatggagtaattggtctccttatatggtcttggccaatcttcaccttttgagctaacttttcgggttgaattaggctcaaggtccattatctttgtaacaccccgtacatttggactaggtgtggatatgttaaatgggtattaatttgatattttagacatatgaagtcccatcaggatgagtatcagtggaaatagttgttttggaatcaagatggagagtgaggtgcataagattcaataaaatcgactaagtttatggaaggtactgccttctagaagaatgtcgtaaatttatagccctttgaaatagctttccaacgatatattatggagcccaaacgtatctctgtgcaaaacgttacgtgtgttttacagagcagtgcacaatatgcgcggccaggtgcgcgcccagagggtcacgtgcgcggccgcgcacttgaggcagtgctactaccattttgcgcagtcaggccttcaggtgcgtacctggggggtcatttttaaagccctacttcgtcccccccaccccaaaacacaaaaatttgctctagaaagggaagctctcaagaagctctcccaaggtccctccaccatccaaggtaagttcgaatggtgattctaagttgatttcaattcctcaataccttattaacatgggtaaaccctccaaattgttagatattcgattggggcatcattgttgagagaaggaaccctagaactcgaattcaagaggattgaacttcaaaaaggtaatgtcttcactctctaattgattataccattgattatagactctatgttcatgagatatgagttgatgagtttaatagaaaagcatgaacggttataggtttgggttattgattgttgattattggttaaagggtgttgtttatcttacgagtgatgaagaatggtattgattataacaatttgagattttaatattatctaggagcaaaagaatgggttgttgggtgtagaaacatcattaataaggactatgaagctttatgcctaccaagtgtttgataaaaggcctaagcggccaaaacataagtattattgctaatatggaatccctttgacttgtattgatatagattaaagttgaaagggttggccgaatgttgtattacgctcaagagaaggaagttaaggtatgcgaggctaactctctacgtttgggaatgttaatgattctccctacgtctcattcttatgactattacaaatttcctcagaaagtaattatgcctcagttccatgaatagttgtagacgttactaattgcctcagaaatatagttaagcctagttccctgaataattgcagaacttctattctctagcttcataattcgttcatgactttcattccgaacgttgtgggcttagtgcgtaatcaatatagacttgtgttaactcccatgagtctcagcctaaattaccagcatgccataaatagttgaaacataagtcctcataatcagttcatgaatacatgtcttagttcaattctattcatcattcaagtatcatgtaactcaatatcgttcagtatcatgtattacagtacgattctcagttcatgtttttaaatccctgaatgttgaacacctgtatttgggcctgaggccgtagtttatgctttatgcatgtttgggcctgaggccgtagttgatgctttatacatttttgggcctgaggccgtagcttgtgcacatatatattgggctcgaggccgtagtttattcagataaacaggttgttcatcattcagaagagggaatattcatatccttacttcctttgttcagttatcatttcagttatcaattctcagttatcagcttagtttcagtttcagcatttataattctttctttcagttgctttacataccagtgcaattcaaatgtactcacgtcccttttgcccggggcctgcatctcacgatacaggtaatgatctacaggttgatgattcagagcgctaggattctcgtaccagctatttggtgatccctagttctttcggggcattatcatcactttgcaatctttcagtatttatagatagtcagtattttcagtacttcattagaggcttcatagactagagtaacagttagacagagtcacacacttttcatgttaagcaatgttggctacaaatacgtttcagatttatgttagtatttccgcacttgatttatatcattcagactttcagtatatcatcatgtgttaatttatcttccgtattcagtatgggcccatattatatttacactccaaatgtccagttctgagcttgtgtgtcggtgtgtgtgcgtagggagggggtgttagagacctacatcagttgtggatgagactcttggtctccttacatggtcttggaaaatccttacctcatgagctaactattggggttgagttaggcccaagttccatttcttatcggtacttgttggaggcataagtttcctaggcttcattcatgaatgtcaaataagagtggcaagtgggccgggcccatgggctaagtgggctaaacggactgggactgttaggcctgctagaggtggaccggttcttagctaggaaccgttaggaccgggcccgtttgacccgccaagagaccgggaccggatcggtcccttggcgggtccaacgaatattttatttttaaaaaaagagccgtttgttgtttgaaaaaaaagagctgttgagggtttaaaaaatagccatttaactcccccaacttagttttaaccccaaactttttattattacactttttccctattttctactataaataccctctcattctttcattttcttacaaaatcaatatcaatctatctaaatctctctctaattttgttctatatttgctacaattgtttactttatcaaaacaaaatagtgaaaaaattgtgaggttgctactattacttactttattccaaaaaatagtaaaacaatattaaggttgttacaatttggttctagtagtaattatttttcacaactaaatgagcttgaagtttatatgtcgcagcaacttgaggaagtgaatcccgatggcacctttaatattttgcaatggtggaaggacaaagaaaatacttttcagttctttcaaggatgacccgagacattttaactattcaagcttcaatcgtgnaccttagatatttttttaacatctttttgtctctaatttttatttaactttttttaaaaaaattgctgcttagccgttggacccatttagcccgttgggaccacgaccccagcccgtttagcccgggaccatgagctcgtgagctcggtccctggccggttcctacaaaaagcacgtttagctcgggaccgtttagcctgtttatttaagacccgggaccggcccacttgtcacccctaatgtcaaatgttattttccagtacaacttgtcattttacaattttgggccatttcatgaaactcgatattttacaatgctgaataattactttgagatgagtaattagttaatgcattttgttcattacggtttagtttacgttttcatgattgcaggaatctgctcaaaattctggaatgaaaataaaagctactttttactagtnctctcattctttcattttcttacaaaatcaatatcaatctatctaaatctctctctaattttgttctatatttgctacaattgtttactttatcaaaacaaaatagtgaaaaaattgtgaggttgctactattacttactttattccaaaaaatagtaaaacaatattaaggttgttacaatttggttctagtagtaattatttttcacaactaaatgagcttgaagtttatatgtcgcagcaacttgaggaagtgaatcccgatggcacctttaatattttgcaatggtggaaggacaaagaaaatacttttcagttctttcaaggatgacccgagacattttaactattcaagcttcaatcgtggcatcagagagcgctttcagtcaagcaagacttcaaatctgtgattatagagcgtctatgagggagaacttggaaaatcagtacttttcagagattggatccgttcggaaagaagaaattttggacttggctgaattacaaccagaggtagacgaagcttacgaagaaatgctagctgaacttgctgaggatgctgcttcgcccgacaatggaagcggcgatgaccaagctacttttccgccaccacctacggaacttcctccgaaccttgaaggatttatgaagtttgtaaaaaatatcatgtaacttgtatgaacaaaaaatagtatgtattatgtaacttgtattttggcacatcttgattggttcctttttcttctcaatggtgttatncatactctttcaagtggcttggccctgtctccttgcaacattcaaatctaggaggtttccagcagttggcagcacaaacttcatgcaaaattgactttgccgatccatttattgcaccacctgaattattttggagtaagagaatgacaacatggataaataagcagatagagatccttgttattgatacctgtagcttttgaatctgaattagaaccattaggcatgatggtctgctcagaattgcattgtacagctgcctctggggccaactttctcgcaggctttaacttgaaattgcaatttcttgcaagttcttcactaatagggaatactatcaagtcacgatcactttcagatgcttcgttcttctccacgtcattgcacctagcagtaagaacacagggtgtttcatcatatcctatcagtttagcttccatctttttagctgtttttaccacttgctccagtgatttagacttgcgcctatagccttgagcctgcacattggaagacattactgaactgcaaaatagctatataagagaagtccgataagggaggtactaagattggaatactaaaatggaaatcaaactgcagaaacaatgataaaatcagataagaagagctgacatctatagtaacgacgcctgatcagcatgcaactgctgagagaagcaaagaacaaaagtagcagaagtgttgtcactgtctcactgaccagagagacatttgaagaagagaagtaaaaatagaaggcaaaaggaacatcgaagcatacttgatattatgtaaaccagtcaataaattatttgccaaaactaggtaatttatcaactaaagtgagaaacttgcgaagttctgcgagaaatgttataggttttagtgataaatcattatctttccttatgcatagagatgcatttctctactctgaatgctcacaaggcaatgtaaactgatccatgaaacttgtccccatttcccattacgaccttgaacattgtgagggtcctagtacccccctaaaaaactttcaagtggaattaatgccccctttctcagtcacctggcatagaaagtgttcatcattctctctttaagagtgtgagggaccaaaaagaagttttataatttgtttttgcaagataattttttgttaaatgcatggcgaaggtacatagttgctcctgaactatgagaaatggtttacttttataagaagttggggttgaaactacccctgttgttaccaaaatggtctacacttgcccttttctactaatggaaaatgattagcaccacaaatcgccacgtgttgaagcttaaaattaaaccacaataattgatttgaattaaaggacatgaatgatccatttcacatagtttaggggtaaaatgacaaaaagtttttaaaatgaaattgacacgtggaagcaagaattgcatgctaagcacttcctttccttaaaacaggggttggccaacaaagggggtactaataccacttagaagttgttcagggagtaatacgacccctgcaaagtttaaggttgtaaggggaaatttgggacaagtttgaggggtccttatgtattatccaaaattatatacatcagcttatgccttgttttattcggnagtcactaagtgcttgaacagcctcttttgagctctgtatctgatgactacctactgtcataatcctttaggcaagactcatcactagtacagagcgtgaatgcaatgagaacattctctgcaacttgatccactacaaagaccaaatataactttgtaacaccacggtggtatcacataaatgaagaaagctgtgagagcaagcagttcagtgatctcaatcatgctcctccagcaaaaccgctgaagagattatcaacataaagcagcaacctgatgttggggtgagtaaagacttgaatgatggaatcaacaagaaagtctgtgctacaagaacagtgatgtcacgagaagaaatcaatttgagcaataagccaccaaaatcgatgttttcgtactattttattgtatgaggagaaagttcgttcataacgaagaagagcacaacagcaggattacaatattttggagctaaaaataattattaaagttacagagatctcaaaattctttaaggattagtcaacagaacaccatgggagaggcctgcaaccatgcaccaaattattgcaaaaaaaaccactctttctcatacatggcatttgaaagaaggtgatcatgacatggtctaggtttgcacccaaattcagcatagtacagccaccaaagcccctcactgtaggaccttagcattctagctagttgctacaaccaatacccaaataccctaaggttaaaatctcactaacaatccacgcagcatcaaaggttttcctgagcaagccaagtgattcaagtcatcagtgacttggcgagtacttggtatgaaaggaaaatgttttccacggaaaatttttcctgaaaaataagtggttttcttacttattttctggtatttcgttagtaagcaaaaaatattatctcaagagcatttataacctagacagacattatgggggtggttttttttttgggtggtgnacatacaggaaaagagctccaacaagaagaaataacagcacaggaactagtaagatgcataccgtcaagcttgaaagtacttgctgggcagccattcttttggcagcttttttattgatattgggagcagaagccgcttcagaaacattcttcccactcacattggcctcaactagatatttgctatctttttttgaaaccaggaactttagatgccagccatgaaactggcaaagttcatggagttctctttttggattatgctgcaacctggtaaagctcatgacgggctttaagaaggagagcattatatgccaagcacggttcaagtcaaaccctgtgtcaagcaggattgcacccatacaagactccaccaggtcgccaagggcctgaaccaaccagtaaacatgagagcacaatgaggattcatctttcaaaggttcagcaaaaaaaatgattgaacaattttctttactgctcaggggagaggggagaagcaatcaaccccggtattacttgatatatacaaatgcttcagcaaaagaaatgattgaacaattttccttactgctcaggagagaagcaatcaaccccagtattatttgatatatacaaatgcttccatgtataccatcagaacccaatttacttgactaacattccttttcagtagccctaatctctttgcttttttactggacaacaaactctcttttaaacaaggaggtgcaatgatgcaagagacaacttaagaaagaacatgaaaacatctgcttttagtaagtttgtggcccaacctttcaaggatgcttatggtataggttcaaatcttattttaggttacgttacctgctaaccatgtctattaccctatcaatatctttatcttgatagggttaaattttttaacctaggatttgcagttcatcgatttataataaatagcacattatttagttaacgtcacctgaataattcaatgattctcattttctttggagccctacccatccaagaagctaaaatgcagaagttcccatattgtttagtaggaatgcgggtaaacatcttgcatgtagtacatagaatttcataatgttctacaaatagaggcaagaacaactccatattgttctgtacgtagcccatggaacaactccatattcttttatattaatggttggaaaatgcctatggctttggcgaatcttctgtggcttctcggtgctttatgcattacattatcaggactcttcatttgtccagatgcacacctgtcaattgggcacggcatgggcattgtacctcgtaagaatccttccatgtaaggttaaggtatatctaaaattgcatgcacctgagttagataaaaacatatatctgatgctcatgttcaagtacctgactctgattgcttcttcttctatttctcactattcttttactccaatagactcgcaataccaaggtattcttcatctttcttcttaatgctttagcaaagatactgtcatgaactattaacaggaccagagcatccctgaattacaatgctagagcccaaagcctccactgctttacccattaatgaactattaacatccaccttttctcctcactatttcagctctaaatatactgctgcaaggttaggcaataaagctcctttctcacctacctaggaaaagaaaaaaagactatgctcaattctaaactggttgcagtcgactgtatgaatcctttatatatcattccgctctagttgggtgcattcattcaaatactcaataatttatcttttattacaacttacagattctctaaaactagaggttcactaaaccttaccctacacgggtgtacaaatttttacacaaactaaaagacactggcaaacattggagtcaaattaggtataccaacacaactaagatttatttccaactagttggtcttgcatatatcaaaagctactacacctaatttatgtttttttctagaatggactttaggactttccctactctagcacaggatctttattgctggtaagcctcgataaaacgctaattgttcagtcgaggcttaccagcaataacaatcctgtgggaatgcagcctcaacctgctggaaaagcatttaactggagtcgtctattcctgatccaacaaaggaaagaagccttgatcccaatactagctgtggattggaatgttattttatccaactgactctaatcatgattattactctaaacaactggattggattttccaactctctttataaatagactgctttcaatttcacttgccctagaacctctttttattgatattgatctagaatcagcttatcctagtttctttattattcaacaccagaagctattgtcacgacttgaacacatgaccttcataaaagaaatgaaataataaatttgttttccaggcaagatttggtgatttgggacattccaggtaaaaaggtagtgcatgtcagaatatagtccatttagtaatgtcagactattttagcttagctaggttcgtcttttgagacttctttcagttacatcgctattctggcaaggccaatgtcagaaagctggagcaggaatagagtttaggacctaaactggaaaacaatcaactcctgtgtgcaacaatatcaaacaattttgcgagaaataagagaaaaggagaatgacagagacatacgccttacacctgaaacctttcacatcactttacaaagtacattcttacccttatttgtggagacaatgtcgttttgcctaatacacaatctgctcctctagcaataatatcaaatgatttgaagactcaaatgtcttatacctggattcttttataggaaaacttacaatgacaattcttactgttagtttacagatatgtatagtgtgattgtcccacattggaagaggagtaatatctccttgtagtgtatcgctataaatagggacctcttgtattgtattttttttttataaccgtggtgtccgggccagcttgcgtgcacctcgactaattccacgggatacctgccacctcccactagcaacaggtaccaggtaactctatccaccaaggcttggatagatgggaagaaatcacctagtatttgcttccgctgggatttgaacctgagacctcatggttctcacccacttcattgaccactaggccacacctttgggtttgtattgtatttatcatccaatatcaataacatattttctcccgtgccttctcacatggtatcagagcattagtgagaaaagatcgctgtgcgtcattccaacgttaaccgggaagaaagaacttagtcatcgtgcaatttttccggtgacctaaggcttgtctaagtgaaagtcactttctgtcggtgttgtgctaaaaccaacatcaccacgaggtagatcaccctccgacgaccaacccctgaaattttatccggcaaaaaaggcgccacgctcctccacgcgccggcaacaacaacttttccggcgagggttccggccactttccggccatttttttgcgaagcttcttcaggacagtgtgctctcctcaaaattccgagcttacccatctaattcaaatcaaattccgatcacttttatatttttccggcgtgaacagtgacctttgcggccattttttgaaaacatttcttcaggacagcttgctcgcaaaggaattccgagtctatccatcatggtttcgacaaattccgacaactttggaattttctggcgagctacagtgtttccggcaaagaacattgttctgttttcctgctgtaaacagtgtttttcaagctatttcttcagttttctaacaggagttacttatttccactatttcaagttaatcctactactacaaattgtagcgacatgggaaccgatgctttgaatagtcggatggtttctttagcagagtatattgagttccttcagtataaagcatgtaagcagacatcttctgagatagcttctgttgttcaaacaggtaatagcgtgacttgtttctcccaatcttcatcctctgagtcttgggtcattgattcaggtgcatcagatcatatttctggtaacaaatctcttttcactactatttcgtattctcaatctcttccaaaagtcacaatggccaatgggtctcaaaccatggcaactgcaataggtcaagcaagcccacttccttccttacctttagattcagtcctttatgttcccaatagtccttttaatctcatagctgttagtcgcttagccaaatcacttaaatgcgctgttttatttcttgatgaccatgtttttatacaggaacgtagtatagggcggatcattggtaccgggcgtgaatcaaacggactttattaccttatccttgctaaatcacatggactcacatcttgttttccttctacaacttgttctgttactgattcaccaaatttattacataaacggttgggacatcccattttgtcaaaacttcagaaaatggtatctggtttatctcacttgtcagctctagagtgtgagtcatgtcagctcggtaagcatacccgctcccatttccctcggcgtcttgataatcgagcagagtcaccttttactttagtccattcagatgtttggggtcctagtcgggtcagttccaccttgggattccgctactttgtcagtttcattgatgattattccaggtgcacttggatatttttgataaaaaatcgatctgagttgttttctattttccagaccttccacgctgaaattcaaaatcaatttggggtttctattcgcacatttcgtagtgataatgcccgagagtatttgtcttccccatttcagcagtttatgaaatctcatgggattattcatcaaacatcttgtccgtactcatctcaacaaaatggggtagctgaaagaaagaatagacatcttattgaaactgctcataccttactcatacaatctcatgctccattgcgtttttgggggatgcagttcttacatcttgctatcttattaatcgtatgtcatcttcanagctcctcgtgctcttaagtgcgtatttctgggtttctcgagaacacaaaaggagtatcgatgctactctcatgacctccaacggtaccttatgtccgttgatgttaccttctttgaaacccaatcatacttcacaggttcaagtcatcacgtagatatttctgaggtactaccagtttcatcttttggagattcagtcactccagctctaccacctacagctccagttgtagctccaccacctatagctccagttcctccacataatccagttcaaccttctgcagcttcaccactcttgacttatcatcgtcgtctacttccagcatcaggcccagatgattcacgccccgcataagattctgcacctactgcagacttgtctcctcttagtcaaccaattgcactccgcaaaggtgtacgatccacacttaatcctaatccccactatgtcggtttaagttatcatcgtctgtcgtcacctcattatgcttttatatcttctttgtccactgtttctatccctaagtctacaggtgaggcactatctcatccaggaaggcgacatgctatgattgacgagatgtctgctttacatgccagtgacacttgggagcttgttcctcttcctgcaagtaagtctactgttggttgtcgttgggtttatgcagtcaaagtcggtccggatggccaggttgatcggcttaaggctcgtcttgttgcaaaaggatatactcagatttttgggcttgattatagtgatactttctctcccgtggctaaagtagcatctgttcgtctctttttgtccatccatggctgttgtacgtcattggtctctttatcagttagacattaagaatgcttttctccacggtgatcttgaggaataagtttatatggagcaaccacctggttttattgctcagggggagtttaatggttgtgtgtgcagattgcgcaggtcactatatggtttgaaacagtcccctcgagcttggtttggtaagttcagcacaattattcaggagttcggcatggctcatagtgaggctgatcactctgtgttttatcggcattccgcttctaatctgtgtatttatctagtggtttatgttgatgatattgttattactgataatgatcaggatggtattactaatctgaagcaacatctttttcagcgcttccagactaaggatctgggcacattgaagtatgttctaggtattgaggttgctcagtctagctcaagtattgttatttcacagcggaagtatgccttagacattcttgaggagactggaatgatgggttgcagacctattgactctcctatggatccgaatgctaagcttctgcctggacatggggagcttcttagagactctacgacatataggaggttggttggcaaattgaattacctcacagtaactagacttgacatttcttttccggtgagtgttgtaagtcagtttatggattttccctgtgatagtcactgggatacagttattcgcattcttcggtagataaagtcagctccaggcaaaggattactattcgaggatcgaggccacgagcagattgttgggtacacagatgctgattgggcaggatcaccttttgatagacgttctacgtctggatattgtgttctagtagaaggtaatttggtctcgtggaagagcaagaaacataatgtagttgctagatctagcgccgaagccgaatatcgggccatggctatggcgacgtgtgagttagtttgggtcaagcagttgctcaaggagttaaagttcggagaaatcagcaagatggaactagtgtgtgataaccaagctgctcttcatattgcgtcaaatccggtgttccatgagaggactaaacacattgagatcgactgtcactttgtcagagaagaaatactttcaggagatattgttacaaagtttgtaaagtcgaatgatcaactagcagatattttcactaagtatctttctggttctcgtattagttacatgtgtaacaagctcggtacatatgatgtgtatgcaccggcttgagggggagtgttagtttacagatatgtatagtttagtcttgtcccatattggaagaggagtaatatctccttgtagtgtataactataaatagggacctcttgtattgtatttatcatccaatatcaataacatattttctcccgtgccttctcacacttacatttatttgatgaagggcaaatactccatactataataagtgtaaacacaaactattataaactctgtataagaaacttcatgaatccatcaatattgaagatttcatcaactcgtaaataatgaatacctttggacaagatggctcttcagccagccttttcatagaatctggtcttccaatgaaattgacatatctagtaattgactcacggaggtcactagaatcacagagtatacggctgtggaagctttgatgtactgcaacaactgcgaaggtgttgttgtttacagaaactgatctcagatcagtcaactgcccaggcttcagatctgggtacacagagtatagataggatgtgatcaaataatcgaggacagcatctccgagaaactccagtctctgctaaaggaatcaaatgttcaaagaaagaagttgctgcaaagtactaaaaatcgcctacgcaacaaccagaaagttacagaaaagtaggctattgaaaaagagaaattccatacctgatagcaacctcctccgtggttgttatatgatggatggatgaaggcctgaatgagcaaacccttatggacgaatggataacctaataaattttcaatggcaggaatatctatttcatcagcaagtggcataaagactttgcttgcagcgcaaatactctttacttgtgattcttcgaaatctgtatagataccaatacatttaagaaatgcaattgctgctttgaagccactgtcaactacaaaagcgccaacaagggcttcaactatatcagcaattgttttcttacgcaaccactgatgacatttgctacacctaacttcagtctttgcaccatcggttacactaccacattgaccgtgtatgcttttttctgtttgtttgttacagattacagggcacggacggcctactacataaaagtggttgggatcaaatgattgatcacgaataaatgcctggcacaaagaaaaacttagagattcaatcaaatcaattgacttagagtataaacatcttaaaaggagaaagctcataaatatatatatttatatataaacacgtaatcaaaacatatttacacacatacagtcacattaatgtatattccatgacctcctccctcataggaaatcacaattgcctctgatttctaaaaattcattagagatatgcggtttcatcttatattcatatttatgttcaattattgagatacagaatgctagctgtcttatttatcacacaaaaagaaaaatgcagaactgtctaactcgagaatccaacaacatagctcattagaaattgaggaagtcattttagtatggtgagtatgttcattttatgtttatggcttgctcttgacaacattcggatatttgtgcagcggacagcatactcgtgacaatcagagtaaaacatttcctcatactaggaaaagaaaatgtcagctactactgaatattaaggtgtatcaagacaacatcaaacaaacgtagtaggactaatttttccaatgatgccacaaaaaaaacattcattatcagctaatcaaatatcactatctttaacatgttaaatgtatgtatagaaaaaagtttactgttccagacacagaggatatcccctcttttacttggttggataggagatcatcaaatcttatagctctcttaatcccaatttatacggtagtctttctatttaatatcttgaaatgttttaacccataccatgtttatacaatttttcacattttcaataccttgaattcattgtcattgtatcaaataataggttactcatgaatgtatcagatatttattactcccttcgtcccattctactttatggttatcgactaggtataaagttttattagtaaatttaacttggttattatattagtttagtggagataatattaaagttatttgatacaatgacaaattattcgaggcctcatattaatcaacacagatgaatttctttttttcctgataagtttcaacaagatatattctatttctgctcttttatttgtttttggggccatgtgagataaaagagagccacacgtgcatctgagtttgtgttaagcaatggcagtgtttgcactttctcacaatgaaataatgtaaaatttgcaaaaattcaaaagccatgttttgaaacaagaaaaagcacgcattctcttatctgaacataaaagatgtgctgtcatatcactgaaaacctgtccatatctagcatttagagttttcagcaacaccatgtacctgtaaattgtttctgactgctatcgtgtacagataggaattgttcacaatgtttgagcgccttctagttaactgtccttcgtcaaaggcatcgtatgtaagaaagacatgcctcccaactgcaaacttgagaaatgcatcaccaagcacttcaagcctttcaagagaaaaaggttcattgcatttctctgtagtaagtgcttcaagaacctggtccagttctaggcaattaatagtaaactaggtactgtatctggcatgattcatatgctatgaacaagtgaaagatgtaaaacagatgtgtttgattgagaaatactactaagctatgttgctcggactctcccaaagtgtcgtcggtgtgggtcaaattctccaaagtagtgcattttaagatccgacacgggtgcggcagcattttggagagtccgcgcaacatagctactgaggatggaagaattccttacatgatctattgttacttcccttccctcagggaaagaagcagatagccagcccttaagttcaatagcgacaagtaagctttcaagacgatgcatgatcgatggcaacagtgataatgagctcccaatatctttggaaaatccaattatcatgagctggcaaatttcagcaggtagttcaacaaagtgctcctctttatcacgcaactctgtgtagagggccagaggaaagtcattcattgcaaaataatacaccacttaagttactgtgtttctcagaataataggcataataaaaattgaacttattttcaggagggaataacaagctgacattctgaaaatctggcaagttaaataaagaagaaagcttatcattttctcatgtcatttcttggtgaagctagtcccaaatgacttactgaagttttagaagaaaatatagacccttcacccttggagctgctcggcacattatgccagtgcagatatcctctaaaaccaaatatccttttttttcaataaataataactattaaaaccatgtatctcccctccattatccattctcctgtcaaatcccggggtttttataagttttgagccaggaaacaggcctcagcattattcacccaagaaaaattctacgcaactggacttttcactgtaatatactaggtggcgattttattatcttctaccactattcctattgctttcaccgaaagaggttacaacacaatttgcctgatgaaaagctgatgttgaagcaaagagaaaaaaaccaatgttcagagactatggaagcagcaatgatactgttcaaagaagtagtgtagcaaaccctttaagcaatctcttgcaatttgcactttagttagtttcatatttatttcatataaatctttcaacttatcttacttagttgatttagcaaccatttgatgtcattttttttaagtagttccttcatcgaatttagaactagggttctttttttctttttgatgaccgtggtgtctgagctacctttagcgaacctcgactaattccacgaggtacctgctacctcccactagcataggaccgggtaactctgtccacatctccattttgatttgaacctaaaaccttgtgattctcaacccacgtcattgaccactaggctgtcctttgggtgccatttagaactaggttttttggatgctctttctcacagataaatagaactagatagaactttatcagcactcaatagtctaatgtcaatgattttgcctctgactttcaagttctatagtagagtttgtggtttatttgcattcatagcagatgcccattagtttaatcaatatgcttcaacatgtatcggagtaaaaccctttctttcaatcccccctcccttgcatcacaagtcttaacgagttttgcaatagaaaccgctcaactatgcagcttgaataataagcaattacctgaatagcctttctttcgtagtaagttgtccaaacagaagagttgttttgcttttattagtggttgctcagggtacgaaagatggataccaaaactgtaacagaaatctctcacccattagaatatacatgatataaagtaatgtagctctaaacaagaagagcacgccagtccctttagaggccagttagggtaacccagaaaacaagcagagttacacaattgcaatgaaaagaagtctctctttcccaaactaaagcaggttgtctgcttcctaaacaagtaaagatacatcaataacgggaagtttcccaaacaagctagtatacgggagttatcccacattaattaaattatttaccttatcaaaaagggaaaaggaagttacatcaagaaattctgttagtactcaaggctcgcaaaaagtataaaatagacatatagtgactgaccataataaaaaaattccagaagaaaaaaaagaacttccaaatacatgtgaggaaattgtttaattggttctccagcagcaagcggttaaatgctaatcgacctcatcaaaaactcttttaactgccattctctttgcatctaatttgtaaattttctccccttttggccacaccagaccaagggctcatttccactttattaatacttgaactcctattctggtttacagtagacagaaaaaaatatctgcgcatgcgtatgctagaatcaacaaaagaacagggaaccaagctggtaagcaaagaacaaaaataaagtcatttacttacgtatcataatagtgtttgacatggttttttgaatctttatatatgctgtatgcattgctttctttgacaacatcagatatgaagaaaaatgtatccttgcatggaacatataccaaactattggcaacatcatgtacgcttttggaaccattggcaagttgcaactgctcatcaaatttggatatctcactattgcctgcatatactgaagtcccaaaaattggtgatgacaagcatcttctgacaagctcccagtctatggaaattttattgtgtccacataaattgacgggaagaagtaggtaaaattttgaagctgagtcaacagaatcttgtttttccaaagatacaaattctgaaatgaactcagagcggtcaagaatgatcttgaggaacattctctggaatttctctgctagttgtacctgctaagagaaagtagtcaaaatattgaagagagcctttctgtttatactagaagagggtcaaatagtttacctcattgttctcaaaattagtagctcctgatggaataagctctgtctctacagatctacctcgagccaagttgagatcaagtttcattctctcagcctcttggggaagaggtgctttcagaaataggccaaactttttataaaccctatcaattggacaaggtgaaaagttaatgtagtacgagttaaggcaaacaggactctctgtctcagtccatggttctttaaaggcagcgggaacaatcatctcatggagctcttctcgtgcatcttcgcctatgacatcaacattacaatgatgacaacaagcaccaaaataacacaaagcatattgaaaatgcttttattagaataattttgcctaaccatctgagctttctgagtcagaaaaaccatgaatcaggtcttcatcttcatctgcttgatccggcaaaagatagtcagtgagtgctccaagttcatgcaagctcttgcaggctcttaaacaggcatctttcttagctgcttctatagaagattgaggagcactcacgatttgatgcattggagcatttgaaggtaaaactaatttgcaaatagttccatctatgtcatcaaagtagaagaactgcggcttggggcagaaaaacctacaaaagaagcaaaacgaacagaaagaaatcgtgttaacccatgaagatatgagttccgaaagcaaaatccacccataagtgatacagaacagaataaactttaattagaaaaagagaaacagaaataacaagtgaaattcagtaaaagatcatactcgtcacgtggaagcttggaacaataatggtgaagcaacgaaatgcttgatgcagaactgatggttgcaccagtcatatctactttgtatatattttcctgaaaatctataactgtcgtgcaagatttcctggaagaaatctcatcattcatccgagcttcatttcttgaaaaatgctcgatcaaattaagctccctttggttatcactgccaagaggtttgtgtcagagatattgctctgatgaacttgacaggtaataaggtacagaaagcataaactttggagatacctatcaaccaaaaatgcatattcagactttggcatgcgggcacgccctctagactgtataaaactggcaacagtttcaggaagatcaaatcgtataacgaggcagcatgtctggatatcaagtccttcctcgccaactttggtggcaaccaataagttgagctgttaaattgagcaatggatatgcgatcaatctcacccaaactatcagatggagactcaaacataaacatggagttaccctcccagaggtcctctggctaagagaatcaagcgagcagagaaagaatctaacaaagaaccaatacctcaccagaacggaacttattgagaatgatatctgtattttttcgtgacatactcttcaatcccgagtgcactcctacaagaaacccacacttccaagaagataaaactttcagatgctgaagcatatatgaaagagatctcgcagtcacgatcctattgacaaaaattatgcacttcatatctggttgtaccctgaaaataaagagagggagataccaaaatgcagaatatgaagttgagtctcgaattccttagcgactgaactcactaagaagaagaaatatacatccatgatatcaaacaaaacgaagcatttattatacgtattgggtaaaggtatggtatgaaagcaagaaacatacatcaatgacttcaaacaatattcttattatttatatcgggtaaaggtacattatgaaggcgaaagatcacgctgaagaataaacttttaggggaagaaatgtggttatcaactaagttcactgccaatactccttcggttgattttattataatcggatacaatgacccataagaacacccctcaaagtggaattgcctattttgtttccagctacttataaatcctgaataagttgcagagaacaggaacaggaaattattcatttagctacataatgactaacccgaagttggaaaggattccaattagacgtaaaagctttttggagaaataaggctccttcagaacttccacaattgccaaatcaggattcataccatctgcatgataaagcatatgttagacttgtaatgtacagaggaaaacaacaagaatatactcgagccgcaacacaagacattcttaaaatcaaaggttttttgatatttctgttggaacagatcattcttttaagcctacatttgcaggagttgacttttatcatatgcccttagtcttcattaaaagaccctaaagcacagcaaataaaattcgaaatcaagtctaactttacacagaggtaaaaaagaggtgtggaaagatttctgttagtctgaaacttgattcatcatacttgttcagtcacaacatgattcccatctagttgtcaagccttttttctttgaaatgctaacatcgcactgaaaactaaggcagcttatattcttgcttcattggtaaattgaacatcaaatgataactttcttccatgcataaaacttcttgctttagtttctccattcttttatttattgcaatagcgattgtgaaaagttacaaattatgtgggggaataaactctattaaggagacttttcctatttttgataaatatcgcatcttcctatcctctccagtctcttagcatttttttcacctcttataatggaattaattccaaatttgtaatatggatgtgtagcccaattgtgtttgaacccccaagtgttctacataagaaatggatgtctccgtttggatcaacaagattaaagtgccatatgcaaacaatagatgtgcgacatattccatgtcatgctctcctccaacaacccgaaattacactaagaattcactagctacagattataacactagctctctattataaacaataataacaacggcaacataggtcttcctgccgaataactttttgctcacaataacccccaccccagcccccccacacacatatacacacgcaatgggtatgcactagttctccactttctacaactttccccaagaaaccattctggataacgactaatctaaaataaaataaaaaatcaattggcaaggtcacatgccttctcatgtctagtttaactagaattcccaactttccacagatcaatacagaattcgtcagttcgtttgagatcacaggagcatccatacccttcaaccagtgaaagcatctttttttttgaaatgataacaggtttcattgatatatagataaatctcagcgccaagatagtgctgagcgaaacggtccctagttacaagacttccaaaaactaagagattgatgaacaaaaacaagtggtgaaaaactagtcttgtggcatttacaaggacccgatgagatcaactagggaatctgcatcttgtactaaagcttctttacaccaaaagtgaaataaaaatatacaattcattttaatcttctgttcagggtttgctgagtcttcaaaacatctggagttcctttccagccagatagtccaccaaatgcatgctggaatgagactccaccacctctcctggctgcttcctgtagctaaaaagtcccaactagacagcaagtccatagtcactctaggcatcacccagcttaagcttttcatacttatgaaaagctgccacaatctattggacacctcacaatgaagaaatagatgattgtaggtttcgatggcccttccgcacagaaaacatctggagcacagaggcattcctctcttcattaagctctcgtgtgtcagaacagcctgtttgaccactagccagcaaaacacattaacttttaaagggaccttcaacctccatatgtgcttccatatccaagcctcttgttgctgcgaacttctgtttaaaacctcatatacagatttcacagtgaaaactcctttgctattgtttttccataacagtttgtccacttgctcagttggacctttgaagttttccaaaagtttcagaagctcaattactctgatcacctcccaatcattcagaaatcttctgaatcttaaattccagccttgctgattccaggtatcagcaatagaagcatctggctgttgcgctaaagagtatagatcagggaaactgtcttttagtggagtattccctatccaaacatccatccagaaagagagtttttgcaccattgcccacctttatgcatgaattgctaattaggttttgccagagagatctaatagatctccacacactctggatacaatgctattcaccacaaatctcaatctcattaacaacacatttaccattgttcagtttccagccaaattgataggtccgaagtccttatttattatctgttgctttcctcttcggaattagaataaagaacaaagaatttaaatatctcttaagggaccaaaccgatgaaaatgacgcacgcactataaacattgtagtttaccactttccattagtgtttaaaagaggcaagagtgaaaccttcaaatccagtgccttgtctcctaaacaactcttcatcccctactccactctttcagcttcaaatggtttctctagctcctctattttcctgctgtcgaatttctagaaaataataccaccaaatcctgacatgtgtgccctttctttaatgaaatttattacttcatcaaaaaaactaccatcaaatcctgacccctactcatcatatttctcataacgacagatatacctgcaaaatctactctttattttcattttccttggagtacctagtcatctatttgcatcttctcaatgagattacatcttccatgtccactatgtgcattggcaggtgaaaagaatttggtcttgttatcaccattattcagctgaaggaaatttggtagtagtgtcacccaagcctatctttaacttcctccctcaaaatctcaacatcattaaaagcaaactataagacagttaagtgtaaaatttgctatgcaaatatagttataatcaatgtaatatagcattagtgaccacaagtgggtaaatatagctatatgtgttgatagtaaatgatgaccataaaaggattcaggcataagatatttgatttcatgtgttccctccattctcttatttctcgcataaaccatcaccttcgaccaactgcaatgtatgtactcctgtaataactctgtaatgtttgtttccactctgccagatatctccatgtttcatttcttgaggtttcttctacttatgagcttcttggctacctaaaatgtggtttcctctcgtgtacttaactatcccactgtcttaaccatctctttaaatccttcatgctgaagccatatgttctcagatttgatggagatggtccttttataaccaccagttctttaagtgaataggacaatgatccaacactaatccgtacgccaaatgggtagagtgacaaaaagaattccctcccattcagtcgataccaagatcaatcctggaaatcgtccttctattcctccaagtgaatcttgtcccaagtgatggcaagtcgatgaccacagttgaagataaagccagagaattcctctggaactttaccaccaatggtgtaaacactattccttccttggcaaaatcagcggcaatcacctatgtgtatttgccgtttcatctaacctcaataattcctcctacagtgtccccattccatttatacagtccccatacagcgttaagaagatccgcgtaaatctggatttaaatttcatttacttgttttaggtgtactagttctcctccctgtatgcttatttgtcattggtgaaatgtgttgtgccgtctaagaacacgaaggaataagaaaccaagtttagctttggcaaaggacagtgtttaaaaaagcgctcgcttcatcgctttaagcgagaagcgaggcgaagcgtgaaaggcagcgcttcagcttgctgaagcgactcaggtaggtaaaagcgaccgcttctctgtaaaaagcgagaagcggggaaagcgaagcgaagagaagaagcgtccgcttctctatattaaaatacccaaccctattttattaaaaacaagtttttttggattaaacactgatcttcaacaacaacgactagtattcttcaacttccaacagcaacgacttcttcttcatcacttctgatttctgaaagaatctcaaatcatcaactagggttgttcttcttcttcttcaacttcaagttcaagactttaagacttcaacaggtatgttctgaacttctgatttccattcttcttcttcttcttcttcttattctttttctttctaaacgtccaaaaagcagcgaaatgctggcgaattttttccagaattttactgcccaatttttccagaattttattgcccccagaattttactgcccaatttttccagttattaacaaagaattcctatgattttttatatcctttatagtttattcttagttcttattgcctttcttatgtgttatgtagttgttcttttaatggcgccaaaagaagataggaaagatccggcttgggcttactctgaaagagtttgcgagaccaacaagatggcaattagatgtcttttttgtgacaagatttcaaatggaggaatctatcgtcaaaaagcgcatctaatcggcggtgatccaaatgtcgcatcttgtcctaaagttccgtcgcatgtgaaggaagatttgaaagcattccttcataaaaagagagagttaaagactcaactgattcatgaacaagaactgtataatcttgatgacgatgatgaaacagaagaaggtgacgatgcttcgtcgcttccaccaaaatcacaaaagcggggaaggatgcaaccaatgtcttctagttgtggatctactggcaagaccaaaggtcctatggattgttacttctcgcaaaaatctggagataaggaaggaaaaagtggtaatcctcaaattgatgccaaaacgattttgagggatcgtgcaattacaatgtttgcgcggtggatgtatgatgcaggtcttccttttaattgtgttaattatactgacactttttctgcttttattgaggccgtaggccaatatggtccaggaatgaagcctccaacatatcatgaagttagagggccatatctaaaaaaagaggtggcagaggtgaacaaaatcgtggaggagcacaaagtagaatggaacaagtttggttgttccattatgatggataagtggacggcgagaaatggaaaaatgatcatcaatatcttggtgaattctcctaagggaagcctgtttcttgagtccgttgatgcaagcgactcttcgactgattcaaccaaaatgtactccttgttcaagagtacaatagactctattggagcagaaaatgttgttcaagttgtcacggacaacgccagtgaaaatgttaaagctggcgatttgatgtctgctgggtacccgcatatttattggactccgtgtgcagcacattccattaatttgatcttcggtgacattttcaaggaaagaccctttagttcaatctttaatcaggcaattagagtgcattcctatattgttcaaaggcctttgttattgaatatgatgaagagattcactaaacaaagaagcttggtgaaacccgcaaagacaagatttgctactgctttcttgactttgcataggatgtatgagcaaaaaagcaatttgaagaagttgtttgtttcagatgagtacactaacagtgcctatggaagggaagctcgagggagagaatctgcagatattatactttctccttcattctggaacaatgtggttcatgcattgaagattggtggtcctttagttaaagtgcttcgtttggtggatggggagcaaaggccaccaatgggctacctgtacgaagcaatggatagggcaaaggaggctattcaagtctcgtttagtgatcaaagaaaatacaaaagagtctttgagatcatagataaaaggtgggatagtaagcttcatagccctttgcatgcagctggacttgttttgaacccggaactgttttatgacaatgaagaaaggattctaggagatgaacctttgtggaatggatactatgaatgtattgagaagttgatacctgaagaatccgtgcaagataaaataacagagcaatttagtatttataggaatgctgagcaactttttggaaaaaacatggcgattagacaaagaaagacgaagtcaccaggtgagcgagtgcttatatgttgttttatagttaataagttatttctttatcaatattatgttttgaaaatttgttctacttctacagttgaatggtggaagcaatatggccattccactccggatttacaaaagttttccatcaaggttctaagtctaacatgtagctcatccgggtgtgaaaggaattggagcgtgtttgaacatgtaagaactaagaagaaagatttagtatattgagataattaaattatatctcaattgtcctaaacttactaattacttattatttgcagattcataccaaaaaaaggaacaaactaaccttgaagcgtctcaatgatctagtattcattaagtacaatagaacattgaggcgtcgttacaacgctcgcaatgtaattgatccaattagtttggacaacatcgatgatgctaatgaatggctaactggagtcccggaagatcatgcagatgaagaagtatttgaggaaacttctgatttcacttggggtgatgttgcggaggcgcgtggaattggggagaggatttatggtttgagggggagtacctcaacttcaagttcacagaggaagggaaaagaggcagctactttgtccctagttgatgaagaagaagaagttgaagaagatgacgagcaatataataatgatagtggaatacaagaatttgacaatcttgtagaagaataggatgctcattttgtgctttaattgatgctactatttagtttttacattgaagtattgaacatttgcttacaattacatgtgttgtctatgcaatatatatatatatatatatatattttaaattttgcttcacttcaaaaaagcgagcgcttcgcttctcgcttctcgctttaagcgaaatgaggtttgtcgctttttctcgcttcacgctcttcacaacactggcaaaggaacaccctgcttaatcagcggaatgctataaattgattcaaacttccaaagctaaacttggcttcttgaaccaagtaatccatgatcaggccagtaagaatagaattcttgttcaagctagaactcataagaatacttgtaatgatggaaagaatacaaatagctatagaacaggaatttacaatagaataaaacataccattagcacaaccagaagtaaacactgcagcgacctgactcagatatctgtcacacaaagagtcatcactagcattaacatcagcttccaccatttgatggcgctcatagtgatccccctttaagaggatgcaactagccttcaccaaaacaagggtgcaacttagaattgaattttccaactgtaagtcttaaaactaataaatagaaaatagatcgtgatgtcttccctgtaaattaatagtgcttaaatcatgctaagcataactagggaaccaacaagattctggagtgcatactctactgaaaataatgtctaaactagggtaatgcatgtgaactctccagctgaagtgctttaacaatacacacaaaataaaatctgaagtgcctaaagatgtccggagatactacttcagcagcagtaagaaagtaaaaacaaaaagagaatggaaggtctacacctaactgatatgcagtcaaaaagaagaagcttagagagttgtacagtgagcttgcggagttgtaactagccgttaggattagttaaaagacgggagtttatagttttgagggtgtgacactttagtccctcttatagtttatttaatacttttgtgtgtttacaattggacccatgtaattagcctttagcattacttttggtcctatagagttggttagctagttttgtcttttagtgagagtggagtatatattcctgtaccttttggtcatcaatcaaagaattatttttttgataagagggaaacccgcagccgctacacttggtgcgcgctgggtaaacctccccttgtgtaatagtctgcaaaccacacaagggaggtaaaccgcactaggcaagccatgtgcaacaggctcaacccagaaggcattgagggggattcgatcccaggtcatccgtatggatgactgccctccaaaccaacagggcaattacacaggtcatccgtatcaaagaattattctcttaccctaattttattcctttttagcttagttttctattccttcttttactgttccattgacggctgcaacagtggtatcagagcacgacctcggctctgtggattaatggcggaattgaatgaagtaatgaaaatgttacaccaaatttctacagagatgacagggtttacagttaggcaagatcagcttgaagctggccatgaacaatcttgcagaaatcaggaggaagccttgaatgaaatcagagaagtgttgaacgaggtcatccatggcggaaagagacctgagagagacaaggacctcaaccatagtggagaggagctgtattcgccaagtgataaaccactcgacctaggtaaggtagctactccttctgttggtgttaacagctcaattcctctaggaacaagtaataacactgttccattgactgtgagcaaccaaattcccaactctcagttacctccacctgttattcctgttaatttcaaccctctcccacttcctgtgaacaccatctctcaaccccaagctcctatgacactaggccgagctgccataactcagttcaaccagctccaaactcaaccaacgcagcaaagcttctctccccaacctagatttaccactgtccaaccccaaccatttcttactcctcaaggtgccaatagaccctactactctgcccagaacatgccaatgtcatatggcccttacccaccaacatattacccacaacctacacacatttcaccatccctgtaccaccaaacaccctatatccagacacctcttcagcctacccctgcacatataccctaccaaaacaatcccatgtatacaaaccagccaaacacacaaaatgtagcctatcctaacctgccttttactaagaacaccaaggttgaattccctaagtttgatggtgaagatttgagaacttggttgtataaggtggagcaattctttgctgaagaggaagtcaccatccaacagaaaatgaagctagtatctcttcactttgaaggtgatgcccttcagtggcacttgggatatatgaggagcaggggacaatcacctcttcctacttgggaggagtatatctgggccttgtgtgatacttttgggactgaatacactgatccaatgactgagatcatgaacatcaaacacacagggacagtgaaggattaccaaagagcttttatcaatgttatgactaggctcaacatatctgtagaacatgctatcagcatattcttaaacaacctcaagccagaacttagccatgctgtgagaattgggaatcattgtacattgccccaggctttctacttagctaggctacatgaatccagctttgcagctcagagtaaggctatgaaaatgggcaactctgtgtcatctgtgaatagagggaacatgagtcatggtcagaacaattggcccaggagcaacttgaatgcactcaagaaaccaatcacagctaaatttgataacaatagaaaaaggaggttgagtccaacagaaatggatgagaagagggctcaaggattgtgtttcttttgtgatgagaagtttgtgcctggtcacaaatgccaggctaagaggcaattgttttccctagaactggaagcaggtgaggtaccaatagaggaggaggaagagatccaaatagaagaagaaggagaagagatagctaaggaactccctgagaactgtgctatttccctacaggctttaaatggaaccttaggttatcaaaccttgaggttgagagggttcacagagcaaagacccctggaaatcttcattgattgtggctctacacacaacttcatagatgaaggaacaacaaaaaggttggggtgccaaataagcaagactaaacctcagctggtacaagttgctgatggtagggaagtgcccacagatagtatatgtaaggggtttcaatggttaatgcaaggggctgtgttccaggatgactttctggtatttccaattggcaaaagtgatatagtattgggaatacaatggctctaccccttagaggatattaagttcaattttaggaagctcatgattgaatttgagtatgaagggctgctgctcactttgcaagggattcagcctaaattcaaaacagtccaagcaaaagcaatgaacaaaatggccatcgaaggatcccagttcttcatggtcaaggtaagagaggcagaatgcaaggaagtggaggtgacagaacttgaaaaagcttaggaatctagggaactcaagcaggttcttgatgagtaagcagggatatttggagaacctacacagttacccccctcaagaggggcgtttgatcaccatattcccttgatggagggcaatttaccagttaactcaagacctttataggtactcagcagtcaacaaacggtctcaatatttgacaattagacccttcattgttaaaacagaccaaagggccctaaaattccttttagaacagaagcttcacactggaacccagcccaaatggatcactaagttgatgcaatatgactttgaaattgaatataagaaaggaaaggaaaacaaggcagcagatgccttatctaggattccactgatccaacttgctgctatgaccttatctactgtgaggactgatctgctagaaatgattatgcaaagctgggagctagatgaagaactgaaggcagtcgttcaatcaatacaacagggagaagctgatgcaaagggatacacttttatacatggccaactcagaaagaaaggaaagatagtagttggacctaatcttcaacttaggaaggatatcttgcaattgtggcttagttcaacagtaggagggcgctcagggatagagaacacctacaggaaggtagctgctcttttctattggaagaacatgaagcaggaagttcaagaactggtcaaggcatgtgatgtttgcaggggcacaagtacgacaatgctccttatcctgggttgctgcaaccactcaaaataccagtagcagcatggagtagtattagcatggatttcatagatggcttgcctaaatctaaagaaaaacagtgatttgggtggtggtggatcgactaaccaaatatgctcatttcatagccctttcccatccttattctgccaatgatgttgcaaaactttttatggaaaacatatacaagttacatggagtacctgaggatatcattagtgacagggatccaatctttaccagtaaagtttggcaagaattgttctctatgatgggagtaacactcaatacctctactgcctatcaccctcaaacagatggacagacagaggtagtgaacaggtgcctagagacctacctaaggtgcttctgctcagactcacaatcatggtcccaattcttagcctctgcagagtggtgggacaatacaaccttccacacttctatacagagcacaccatatgaagctctgtatggacaaccaccccgctacatttaccctacatggctggagattctcaagtagaggaagtggacagaagcctattgactagagaattcaagacacaattgctgaagtatcacctcaaaagagcccaacagagtatggaagaacaagccaacaaacatagatccgacagacaattcaaggagggagattgggtttacctcaaaatccaaccatacaggcaactatccatgtcaggcaggcactttactaagttatcagcaaggtactatggcccttaccaggtgttacagaagataggaccagtggcatacaagttgtcccttcctgcccagctcttgttgcacccaacctttcatgtttcccagcttaagatctgtcatgagttaccccctactataacacaccctcctgtcatcgagttgtctagcccttattgcccacaaccagagaaaataatggatagaagactgattcaaaagggaaacaaggcagtggcacatgtgttggttaaatggaaggagttaccatcagaattggcaacatgggaggactattcagctctaaagatacgctttccatctttccttccttgaggacaaggaagatttcaacaggggagaactgatatgcagtcaaaaagaagaagcttagagagttgtacagtgagcttgcggagttgtaactagccgttaggattagttaaaagactggagtttatagttttgagggtgtgacactttagtccttcttatagtttatttaatacttttgtgtgtttacaattggacccatgtaattagcctttagcattacttttggtcctaaagagttggttagctagttttgtcttttagtgagagtggagtatatattcctgtacctctgcagatttggtcatcaatcaaagaattattctcttaccctaattttattcctttttagcttagttttctattccttcttttactgttccattgatggctgcaactctgccaaaaactctatgccaggtcaagagagtcgattaaactgcacaagcttttcaaaccagtctgcactagcttttcaaaccagtctgcactctcttgttttagaagagagctaggagtctggtcaggtcttgatcctaaatcatgggttccatatggtacgcaagtttctcctttttaatgaaattctaaatgatactcaagtaaaaagaccatttataaagtttgcttgttggaggtgacggcaaggcggggcaggtcttcccctgctaatttttttctaatataattgggaacacaacaacaacaacaacaaacccagtttgatctcataaatgggatctggggagggtagtgtgtacgcataccttacccctacctcatagagatagagaggttgtttccgatagacccttggctcaaggaacagacctacatcaatctattaatataaaaaaaacttatagaaagaataaattctaccccctaaacaaaactccctaaaagactatattgtggacgctatgttgttgtccttgttacgagaagaaaacgtcaatttatagaagtacaaaatttctcctccaagaaaacgataaggcatatgaaagagatttaacccttttaggagtctttttcctttccttcaagaaagagagtcctaatagaatagaaattcagggcaaaatcctaacaattagatctcctggtagtgcttcagataactttacttcactaatctttcaatttcggttttgccatttccccctatctctacttcttatgaagcttcagcccatagaattcgtaatgtttgagacaatggaatgtgcgtactataagataggttgttaatccaatggagagaagtgataaagagataaatagaagaaagctcaaaaatgcagcaggaaaataccttgcatccgcaagagaactgaacatatattggagtaaaattttcgcatcccccatttaacacgaataaaatattaaatgtgagcaataaaatatctgataacctaagagtcatgaaagatcagggcttgtgaaaacggattaggggacaaacaagaagaagtgcaacaaaacctgtaatgccccaaagacacctaggttttccagagaaaaaattagatgtccatgcagtctcttgagcatcttttttgtgttcctgagagtgctatgatcagctgctttcttgtgaagcaccatcacacactgtcaaatcatgagaacaccagggagtatttagtcgaagactaattagaaaatagccatctcaattactgaccagataatacctggcttttaatctcttcaagcttctgaaagtaagctttagttaggcacgcggtgccaggaccatagtagtatacattcaccttaggtgatgcaacaaactgttccagttcatccttgtcttcaacagaatacacctgtgatcatacatagacagaaattaaatatagaaatattaatgttcctatttccttctacgcttctacaacagtatgggtatgaaccaaggttagcagtccagctcagcccagacaggtaaaatgccgccaagctagtcatttgttacctaacaatgaaacacccacagcaagagacccaggatacaaaatgatatctaggaaattggagtcgcaatcttatgacagttcttaacaactccaagaactaataaaccaaatgacagaaacccatccttcacaagtaactgtttttggactttattgttcccataataatgatgtgaggaaactgtaatcttttatagtcatccttttttttttttctttttttctttagaaaaataatttattgatgatggaaaaaaaggcttatgtgtacaatagtagcaccttagagcgaagtaaggtctcaaggccttcaacagtagcacctgcaatagtaagaagacaacataatatctgagagttgaggagcatccaaaaacaaatgtagcttaaaggaaagatacatcagtttcgcctggcagaatgattagtattgaaagcaaagataagatagcaagaactatcaatctgtcacagcaattgaccaatgcatgtcatggcaagcagatttctttccaaaaaggcacagaccccagagatcagatgagttttttaatacaactacaatagacccagtgtaatcccacaagtgggtctggggacggtagtatgtacgcagaccttacccttaccttcaagaaggcagagaggttgttcagattagttttttttataagtccaaaaatcagataacttgaaggcattttcagatttattttctgataatactcctgtaacttctgttggtccaatttctaaatagaattcatacaggtcatgactttctctagaagaatcaactacgtaacgaagatacatatattgtcatgcataaggaggtaaaattccacatatgcagatgagagacacttaaagaaaaaggaggtaaaaagggtgcgcagggacattttcccagtcatacaaggctatattagacagccaatgatgtttgatcattaacatagacaaagaaatatcataacaacaattcctctgcttctccagtcagttttcaatcagtttgaccaattttacatggataggagacggggataaattcatcatttcgaagtaataattaaagatttacagatctcacaagatggaaaaactttttcgtttgtaacaagtaagaagttgtccagagaattgacccctggaaacccaagaaagagacacaaattatcaagtaacctcaagtgtcagttccatgtcaatcccatgtgacattatatcattctgccgaaaattactgtgttgaatctcataacagttacattccatgacaatccctaagcatcttacaagcactgtgaagtactatttgtcaattttccctagattaataactatatttacattataaaaaacgtgaagggttgtgaggctggaccaggtacgaagaatgaatctatatctgtgcacggaaattgctggcactagctagcgccttccttatactaatataaaacgttgggcctcttccccttatttattaatattaggcaagttgctcgaaaattgtcaagtttaaaacacgaaggtaacatatgaaattactgatgagctatctgattggatccagctagttggagaaagttacaacatattgtcaccgggtgaatccatcccaaaaaagcaacaatacgcatgaagagtacagcacaatatattaccttttcctgatattggagatgcagtcatgccaaatatacggggcagttttactacatcaggtttgtaaaatatctgttaaaaataatatacatgaataagaaggctactacatacaaaacaaagcatgtttcccagcgtataacaccagctcagctaattaccttcataatctcagcataaggatgatcactctcaacttgtgcatagtggcactcatcaaagatcagaagagcaataaattcaatcctgatatagcagtgggaaagattatgtaatagtatttgaggggtcataacaaggacctgaacaaacaaagtgcaaatttgttggtcaaaacattcacatagtgtcaacaaaaaaataatttacagtataacagtcagtcatcaccaatatccacactcatgcacagtccaaaaaagggcctaagatgagaagaccacagtatataacacataacatggttcagtcaatctactacatatagttaacagaaaccatgttgattcctttttttattcttagtgactataaaggataatttaaattaataaaacaaaacaagcaagtgttgacgtcaacatttacgaaagagagaacaaaaaattgtaggtccctaccaaaaagtgaaaggatccaacaaactccatcaaaccatctatgtcatgtatagatgtacaatgacactaagctaagtgctgtaaacaaaaaaaaatccaaaatgtgtagactttctatttctttcaaagtatccgtgacaccctcgaaataacccatgtaacacaggaattccaacccgtacttgtggctcttctcagaatctccctttctcaagcacataagcatcatccactatcttcaaaaaggctcagaaaattatgtaagatttcaattgtaattggacaagataagaatacattaatttagttgaaatcagtaattcaggttatttttatccctaacatcagttattcaagtttcaaagaatatttgcattaaaatctgtaattggaagtttgcataccaagaatgcccaaggttctgtctcagacaaataacattcaggagacgatgcccatcactaacaaaaaaaggtcatgcagacttacctcatactgttccatttctttttcccagtcttcgtggctcttcaaatgcttggatttcccgcagtatgtcccaactttgaagtcgacagagtcttctatgaccttggcttgctattccaatttcaaaagcacatgaaaaagaaaataacttttatgcccatgctttatcaagtttcagacacactttttaattccccacgctgccccaagttgtaagagtacactcagaaaacggaatgggtttaataggagtaataagattctaacgaagagaaaattagttaaacataacaaatgacattctgaattgaagagcgctttcttgttctctacgagatgtaagactataggccttttcatgctaatcatagaccaattttcagcaaaatcgaaaaactctatatacttaattattttgtctagactgaaacaattagtcttcgattatttaataaaattcctttcgtcatatatgcgaagaatattgcttacatactagccacaaaatcaataaaaatttcgatggacaaatacgacaagtaaagagataagttgaaaactcattaatatgatgaatacaaagctggaacagccactggacaaaaggagcgatgccatcagaacagtagccgcaaagatatgtggtgcctttgaagcagcagaaagcgcaacggcatcaaagtggtcacccaaaagaagggctttgattgggccggtgacataagagaataaaattgtcacttctattttagtgtccccatgaatcaacaaaattattgctttatcaaaaaagagactcaattgaaaggagaaaagacaaaaagggaattgtgtaaaatagctggcggaaacatgcttaaagctctcatataccacctcaatagcaaggaagatatttgatatttcagccatctgatttcatgtaaattagttttcatctttgatatcataaaatttaaaagtttgagcaaaagccaaaaaaaaaataaaaaataaggaaacctgttgcaccaacgccacagtgggagcaagaaacacgcaaatgctcttctggggtttccttatcagctgtcccatctcatagataagcaagacggcaatatgagttttcccacagcccgttcccagatatacaacaacattctcctctaatgcttttttacaaagatccatctgatacctgataagaaataaaatcagcaaagtcttgctataaaatcatcaaagcacctatctcattgtttttgccattacttaaacatcattattcagtaaaatagagtagttaaattctgcaagcgtttgataattataaagaacttcagctgtttgaaagttcaaaggttttattgtaacaacaccttagcaaaaaaaagttcttttttttataagaaaatgtagagattttattaatgagtaacaagaagagagagtacagaaggaatgagcttttcctcctaaatattgaaggaatatatcagatcgtgagaaataatacactggaaaatattatgttattgatgtcccctaactataatacaagtacccctattataacaatatacaatacctacgctactcctattccatgtaagactacgcttatttacataacaactaactaactaacaaaatagtgaagcaaaatcagttacatctttcttacaccacaaatagagaatatttgaacacctagactctaaatgagaaatgaattctttttttccggggaaccatttctctctaaccaggtgttccagaaaaatacaactagacactgcattccagtagaatctgcttcccatcactaaagtcttattataccagctatttaaggcatgtattgtgcttctgcatgacaatttattctcagaaataaacatagaccaaatctgcttgatgaaaatgcaatggaacagtaagtgatcaacagattcagcctccttcacaaaatactgcatatagtaaagcccctattttgtaggttttcatgagtgagataggcaccatgatttgcagtccaggtgaagaggcaacctggggctgcgacagtcattcaaattatcctccatggctaatttacatggcttcctccaatttttagcaaaagagttttgttgataagaatggaccttgggcctaacttaaccccaaaagctagcttatgaggtgagaattgcccaataccatataaggagaccaagacacccattacccaccgatgtgggacacaactcaacaccccccccccccnnnnnnnnnnnnnnnnnnnnnnnnnnnnnnnnnnnnnnaaaaacccccccccccatgcccaggcctgcaactagagcgtggtcaatataaatgggggcccaacatcggtacaataaattaggatgggcctagctttgataccatgataagaatggaccttgggcctaacccagccccaaaaactagcttgtgaggtgaggattggccaagactatataaggagaccaagacacccattacccactgatgtaggacacaactcaacagtagcatgccctgacagaaaatgtcttgcccttccagagtacagaatttgctatatgttcatcaatccttacagtttcaaactttaaattagttcctttacatgctcaaactcccaatcattgagatttcttctcaatattattttttataaggtagattttttctcaatataatatcaataaaccagaagaatagcagtcaacaatagtgcaattcttgtgggtaattacatatggtcaggtaattgctcacgtagaggagtatcaccaaagcacttctcggccacattttaactctcttgctatttctaactttttactaaacattatcaaagaatcatcccatagctttctagtatatttccacatagaaactccatatacaccatttgcactatcagtgcaccactcttcggtcttcctatatttaacaataacaagattcttccacaaatcatttttgtcatcattgaacctccggtgccatttcatcagcaggcttttgttttgaagttctaaaatctttgagtccaagtctccaaatttcttatcccagcaaactgtacccaacttgatcaaatgaattctccgttgtcttgacatccttcccacaagaaattgcttctcaacatgtccaaattgcctttccacttttgaggcatgggaaataaacacatatatgagtaggaatctcatccaacacgttatttattaatgttaatcaccctccaaaggatatatacaaattcttccagggtgccaatttcttttcacattttcaatcacatttgtcatatattaacatccgttcctttagcacccaagggtataccccaaatatgcggtaggaaagcatccaattttgtagccaaagacatctgctagcatgtgtataacatggtcaaaattgacagatatagcttgtgcattacatgtaacccagctcctagtcaaaaatatgtaacccagctatagcttcaaaggttagatgtggcaaatgactgcctaccgataatcgccaaaataaactcttgaagtggtcacattaagaaatatctctgacttgggaaatgtgcctactttttcattcaccttccggcacccttccggtggaatttcactccttttccggtgaatttcctccttcgcataacatgggggaaaagcttttctttatttcaggtgacaagtcctatgagcttatcgacatcagtgaagacaatgaaaggtggttcttatggatcgaaaggggcagtagatttacaagtaaaattaagattgatgaggacaacatcagatgggtatgtgagcaaatgtaccaagcttcacggggcgccgggaatctttacaaaaggtgggggagaaagatacaaacctatttatacagggggtatcaaaattacaatgtctatggcagattcatcagaattgaaacttgggttggagacaggaaatttgcaatcataataccagaaaacgactacaacagggggtggggagatatagcaggaagaatattacaatttttggggaagcctgccaactacaagttcaaaaagtttgcagatgtgcaaaagagatcctttctagtggctgcaaagattcaccacacaagtcaacgagcaagaagatgtagtggctgctactggagatgttgctgctgccgatgaaagctcccagtttctatccaagtgcctgattggttgcttcaacgaccctttcaatcaaagccccaaagctgaagtcatccaaaaatggtttatcacaaggtggaaagtaacagcaggtctcatggtcactccgatggcccataatttgttcctcttcgaactaccatctagacaggaagcagctagggtaaaagcaggggattggttctggaatggtagacacttactcttgagtggtggttgccggaatctgggtcatcttcaaccaaaggtgtaatgggacagaagtggatcaaagcctttggaatccctctgcatggctggacatcaaaaaccttcagtttcataagagataagttcggaggattcataggcaccgatgaggacatgaaaaacagggcacatatgttctgggccagaatctgtgtaaaagaaaaagagatgagttacccagggatcgtctagttaaaaataggcaacttgagctacaagatttcagttttggaggacgtactcacaaaatctgcaattttcggcgaggtttcagtcgccgtcagagtagagaaggcggcagtctctgataagccctaatcgagtcaagcgaaggaaccaacgaacaacccagtagtctttaaccaaaatagacccatcatctttaattcaaaagaggctgacgtggctgcctcaaactcggggcatacgtacaggatgccagttggaggcacattaaatcaaacggtggggtcaaagagaggactcaaaatttgggcctcttagtggaccgtgaatattacaaaaagggcctaaaaggcaagcatgcacagaggggaaaattaacaaagcaaaagcaaatctggagagcaactgggcctaaaattgacccatgtgttttgaatcccaacccctacatctctgtaaattattttgacccactaatgatagaactagaagcctccttctcagaagggcccagtgttaaaccttcttcaatgacgcactgcgactcagaggcagatgatgaaggagaaaaggttctgcaaaatatttgcaggaaaatgccacaaccaactgaagctgtagacagttcagatgtcgagctctcttcttgcgacacacttgctctcccatggtatgaacaaggctcttcaaacatgcaagtaatcgataccccaaatatctacaagcatacaagatggacaaaggttgtgatgttaaaggcatgcaaggcttttggggttaacgtagaagggtttgagcatgagattatgggcatcatcctccggatggatcaaaagaggcaaatgcagttgcagaaacaaaagtcaccttccggagaaaaaaaaaaaagggtaagaagaagcaagaaacagaactggaaagactaaaatggggcctaaattacgagggtaaaaggaaaggggatatggacaggttttacaaagctttttctggatgaatgttaaaatattaagctggaacgtgcggggattgaatgaagcagataaaaggactacaacttcatcactgattaagaagtggaagccagatattgtttgtttacaagaaacaaaaatagaagaattcccagcatcatggatcagacaaatatggggcaatcgttgggtagagtgggcagagttgaaatctgtagggaacagtggaggtattatcatcttatgggacaaaaggcaatgggcaaacagggatactcatcaaggttagtaaactctttctcgcatgttagaaagcttgcatgaacagttcagatggtgttttacaggagtgtacgggccacacacaaatcctgaaagagaggaactatggttagaacttgcagcaatcagatctctctgaaatgagcaatgggttatagggggtgatttcaatgtttgtaggtatgaaaatgaaagatacaattgtgttagaaggtccagggccatggtgtccttctcagacatcatccaagacctaggaattatagacttgcctctacaaggagcttattatacctggttcagaggtgaagactcattgcaagtttcgagaatagacaggttcttaatttcccctgaatggtgcgacagtttcaaagctatcaagcagctagccctcccaagagtgatctctgatcataggcccctactccttgagtgtggtgactgggaagccaccccccttttttattgataaggtaaattgtattaatcaaaagggagtgaactcacgtatacacgaagtataccaaagacgtagagaatttacatcagaacatgattctctacaaaagacgctcaatcttctatacaagtatgggctaaatgagtgcacaaaaaagcgaccaaagataaaagactattcataagatgtgaaaaaggagattcaatctcctcaaaagctttcctatttctctccccccatactacccacatgagagctaatggggcgaactaccacgccttttgctttcttcttctacaaaaaccgccccaactatataacatttcctttacagtgctaggcatcatccattgaacaccaaaaagattcaggattgccctccacaaagcagaggacacaggacaatgcaacataaggtgatcaacttcttcccctgagattttacacatgtagcaccaactaacaagtgtaatttctctctttcggagattttcagcagtcaagatcactctcctcgccgctagccatgcaaaaaagcacaccttcgtgggcgcccaaggaatccagatcgaggagtatggaaaagtggtctcctctctcaccaacatactctggtaaaaggactttacggtgaacaaaccatcgccacgcaagccccatctccaagagtcgcaggatggctggagcctgtcttgcccgtatagcagtgccaacaaaacttggagctccgcaatctcccaatgttgcatgttccttctgaatgagaggtcccatactacccccttcatactccttgcgaatctgttggaccatcagttcttctggtttgaaactctgaagacgtgggggaaggagaccctcagagtatcctctccacaccacctatgattttaaaagctgattctccttccatctcgcaccctgtaagtgatgttgccactgaatgcttcccaatttttcatgatgctcctccacaagccacacccgaaaggcgttgtgatcgccttggtcctccaaccccctcccgttgaatcgtacttttctactatgacctcactccatagagcatgctcttctacctcgaatctccacagccactttcccattaaagctctgttaaataccctaagatctttcactccaagtcaactccacttctttggggaagtgactgtctgccaattcaatagatgaaactttctagttccatctgccgcatcccatagaaagttcctttgaagtcgctccagtttttctgtgatgctcaccggtgcttgcaacagggataagtaataggtgggcatactcgataaagtgtttttgataagcacctccttacctccttttgacaaataccgtttctgccaccctgctaatcttttttcaacccgcttgatcactagattccaaaccgtagtatccttatatgaagcgcccaaagggaggcccaggtaagtaatgggaagggagcccacattgcatctgagaacacaagacaaggcatcaatattagtaacctcacctaccgggaaaatctcacacttgccgatgttgattttgagtcctaatactaactgaaaccactacaggacctgcttcaggtaggtcaactgatctatatcggcatcatagaaaaccagtgtgtcatccgcaaaaagcaaatgtgagactcttcgggcactgagcaccccaatcggagctgagaatcctctcaaaaaacctctgtccgccgcacgatacatcattttgctcagagcatccatcactagaatgaataacatgggggatagggggtcaccttgcctgagacccctggaactgccaaagaaaccacacgggctaccattaaccaggacagagaatctgactgaggaaatgcagaacttgatccatcccctccatctttccccaaaccccatctgcatcataatgaagtccaggaactcccaattgacatggtcgaaagccttctcaaggtccaacttgcatagtaagccgggttctctatttttccttctggagtctacaagttcatttgccaccaaggcagcatccaggatctgcctaccttccacaaacgcattctgggaggacgaaacagtcatgtcaagaaccttcttaagtctgttggaaagcactttagaaataatcttgtaaatgctccccacgagactaataggcctgtagtctctgatacaagatgcaccttctttcttaggcacaatagtaataaaggaagcattgatacttctctcgaaaacaccattcgcatggaagtattcaatggcttccatcaactccccattgatggtggcccgggggccttatcaccagcacaactcgacacagcctcgtgcaccccctcttatttcaagttcgaaaatatgtggctacaggcagagggtttcattgataaggttaaaaactggtggcagagctatgtgataggaggcaacccagattttatacttgtgcagaaactgagaaggctcaaggcagacatcactaattggaacagagaggagtgtggcagactggaaacacaaaagactaaactactggaggagctagcaatgctggagcaaacaacagagaacagggcccaaacacaggttgaaaaggagaaactaatgcagataatagtggagttacagaaaatagcaaaggttgaggaggtctcatggagacaaaaatcaagatgtctatggcttaaagcaggtgatagaaacactaagttctttcaaaaaatggctaactcccacagaaggaacaattatattaacagattgaagataggggaggaaattactgaggaccaagaagccatcaaaactgaaatcctaagcttttatcgggggttatattcagagagtgagcaatggaggcctagtgtcaactttgaaaacttagccaggatctcgatggaggaagaagtatggttggaaagagcttttgaagaagaagaagtgatggcagccatcaattcttgtgcaccagataaagccctagggcccgatggctacacaatggctttttaccaaaaaacttgagaatttatcaagccgaacatcatggcagcaatcaactatttccaccataactgtcatatggtaaagtccagcaatgcctccttcattgcactggtccccaagaaaaaaggtgcagtagagctcaaggattttagaccaataagccttattggaaatgtgtataagatagtggctaaacttctgcagaaagaatgaagacagtgatggggaagctaatctcaggggaacagaatgcattcctaaaagataggcagataactgatgcatcacttattgcaaatgaactgctagatgagagacttaaaagtggagtccctggaattctatgtaaactagacatagagaaggcatttgatcaagtcaactggtcctatcttctgtctactctaaggggaatgggatttggtgaaagatggataaggtggatcaagttcagtctaaccactgtcaaatactcggtcctcatcaacagatctccagttggctttttctcccctcagaaagggattagacagggagaccctctctcaccttttctcttcatcatagcaatggaaggtttgagcaaaatgcttgataaagcaaggcaattggaatggattgaagggttcaagataagcaacaactcagggatttcaatctcaatctcccatctgctatacgcagatgacacactaatcttctgtggggctgagagactgcagctgcagtaccttaatctaacccttcttatttttgaatctatttcaggtctacgtatgatatatcctgtcaatgtggtacctgacttagatgagccggtaggtataatgagttgtggcattggctcatttcctactacctaccttggactacccttgggagcaaaacataagtcagtggaggtgtggaatggtgtgattgagaaagtggagaaaaggttagcaacttggcagatgcaatatctctcaatgggtggaagggtgactctgatcaatagtgtcttggacagcataccaacctacatcatgtccctcttccctatgccaagtaaagtccaaaagcagctggacaaattgagaagatcctttttatggaaaggtaacagtgaaggtcacaaattccatttggtgaagtgggcaactgtaactcaaccaaagtcactaggtggtctgggcattagagatctttcaaaacacaacaagagtttgctaatgaagtggcattagatatatgggcaagaaggcacaagcttatggaaagaaacagtgaaagccaaatatggtcacctgaataattggagtaccagaaccacaagagctcctcatggggttgggccctggaagcacattaacagcctaaaggaagtcttccaacaagaagtatccttcaaagttggtaatggagcacatgtgaaattttgcaaggataagtggctagggaacttcacacttcaagaaacctactccactcttttccacctagctactgatcctaactcaacagttgcacagaatagatccaacaacacttgggaggtccttttcaggagaaacatccaagactgggagatagacgaagtgcttgatctacttggaaagttagcaaattgtacaacgagtatccagcacacagataagctaagatggagcacttctaaggaaggggcatatacagtgaaggcaggttacaataaactttgtgctttgaatgaagttattgacatgtggccttggaagctaatccggaaaaccaaactgcctacaaaggtcagctgttttgtatggactactcttaaaggtgctattttgacacaagacaatctctgtaggaggagcttccagattgtgaatagatgccacatgtgtaagctaaactcaaaatcaatcaatcacttatttttgcattgcacagtggctactgatctttggcacatgttcttctcaatttttggtatcagctggacaatacctcagaacattaaggaagcagttgaaagctggagcttttggaaagtcagcaaggccatcaaaagtatctggaaaatggtccctggttgcatattttggtgtctttggacagaaaggaatcgaagatgttttgatagcatttcaactcctattcactttttgaaagctagatgtctagtgtatctttttagttggagtaatacctcccctgtaaataatgttattccctttctagacttaattggctcaatgagcctagcctgatggcccatttttttgtggttagctccctatgattagaatagcacatgatgtactggagctaacatcccttcttttgtacttattgcatcttcctgatgccagcaatacaacacaccagcaatacaacaccttacttcatcaataaaaggttagacagatagctttcagatgcagcaataggtcttcatcagcctcacataatcagcgtgtcatctgcatatagaatatgtgacacttcaacactgcagaggagcatgcatttggaaggtggtagcacagaaatatgacaccatggaaaggaggtggatagccaaaaacatcattgttccttttgagtgcggcctttttaaggaatattaagaaaacatatatttagtggttgtatctcatttaaggtaagagagaaacaggacttttaggggacaggtagtccagggacaacaggctgaggatcatttttccaaacatctgtagagattcatgccatctacagatgacagtccaacaagtctggatataacatgagggaagtgtatttgggatcttagattaaaaaggaaccttctaagatggaaagacgacaaatgttgaaaatctacttgaaatgctctataaaaagactttactgctagacaaccaagattactggaggtcggagttagggggtggtcaaaggttagagtcggatatccgtaaagagctactattgtaagtttcttgctagacaaaagttgggtttccctcgttttccagtttggatcccaaggactccaagaaatgtgcgctttttttgaatggttggtggctaaaagagcgattttgagaaccgaaaatttgaagaagaggagaatcacatgtcagttggtatttcatgtgcaaagattctggagatgacatggatcatatcctactgcattgccaggtagcattacgttagtggtagagtattaagatggttcgagatacaatgaaccgtgctggataatgtgaaggctggttctagttagttgggtctaacacggggcatggaatgttgctctattagcacttatatgggtaacatggagagagaaagagcagagctcttaaaggacaaagcttgcttggtacgtttaaggaatagcctcttgtttttaatgtgtttttggtataccaatgagattccttgttttatagaagaagattggtgtctttcctaaataatcacatctttatatagattctctattttttacttcctttggcattaaaaaaggcagcccggtgcactaagctcccgttatgcgcggggtccggggaagggccggaaccacaagggtctattatacgcagccttaccctgcatttctacaagaggttgtttccttttgcatatgggagctttttcctttttcgtaaaattacactttacttgataattaaataatatacccaagggaatggtttagtggccaatgaagtgggaggagaatcatgaggtctcgggttcaaatctcaatggaggcaaaaaaaactaggtgatttcttgccatctgcctaaattttggaagatagagttactaaatgtttgtgctggaggtaccaactatcccataaattagttgaggtgcacgtaacttggtccggacaccaccggaataaaaaaaataaagataaaaataacaataatagtaatattataaaatagtatgagataaggcgaagatcctatattcaagctatgttcctcagactctttacaaaatattgtcgggtgcgtattggatcctccaaagtagtgcattttcggaggatccgaaacggatgcaacagtattttggagagtccgagcaacatagaattcaagtcaatacacccttataaacaagaattttcacgtgtttgatcccaaaaaaaggtgctttagcctgctcgccttccaatcaaagaggctgagagactcgaaagcttcacttcattcgcttctcggaagctgcacaactctgcacgtgacaaatgctgagtctcattgtcacttgttatcaaaaacaatttccttgtgttcaatcccctgaagaaaaagggcgcaagcccgcagtgaggaaacattaatgtgtcccttctttaacaagcttacactttttgagttggtcaaactattcaacatcatccacctcgcctttgaatgatatatacacttgaaaaacctttatttaatatatattactataaatttcacacaaaccaccgcacagctcaactatatgcgtacacacaaataattatttatatacacaaataatctataaaatatttctttatctattttgacgaggagctttagctcaacgataaaagttgttgccacgtcactggaaggttgcgggttcaaactactgaaacaacctcttgtgaaaatgcactgtaagcaggggcgactctaaaggctcgggcagtgaggtcgttgccttaggccccaagaatttgaaggccccaaatttattttaaattattattattattactctatattatataatttatataaataattactataaagaaaagtgttacagtatattttttcattattagattgaggttattttataccaataaatttataaattatgataattttcttgtcagttaggggtaggtataaaccttcattcactgattaattagtatatttgtttcattcttcaattttaattttatcctttttatatatgaactaagttatatatatcgacaacataataaatattttttacaatgttctctaaaactacatgaatacaaaagtattcatggaccgtctttttagagtaattcgacatattatattaggttgtttacaatctattttaaatattcaccaataagtgctacttaatagaatgaagtataattattgcttaattgatcagaaggagtaaatatttttgacaccgttaatgctcaaaatttaagatattacgttatgtacgtttatttctttttcttgtttgtgatgatagccaaatcaaaatttgtgcaccagtccaccactttattttttgtgctcttcttcctattttgttgtttaaaaagcaaattttattgttagtgtaaatatggcagcctatttctctaaatttttaagttgggcaaactatttaacatcaccacaaagttttagttgatagtgtatgattctataaaatctcacaaaccatgacacaacaaaaatatatgtacaaaaataatcttcaaaatatacatttagcaattttgcacccacaagagcaaagaacttactttctagcaatttttctggggtctttctctggtttctgaactgaaacaggggaatctgtgtcgccattgagagacagagcagaaagctgctcagtgatcggtgaagttgcagcacttcgtggactctcagtgtcgttctcaaaatcgccgccttccatgacggtggcagtggcggtggcggtggcggaagtaccggtagtgtgtcggcggcagaaaaaaaggtgattgactttcagttgactgggagaagtgaatgttttctttttgtttagcttacttggagaagtgggtttcttcaggtgcgccttttgaaaaagaaaaaatgctgacactgttcaatgatgtatcaaacgcatctcacttcctaccaaactcctcaccttttttattttggtttttgtctatttttttttaatttaagtataaagtcaccataatataatttttgttacgtggtgaggctttggattttcgagaacatgacttttgataggaagttatgaagattgagtattaggattgtaggttaggaggtagttgagtgtaccttacttcgtacctttgtgagactagtatggtagggttttttaccgatgtcagctagtggcaacgttgtgtcttactactcttttattttccatagtgtcgcgtctatgtactgtctatcacgtttactttgcatctactttctcatttccatgatgttattttttcatattgtctcttttaggcttcttgagccgagggtctttcggaaacagtctctctactccctcggggtaagggtaaggtctgcgtacacactaccctccccaaaccccactagtgagatttcactgggttgtt

SEQ ID NO: 64 - DNA, 27271 bp, Nicotiana tabacum
  misc_feature (1406)..(1417): n is a, c, g, or t
  misc_feature (10706)..(10706): n is a, c, g, or t
  misc_feature (15092)..(15092): n is a, c, g, or t
  misc_feature (18008)..(18008): n is a, c, g, or t
  misc_feature (24423)..(24423): n is a, c, g, or t
ctacaagtgctgcaatataaactaaacagttagaaatagtaggataatattggtcagataatttatttgtagcaatatgaaattgttctaaaaaatctacaagcattttaacactactccaatcttgatttgtaaggtgctcatcatcatcatcatcatcatcaccatcacctacactagcattatacgttgcgcttattgggttcctatattcatattcaataactaaactttcatacatgtaatttcatctagtcggacaaggtttaggaacttttctttctctaaggccaaattcatcacattttttaaaatagtctctaagtctacttctacggtttgaataaaaaagccaattaagagccattttaaccttttcaatttctacatctaaaactttcataccagcaccgacgattaaatggtaaatatgaaaaatattactaaatgaaggatttagtgtagttgtaagcaagcatatagcattagtgttactagaagcattatccattgaaatcgacataattttatctctaatgcaaaaatatctacaaatatctgcaactgtgttagcaataaacttacctgtgtggtgtgaattaattattttataagcaataatgcgcttttgcattatcactcctcatctatccaatgacttgtaacagttatataatcacagtcattaccacttctaccaatatcagtagtaatagcaatgcgacaatctatatgagtaaataaatagcggaaatattgttcatattcatgtttatatttataaatatcgctctttacggttgcgcgaggccatcctttataagtaggattaaaaactcttctaatataatgcacaaaatgagggttagagggaaaactataaggtaagcacataacagtaaccatttttgtcaattcttcacgatctttatttggatcataatataaaatgccaccggtaacagtgttaatttccggttgaactagatttgaaactagggttaaccgtactatctacacttgtcctctcttacacagctttcatttgtaaaaatctagctttatctctagggtgtttcaatatgtgtctagtcaaactacctgtaccgctacggtctccagtaaatttatgaactagttgagacccacaagttttacatttagccttattttgttctcttagttgagtaaaaaaatgtcaaacaagagatgtttcgggccgtttagaaggtaggttgtttattaaaagtaggggttactataggagggtcatgcggggcatcagttgggttattaacaggactagtaggtgtatcatctaaatccggttgcgtttcatctaaatcatcattttcctcatcattttcaaagatagtttcattaggataaagagcattcataacttnnnnnnnnnnnntcaccaattttagatttttccttacccttaccaccaaaaatatttttcaaagaaaatgccatattaattataattatactaaataaaactaacaaaactatgatattaaaacttaagagttggaacgagtttaccgaattgacgaacaacttcttgaaaattaaatatcgttgaagacttgaatacttcaattcaccaacttcacaatttttcacgaaattgcaataataaagtaagcaattatagaagaaaattagagagagattaataaatttgtgagtaaaaatgaaagaaagggggtatttatagttgagaattgggaaaaagtgtaattataaaaagtttgggattaaagcaaagtttgggggccaaatggctattttttaaagtgcataacgaaccaattttgaagcccaatggtcagattttaaaatatagccgttgggcatttccgttgggccccgtctggcccgttgggcccggttgaaccggcccaagcccgcttgccggtcccgggcttaacgatcccgggctcacgggctatttgtgtttaaccgggccgtcacgggtttttgcaccacttaagctcaggcccacttgaaccggcccattaggcccgtttggaccgcggtcccgagccggtcccgagcccagaccggcccacaatacagccttaacctcgtatctaggggtgtcaatgcatatttaaaagccgactaaattgaccagaacgtactgcaccaaaccaatttttagatttcttttaataaaatcgtaggtttttctataataatgacaagttttttgccttctctctaggaattatgacttgcatttatgaacctattattatgaagttcgaattcatatatgtttttaaccattaaatatctgaaattcatcaaaaagtttaatcagaattcacgttgctgaatacttattatttcctaaatattactgacaaaaaatttaacatttctctacattccttaagaggttagctatctcaccttctatttggaatagttaatcgtaccattttagtacaaaatggttattgaaaccaataactacccattaccaaatgggggataaaataaaccggcattttattcagaagatgaaatttcttacatcaccaaccaaatggcccctaataatattattttttctttttaaaaagaaaaattaatttgaatcgtacagattttgaagaaaaagaaaaattaatttgaatcgtatagattttgaagaaaaaagaaatttattcatatcagatttattcattctttgtaaatctttttggattttaaagaatttgaatcgcatagatttttgaagaaaaagagacacgtcgaaagaagcataacacattttcaaagaaattaagtcccataagcaacctataaaagaccacaagaaataaatagataactatcattatcttctagatgtaaaggaataatcaccaaatacaagtgtcatttaccaatttatagtcaaagaattctacaactttctcaataaaatgaagcaaatattaatacggtgttatggtaagatggatagaattcctttaatctaaataagagtctttaattcgagtcgtgaaataaaaaaattctgataaggagcgttttttttataatgaaactaattaagctccagaatagtatttacacaacatgtgatatgttattaagctcatgttataagttcgtactctgacacaaacaaaagtgatatataagtgcagaaagacagaagggcgagtccattagccatcattgagcttcgatccatacgtcattaaccctcgaaagtttctccattgcgcagataaatataatatgtgaatgtcctttaatacaattatatatgaagtaatgaaagcatccaaaaacggatatccttcgaaattataatatttacatgtataatgtataatttggaaaaggacaatcatgaaagagtataataatgctcccaaaagaaaagtcttaacattttaattgtcaaaatgatgagtttatattacagaaaaataatacacaggtatgcagcagactcaaataccctaacaaacggtttagccatgaatttaattcaactaggtaacaccttgtttagatgattgttacctattatattatatcgtagtgttaatttaaatttaatatttattttgattgttaaattttattatattgtatcgttaaatcaaatgttacaatacaacgaaaactatcacggacaaatttagtgtggtcacattgttacattttttttctctcatcttgccctttcttattactaaataatcacattttatcctctgcatattgctggcgcatgacatcataaaacgacgacaaacaatataatctattcttgtatatatcaaaataatatagtacaatacaatacaatattatacgatacattatgaaactatacataacaaccatccaaacaagctgaaggatagagttaaaaagaacttgtttaaggataaaaatagatcatcaaataataaggcaacatgagacaaagaaataacataggtaccccacaacaccaaatttgttcataaaatgagcatctttattgtcacataacaatgaaaccaaataatacatcgcaataaattttaaatgacaatcaaaacaaacatcgcacttgaaataagtgcacagtacgaaaggaacacacaataagggcagaaaccaaaacatatgaaaattttgtaacctgaagttgcagaaaagcaactaaagctaagcatcagatgcacattccttcagcaatttgagtacatttttgcaggccaccttctcggccatttttttgtctcgttccgagcacgtgaacttgtgactaacaccgtcagcttcaacctctactgtaatatacgccacgccattcacacgtgagacaacattcttcttctttatataacctttctggtcacatagttcactcaactctctgactggatggagctttactgtatccggtgtaatcaacggttccagaagtgttcgtacgctctgaaacacaatatctttgttgaagccagaatcaacaaagatcgccccggcaagagactcaataacatctccaagcacctacatcgaaccagaaaagcaaacaagttaataaacctaaaatagaaaatcaaattagattattggatactctatctgtcccaatttatgtgatacactttcctttttagtatgtccaagaaaggaatgtcacctttctataagtagaaacaatttaacttttaaaattttctttttaccgttaatgagatgctttactgccacaccaatgtctaaggtttgtcttagaatttagaccacatatttcaaaattcttcctttcttaaactttgtgtccactcaaattgtgccacataaattgggacggagggagtagtattaatccacattgatcttaagttgcttccgtcaattttttccttccgtttctaaaatttatggttttcagataaagtgttatttctaactatgaggcaagtatgcaatacctttggaaaagtagtttcggactcccatccgaaagtggacaccggatccagtttctcaaaattcagaacagtattaactatctgtctctgcagatcctgtgaagcatgaagaatgtgcttgtgtaggccagccctaacagcagcttgagcgtagcattcattatttacggaggcagacctcaaatcagtaatgagtcctggagacagtcccggatatttgaaatacagatgcgctgtaacagcataatctagcactgcatctccaagaaattccaatcgctgcatagccaggtaaatcatcagcagttgaatcaagaaaggtaaaaatgtacaaataccaatggaagaaaatgatttctttcattaataaccctttatttaggaaacatcataattgacttttcattgttcatataacaataagtaaacaagattgctaatggaagactagaagtcaactggaaacgacatccgaagtactgcatctgtttaggatacttattctgagctaatgggacatcacatgtgattgtaagaactttaaccttaaaacagaacaggaaaagggaatctgaacttagaataagattccccgaaggcaagccagcattccaaataaaaccatgaaaaattagcttgaaagaatgaaacagtgcacttttttcctttgtcactagaagttttatgaagaatcattaagtactatcgagccgcttaggtcaatgaaatgttcaggttcacaagacatagatccaactatttaaagattcaatgatagagcaaccacgagacgcagaaatgcaatcaacatcaagatggataatagttatttacgagactaacctgatagcatcgtggaatctcaggtagcatgtaagatccgtgagttagagcctctacaagcagagaaggatcatggaacttgtaatgtagcagtgattccaagtatctgacattaactagcttctcagcattcatagggaagtgcctcggcgaaggtgcatctacaaaatcaatgccgatcccaagccatttcataaatgacaaagccgctacttccccgcctgagctgaggaaggcacctatcagtgcctcaacaacatcagctaccctcttacttttcatcttctgccttcccctactatacatttttactgaaggcatcagcacttcttcatcgaaattgtgaacttgggaattatcaccaggaatgagccaccctttaagatcgaatggttcattacgaatgaatccctgtaaagaaaggtatttattgggatagcacacatcaagcaatccagaagtgttaaataaataaaatgttaaaactcgactataggaaatctttaagaatactcgggcatatttacttattcaaaggtccaaaaacagtctgtcaaaattgacatgtcctacgacaaacatactccaattgatgcactgacaaagtagacattttttacaaacataggtaacagcaatggccaagaactactaattaacattataacaaacactagacttagttgtgtgggcgagggacagtagtaaagaatgttcatacgtgatagttggcaacctttaggtcaacaaaggtctttaattaaagtgcacgtgcatataagtcagcaaagattcatatagacatggagtttatgctatccaaactcctaacgaaaacccaactaaattatcaccaaggaatacctaccggtattttacgagcacatccaagcttgcacaatgcagcattggaaataattttgtttttcttcacagtgagtagaccctcgtgatgattttcataagtcttgaacagctgtatactcgcagcatatttgagaaaagagtctccaagtgtctcaagtgattccaaatgaaacttctcgacgcatttctttgttgtaattgcttccagaacctaaaataaccaagcaaggaccacaaatcagctaaaacacaaggaagtcgagaaaaagctagtctactgatggaaactcaatccctagagtatgtgtaaagctcagagcttaagaagactgaataaaatggagctaaatctgtgatgagataaatgcagcacataccttagcagttggaataaaaatgttttgagtgcactgatctgagtgcatccttttcaagtttgaagccataatcaaggactcaacccgatgcatgaccgaaggaagatatgaataggtataaagggtagaaatagagacaggtgacatgatgatggaacaaagttcaggaggcaattcaacagatgaatcagttgaatctgcaaaggcggacaaggaggtcaaataaaaaagaatagtgattttccattaaacagtgaaactaagttctgagttctgcattataacagaaggagagccatttgatcaacctttagccttctgacttctacatctttgaaggtaattgtgtactttgaaaatgcgtttccccctaagaagtggttctccttcaaagcataagttgatcccatgcctgtaaaccgcaccttacttaatattttgaaaccaaacattgatataaggaaaagctgcaccaaggaattagtagttaattgtacaaaccgctttttgtagtattctttgtaggtaacggattctccagttctctgttccagcaatgagttgcaatccaaattatccaagaaaccggtaacgcagtacaaatagccattatgaggcgtacagaccaatgagttctccaacatgcagctgcataccaccccagttttggtgttcacactgcgtttgcaaccttgcgtagaacaacaatcaatatgcttatccccaagaactagagaaggaaatagcacagaattcacacattcccaatttatttgagggttttgagttgaacccaaagatggaagcaaaagataatcagaaactgcagaaccaacaggcagttgaactgcagccaaagcgtcctgcagtttactcagatcgcggtcaagaagaattctgaatagactgacttgaaacctctgacatcgtaaaatctggagagagaatataaaaagtattacctacttttttacaggtgacaagtaaagagaggataaaagtcataaaaattatagtcacctcttcagaagtaagtctaacaactccactataatttacttgaactagcaagcttcccttatcaacatccaagtcaaaagtcagtctttcatcgtcaaacttgagccttgtcctaacagcaagtactatcccacgaagctggtaactgctgtaagagtcatgttgcaaatcaactacgtagcagtagtacactgtttccgtatcattggcacaatgactaaccaattctggtggaaagtactttgattcttcatcggtagagatttgacatcctgcaaaatataatgggttagaaggcaagtaactttagtccaaaaaacaacctataaccaaaagtgagaattacccaattctttgatgatggcttcttcctccacgatatcaggaacaagattgtcggtcaaggcaccctcacgatgaagttctttacaagcttcgaggcaagcaagttgcttcaatattttggtattccctttcacactaataactttttgcagtgggcaatttttgggaagatgtagggtgcaggtttctgtttccttctcaataacaaacctaggactgggtttatagtacctacaatcacattgaacggcggtcagtaatgcaacatttaatgcctgaagtactcaaagataccaaatcattagaacgtggcaaggattatgcagtacaaacccatccgaagatagccttgagcaatagaagtataacaatgacacgctagaactgagggtgacaactgctccagttgtttcaactttatagtgcaattcattttgcaattcatcatcaagaggtgaacacggaatagaagcatggcgtaatgattcttgccgcatgatctccccactttgcatataattttgcatgcgagtaagagttgatgcatccccgctaggaattaagccagaaatagaaggataatcagtccgttgttataattaaatcttccaaattttatcaataatgattgcacaaaatcaagagctgaagcagatttaatcttttcaatatgtgcacaatcgacaagagcataaatagaatgttttaagtgaaaaatacaccaaaaacaataatgacagaaccctttaaaacattcatatatcaattatatccaagtgatagtatttgtatttgtgaggagggaatggatggaaaagcatacctcccaaccatcagaataaagtgtgagttctgctttcgagctcgaccacgagactgtataaagctacaaacggttgctgagggatcaaatcgaatgacaaggttgcaactttgaacatccaagccctcttcaagaatagaagtcgctacaataatgttgacctgcagcattttgcatggaaatgagactttcaatcttcagaaatcatctacaaaagtagggaaaaataaaactggacttaccacacccttgcgaaattcttcaacaattttattttgaacatttctactctgtgactgcagcatagaactatgtcctgctgtgtactcagttttccaaccacttaattccggaagcaactcatttaacaaagaccgaagaacaatagctgtaatgattctctcaacaaatatgatgcatctcaaatccttcaggtctctattggattggtaaaaaaatgtcatcaacaaaaaaaaagatgtcagccagtgaatcactaaatagaaaatcctatttccccacaagaactacattatttaagacatatcgcaaaatcaaggagcaaatagagtgatagactccagcatcacatttaatcaagataagatgagacaagaagcacaggattacagttttaacattaaaatgtgatcattaaaaggaatagcgtaaaccattggtaaactaccatgtgtaatgttatttcaaaagcatagatgaaagtacacagtcatgtaagggagtcacttaggcagcataatgctccctccattacagttagatggcttgcgagttcaatgactcaagaattgaaattttttagcactcatcttgtgcttccaaaatatttaaatccgtcaacacaaaattgcatgtcattctctcgttgccacaaatcctaagaaatctatcggacaacttttttttccatttattctatggaacatccaatagattgaattggcattgatgcttttaacaaatagagttaatagtcagacctcttacctgtactcaagaagagactctataagactattaactttggatgtcaaatatccagcatcaacatttgcatgaatatcaccaccaagtgaccaatgtgaacctatgtagagagagagagagagagagcacaaaaaaacaagaaagaagaaaacatatttacagctcaggaaaagaaaatggatgttgctaattatgataccagatggtatgtgagccgagaagaccttagatgcacccaaactgaaatctctaacaatcgtttgagcacaaacatctaattctccccatgaaaagaaggtgtcttcagaggacaagaactcggctgcctgattcaaaacgaattcatgttaggaaaatgatcaagaccaaactgattaatttttttatgcacaatcaaggaacgtatttaccttgaaagccagccaaacacccagctcactcaaacaaaangcagctcaggataagaaaatggatgttgctaattatgataccagatggtatgcgagccgagaaaaccttagatgcacccaaactgaaatttctaacaatcgtttgagcacaaacatctaattctccccatgaaaagaagtcggtttcttcagaggacaagaactcggctgcctgattcaaaacgaattcatgttaggaaaatgatcaaggccaaactgattaatttttttatgcacaaacaaggaacttatttaccttgaaagtcagccaaacacccagctcactcaaacaaaatatgaaggacgaatatagttttgacaatctttttcttgcagaaccagcacttaaatcagataggcttgactttgaaatcaaatcttcatactgcaatgataagaaaacacaaccagaattgaaagagcaaaaatgtagaccccatatgttgaacaagccaacatgattgtttaatcattacaagaatagattaaggtttagttctaagttccactattttctcagtgcactacccattttccgctcgcacacaactactatatagagaaaggaagagccaaaataaactgaatgataaatgacactgtatataagttccaagggggaaaaatatatgatataaaggggagtaaatccagagagaaagacgaaaggtatgtatgaacattctattaatagaaaggcataatccattggctgaagaattgatgcttaccttgtctttcaactttatcaggtcagaaactaaactaacaaacaatgtgcacggaatatccacatgttgatatatcttcagcttcggagttgagaatgggatatactctgccaaaacagattcactgacacatgtgtataccttcatttgcaaaggcagatcacaatcagacgaactaaatatatatctacttgtgtctgtctacttgcatacattaaaaagacatgctacccaaaaaataagtgcaatgctcagcgatgtactgacgacaagcatcagaagcaaaaagtttaatgcttccacagtccggtaataaacacaaattacagaaagatacatttgcaaaaatcatgttgatgccatcaccaaaaaaacttctttaaaaaagaaaataacatgcttctatcctttgaactacatttaagaccatattgcatgacgtgaatggataaaaggagaagatttagagagaaccttggaatgcataagattctcgaggtcacgaatcatttcccaagtaaattccgcgctagaacctgcaataaaagtatgactatgacaatccacagaaagacagtttgctaagagtgtgatgtaaaagcatctcaaagggttaattgatgatgataaaattaaatgtcagtaacctttagtctttataggggaagcagtcattccgaatattcttggaagttgtgcgctttcaagggtgaattgacgatgataaaattcctgaaattaaaatcaattctttgacgaaaattgttcaagcaccataataagtcatgctcaagcaggattatagtgggtcttttctaaggtgcggtaagattgttggactagttagacttaaatcacatacgtacaaaagcaatgatgtgagcattagagtgccacatcgggtaaatgtacacactgtctactgtgcctataaaagaattcagccataagacatgtattcccatattctctcaaatctcttttttatctaattcctgtattctctcaaatctctttttttatctatctttttacaggtggtatgtgacatgcaacacacactcaagagacaggaagtactgaaataaaatactgttgaagaagtggtggtaagtatacaatagtagtcaatatccattgagacagaaatccaatgttgttgataacattttgctccattctttcttctttatgatgtaagtagggcactctccctaagacgagcacaaaaattaaggtccagcatgataccgatagaagatgagtacaaaaaatataccttcattatgcaggcataagggtgtttgcctcttgcattgtgacattcatcaaatattagaactttgatcatttcaatttttaagaaactatgcctcaaagcagcaagtagtattgcgggtgtcattacgaggacctgaaagacagaaatgataattacttatcaagcatccaggtatatattttttgataaggacatccaagttatatatatacaacttaagtcctagcatgtatattctgaaactcaaatctaaaatatttgccgagagccatgccaaaacaaaaaggattgtaaggcaaaaaaacagcagctttgtttgcgaattccctcggagagaagctattgcttatactccatagccatttgttttgccggcaggaaaaattttaaggattccattaccgaagttgacatgaaggatttaggaattgcaaagaaaatccttggtatggagatttcaagagaagacggtgttgtacatctttctcagaagaggtatattgaaagggttctcgagagattcaatatgcatacgttcaagcttgtaagtacaccattaactcctcattttaaactttcagagttacaaatgcctcagtccgaggatgagatggagcatatgtcaaagattccttatgccagtgcagttggtagcattatgtatgctatggtatgcacacgtccagatattgctcaatctgtaagtgtggtaagtagatacatgtccagcccaggaaagaggcattgggaagctgtcaagtggatattgagatatctcaaaggagcttctagggttggtcggaccttttgactaaatggaagtgcatgtctactcaagcaagtgttaataatatatcactaaagctaaaatatagttcacgcatacctcatgatcgtccacctgtttttgccatgtagcagcatcccaataatcgacacccatttctccccaatacttgcccactttcaagtcggtatgcatgatgagagcatctccttgctttgtaattatcaatcgcaagatcaacaaaattaggacaagtcaaatagacagtaatgcacctaaaaccaaggcccttagaaaaaacacaaactaaaggggtactcctcaaacaatatcacaagtcacaaaatgtggtaaaaccgtaaaactaatgctcaataaaagagttccacaaactatagccagaagtgaaaaatatacacagtaagatcatgaagtttaaggactagattaagctaggagtagtacctgagccaccagaacaaccgtcgggaccaagaaaacagctatgtatggagaaggctttcgaagaagataagcatagcttctcaaaagcataatagcaataagggtctttccagaaccagtctccaaataaacaatagtgttccgctttaatgccgtctccaatgcctctaactgataactacataacaaaatcatacataaaaaacacacatattacaaaggttccttccttttctatttattttttccaactccatgatacattaaaagagaaaataaatgtaaaatgccaaccttcttgcaaaaggcagagagtcagggcagagctgttggttttcagagactgtaacttcagctggctccattgatagttatctacattaacatcagaccaaataaacataaggacaccaaccaagtaacaaagatgaaacagaaaaatctacaagaaaatgacaaccagttgatcatacataatagcagcctatttatcttacataccctttttaccattttaagcaaacactaagaaaaaccaaggatttcacaaggcacaaataattaaagatttaacaccatggaaacacccacttcataaaaaatccaagaaaactctatgagtaacaatgatgaaacagtaaaactcctaaaggaaatgacaatcaggtgatcatacattaaaaaaaccacaaaactcctgcataccctttttaccattttttaaaaaacacaaggaaaaaccaaaaatcaagaaaagaataccaataaccaccaccaccaccacccctctaaaaaaaaaaaaagacatcacaagtaaatcacatatgcataaaagagtaatctgaatgaaaaacacccagttgaataaaatgcataaaagagtgattttatgaagaaaaacacacataaatagttaagaaaactcactggctagagttgaattgcaggattgtgaataaaatatttcaaacagttttgagtgtttagtgaagaagaaaaagtaagagagatacaagggaactatatgtagaggtttttagcttctatggaaaggaaaaatactctcccccaccccaccacccaccaccccttaatcagagtaactctgcaacaccaccaccngatgaaaagacagaatctttttgacctattaaatataggaaatccaatttgaaggaatataaatcagaactcctttaatactgttctgtttaaattcctctaaagagaaaagcactcagtatattcatttgttttttatttcattattgacccttaagaatctgtttaatgacaatagaatttcttgatctacggaacgaaatttgataatgtaagggtaatttagtcattttgtattaagaaaatggaccaccttaatacggaattctgtttctcgtcactgttacgaggaaataggaataacgctgatggttactgttaaggtgttcaaaccgaaccggaaaatcgcaccaaatttaaaattcaaacgaaaccgattaaaaaactcaattaagtttggtttgacttgatttggtattgagtaaaaaaaaccgaaccaaaccgacatataaatatataaattttatttatattttcaagactttatagtgaattttcttaagaaaatgtagaaatatttggatcctctcatgtattaaccttgaaagtagtaaattaacgaaaaattattgttagatgactaaaaaaataactatcacgtgttactaaaaaaattctctcattagaatattttaataaatcatatgtttgtcaattttttttccatatttactaaacatatattcacttattaaagctttatctataattttaacaaagtaagactgaaataatattcatgtaacaaaaaaactcgaaaacactaaaaacccgaaccaatccaaaccgatatagttggttaggtttgattttggtaaaaactaaacaaacccggtccatgcacaccccctggttactactattttgcttcagtgtgacgtgcacgtgaccgagaataatgaggggaaaaactaaacattttaaaagttcaaaataaagttggtttctatcaaaatataattatatttatttgtttggctgttagtgggcgtttggacaataagaatttgaaatttcagaaaaaaataaaatattttttcaagtaaaaatagtatttgaaaattagagttgtgtttggacatgaatacacaaagaagctaattttaatttttgtgagtgatttgaagtgaaaaaatttgaaaaataattttttgaattttttcaaatttcaaaaaaaaatccaaagttcagaaaatgaaatttaaaattttcatggtcaaatacttattccgataaagaaaaaatatattcaaaaaaataatttttttatcgccaaacgggccttaatttagtatgttgtatgattgactacagataaattgtcaagaaaatttgtaatttatatcagagttaagtaagttatttatcgtttaaaagaggaataaatagaagtgcatcttatcaaggattataaataacaaattactaacaattaatgctaatctcaaaaaaaccattcaaagagattaaaagttgttactattggactatggaatagttatacttccattggcagagcacttggtttacacgcgtgccaatattttacttgtaaaatggcatcgagaaattaattacatactgacttttcagcaaaatttatcttaaaattaaataaagattatcacttgattcaagaaaataattaacaataaagtcgagatcagttgcaacttgcaaagaatgtcatgaattccttttgggcgttttgacataagaattgtaaaattccaaaaaatggtgaaaacaaatttaaagtgaaaatggtatttaaaatttagagttgtgtttggatataaatataattttgggttgtttttgaagttgtgagtgatctaagtgaaaattttagaaaacagctttttggagtttttcaaatttcgaaaaataacaaaatgcatcttcaagtgaaaattgaaaattttatgaacaaatactgattttgaaaaaaagtgaaaaaattttaaaaataaggaaaaacttcttatgtccaaacgggcttacgcagcgtatagatagtgtgacaattgaagtgcttcgttttcttctctttttgcattttcatgtaaaaaattttagtgttatttcttctaaaacagtttgttacagtaattggttcatagagtaaaaggagtagtacataatttactgtaattgttaggcattcgaatcaaagatgtgatattaagtaggagtaataaatatacaaatcaaacatattatggatgagcatgcctttaaacggtttataatcctgatgcaaaatcccgaaaaatctttacactttcgaaattttatattattatttgatgatatgtatgatcaaaatttatttttttatttcctacgtaggtaaatatattaatatagttgttagaaaataatatataaatgagtgagatgattatcttagttcaaataaagttagattaaatgaaaagaaaagggaaaaactacaaaaatcccttgcgatgagctcgtgatacgagattaccgcattggattcaccagcgtgagaagagcctggtttggtcacgaggtctcacgtcagctcctttaccattaaaagaaaaaagatacgcaaaaactaaaatccaagaaaagatacaaatccaactagaaaataaagaagatttaacaaataagtcacatagaatcaagatttaactcaatgtaaataaccaaatcaacaaaaaaaaaatactacaaaaaagagtaacatttatgaagaagaaacacaaaattggttaggaaaacttactggcaagagttgaattgcaggattgtgtacaaagtaattcaagcaattttgagtacaatttataatcaatacaaaatcttttttcagaatgaaagagagagggaaatgaaaagacaaaatcttttggcctattatatactagtaaatccaattantagttgaattgcaggattgcgtacaaagtaattcaagcaattttgagtacaatttataatcaatacaaaatcttttttcagaatgaaagagagagaggaaaatgaaaagacaaaatcttttggcctattatatactagtaaatccaattaaaggcaaaaaccagaactccttttaatactattctgtttaaatttcataaggaaaaaaaaactaattaaataaatttgttttttatcccttattaagccttgataatatgcttaatgacatgtgagtctctcgttgtatagccggaattatgataaagacaagggtatttgtgtcaatgtttataagaatatggaccgttgttagttgttacaagggaaaacgaaaagactgacgctactagggcttgcttccgtgtgcagtgcacgtgaccatgaaataaaagaagaaaaattaaattaaacatattaaaaggttcaaaagaaatattggaaaattatatttatttgtttggttcttggtcatttgcatgaatgacttccaaataatatataatgacaagttgttctaaatttatatatgagttaagtaagttattatcgttttaattaaaactaaagtaagggtatccggttgaggattataataaatatcggatatcgtattacttataactaattactaaactaaagaataccaattaaagagatgcatcataccagatcatgcattatttgtaatgtaattggcagagcgcctggttactcacgcgattgtttttccataaagtggcattcagaaattaattatttacttttagacaataataaacttgattttcttgaaataaataaaaattgtcacaagattcgctattttgcatcccaaaaaaaactttactaaagatgatatcaaatgcaaggaatgtgaaacctttaatttgtcttagcataggtggtgtgacaattgatttttttttttttttactatgtgacataatggattaatttgtctgttcctaattagtgtttggattctaagcttataatgaaggaaataagccttacgcggcgctacttcggattagttaagccaataatctcttatagtagatgattaaacaaaaacaatcaattttattttttaaatttcgtgtatagactttaagtggtactagtattgttactccctctagtccacaataagtgatcaatttacctttttattttggtccaaaataagtgtccatttatataatcaagaaaaaattcaatttgttttttcaaaattacccttatgtatgtattcctaaaaagtcttttactcctcacattaaattaagctgcaacatttaattaagggtagtttagtcacactaactatttttgtttagaatttagtattttcttaatgggtgtgcccaaggcaaactggtcacttattgtggaccggagggagtagttactaatgaccattaattgtttcatgacggtataattttgtactcaaaaaatataggagttataaatatacgagccaaacataaatgtgtatgttgtgtataattttgatgacttaagttttttttttttttttcaatcaaaaatgcatgaaataacatttcattttgtgtgctctacccatccaaattcgaaatagtagtatattttgaatatgattttttttttaaagttcactaatctagtataagttgtgacgtacagacagattctagcgtcctttaattaaatgacctattctaaaacttcgtaaatgagttcaagctgttttttattgggtggatggccctttaattagattaaagaattttaaaaaacaaataaaatgttgttcgtttgtttattttttctattcctatttcaaatatattcgattaaatcaatcgaaagttgattaaataaaaagttaattgacattgagaaaattaattagccgctttctaacaaaataaaatttgaatttcttgaaatgaaataaaatggtgacaaattcatattgattttcttttacaatattattattaataatttgaagatattactatgattttacaaatttgaagatcttattatcttggtactaccagagatgccgatgaaaatattatattacattcatttattaatagagacaacttattgtgagcacgtaatttttgtttacacgacaattactccaaaaaaaatcgaaaaataaaacaaatggttttgttgtacaatttttttggaatttacgtggcatttttggtggttatttgtagttttgtctgtgattgtttagttttgtcaaataaaaatacaaaaaatatatgtcgcgtgtaagtttaggatatcgttctactattaggaattaatcaactcataatttggtttaaaaggaaaaatcacaaaaaaaaaaaacatatgcatcttttattctagttgttgtcattgagtggttttatttcagttaaatgtttaatgtgtgtgataattgttgtttaagtattaattaatatttgtatagctttatttttgattttacaaattaggtgggaattttgtttaaattagaaatttaaaaaaggaaaagaaaggagtttaaattgaagaaacccgggtttgggctcaaatttgagaccaaggaccaggcgcaattcatttcatgacccagtctggttccctggtctctatggcctcaccaaacgacgtcgtttaaggcctatcggatctggaccgttgatcaaacatatctaacggtccaattcaactcccattttaaaaccaaacgacgccgtataagagtttcaatcaaagccgtccatcttgttcaatcctacggtccccaacacgcctcattagcccaacctattcccgtcaaaaactgacccagtcccctatgggaccaaacgtcgtcgttccccctaaacgaaagatcctgtccatcaaccacacttcatccgacggtcaggatcaaaccacccaccccatatagaaatctaaagcctaccccagcccccaaaccaagcacccccccttctctcactgttcctctgtctctctcattcagagacggacctaggtttaaaccctagccgccttagttcccttccttttgaaacccggcggcatcaatgccgccggccaccatcttaacatcctaggaccactcaaccaccctgaatccgaatccacaaaccttttggctcgaatcaattcctagattctcgaatcttcatttggagattcgaaccaaacctcaacctacaccaaccgacctcaaattcacaccagttgctcccctgacctccctcataccctaatcaacggtggtaccgctcgaatctaggtagaacccttcgagccccaaatcggcctgtatgaaccctagaactcaagatcctgggggtttgtccaattaaaagaggagttggggtctaatagacctcaatcgaggtgttttcagtagagaacatttcgattgaagtccgttcgacctcaaagaagtcaagtccagttcgagtctgggttttctttgttcttaagcttccaaggtaatttttcctttccttctgttctgttagtgattctatttgtatctctttatgtgtttagtttagattatatgattttttttaaattaattctgtctgtcttcataagaccttttatttgatccttctgtttgtttgttaattgtacatgttgtgagttatgtgtttaacctattcagagtcgtcaaatagttagttcatatagttttcatgttatgtctaaagtcactgaagcctcatggtaacctagtttgtctagcttgtttatcgattgtttgctatctattatagctcgtatagtcgattatataaatgtcgttaactaaaatcatctgaaaaactggagaagcatgagtttgctcatgtcagtgtaattacttgcgtgtttctcttgtctgtgctaagacctgattgatattgcctcattctttagtttgcattatcaactctttgttgatcaagtttggttttgagttggtcaggattggttcccaatatgacaaattcatgccatttgattaacaccccttatgtgttatgatgtgtttgaacctaagttgagaattggatatagctgtagtaatctgatagttcaacctgaaatcagtgtttagtgtaagtttatatagactataacctcaaacctctaagcttaagggtaatacaataaatcacaggagtttcaagggtattctagggttttaaaggttctgaattgcttaaaggcaagtgagctgacaataaggtactaaaatattaattaaactaatgaagttaattgaccaatagtgggattgatgatatgttaaacacctttagcatctggaaatcagtaacaacatgggcttgatgataaaaagttaaaaatgcacatgagaatagtagtggaacctgctgtacagtaggatatccactgcatttttaaagttcagcagattaaagtagaagaaaactccatgcctaaacaatcctaagtggaaaggacagcctttagggtttattttaagccatgggcagcctgcctataagaggggggttcttcttttctagggaggtcagacaccaagaggttaaaaaacagaacccaagaactgaagaaaaggaaaaaaaccagaaagagagaccaagagtttagaacctgaaagagagaccaagagtttagaaccagaagagagggaaggggggtttgaaccaaaagaaacttgagaacaaagaggaagctgaaaattcagcttttcttcgcctttcttttgttttgaaatcagcattaactgttttgttctatccgaaacttggacttctttcttcgagtgtcaaaaaaccagaaagctgtggttccactgtcattgcggatccacctatttctgcttggcctccgctgagtctccggagtttgttttggtttggattttgagcactatttccattggttgtggctgaactcttttactgggatttatcctgcttactttattgctaaaattgctgtttctgctgtttgctgttggtcattgctgctgcgctgatcactcctcttttccttcttgtgtccaatatccaggtacacacttgaatttcacgctgatgtaacagtaacaatgagtatgaaatgaaagacgtgaagaatttcaatcatttggtgctgcaactatagtcttataagttgttagcagattgtatgattcttcaaggttataactcaatagaaatacattaggtagtttgtacttaattgaaacttagtttgtttaatactgtgattttagttgtttagctgtctgtatgatatggaatgcacaggtggttagtatatcgataattaaatctcatctctagtcctattttacatacatcaggcagattgcttctaattcggccaaaatgcaacagagttttaagtcatttgagctgactctatcaaataggatttcgttaggcagtttatagaacgatgttcaaatcccattaataacatcctctagtagttaattccattaatctcattaaaaagagttagtttatattcaacttggagaacgtttagcgtgagtatagcattttatcaatcttgtatgcatttttttttaattaaattaaaagcatgttcgctcatggaataaggcatgaaataattatccgtctcacaaataatcaatctgataattaataagaggccggagctggccaagcatgtaatttaattaacgtatattttctttctttcatttttagagacaaacataatagaaaatgtagtcactgtaggtttttcctttcaaaaaaaaaaatgagcctagcctcgccaaaaatacaaaaaaatgcaaattgcggggccctcaataattggtcgtaataaatatttagatttctcgggacggactgtttagtgaatttcactaccttccccaaaaataataactcgtttgactctttaggcgcgatttaattaaattatattcttaaattcgggtgcgcatttatgcgacccaaatccaaatctcgacagagtcgaaatgtattaacaaccacgagcgcattgattgtgacgtggttcgaaatgcattttcacgacgttgcaattccataaaaaataataataataaaagcggtttaaacttaataaaagcacataagtcataacatgtatttaaatcagatatttagccattataacaatttaagcgatcgtgctagaaccacgggattcaggggtgcctaacaccttccctcgggtcaacagaattccttacttagaatttctggttcgcagacttcatttggaaagtcgaatattttcctcgatttgggattcaagataaaccggtgacttgggacaccaaaagccaaacctttcccaagtggcgactctgaancacgggattcaggggtgcctaacaccttccctcgggtcaacagaattccttacttagaatttctggttcgcagacttcatttggaaagtcgaatattttcctcgatttgggattcaagataaaccggtgacttgggacaccaaaagccaaatctttcccaagtggcgactctgaattaaataaataatcccatttcgaatattgtcacttaaattggaaaaactctctcgcgcatttaccctccgggacgggcgcgcaaaaaggaggtgtgacactaataagttaagatattaaccaacgagtaacatcactttttgaaaaattgtagtgtcatacaattatttgaggagaaacgggaagaaaaatgttctcatttgttcaaccgcaaaaaggaaaaatagcaattgaattgatctcaaggaaaacgaagttgatcttaacaacttataaatggataacataagtatttcctcattttaaaatttggtttaggactcgagctttgaatatgaatttacctttattacagaatatttcaccctttaatgtgaacttcgaggcacaaattcagatttagtcgagtttcaatacggacagtcaatatgtttgagaaaccaaaaaaaatatatatatatatatatatatatataaagggcttatagctagtgaacagtatacttccacagtctattcagagtacgaggatggaaacatttaattttttgtgtgtaatttgatcaacataaattttaaaaatatttaataagaaaattttgcgtatttgaaaactaaaaattacatgaaaaaactgtaagcctacatagttaataacttaaaatatttaaaaaatcataataaaatattatttaattccctaatatataataacatctccacgtaagagggagtaggatttttcagtgatgtggagaggaagaactgttgaaactttggtttggtctccaaggaagaactagtcactactcactactcactactcactagtggaaaaataataagtacatttttctttttgaaagcttaatccctaaggaaggctccattattatattacaactgtgataaagcaaatggacaaacaactttttgtttttgatgtggacagggacaactgtagaaaccttggtctccaaggaattagtggaataataattcatttttgtttttgatgtggagaggaatacttttgaaagctcactgggcgtttggacataagaattctaaagttttaagtgaaaattgtatttggaaattagagttgtgtttggatatgaatataatcttgggttatttttgaatttttgtgagtgatctaagtgaaaattttgaaaaatagctttttggagtttttcaaattttcgaaaaatttcaaaattcatcttcaagtgaaaatttaaaattttatgaacaaacgttgatttcgaaaaaactaaggaaaaatttcttatgttcaaacggactcttaatctctaagtaaagcaaatggacaaacaactttttatttttataataggataaaaattaagagaccgtttggattggcttattttaagtacttttaagctaaaatagcttttaagtcattttgtagtgtctgaataaagtaaaaaagtgcttttaagcacttgtttttaagctaaaatgataaaaaaagtcaaaagccaaaagctaaaattcctaacttatgacttttggcttaaaagtcacttacaacaagcccgtccaaacgggatctaaaggacattatcgaccaatagtactagtatgtgtagctccttagttacatattcatttaaacccataacttttgacatatatatatatatatatatataagatttgaaccgggtgcggctcaacactattggagacctaaaacccaactagcataagaagcctttaaccttcccccctcccaaaaaaaattcttatgtatacaatttttttaaaatctaattttctagctttttgagatgcaaaattatgaataattattataatttttttctaataattctttttcaatcggtaatataactaatccatttaatcttttttgcgccatggttgattttaagtaagaatttatcaattttaattttaaaaaacttctttcaagtttctagtttgtggtcgagggtggccagaggcaattgttatgggaactagtattattattctataagcaatataagcatttgaaaaaaaaagtctttttatctgattaagtgtatcaactaaagtactatgatctcttctttttttggggattgtatcaatgattcatttttttttctcttaagttttgaataatcggattcatactttccatttgacatatttaaattttgacaaatactaagaggataatatctgcatgcgtattgaaatatcaagaaaaacaaattttagatgaaaattaatattgaaaagttggaatagaaaaacctgagttattaacttaatgtgaaaataaatatttttgctttgacaatatacttctcgtaggctttagcttgatagatgaaaaatgcagaataataggatctctgggagacacaaagaaaaatataacagtaataacgtttggtgagaactgagaaacttaaaaaaaaattgaatattgtaaaaaccaagaaagaaagtggaaagatttacataatatttatcaaagacataataacttgattcctttcataaattttacatttacatgttaggggcccaattttaaggaatatagatatttttccttttcagtttttaccaagatatgtaataagaataaaaatacttaatttttaaaaagggtataggttaaaaaggt

SEQ ID NO: 65 - DNA, 52281 bp, Nicotiana tabacum
  misc_feature (11517)..(11517): n is a, c, g, or t
  misc_feature (52150)..(52150): n is a, c, g, or t
ctctaaaattaaatttcaaacccagaatttgaagtaaacgtccagaaactaattcaacccaaataatggaccagacagaacttcagccaagctgattttagttcatttctaaggtcagctagtcagaacttgttgcaccaccaggaaaactgcagaaattaatagaagactcaatgaaacagtaaaatttctagcatttttgtatttttctgagcttttcttctttttaccccttgggtctgccttgaaaagcaagaaaagctgcctttataggcaagttattagggaagctttcagatattagtctttgcacatttgaaccttttaaaattttcattatagctcagatacccttattaaaagaacagaaaatcagaaccaaccccttccccagcttcctagaagcttcctagtctgttatagcaagattcccttaagtgacttacccaatttaccaccattacccctgtattttaccttgaagcccggccagacttgaacatgggctttggatccccaaacaagctcaatgcttaactagacatgggcttagactcagaccaaacagaacacctttaacatgagatcttagactgtcctaagagcccacagacgaaacccaaacaaattcagaagactaaaaaaccaaacaaatcaattaattaacaatcacaaacaggcagcgaattaattaagcttagtgaccaaaacaaagacaaaaatgagaaatttaagcatggcactaatgaaagttaaccaggccataaacttcaacaaaagaaagctgaaaaaccaacggaacatatacgaacgcaaaatcaatcttaagggaaactaggtccagaaccttgttcaaatttcaatcaaggtaaaaaaaacttaaagaaaagaagaggaacaaaaggtaaactcggatagaaaagaaaaagggaaggaaatttaccctagcaaacccgaaatggcatcggacaccttgatttaaacggaactgatgctaatcacttattctttgtcaagaacaaatgagcagcatcaattcgtgttgaatcagacttcaaactcgagcaaatcggaggcttaaggtcatgcatgcccacaactttgatggaattttagggttcgaactcaaatttgaaattcggagagttctggggtgattcaagggaatctaagtgggcatttagagtgagggagagatgggggttcaagggtgtgattttggggtcgaaccgaggtggcgccaccaccggagaacataggggcggttctaaggtttttctttcaatccaagattcgagacgttctgggcttgttcgatgtaaggtgcttgtagatttggattgagggagttgaggagggtgtttggtgttaatttggggtcggttcgtggtggcgccgccaccggaggtggggggatttgtggcggcggcgcagggtttgcaggggcgggttgtgagggagatgagtgagggttatggggcaggtttggatatttaaatactaagtcgccctgtcttcaggactatccaatcaagaaacctcgacggtcctgaactgttgcccattaaacgacgtcgtttggtttggggggaaaccgaccgggtcaaggcgtgggtttgtgccgggtactgggtaatttcgtttgggctggggacagttaaatgggttttagcccaaatctggatttctttctttttcttctttttttcaatttcaaaattcttttcttttcaaaattaaaataaaaactaaattaataataataatttttaaactaaattaacctacccaattagattaatcactcaccatggtatttacaatacttaattaaatcctaaatttaaagaaaactatacaattcaaaatcaaaaagtaaaaatgcaaaaatgaattatttttgtgatttttattttttttataaacaaactaatttactaattaatctaaaaatataaaattaaatcctaaatgcatatgcaacatattttttatatttttcatgaattaaataaaataaacgtgcaccgacgaatgcaaataattaacagaaaatgccacgaaatccaacaaaattgcaaacactgaaagaatttttttttttgaatttgtcggagtaatacatatagggcaaaaatcacgtgctcacaattgcccctctttgctcggaaatacgaagagttttcgtgcaaagataagtgaacggatacgagcaatttttgcccatttgaatactccatgggaagcatttttgaaagatttgaccgcaccctacttcgaggttgcctacatatccttggctaaaaaggaatcaggtcagtatagttcagaaatgtctggtagctgggactaccatggagctgtgatttcactgttactgctgctgctgctcactgaaccccttattacaccatgtcaaaaataaaataagctagactagactatgatctatgctttacaaaatcttgtcttggtcttcaaacttgatctttttttttcttgttgccttgttgccttgtattctcttggtaaaattcctttgttgccgtcttgaattgtaaactgagatgcttccctttctccaagtggatgtctgactgctgaacttgatatgtattcccatgctatccaggcgggctcctggcttcaaaatgtgaatgtattctcatgctatccatgcgggctcctgactgctaatctgaaagtgttccctgcttttcaggcgggctcctgactgctaatttgaaagtactccctgctctccaggcgggctcctgacttcaacaaagaattcgacagctaaaatttaaattgtactcccttgttctccaggcgggctcctgattgctacgcttgaaagtattctctactctctaggcaggctcctgactgctaaacttgaaagcattccctgctttctaggcgggctcctgacttcaacatttgaaagtattccctactctctaggcgggctcctgacttcaacttgaaatatattccctgctttccaggcgggctcctgacttcaacacttgaaagtattccctactctccaggcgggctcctgacttcaacttgaaatatattccctgctttccaggcgggctcctgacttcaacttgaaatatattccctgctttccaggcgggatcctgacttcaacacttgaaagtattcactactctccaggcgggatcctgacttcaaaatagacaaaacaaagaaaattttcctgcccctgtttggtggctgggcataggtatgagtgtaaaactaaatcatattaccaagtattactaagaattgaaagctagatcccattattcaggaggtcctgacaactcttaactaaacgacaatatttaaaacctaagttatgtcttctacaggtatgatttctgctaaatcttgtaatctaagagggtcttaaactactctccattatccagcagggtcctgaaaatcaaaagtcaagttttatcttaagagtggcaactttcatggctgaactatactaccctacagcagaacttacgctaaatgttattatctaatcgaaaatttaaagctaagtcccattattcaggagggtcctggaaactcctaattgaatcctatctcgaacctgcaaacttctaagctaacttatattcttttcggatacatttatgctagattatgctatttctgaactttaaactaggtatcattttccaggagggtcctgagagtttacggtcaaaccttttttgatgcttgctctttcccctgcggagagtttctccgaaacaaatgaattttctgcccctgtttcaatcaaagagaattttgtcagtttaaagtggtggttagctgatggctttcttgctggagatctttccactacattattttgttctgaatggctttctcgaactggccctgaaccgctttgactttctgactgactttcaaatcccatggcctttaacaaaccgagtgttctatacacatgctgctgtttcacacccctgaccccattatctgaacctttgtatctcccatgacattaccaaatctttccaccggtgaaacttctagtgatcccctgtatcccgccttacatcatgctgtctttactaggctctggtcgcgttgtgatttgcaattctggaagttggtagtgagttttgaaattcttttccacttacttgggacaaaactgatattaaaacatcttaaacaaataaacccaaaagaaaacgaaatgcaatgaccttacgggttaaggataagaagaatccaaaaccagatgactgctacaaagggaaaaaagaaaaagaaacttatctgagtggaacagctggcaccaatgatcatgacatgcatttcgtattaattggcccaatctgtccaaccgatccactttcagttgtcgtacttcatcgcctcagcatttccatgacttgattactttacccaaaccttaaccttgttcatcctgcggtgccttaaaacagttttccaccaacagacctttctcacttgtcaactcactttcgcctcagggtgcccgtgaaggttttcaccaataagactctctcattttaatttctctcaactcccattgtcttacggtgcctgtgaaggttttcaccaataagactcttttatttttatttttcctacttgaaccagagtgctacccctgacatgaattacttttacccgcttgacttggcatttctcaaagactgatcagaaggtctttctttggactgtaatgtgggcttttggacatggttagaaagaaaaggtataaaaagctcaaaataattcaaatgggttcaaattacaactttcggaatcagatttctcacaacgatcatgacttttgccccacttttcctgcttggggacttttggatttttaatttgatgagaacgaaccgtgaggctgcctacgtatccttaaaaggaatcaggtcgaacgtagttcatgttataggaattactttgttgttgtgattttctcttttctccttcttttctttctcttttgcttttcttttttcctttttattgtttttcttcttcttttctcttttcctcttcttctcttctttttttcttctctcttctcttttttctttttttttttctttcctttctttctcttttccttttcttccttttcttatctttcatgcttgtgcttctgatattcactactgatttcgaaggaggggtaagaaagaaaataaataaggttcaaaagaggtgacgagggataaagtgttttagatagcagaacaaaatgctttcatcatttcaatcttcaaaatatgacaaatacaaacaagtacaatcaacaaagaaatcatacatagtatctcttgactgtatcggaattgatagccatttccacacatttcccttccacatctgtcaaatataacgcgccattggacaacactctagttacgacaaatggcccataccagtttggggcgaactttccttttgcttcaacctgatgaggcaagatacgtctcaacactaactgacatacttcgaacttctgtggacgtaccttcttgttatatgcccttgtcattctctgttggtacaattagccgtggcatactgctgtcaatctcttttcatcaatcagacttaattgctccaagcgggttttgacccattcaacgtcatcaatttctgcctcagcgacaatccgaagggataggatttcaatttctgcgggtattactgcctccgtgccatacaccaacaaataaggagtggcccctactgaagtacggatggtagtgcgataacccagcaatgcaaatgatagcttttcatgccactgtctagaaccttccaccatatttcgaagtatcttctttatattcttgttggctgactcgactgctccattcgccttagggcgatatagggtggaattgcgatgtatgatcttaaacagctgacatgtctctgtcatcagatgactattgagattagcgccattgtccatgatgattaccttcggaatcccaaaccgacagatgatatttgaatgcacaaaatcgaccactgctttcttagtcacagatttgaaagttttcgcttcaacccacttggtgaaataatcaatggctaccagaataaacctgtgtccgtttgatgctgctggctcgattggcccaatgacatccatgccccaagcaacaaagggacatggtgccgacattgtatgcaattcagatggtggggaatgaatcaaatctctatgcacctggcactgatgacacttgcgcacaaaattgatgcaatctcgcttcatagtgagccagtaataacctgctcggagaatcttctttgccagaacgtacccactcatatgtggtccacaaactccggagtgtaactcaatcatgataattgtggcctttctagcatctatgcatctgagcaatcccagatctggagttcttttatacaatacccctccattgaagaaaaatccacttgccaatcgtcgaattgttcttttctgatcacctgtggcatgtaccggatatacctctgttctgatgtactctttgatattatggaaccatggctccccatcaagttcttcttccaccatattacagtaagcatgctgatcacggacctgaatcagcaaagggtctatataagccttatctggatgatgtaacattgacgccagagtagccaaagcatcggcgacttcattatgtattcttgggatatgcctgaactctattgactgaaaccgttgacaaagatcatgcaaacattgtctgtacggtatgagtttcaaatcccgtgtttcccattctccctgaatttggttcaccaagaggtccgagtcacccaaaaccaagacttcctggacactcatatccacagctaacctcaaacccagaatgcatgcctcgtactcagccatattgttgttgcaataaaaacgaagctgagccgtaacagggtagtgttgccctgtttcagaaataagcactgctcctatccccacgcccttcatgtttgcatccccatcaaagaaaagtttccatcccggcttctcaacttgctcggactcatcaatgtacatcacctcttcatcggggaaataagtttttaaaggttcataatctttgtcaaccgggtcctcggccaaatggtccgccaaggccggtgctttcattgtggttcgagtcacgtagacaatgtcaaactctatgagcaagatctgccactttgcaagcctccctgtgggcataggcttttgaaagatatacttcaatggatccaagcgagatatgaggtaagtagtataagaggacaaataatgtttcaacttctgggctacccaagttagggcgcaacacgtcctctcaagatgagtatacttaacctcgtacggcatgaatttcttgctaaggtaatagatggcctgctccttcctaccagtgatatcgtgttgacccaatacacaaccgaatgaattgtccaaaaccgtcaaatttagaatcaaaggtcgccttggttctagcgggaccaacacgggtagatttgacaaatacccctttatcttatcaaacgcctcttgacattcatctgtccatttgaccgtagcgtctttatttaacagtctgaagataggctcgcaagttgtcgtgagctgagcgacgaacctgttgatataattcaatctccccaacaaactcatcacctcggtcttgttcctcggaggtggcaattcctggatggccttgactttcgatggatccagttcaatgccccggcggctgactacgaaccccaacaactttctagacggcaccccaaaagcacactttgcaggattaagcttgagattgttcctgcgaagccttcgaaagaactttttcagatcactgacatggtcagactgctttctaaacttcacgatcacatcatccacgtacacctcgatttactagtgtatcatatcatggaatattgttgtcattgccctcatgcaggttgccccagcattcttcaagccaaatggcatgacccgatagcagtacgttccccacaatgtgatgaatgccggcttttctgtatcttccacgtccattaagatctgatgataagtcgtgtaacaatccacaaaagaaccaatctcatgtttggcacaattatcgatcaagatatggatggtcggcaatgggaaattgtcttttggactcgccttgttgagatcccgatagtcaacacacaccctggtcttgccatccttcttcactacaggcacaacattggctaactaagtgggatgtcgggtgacccaaatgactttggcatcgaactgcttggtgacctcttccttaatctttacactcatatctattttgaactttctcaacttctgtttgacaggagggaattccgggtcagtgggcaacttgtgaaccactaaatcagtgcttagacccgacatgtcgtcataggaccatgcaaaaacatctttgtattcgaacaataatttaattatttcctccctgagttgaggttccatatgtacagttatcttagtttcacggacattttcttggtcccctagattaactgcttttttatcgttcaaattaggtttgggcttttctcaaaatgacttagttccttactaatttcttcataggcctcatcatcatcaaattccaactcgtcatcacactcaatttcttgtattattatttcagagttagattggcttttaaaattgggccgaagattcctcatgcatgtcatgtcattgatatcagcataaaaagaactgtacaaaagaacaaaacaaaaataaaattagaagaaatgaaggaaaaggaacaattgcatttcattgaatttaaagataagagggttttagcacatccaactggagggaacataatatctgaattacagaccctggaataatccagacaacaaaagaaaatcaaaacccactaccaagactccatccggataggaagaggagtagcttcccagttgttgacttttgcacgtggtcccacgaattgcacatctgccttgcctttcccagcctcaaccatgttaacctcagcgaataacctttcgaaccccttctccaagtctccgttggcaccaatcagtgatccaggaacctttgacaacagttgctttctgctacccgacctgatgaatgatctggacagacgcgggattggctttggcaatgcccatgctttctgtttcattttcctggctcttctcacatccacaactgtgggcttgaaccctaggccaaaagtatctagattccttggaagagaaactggctacacaataccttgcaaagatgcacccaaacccttgcccggtacgaaaccatttttcaacggctaccatgactgatgcagtagctacctttggagttggaatgcacttcccttctggaattttctcatcctatactgtgtcaaaaacctgataaacccatggccccttgtcgtcttcaacctctatgagtggaacaatggcatcattgggcacacacaaattatccttgtcgtgtatgacaatttcctatcgatcccattcaaacttgacaatttgatgcagagtagatgggaccgccttggcagcatgaatccaaggtcgtcccaacagcagattatacgaaacagccacatctagcacctggaattccatagtaaattcaactggccctattgtgagctcaagcactatgtccccgactgaatctttccctccaccgtcgaatccccgaacgcaaatattgttcttgtgaattctttcatcctccacttacagcttgttcaatgtggagagaggacaaatgttcgcgctagacccattgtcaaccagtacccgagtaaccacggaatcttcgcatttcaccgtcagataaagggctctattgtgctcggtaccctccacgggcaactcatcatcagaaaaagtgactctgtttacctcaaatatcttgttagctatcttttccaagtggtttactgagattttgtcaggaacgtggtattcgttcaagatcttcatcaaagcacgacaatgctcgtctgaatggatcaataatgacaagagcgagatctgagccggtgtcttccttaactgttccacaatggaatagtcttgcactttcatctttctcagaaattcttctgcctcttccttagtcaccgctttcttcaccaacactggattattttttgaggtcttagcttttctcagctcgtcgggggcaaagcatctccccgatcgagtcaaactatgagtctcacatacttcttctttaacctctttccctttgtaagtcacggtcactcgttcataattccatgggactgccttgctgttgactatcggtaattgagttaccagtttgataacgacaggatctgtgggggcgcccttcacaatgaccacaagattgttcgtcactcctggcacaaccactttttctctttcatgttttcctacgacgtcactagtggaccccttcttgacctccacagatggttcattatttgcgctgttcaacttgatcatagacttctcacttgttgacttttcaaatggtctggcttcactagcccgaatcatcatgacggtttgcgaaggcttcttaggctcccctcccttatgcactatctcgatcatatttgtttcatgatgggctggcaagggattctggttgatattaggtgcctccgaagcttggacctcaatccgattggtgtcaatgagctcttgaatagctgttttcagtttctagcatttctctgtatcatgtcttagggcccctgagcaatactcacaactcatcgagcggtcaagatttctgggagggggatttggcaatttagcctcgatcggattcaacatacccaactgtcttagtctgtggaataagttagcgtacgattctcccaatggagtgaaagtcttttgcttctacaacctctcattcttaaaggcttggttgggtcgaaaacccgttccaggaggatttctgtgggcttttgggggtaggtagttattttgtggagcttggtatgggttttgtggagccggcgcatggtattgtgcatgagtgggaggttgggtataggtttatgcatgatggacagaaaaatagggatctggcggtgggtaatagtgttgtggtgggttatatggagtttgggggtaagtttggggatagggtcgcggctggttgtagtaacggggaaggttcctggatccaacccaagctctcgactcaattgtcgcaacatcctccttcttcttcttcccgagcacacccccagtaccgttttgaatggcctgagtggtcgccttgattgctgaataactcatgatcttgttggacttgagtccctcttcaaccatgcctcccatttttacgacttcattaaaggacttacctactgttgacaccaagtgaccgaaatntaacttacctactgctgacaccaagtgactaaaataagtgggttccaaatcctgcaagaaataatcaaccatcttgctttctttcatcggagggtcaaccctcgctgcttgctctctccaacggaatccatattccctgaagctctcaccgggcatcttctcaagctttgtcaacgatagacgttctgggataatttcggggttgtactgaaaatggcaggcgaaggcttgggccaaatcatcccatgtgtaccacctgttgtgatcttgtctggtataccattctaaagttgatccacttaaactttggttgaaatatgccatcagcaattcatctcttccacctgctcctctaatcttgctacagaatcctcttaagtgtgctaccgggtcaccatgcccatcgtacagatcaaacttgggcattttgaaccccgctggcaactgaacatctgggaacagacataaatccttataagccacacttatttgacatcccaaccccctcatatctcagaatgactgttctaagcttttgacatttctgatcatctcttcatgctcgggagttttgggcggtttcttggtctttgtcgggatatcaagatgaggggtgtaaggatatggttctggaactttgaaggtgggtttagagggatagtactggttttcgtgagtttagaacaggggttcactggaaaatcgatgtaatatggcaggtggaggtgccacaaaaacaggggtaattggtgggggagggtatgagacttgtttgggtggtggagtttgagaagtatgggaagtggtgccatagcattgttggtagaggggaaaggctggagatgagttcacagtgggaggatccggaggttgggctgatggtgggatataagcagggttggcaggataaaccggtggtggatgccccttcgcccaagcttggtacatttaagccatctgttgcttcagcttatacatttcttccctcatcgcattaacgtccatttcttccacctctttcgatgggtcgataatacttgtttctggttcctggttggccatatcagcctgtctttcgatcgggtattgtatgagtgagttgccaaaatgtcgatcaactaaccacctgcctgagctcaatacatcaacaacaacattgttagcgattaggctttaacagattggtaaccgcacattgggcatgaatgcacctaaatagttaaccgactctattatgtatttgattggttgcatgcgtcatcccggccttttctttcttccgattgcaaacttccctctttttttcgtttccttccgttcactcttgcctctgttctctctttgtttttactctctcttgatttcttattttctctctcttttttgtttttccgctcttttcttttctttactctcttgtattccctcttattttctttctttctcttgtttttccgctctttctttctctcttttatttggttacggtcgaatcgtatggagattgcctacgtatcatgaccccgcatgaatcagaccaagcgtagttcttggggataagtgtgaaataaagtaaaaacattttgggattttcagtttttcatatataaaaaattgctattacaaaggctaaaattaacagacgggaaagaaactaacagactctgatgaaagaacgagatacagactcaaaaacaaacaacccacacattctgataacagaaatatcgactccacaaaaactggcagactttaacggaaagaaagtacagactcaagaaatcacgaatacatcaatgcctcaattgtcctcgggacctgcggggcatcattcggccttgttgcgggccgactcgctaaatccccctgaaggtggtagagatcttccattacccggcgaacaaaaatcatcacagtagcaaagaacatagatcgagtcatatcctcacagctactgcacttcatcgcgatatagttggcaaccattttgactcgctctcttatgacgcccttctctcgcaacaaacgcccaatttgtttagttctggcttccaaagttcgttcggccacctggttctgctcttggagttgttgcaagtccctttctagtcgtaccattaaggcgtaacaatgttctctttcagccttaaaatttttggcttgtttggccatttcaccctcgaggttgccaattgccttttcccaacccgtgacccctctttcatatctttctttcatctgttgaacgaaagttgcacgcccttcagcacttttagccaaccttgcccgtgcttttgctagttcggactcagctttctgcatgtcatcttcatagtcacgtaccttttgagtgaggttgtaaatgagcctctcatctttcctacttcggcctgggttctcggcttccattttcaactgtcgaacctgagctctaagggcttcattttctcgcaccaatcttctcctttcaccttcggcctcttgtgcctgcaaatcattgttgaaggtgacatttctcagttcttctcgtaaacaatggatgatggccttatattccttttccttctcccccaagctaacctctcttgaactttatcatcaaaggcttgaacatgaggccttttgggctctggctcgttgtttacataatacattttatcaaaccaagcagcataatttggatctacttcccccttggcgatgtctggtacctgggtgccattttttagataccgacagctattccaatcctactaaactgcagcctcgggcaatgcagcttctggatgtaactctatgacctgggtgtttagatcttcatctttaggcacaatctgataccttcccaattgccttaagaccctctgcggtgcatatggctgaatactccttactcccattatcctcaggtatcctttggtagcagtcatatagatggcctctatcttcggaagcaatcctatagtccactcaacctgacctgcgtcaagaactttcaaatgagaaactcatgcttcaaatccttctggtgtgttgtaatattttatcctctcctcataactaatgatgaagttgtttgggctcgaaccgtaactcatgaatctgggatgatggcgaatatgctcgatcaaccacatttgcagaatgatgcttcaaccttcaaaaaattgagcccctactttgcataaagtcaatgctcgatagatatctgccaccaccaacgggacaagtgtatgatcttccttggtagtgaggatttgtgcaattctagccatacgaatatccatcgttcctttctcatttggaaaaatcatgatccccaagaatgccacaataaatgcgaaccggagatgaacttgccaaagacccttgttttgtttattgcttagaccattttcatgcatttcaaaaccggcagaatgcccgaacctgaagtataagaaatggaaagcacaacacccattgctcacacgttccttatttgtctgtttactaatattcaggatgtcaaagaacttgtgcacagatggagcccttggaaatataagttgttgtttcctcaaatcccgtctgaattcaatgtacccggctatctcctccaaagtaggggtgatctcaaaatccgaaaaacgaaagacattgtggacaggatcccagaaagttaccaaagctttgatcagatcttcccgcggtttgattccaaagaagtctataagagcacccaagtgctttttcactcatttctgaccatcttcgtctagatcattctactacatacgcaagtgtagccgagactgttccctaaccacttctggcggttcctggatggtattcatttgttcttgcggaagattaaggttaaggttgactcgagttgacccttttgtaaacagtttttgaagaagaaaggaaaaataaaaaagaaaagaagaaaataaccataatttcttcccctatatgccaactttagccaaatggctgtttttgcaaatgcggcccctttccaatttcgcgcgaattttgaggccggggggaattatttttatgacccctcacaaactggtccattctttcacggaaatagcctttcgacaactgaagcaatattctgaggctatcttggcaagaacggtttacaacattactgaagctgaatcggcttattttgaccgaaaatcaagattgcgttcactcgaccaccgactctctttccttgttttttttttttttttttcaaaaataaggccgaaccttatgtaggttgcctacgtatcccacatccggtgagaatcagacctgcgtagttcggtcagttttgacacatttgaaagaacatcatgtttgactcttttggaataactctttttttgaaaaaattttgggcagagtttcaggacgcttttcaaataccgagatgtttagaaccgggttttacttccttccctatatcacttttggttttcctctcttttttcgctttattttcccatgtcggtcagcatacaagctaaaataaatgttcacatagcacgtagaatgcatcaggatggtctgttatttcgggcatacctgtcctagacggacccaatccctgtgttgagtcccctaagtcaaatgcatatgatgcaaacaaacgctcctactagggattcggcatgaggctgtgtttttctaggtttaaatcctggggttttggtctagacttggggtacccgagcagataattggagccgaggagggggcgacgtaccgggatcattgaagtctacctggccttgtcgttggcccagcctcgttctatttggtataatttctatagaaaaagtgggctacgcgaacgtatgcaccatttccagaagactaagatgggtggcgtaagaagacagttatatacaattcagataatattaaagcggtaaacagatagcatttagcacaaaagattcacagaatacagtaatatcaacaataaataaagccaagtaaaaaatatgttaacaagctcgaattcttgaaccctgaaccagaaattctgggttcgatccccagcagagtcgccagagctgtcacacctcctttttactacaccccgtaaaatgtataaattcaagggagtttttccaattaaaggacaatcgaaacatgattgtttatttattaaaaatcagagttgtcacttgggataatttagggtgtcccaagtcaccggtttaaaatcccgaatcgaggaagttgactcttatttatggttcgcgaacacagaaatccgggtaaggaattctattaacccgggagaaggtgttaagcattctcgagttctgtggttccagcacggtcgctttgatcatacttggcttattaaaattatttaattattgattttagatcctatgtgcatttaccctttaccgcttttaaatcacttgattattcgtagttaaagaattgagttacgcgtacgtatactcttttcctttggcgcgtcaaaaatcatgtcacgcgaacgtgtccataattaataacgctttgtattattaagaaagtttagccgaagttacgcaaacgtatacgaacatttgttaaagaaatcgtaatcgtgtcacgcgaacgtgtccacaatcatgatggtattttaaacggccctaaaggtttctctacaaacatttgttattttacatctacattatgaaattaacacaagagccatttgttactaagtgtctaattatggcttgcctccaatttccttttcaagaccctaattatttaacactaaagtacttgacaaatcccttccaatcaacaaggcttcattgaaccattttattagcggaagggcctgaattaaacaggcccaaacttctgagaaacaaacctgcctcaacgtaggggaaatcttttggatttaagagactccaataatattcaggcccaaaggcccaaatattttggcagcgctggctacaagcaaggacttcaggatcgaatccctgaagccaaaatccaacaatgaccttgctcatacccttttcgaccattgaggaggggagaaaacgagttgggggcattgataagaaccttatacagcaatcactaataatcccgtttaaactgaggcgtaaactgtacggaaacatcaattggctacaattaaaatcaagcaaactatacatacatagcttaacacatgaattatatcaagaacacaaaggtataaacaacagaagctaatagaatctaacatctaaacaagatttctattcgaattccaatttgaacatccgaatctgcatacaaattcagtctggctccaacttgtggcacttcatttgttagcaaaggggataagaaaatacctgagagaacaaataagaaagagcaagggtcagcagcaaaacacaatcaacagaaccaaccagctgctctaaaattaaatttcaaacccagaatttgaagtaaacgtccaaaaactaattcatcccaaataatggaccaggcagaactttagccaagctgattttagttcatttctaatgtcagctagtcagaacttattgcaccaccaggaaaactacagaaattaacagaagactcaatgaaacagtaaaatttctagcatttttgtatttttctgagcttttcttctttttagcccttgggtctgccttgaaaagcaagaaaatctgcctttataggcaagttattagggcagctttcagatattagtctttgcacatttgaccctttttaaattttcattatagctcagatacccttattaaaagaacagaaaatcagaactaacctcttccccagcttcctagaagcttcctagtctgttatagcaagattcccttaagtgacttacccaatttacccctattacccctgtattttaccttgaagcccggccagacttgaacatgggctttggatccagaacgggctcaatgtttaactagacatgggctcagactcagaccaaacagaacacctttaacatgagatcttagactgacctaagagcccatagacgaaacccaaacaaattcagaagactaaaaaaaacaaatcaattaattaacaatcacaaacaggcgaggaactaattaagctaactaattaagcttagtgaccaaaacaaagacaaaaatgagaaatttaagcatggcactaatgaaagttaaccagaccataaacttcaacaaaagaaaggtgaaaagccaacagaacatatacgaacgcaaaatcaatcttaagggaaactaggtccagaaccttgttcaaatttcaatcaaggtaaaaaaacttaaagaaaaggagaagaaaagaagaggaacagaaggtaaactcggatagaaaagaaaaagggaaggaaacttaccctagcaaacccgaaatggcgtcggacaccttgatttaaacagaactgatgctaatcacttgttctttgtcaagaacaaatgagcagcatcaattcgtgttgaatcagacttcaaactcgagcaaatcgggggcttaaggtcatgcatgcccacaactttgacagaattttagggttcgaactcagatttgaaattcggagagttctggagtgattcgagggaatctaagtggggatttagagtgagggagagacgggggttcaagggtgtgattttggggtcgaaccaaggtggcgccgccaccggagaacataggggcggcgctagggtttttctttcaatccaagattcgagacgttctgggcttgctcgatgtaaggtgcttgtagatttggactgagggagttgaggagggtgtttggtgttaatttgaggtcggttcgtggtgacgccgccactagaggtggggggatttggggcggcggcgttagggtttgcaggggcgggttgtgagagagatgagtgaggggtagggggcaggtttggacatttaaatactaagtcgcccgggcttcacgaccgtccgatcaagaaacctcgacggtcctgatatgttgcccatgaaacgacgtcgtttggtttggggggaaaaccgaccgggtcaaggcgtaggtttaggccgggtactgggtaatttcgtttgggctggggacaattaaatgggttttagcccatatctggatttctttctttttcttcttttttttcaatttcaaaattcttttcttttcaaaattaaaataaaacctaaattaataataataatttttaaactaaattaacctacccaattagattaatcactcatcatggtatttacaatatttaattaaatcctaaatttaaagaaaattacaaaattaaaaagtaaaaatgcaaaaatgaaatattttttgtgattttcattttttataaacaaactaatttactaattaatctacaaatataaaattaaatcataaatgcagatgcaacatatttgtttgtatttttcatgaattaaataaaataaacgtgcacagacgaatgcaaataattaacagaaaatgccacgaaatccaacaaaattgcaaaaaatgaaagaaattattttgttttgaatttgttggagtaatacatatagggaaaaaatcacgtgtcggagtaataataatagaataatagaaatattgaacataattaaacactcaactaaaaataataatcacaacctcaagaataaaccagcgtcgcccagaattaccaatctttaacagttcaaatataaataccggaaaaccaacacagctcaagaaatggaaacatataggttgttgcggtgtgcaacccgatcccactagataacacataatcctcccttatttcgccgtaacaatatcaacaacagtaaataatatatatatgttgtggcgcacaacccgatcccaccatatgtcaatatcaatatcataattcacccttacttcaccatatcaatacacctattacggtgtgcaacccgatcctaccttacaatacaaattcacctgtattttgccatatcaatccacccttattatacctgctgcagcgtatagcccgatcccaccatatcagtactaaatgctcaaagcacagtcaaatcaatggttcaaactacaaaggctttacaatcagtaaatgccaaactgaaccaaaaggaaaacttgcacattatagaatctacacaagtaatactacacagctagaccaatccagcaatttacaattagaaggtgcaaacaagtcaatttaagcaaacaatagggattcgtgaaactatgaaaagaactaacattattaacagtagaaggtattgattaacatgtagcagttaagcatgtaaaacatttagaccatataacagttaagataggaaaggaaacaattaaggaaaaaaggtaaatcgggggaaaatagataattcagcggcgtataagcactcgtcacctcgcatatacgccgatcgcatgagaatcacataacatatagtctgagggttcttaattccctcaagtcactgttaaacataacacttacatcacttcgcggccaattcaaagctctaccggggcctttcccctagaatctgcctccaaaccactcgtatctagccacaactaactcaataacatcaaatattgctaaaggaattaattacaatgcataaaattagatttcccaaacttttccttaaaaagtcaaaaatcgactcaggcacgcttggtcaaaacccgatgtttggaccaaaatccatttacccattcacccccaagcctaattatgtaatttgtctcgaaatccgacctcaattcgaggtctaaaatctatttttttacaaaaatccataattctacctaaacccccaatttctaccatgaaaatcatagattttaggttgaaaactcatggaaattaatggctaattgaaagaaagtaggttagaatcacttatcaacaaaattggggaagaaaatatcttggaaaaatcacctctaggtcttctagttttgaaattttgaaataatggccaaacatcccataatgggactgtttaataactgggcgacattgttcatcgcgttcaagtgaatactgtcgtgtttgcgaagagtagggcctaagtggcctacgcgatcgctagtagttgtacgtgttcgcgaaggtctatccccccatgacctccgcgttcgcgagactgggctcgcgttcgcgtagagttacctcaactccactacccagcctagctaaatctacacgttcgcgtgagatgggtcgtgttcgcgttcgcgtagagcaaccccccaacgctccacgttcgcgaccatggtctcgcgttagcgtagtgaaaaatcctcctctaacccagatccctttcgcgatcgcgagaatgacttcgtggtcgcgaagcacaacacaccagaataccagctgcaacaaacacaccagattttctaagttcaaaacacctggtagcctatccgaaactcacccgagccctcagggctctaaaccaaacatgcacgtaagtcctaaaatatcatacgaacttacttgcacgatcaaattaccaaaataacacctaaaactacgaattgaacaccaaatcaaatgaaattttcaataaagctttaaaacaaccggacgttcgaatcacgtcaactaactccatttctcaccaaatttcatggacaagtcataaatattataatggacctgtactgggatccgaaaccaaaatacgaacccgttatcagcaagaccaaacgtcaattagttcttaaaaccattaaactttcaaactttcaattttctacaaaaattcatatctcgaattagggacatcgaaattcgattccgggcatacgcccaggtcctaaattacgatacggactcaccgggactgtcaaaatacagatccggatctgtttgatccaaacattgaccgaagtcaactcaatttaatttttaaggtaaaaattcttatttttatcaatttttaacatataagccttccgaaaaatactcgtaccgtgcatgcaaattgagaaaggctaaaataagatacttaaggattcagaacatgaaatttagttttaaaatatgagatgacctatcggatcatcacattcatgctccttgtagtaatttagatgttaatgttatgtcgtcagcaaaaataaggtgagatattatgggaccttgtctggatagttttatagggtgtcaattcctataatccacttctacgtatattttacgggggatcatctccatgcatatgatgaatatataaggtgggagggggtctctttgacggatgcctcttgagggagtgaagaaataagtgggtgctccattgattaggattgagatagaggattgtatcatgtgttttggaaaattaaagtagtggagagtgtcatgtataaagccccattccaatttatcaaaagctttatctatgtctagttttaggagaaaagatgattgaccacccttctttccttaaatggtgcatgatctcttgcacgatgattgcattatcagatgctttacgattttttagaaaaacggattgttcagggcttatgaggcttaatagtaagggtttaatatgattgattataattttggtgattagtttgtaaaaggtgttacaaaggcttatgggacgatactgagtaatagaggtaggatgttgtactttaggggtaaggcataggtacgttttgttcacctctggtggcatttgttgggtactaaagacctccatgcaaaaggaagttactgatgaacccactttgttccaataattttggtagaaatatgggtatagtccatctggcccatgagcctttaggggttggaaggatttgatggcattggttatttccagtgtagtgatgggggaagtcaaattgccagattcccgattattgatagtattgtaagtggctatgtaattaggatacgtgtagtgggtatggatagtagtaaataagttgtgataattcaattgagagatttatttcttgttagtttgttcatagtaaaacatgtttgaatttttataaaaataaataatttaaagttatactaatatggtaagaaatttataaacaatctcactaaaaggtagtagtaatttagtaagcagtaatatgcactaattagtaactagttaactgaaacatatactgtaataatatgtttagaataatgctgaccactgggtgacataattttataacacgataattagtgactattcaagttcataccggacaagttttgacacgcacgaaaaacataacgatttaagcattatcttgaaaatggattaatagcctgtttggcgtgcttctagaattagcttattttaaaaaatatttttggtgagtgataatttgtgtttggctaatcaatttgaaaaatatttttgagcaccaattagtgtttagccatgcctttaaaaatacttttaagtgtatttttttccaaagtacttttcaaaaaagtatttttgtggagaaactaacttttttacttttcaaaagtcacttttatttctactcaagcatttttccttctaaaaacatggccaaacatctaaattttgccaaatattttttttgaggaaaaaaatactttcaaaaaagcttagccaaacaggctataggtgaaatagagttataccaataatgtaaaaaaaattatattatggtaactctctttatatactttatacttaaataataataaaaaaagataaaaaacaaaataaaaaaggtgaggagtttagttggaagtgacatgtcacatgcgtttgatacatcattgaacggtgtcaccattttttcttttttcaaaaggtgcacctgaaaacccacttctccaagtaagccccccccaaaaaaaaacattcacttctcccagtcaactgaaagtcaatcaccctttttttctgccgccggtaagacactaccggtgcttccgccaccgccactgccgccgtaatggaaggcggcgactttgagaacggcactgagagtccaccaagtgctgcaacttcaccgatcactgagcagctttctgctctgtctctcaatggcgacatagattctcctgtttcagttcagaaaccagagaaagaccccagaaaaattgctagaaagtaagttctttgcccttgtgggtgcaaaattgctaaatgtatattttgaagattatttgtgtacatatatttttgttgtgtcatgggttgtgagattttatagaatcatacactattaactaaaaccttgtggtgattttaagtggtttgactaacttaaaaatctagagaaataggctgccttacagttcgtttttgcaagaggttgtttcagtagtttgaacccgcgaccttcgagtgacatggcggcatagatgaatatataattttatagattatttgtctatataaataattatttgtgggtacatatatagttgagctcattcgcagtttatgagaaatttatagaaatatatattaattaaaggtttttcaagtgtaatatcgttcaaaggcgaggtggatgatgttgaatactttgaccaactcaaaaagcgtaagcttgttaaataagagacacattaatttcctcaatgcgggcttgagtccttatccttcggtggattgaacacacgtaaattgtttttgatagcaagtgacaatgagatttaaagcatttgtcacgtgcagagttgtgtagcttccgagaagcgaatgaagggaagttttccagcaagcttacgcttactctcggcctctttgatcggtaggcaagcaggctaagccacttttttttttgttgttttggaatcaaacacgtgaaaattcttgtttataagggtgtattgacttgaattctatgttgctcgaactttccataatattgtcgcacccgtgtcggatcctccaaaaattcaacacgcatccgacaatatttgtgaagagtccgagcaacacaggtgggagccttggtgtagccggtaaagttgctgccatgtgacctggaggtcacgggttcaagccgcctgccctttttagtccgagcaacatagcttgagtataggaccttcgccgtatctcttactatttgtaaataatataatattattataatatttttattttttaattttatcttcatattttttattgtggtggtgccctaaccaacttacgcgcaccttgactagtctatgggataattgacatgcattgagtaactctacttactaagatttaggcagatggcaagaaatcacctagttttttttgcctccattgggatttgaacttgagacctcatgattctcctcccacttcattggccactaaaccactcctttaggtatatttttttcattatcgaggaaagtgtaattttacggaaaagtaaaaagctccgtgcaaaaggaagtaaaaaatagagaatctatataaatatgtgattatttaggaaagacgccaatcttctatgcaaataggaatctcattggtgtaccaaaaacacattaaaaaaagagaggctattcctcaaacgtcaaaacaagctttgtccctttaagagctctgctctttctctctccatggtacccagataagtgctaatagagcaacatcccacgctcggtgttagacccaactaaatagatcagccttcacattattgtatctcgcaccatcttaaagctctaccactgacgtaacgctacctagcaatgaagtaggatatgatccatgtcatctcctgaatctttgcacatgaaataccaattgacgtgtgattctcctcttcaatttttcggttctcaaaatcgctcctttagtcatcaaccaatcgaaaaagcacacatttcttggagtccttgggatccacactggaaaacgagggaaacccgacttttgcctagcaagcaacttacagtattagctctttacggatatctgactctaactattgaccactccctaactccgacctccagtaatcttggttgtctagcagtaaagtctttttatagagcatttcaagtagattttcaatatctgccatctttccatctcagatggttccttcttaatctaagatcccaaatacacttccttcatgccgtatccagagacttgttagactatcatctctagttggcatgaatctctatagatgtttggaaaaatgatcctcagcctgttgtccctcgactacctgtccccctaaaagtcctgtttctctcttaacttaagtgagatacaaccacttaatctttgttttcataatattccttcaaaaggccgcatcgaaaaggaacaatgatgtttttggttatccacctcctttccgtggtgtcatatttctgtgctaccaccttccaaattcatgctcctatgcagtgttgaagtacacatattctctatgcagatgacacgctgcttaagtgcaggcctgatgaagacctactgctgcatcggaaagctatccttctgacctttgaagctatagctgggttacatatttttttgacaagggctgggttaacaactatgggggcctagaagagtggtgaggatgctagcgttatgtggacggcaacgacagaatgtataagggaggcggcgatggaggtgttagggatctcgaagggctactctagcgggaaccgaggtgaccggtggtggaatgacatggtacagggtaaagtggaagcaaagaaagcggcataccttaagttagtggagagcaccgacgaggagcagaggaaagcgaacaaagaaagatataatgaagctaggaaggaggcaaaattagcggtcacggaggctaagactgctgcgtttgattggctgtatgagtaactgggggccaaaggtggggataagaagttatttcggctggccagagcgagatagagaaaggctcgtgacctggatcaagttaagtgcatcaaagacgaggaaggtcgtgtattgatggaagaggcccagatcaagcaaatatggcaatcatactttcataaattgttgaatgacgaggaggacaagagcatcgtgttagggggattggggcactccgagagtcaccgagattttaggtactgtagacgtattagggttgaggaagtcgtgggagcgatgcgtaagatgagtcggagcaaagcgaccggaccggacaagatcccagtagaattttggaggtacgtgggtagagcaggcttggagtggctgactggctgtttaatgttattctcaggatgaagaggatgtcggatgagtggaggtggagtacgatgagtccgttgtacaagaacaaaggtgatatccaaaattgtagcaactataggggtatcaagttaatgagacataccatgaaaatttgggagagggtggtgaagggagggtaaggagagcggtgtctatatcagagaaccagtttgggtttatgccgggtcgttctactacggaagctatccatcttattaggaggttggtggaacagtatcgagataggaagagggatttgcacatactatttattgacctagagaaggcgtatgacaaggtttctagggacgttcgttggagatgcctggagatgaaaggtgtaccagtagcttatattagggcgataaaagatatgtatgatggagctaagactcatgttaggatagtgagaggtgacttggagccttttccggtggttatggggttacaacaaggatctgcgctcatttcgttcttatttgccccggtgatggacacactgacacaccatattcaaggggaggtgccttggtgtatgctgttcgctgatatagttctgattgacgagacgcgaggcgacgttaacgagatactggaggaatggatgaaggccctttagtctaaaggtttcaagttgagtaggactaagacggaatatttggagtgtaagttcagcgatgtatcggggaagcggatgtggaagtgaggcttgactcacaagtcatccctaagagagagagagtttgaagttcctagggtttattatccagggagatggggagatcgacgtggatgtcacgcaccgtatcggggtggggtggatgaaattgaggttagcgtctggagtcttgtgtgacaagaatatactatcgaaacttataggtaagttctacagagcggtggttagacccgccacgttgtatggggctgagtgttggccagtcaagatttcacatacccagaagatgaaagtagcagaaatgaggatgttgagatggatgtgcgggcacactcggatggataagattaggaatgaggatattcggaagagagtgagcgtggctcccgtggatgacaagatgcgggaagcgaggctgaggtggttcgggcacgtgcggaggtgtgagcgcttggctctggcagttacgaggcgaggtagagggcggcctaagaagtactgaggcgaggtgatcaggcaggacataacgcaacttcaaatttccgaggacatggcgtttgataggaaggtgtggaggtcgagcattagggttgtaggttatggggtaggcgatcatttctcctcgctgcgccggctagtctgatagtgtttcgtctaggactgctagcggtttttgttgtgtttcacataatcttttttgttgtccgtcacattttggcctttccatttatttgttgtattttaagatggtgatactatttttctggtttctgttgtggttatggatctactatctctgagccgagggtctcccggaaacagcctctctatcccttcggggtaggggtaaagtctgcatacattttaacctctccagaccccactagtgggattcgactgggtggttgttgttgtttgttgttgttgaccatgttatacacaagccagcagatgtctttggctacaaaattggatgctttcctaccaaatatttggggtatacccttgggtgctaaaggaacggatgttaatatatggcaaagggtgattgaaactgtgaaaagaaattggcaccctggaagaactagtacatatcctttggaggatgattaacattaatacatgacgtgttggatgagattcctactcatatatgtctttatttgccatgcctcaaaagtggaaaggcaaatggacatggagaagcaatttcctgtggggaggatgtcaagacaacggaaatcaagttgggtacagtttcctgggataagagatctggagggttggactaaaaaattttagaacttcaaaacaaaagcctgctgatgaaatggcactggaggttcaatgatgacaaaaatgatttgtggatgaatcttgttattgttaaatataggaagaccgaagagtggtgcactgatagtgcaaaaggtgcatatgtagtttctatgtggaaatatactagaaggctgtgagatgattctttgataatgtttagtaaaagttggaaatagcaagagagttaaaatgtgggctgagaagtgctttggtgatactcctctatgtgagcaattacctgactgtatgcaattacccacaagaattgcactattgctgactgctattcttctggtttattgatattatattgagaaaaaatctaccttatcaaataaataaataaattgagaagagatctcaatgattgggagtttgagcatgtgaaggaactaatttaaaatttgaaactgtaaggattgattaacatatagcagattctttactctggaagggtaaagcattttctgtcagggcatgctacaaaactgttgctaaaaattggaggaagccatgtaaattagccatggaggataattcgagtgactgtcgcagccccatgttacctgcttcacctggactgcaaatcatggtgcctatctcactcatgaaaacctacaaaataggggctttactgtatgcagtattttgtgcaggagcggctgaatctgttgatcacttactgttccattgcattttcatcaagcagatttggtctatttttatttctgggaataaattgtcatgcagaagcacaatacaggccttaaatagctggtataatcagacactagtgaggggaagcagattctactggaatgcagtgtctagttgtatttttctggaacacacctggttagagagaaatgtttctctggaaaaaaagaattcatttctcatttagagtttaggtgttcaaatattctctatttgtggtgtaagaaagatgtaactgattttgcttcactaattgatctgatgtattccttcaatatttaggaggaaaagctcattccttctgtactctctcttcttgttactcattaataaaatctctacaacaacaacccagtatattcccacttagtggggtctggggagggtagtgtgtacgcaggccttacccctaccctggggtagagaggctgtttccaaatagacccccggcatccttccctccaagaacttaccaccttgctcttggggagactcgaactcacaacctcttggttggaagtggaggttgcttaccatcagagcaacccctcttgtccttaataaaatctctacattttcttataaaaaaaaagaaccttttatttgctaaggtgttgttacaataaaacctttgaactttcaaacagctgaagttctttataattatcaaacgctttcagaatttaactacactatttaactgaataatgatgttttagtaatggcaaaaacaacgagataggtgctttgatgattttatagcaaggctttgctgattttatttcttatcaggtatcagatggatctttgtaaaaaagcattagaggagaatgttgttgtatatctgggaacgggctgtgggaaaactcatattgccgtcttgcttatctatgagatgggacagctgataaggaaaccccagaagagcatttgcgtgtttcttgctcccactgtggcgttggtgcaacaggtttccttatttttgaatttttttttggtcaaccttttaaattttatgatataaaagatgaaaactattttacatgaaatcagatggctgaaatatcaaatatcttccttgctattgaggcggtatatgagagctttaagcatgtttccgccagctattttacacaattccctttttgtcttttctcctttcaattgagtctcttttttgataaaacaataattttgttgattcatggggacactaaaatagaagcgacaattttattctcttatgtcaccggcccaatcaaagcccttcttttgggtgaccactttgatgccgttgcgctttctgctgcttcaaaggcaccacatatctttccggctactgttctgatggcattgctccttttgtccagtggctgttccagctttgtattcatcatattattgagttttcaacttagctctttacttatcgtatttgtccatagaaatttttattgattttgtggctagtatgtacgcaatattctacgcatatatggcgaaagaaattttattaaataattgaagactaagtgtttcagtctagacaaaataattaagtatatagagtttttcgattttgctgaaaattggtcaatggttagcatgaaaaggcctatagacttacatctcgtagagaacaagaaagcgctcttcaattcagaatgtcatttgatatgtttaactaattctctcttgtctcttcgttagaatcttattacttcgattaaacccattctgttttctgagtgtactcttacaacttggggcagggtgggaattaaaaagtttgtccgaaacatgataaagcatgggcataaaagttaattttctttttcatgtgcttttgaaattggaaaagcaagccaaggtcatagaagactctattgacttcaaagttgggacatactgcgggaaatccaagcatttgaagagccacgaagactgggaaaaagaaatggaacagtatgaggtgagtctgcatgacctttttttgttggtgatggacatcatctcctgaatgttatttgtttgagacagaaccatgggcattcttggtatgcaaacttccgattacagattttaatacaaatattctttgaaacttgaataactgatgttaggggtaaaaaaaaacttgaattactgatttcaactaaattaatgtattcttaccttgtccaattacaattgaaatcttacataattttctgagccttttgaagatagtggatgatgcttatgtgcttgagaaagggagattctgagaagagcgacaagtacgggttggaactcctgtgttacatgggttattttgagggtatcacggatactctgaaagaagtagaaagtctctacacattttggattttttttgtttacagcacttagcttagtgtcattgtgcatctatacatgacatagatggtttgattgaggttgttggatcctttcacttttgggtggggacctacagttttttgttctctctttcgcaaatgttgacgtcaacacttgcttgttttgttttattcatttaaattatcctttatagtcactaagaataaaaacaggaatcaacatggtttctgttaactatatgtagtagattgactgaaccatgttatgtgttatatactgtggtcttctcatcttaggtccttttttgggctgtgcatgagtgtggatattagtgatggctgactgttatactgtaacttttttttgttgacactatgtgaatgttttgaccaacaaatttgcactttgtttgttcaggtccttgttatgacccctcaaatactattacataatctttcccactgctatatcaggattgaatttattgctcttctgatctttgatgagtgccactatgcacaagttgagagtgatcatccttatgctgagattatgaaggtaattagctgagctggtgttatacgctgcatgctttgttttgtatgtagtagccttcttattcatgtatattatttttaacagatattttacaaacctgatgtagtaaaactgccccgtatacttggcatgactgcatctccaatatcaggaaaaggtaatatattgtgctgtaatcttcaggtgtattgttgcttttttgggatggattcacccagtaacaatatgttgtaactttctccaactagctggatccaatcagatagctcatcagtaatttcatatgtcaccttcgtgttttaaacttgacaattttttttataatttaaataaatttattaatctagggaaaattgacaaatagtacttcacagtgcttgtaagatgctaagggattgtcatggaatgtaagtgttatgagattcaacacagtaattttcggcagaatgatataaagtcacatgggattgacatggaactcacacttgaggttacttgataatttttggcaaaatcatcttaagtatagatgtcatccagaaatcaccatctgtcccagatttgtgtctctttcttgggttttcaggggtcatttctctggaccacttcttacttgttataagcgaaaacgtttttccatcttgtgagatctgtaagtctttaactattacttcgaaatgatgaatttatccccttctcctatccatgtaaaattggtcaacctgactgaaaactgactggggaagcagaggaattgttgttatgatattttctttgtctatgttaatgatcaaatatcactggctgtctaatatagccttgtatgagtgggaaaatgtccctgtgtactctttttacctcctttttctaatagtgtctctcatctgcatatgtgtaattttacctccttatgcatgacagtatgtgtatctttgttacatagttgattcttaacgtcatgacctctatgaattctatttagaaatgggatcaacagaagttacaggagtattatcagaaaataaatctgaaaatgccttcaagttttctgattttttgacttataaaaaaaactcatctgaacagcctcgctgccttcttgaaggtaggtaaggtgtacatactaccctccccacatcccacttgtaggattacactgggtctgttgttgtataaaaaaaactcatctgatctctggggtctgtgcctttgtggaaagaaatctgcttgccatgccatgcattggtcaattgctgtgacagattgatagttcttgctatctgatctttgctttcaatactaatcattcttccaggcgaaactggtgtatctttcctttaagctacatttgtttttggatgctcctcaactctcagatattatgttgtcttcttactattgcaggtgctactgttgaaggccttgagaccttacttcgctctaaggtgctactattgtacacaagctttttttccatcatcaataaattatttttcttttgaaaaaaaagaaaaagaaaaataagaaaaagaaaaaggatgaccataaaagattacagtttcctcacatcattattatgggaacaataaagtccaaaaacagttacttgtgaaggatgggtttctgtcataggtttattagttcttggagttgttaagaactgtcataagattgcgactccaatttccaagctatcattttgtatcctgggtttcttgctgtgggtgtttcattgttaggtagcaaatgactagcttggcggcattttacctgcctgggctgagctgggctgctaatcttggttcatacccatactgttgtagaagtgtgaggtagagggaaataggaccattaacatttctatatttaatttctgtctatgtatgatcacaggtgtattctgttgaagacaaggatgaactggaacagtttgttgcatcacctaaggtgaatgtatactactatggtcctggcgccgcgtgtcttactaaagcttactctcagaagcttgaagagataaaacaccaggtattatctggtcagtaatttagatggctattttctaattagtcttcgaataaatactttctggtgttttcatgatttgacagtgtgtgatggtgcttcacaagaaagcagttgatcatagcacactcaggaacacaaaaaagatgctcaagagattgcatggacatctaattttttctctggaaaacctaggtgtctttggggcattacaggttttgttgcgcttcttcttgtttgtcccctaatccgttttcacaagtcctgatctttcattactgttagattatcggatattttattgctcacatttaatattttacttgtgttcccatttatattagaaacaattatcaggggaagacctgcccccaccttgccgccacctccaacaagcaaacattataaatggtctttttacttgagtatcatttagaatttcattaaaacagagaaacttgcgtactatatggaacccatgatttaggctcaagacctgaccagattcctagctctcttctaaaacaaagtgggcagacgggtttgaaaatcttgtgcagtttaattgactctcttgacctggcatagagtttttggcagttaggtgtagaacttccgttctctttttgtttttactttcatactgctgctgaagtagtatctacggacatctttaggcacttcagattttattttttgtgtgtatttttaaaaaacttcagctggagagtccacatgcattaccctactttagacattatttcagtagagtatgcactccagaatcttgttggttccctagttatgcttagcgtaatttaagcactattaattcacagggaagacatcacgatctatttttatttattagttttaagacttacagtcggaaattcaattctaagttgcacccttgttttggtgaaggctagttgcatcctcttaaagggggatcactatgagcgccatcaaatggtggaagctgatgttaatgctagtgatgactctttgtgtgacagatatctgagtcaggtcgccacaatgtttacttctggttgtgctaaaggtatgttttattctattgtggattcctgttctatagctatttgtattctttccatcattacaagtattcctatgaattctagcttgaacaagaattctattcttactggcctgatcatggattacttggttcaagaaatgaaattagcaagctctaaccatatcttactgacttgttaatagagatcaagacatttgaattggacgtctaaatcaatttatagcattccactgattaagcagggtgttcctttgccaaagctaaatttgggttcttattccttcgtgttcttacaacacatttcaccaatgacaaagaagcatacagggaggagaactagtacacctaaaacaagtaaatgaaattttaatccagatatacatggatcttcttaacgctgtatggggactgtagaaatggaaaggggacactgtaggaggaattgttgaggttagatgaaacggcaaatacacataggtgattgccgctgattttgccaaggaaggaatagtgtttacaccattggaggtaaagttccagaggaattctctggctttatcttcaactgtggtcatcgacatgccatcacttgggacaagattcacttggaggaatagaaggacgatttccaggattgatcttggtatcgactgaatgggagggaattctttttgtcactctacccatttggcatagggatcagtgttggatcattgtcctattctcttaaagaactggtggttagaaaaggttatctccatcaaatctgagaacatatggcttcagcatgaaggaagggatggttaagacagtgggatagttaagtacacgagaggaaaccacattttaggtagccaagaagctcagaagtagaagaaacctcaagaaatgaaacatggagatatctggcagagtagaaacaaacactacagagttattacaggagtacatacattggagtaggtcgaaggtgatggtttatgcgagaaataagagaatggagggaagcgtgaaatcaaatatcttacgcctgaatccttttatgtgcatcatttactatcaacacatatagctatattcacccacttgtggtcactaatgctatattacattgattataactatatttgcatagcaaattttacacttaactgtcttatagtttgcttaatgatgttgggattttgagggaggaagttaaagataggcttgggcgacactactaccaaatttcgttctgctgaataatggtgataacaagaccaaattcttttcacctgccaatgcacgtagtggacatggaagatgtaatctcattgagaagatgcaaatagatgactagatactccaaggaaaatgaaaataaagagtagattttgcaggcttatctatccgttgttatatgaggaataggatgagcaagggtcaggatttgatggtagtttttttgatgaagtaataaatttcattaaagaaagggcacacatgtcaggatttggtggtattagtttctagaaattcgacagcagggaaatagaggagctagagaaaccatttgaagctgaaagagtggaggtaggggatgaagagttgtttaggagacaaggcactggatttgatggtttcactcttgcctcttttaaacactaatggaaaatggtaaacgacactgtttatagtgcgtgcgtcatttttatcggtttggtccctttaagagatatttaaattctttgttctttgttctatttccgaagaggaaagcaacagataataaataaggactttggaccaatcaatttggctggaaagtgaacaatggtaaatgtgttgttaatgagattgagatttgtggtgaatagcattgtatccattgcacaagattctttcgctggttgaagggtatggatgttcgtgtgatctcaaacgaactgacaaattctgtattgatctgtggaaagttgggaattctggttaaactagacatgagaaggcatgtgaccgtgcttgtgggactacgctgggtatgttgttgttgttgttgttgttgtattagtcgttatccagaatggtttcttggggaaagttgtagaaagtggagaactagtgcatacccattgcatgtgtgtgtgtgtgtgtgtgtgttgttgggagcaaaaagttattcggcaggaagacctttgtgccgttgttattattgtttataatagagagctaatgttataatctgtaggtagtgaattcttgagtgtaatttcgggttgttggaggagagcatgacatggaatatgtagcacatctattgtttgcagatggcactttaatctttttgatccaaacggagacatccatttcttatatagaacacttgggggttcaaagacaattgggctacacttcaatatgataaatttggaattaattccattagaagaggtgaaaaaaatgctaagagactggagaggataggaagatgcgatatttatcaaaaggaggaagagtcttcttaatagagtttattcccccacataatttgtaactttgttcacaatcgctattgcaataaataaaagaatggagaagctaatgcgagaagttttatgcatggaaggaagttatcatttgatgttcaatttaccaatcaagcaagaatataagctgccttcgttttcagtgtgatgttagcatttcaaagaaaaaaggctggacaactagatgggaatcatgttgtgactgaacaagtatgatgaatcaagtttcagtctgacagtaatctttccacgcctctttttacctctctgtataaagttagacttgatttcgaattttatttgctttgctttagggtcttttaatgaagactaagggcatatgataaaagtcaattcctgcaaatataggctcaaataatgatctgttccaacagaaatatcaaagaacctttgatctgtcttgtgctgtggcttgtgtatattcttgttgttttcctctgtatattactagtctaacatatgctttatcatgcagatggtatgaatcctgatttgacacttgtggaagttctgaaggagccttatttctccaaaaagcttttacgtctaattggaatcctttccaacttcgggttagtcattatgtagctaaatgaataatttcctgttcctgttctctgacttattaaggatttataagtagctggaaacaaaatagtcaattccactttgaggggtgttcttctgggtcattgtatccgattataataaaatcaaccgaaggagtattggcagtgaacttacttgataacctattttcaataaattcagtttatgcatgtaaatttactattagaaaactggaagcatagacaatatttcctttaacagttggtgtgttcttttcttatgaaaactttatattcttttgattgccattttcaagaatagtcccaaaatttccattttgcttcaatgcaactactcaaagagtttaggggtgagggagatgcatcaatgcaaatgatctgtaacaatcaggctacagtgttaaaaatggagcatgtaaaagtaggttgtcatttcgttccacaaatgattaaaccgggtgaattgcggcatgttttgtttgctcggatgattaattaatagatattcttacaaagccactcagcaactctggaattgagtatatttttagcaagttaggaatatctaatatgtatgatccaacttgagagggagcgttaagaatgtcaatattacgaaattgttaatatgacaagaatacgtaccttgtaagtataggaaaagtttagaatccatataattgggtaattaacaataaggattattttcttttccttttttggattacttgtagacattattcaaatcccagcatgtttattcctcaaataagcactatctttttcgacttcttccagtgttgggttccaaaattatgtagtcgatgcaccaaatgtccaggcctggctgcacggaggtgttagagtgccccattttggttgtgaaataggttgtagtctccttttatggtttcaagcaaccctcacatcaagagctgacttttgggttgagttggacctaatgtccattttcttaacgtaaagtttctttttcactttctcaatgacctcgttccatatctctgtcgatttttgcaccatacccaatggcattcccaataagtagaaataatccctaactgcagcctaagatatcagccaagtgctctggtctcaaaagattctgctcagttatttgtcctatttcacctgatgacatctttccttgccacttggttttttccatgtcctttgtttgtcctatgattagtggaaaaagggctataggaggttaaattatgttataactggggattctagaaagcggcacaaagaatgacacaggagatctataacggtttaaatgtgaccttctctgtacattttacattatgctataactgagatgttgcggtgatttgcagaagataatgaatttgaagttaataagatcattagaacaagagaaacataatgtattagtctgaaccaccctaaatgtactgttaatcatccagcagctataagcttaatgctgtttgttccaaataagactctttgaatagaactataaccgattaaacctgacgtattagtttatacatttgtacatgggaattagaaatttcaatttctcagtttatcaaaatatgtttggagcagaagatcattctacttgtactgatttttgttcgtcatgattttaatcttaacattttattctttgcccttacagagcacagtatagggcctatttttaaagagcaccacaatggggcctctatggagaattatggaattaatgccttgaatatgagtgtttatatttatggacctaaccagctagttagcccctcagtttgctctcagttactctaaaagtgcacccgaatagggccctgatggagaaacatgaaatgaatgcattgactacaattgttcatatttttggattagcctctgtgcatttgctgccaattacggtggtaccatctgaaattaaatgttcaacaaccatctgcttttgatcacatttcttcccctaaaagtttattcttcaacgtgatcttttgccttcataatgtacctttacccgttagaaataataagaatataatttgaagtcattaatgtatgtttcttgctttcataccatacctttacccaatacgtataataaatgcttcattttgtttgatgtcatggatgtatatttctgcttcttagtgagttcagtcgctaaggaattcgagagtcaacttcataatctgcattttggtatctccctctctttattttcagggtacaaccagatatgaagtgcataattttcgtcaataggatcgtgactgcgagatctctttcttatatgcttcagcatctgaaagttttatcttcttggaaatgcgggtttcttgtaggagtgcactcaggattgaagagtatgtcacgaaaaaatacaaatatcattctcaataagttccgttctggtgaggtaatggttctttgttagattctttctctgctcgcttgattctcttagacagaggacctctgggagggcaactccaagtttatgtttgattctccatctgatagttcgtatgaaattgatcgcatatccattgcttaatttgacagctcaacttattggttgccaccaaagttggcgaggaaggacttgatatccagacatgctgccttgttatacgatttgatcttcctgaaactgttgccagttttatacagtctagagggcgtgcccgcatgccaaagtctgaatatgcatttttggttgataggtatctccaaattttatgctttctgtaccttattacctgtcaagttcatcagagcaatatctctgacacaaacttcttggcagtgataaccaaagggagcttaatttgattgagcatttttcaagaaatgaagctcggatgaatgatgagatttcttccaagaaatcttgcacgacagttatagattttcaagaaaatatatacaaagtagatatgactggtgcaaccatcagttctgcatcaagcatttcgttgcttcaccattattgttccaagcttccacgtgacgagtatgatcttgtactgaatttcacttgatatttctgtttttctttttctaattaaagtttattctgttctgtatcacttatgtgtggattttgctttcggaactcatatcttcatgggttaacatgatttcttttctgttcgttttgcttcttttgtaggtttttctgccccaagccgcagttcttctactttgatgacatagatggaactatttgcaaattagttttgccttcaaacgctccaatgcatcaaattgtgagtgctcctcaatcttctatagaagcagctaagaaagatgcctgtttaagagcctgcaagagcttgcatgaacttggagcactcactgattatcttttgccggatcaagcagatgaagacctgattcacgttttttctgactcagaaagctcagatggttagacaaaattattttgataaaagcattttcaatatgctttatgttattttggtgcttgttgtcattattgtaatgttgatgtcataggcgaagacgcacgagaagagctccatgagatgattgttcccgctgcctttaaagaaccatggactgagacagagagtcctgtttgccttaactcgtactacatcaacttttcaccttgtccaattgatagggtttataaaaagtttggcctatttttgaaagcacctcttccccaagaggctgagagaatgaaacttaatctcaacttggctcgaggtagatctgtagagacagagcttattccatcaggagctactaattttgagaacaatgaggttaactatttgaccctcttctagtataaacagaaaggctctcttcaatattttgactactttctcttagcaggtacaactagcagagaagttccagagaatgttcctcaagatcattcttgaccgctctgagttcatttcagaattcgtatctttggaaaaacaagattatgttgactcagcttcaaaatcttacctacttcttccactcaatttatgtggacacaataaaatttccgtggactgggagcttgtcagaagatgcttgtcatcaccaatttttgggacttcagtatatgcaggcaatagtgagatatccaaatttgatgagcagttgcaacttgccaatggttccaaaagcgtacatgatgttgccaatagtttggtatatgttccatgcaaggatacatttttcttcatatctgatgttgtcaaagaaagcaatgcatacagcatatataaagattcaaaaaaccatgtcaaacactattatgatacgtaagtaaatgactttatttttgttctttgcttaccagcttggtttcctgttcttttgttgattctacctgcattcgcatgcgcatatgtttttttctgtctactgtaaaccagaataggagttcaagtattaataaaatggaaatgagcccttggtctggtgtggcaaaaaggggagaaaatttacaaattagatgcaaagagtatggcagttagaagagtttttgatgaggtcaattagcatttaaccgcttgctgctggagaaccaattaaacaatttcctcacatgtatttggaagttctttttttcttctggaattttttttattatggtcagtcactatatgtctattttatactttttgagcctcacttgtgggattacaatgggtttgttgttgttgttgttgtgagccttgagtactaacagaatttcttgatgtaacttcctttaccctgtttgatagggtaaataatttaattaacgtgggataactcccgtatactagcttgtttgggaaacttcccgttattgatgtatctttacttgtttaggaagcatacaacctgctttagtttgggaaaggagacacttcttttcattgcaattgtgtaactctgcttgttttcttggttaccctaactggccgctaaagggactgtcatgctcttcttgtttagagctacatttgttttatatcttctgatgttgcttattgaggacatcttgtcctcttcctttgcgcttagcttaataatacttttctcataaagaaaacatatatcttagttgaagatctgatcatttttacaaatatgtatcatgtatattctaatgggtgagagatttctcttacagttttggtatccatctttcgtaccctgagcaaccactaataaaagcaaaacaactcttctgtttggacaacttgctacgaaagaaaggctattcaggtaattgtttattattcaagctgctaagttgagctgtttctattgcaaaactcataaagacttgtgatgcaagggagggggattggaagaaagggttttactccgatacatgttgaagcatattgtttaaactaatgggcatctgctttgaaagcaaagaaaccacaaactctactatagaactcaaagtcagaggcaaaatcattgacagtagactattgagtgctgataaagttctatctagttctatttatttgtgagagagcatccaaaaaccctagttcttgtgagaatgcaggggagaaaaatctactattgatatttgttttaattataatacaatgagccctatttatacaatacatatcatactcctattctatgtgggactaggactaattcatattatttacataactatttaacaatcccctcaagccggtgcatacaaatcttatgtaccgagcttgttacatatgtaactaatacgaggaccagtaagggacttggtgaaaatatctgcaagctgatcatttgacttcacaaactttgtagcaatatctcttgagagtatcttttctctgacgaactgacaatcaatctcaatgtgtttaattctctcatggaacaccggatttgatgcaatatgaaggacagcttgattatcacacacaagttccatcttgctgatcttaccaaatttcaactcattaagcaaatgtttgatccaagctagctcacatgttgccacaaccattgctcgatattctgcttctgcactagaccgagcaaccacattctgtttcttgctcttccaatacaccaaatttcctcctactaaaacacaatatccagacgttcatcctgcccagccagcttctaattacgcaacaatttgctcatggcctcgatcttcaaacaataatctttgcgtggaactgattgtatatatcgaagaatgcagacaactgcattccaatgactatcagagtgagaatccaggaactgacttaccacatttacaggaaaggaaatgtcagatctagtcaatgtgaggtaatttaatttaccaactagccacctatatcttgtaggatcgttaagcggctccccctgtcctggtagaagcttagaattcagatctataggagtgtcaacatgcctacaacttgtcattcttgtctccacaagaatgtctaaggcatacttccgttgtgagattacaatacttgagctagactgagcagcctcaatacctagaaaatattttaatctgcctagatccttagtctgaaagtgcttaaagagatgttgcttcaacttagtaataccatcctgatcattgccggtaatgacaatatcatcaacataaaccgctagataaatactgatatttgaagcagaatgccgataaaacatagagtgattagcttgactacgagtacttaccaaaccaagctcaaggagactgctttagaccatagagggactggcgcaaccgacatacaaggccactagactccccctgagcagcaaaaccaggtggttgcttcatataaacctcatcctcaaagtcaccgtgaagaaaaacattcttaatgtccaactgatagataggccaatggcgaacaacagtcatggatagaaaaagacgaactgatgctattttagccacgagagagaaagtatcactgtaatctagctcaaatatctgagtatatcctttggaaacaagacgagccttaagtcgatcaacctggccgtctggaccaactttgacggcatatacccaacgacaaccaagagtcgatttacccgaagggagaggaacaagctcccaagtaccactcatatgtaaagcagacatctcgtcaatcatggcctattgccaccctggatgagatagtgcttcaactgtagacttagtgatagaaacagaggacaaacatgatacaaatgcacaatggggtgatgacagacgatggtaacttaaaccgacataatggggattaggattaagtgtggaccgtatacctttccgtcgtgcaatcagttgactaagaggaaacaaatccgcagtattagcagggtcaggtgcaggacgtaaatcagctgggcctgatgctcggcgcggacgacgatgataagtcaggagtagtggagttgtagaaggttgaactggactaggtggtgaaactggagctataggtggttgaagtgcagctggagttgtaggtggtggagctggagttatagaggaagatggatgggagatagtgactgaatctccaaaagatggaattggtagcacctcaaaaatatctaagtgattacttggaccttgtgaagtatgattgggtttcaaaaaaggtacatcaacagacataaggtactgctggaggtcagtagaatcgatatcccttttgtgttctcgagtaacccaaagatacgcacttaagagcacagggagctaacttatcttttcttggagtaaggttatgaacaaaacacgtactcccaaagacacggggtggaagagagaacaaaggtaagtggggaaacaggacagagaatggaacttgattctggatggttgaagatggcacacgattaataaaatagcatgatctaagaactgtatcaccccaaaagcgcaacgaaacatgagattgtatgagtagggtacgagcatgtctattctttctttcagctatcccattttgttgggatgtgtacgaacaagatgtttgatgaataatcccatgagagttcataaacttcttaaatggggaagacaaatactctagggtattatcactacgaaatgtgcagatagaaaccccaaattgattttgaacttcagcgtggaaggtctgaaaaatagaaaacaactcagatcgatttttcatgaaaaatattcaggtgcatctggaataatcatcaatgaaactgataaagtagcggaatcccaaggtagaactgacctgactaggaccccaaacatctgaatgaactaaactaaaaggtgactctgcttgattatcaagacgccgagggaaatgagagcgggtatgtttaccgaattgacatgactcactctagagtggacaagtgagataaaccgggtaccatttgctgaagtttgacaaactgggatgtcccaaccgtttatgtaataaatatgttgaatcagtaaattgtgctttaacttctttcggtgaaggcaataggaatagtggtagaagataataaaatcgccacctagtatattacagtgagaagtccagttgcatagaatttttcttgggtgaataatgctgaggcctgttttctggctcaaaacttatagaaacccagggatttgacaggagtatggataatggaggggagatacatggttttaatggttattacttattgaaaaaaaaaagatatttggttttagaggatatctgcactggtgtaatgtgccgagcagctccaaaggtgaagggtctatattttcttctaaaacttcagtaagtcatttgggactagcttcaccaagacatgacatgagaaaactataagctttctttatttaacttgccagattttcagagtgtcagcttgttattccctcctgaaaatgagtttaatttctattatgcctattattctgaaaaacacagtaacttaagtggtgtattatttttcaattaatgactttcctctggccctctacacagagttgcgtgataaagaggagcactttgttgaactacctgctgaaatttgccagctcaagataattgggttttccaaagatattgggagctcattatcactgttgccatcgatcatgcatcgtcttgaaagcctacttgtcgctattgaactaaagggctgcctatctgcttctttccctgagggaagggaagtaacaatagatcatgtaaggaattctccgatcctcagtagctatgttgcgcggactctccaaaatgttgccgcacccgtgtcggatcctccaaaaatgcactactcttggagaatttgacccacacccgacgacactttgggagagtccgagcaacatagcttagtagtatttctcaatcaaacacatctgttttacatttatcacttgttcatagcatatgaatcatgtcagatacagtacctagtttactattaactgcctgaaactggaccaggttcttgaagcacttactacagagaaatgcaatgaacctttttctcttgaaaggcttgaagtgcttggtgatgcatttctcaagtttgcagttgggaggcatgtctttcttacatacgatgcctttgacgaaggacagttaactagaaggcgctcaaacattgtgaacaattcctatctgtacacgatagcagtcagaaacaatttacaggtacatggtgttgctgaaaactctaaatgctagatatggacagactcctacagtgatatgacagcacatcttttatgttcagataagagaatgcttgctttttcttgtttcaaaacatggcttttgaatttttgcaaattttacattatttcattgtgagaaagtgcaaacactgccattgtttgcacaaactcagatgcacgtgtggctctctcatctcacatggccccaaaaacaaataaaagagcagaaatagaatatatcttgttgaacttatcaggaaaaaaagttcatctgtgttgattaatatgaggcctcgaataatttgtcattgtatcaaataactttaatattatctccactaaactaatataataaccaagttaaatttactaataaaactttatacctagtcgataaccgtaaaatagaatgggacgaagggagtaataaatatctgatacattcatgagtaggtattggacgaagggagtaattatttgatacaatgacaatgaattcaaggtattgaaaatgtgaaattttgtataaacatggtatgggctaaacatttcgagatattaaatagaaagactaccatataaattgagattaagagagctataagatttgatgatctcctatccaaccaagtaaagaggggatttcctgtgtgtctggaacagtaaacatttttttaaaatggaacagtaaacttttttctatgcatacatttaacacgttaaagatagtgatatttgattagctgttaatgaatgcttttttgtggcatcattggaaaaattagtcctactaagtttttttgatgttgccttgatgcaccttaatattcagtcatcgctgacattttctttcctagtatgaggaaatgttttactcttgactgtcacgagtatgctgttcgctgcacaaatatctgaatgttgtcaaaagcaagccataaagataaaatgaacatactcaccatgctaaaatgacttcctcaatttctaataacctatgttgttggattctcgagttggacagttctgcatttttctttttgtgtgataaataagacagctagcattctgtatctcaataattgaacataaatatgaatataagatgaaactgcatatctctaatgaattttagaaatcagaggcaattgtggtttcctatgaggggaggaggtcatagaatatacattaatgtgactgtatgtgtgtaaatatgtttgattacgtgtttaattttttttttttttgtgagctttctccttttaagatgtttatactcttaagtcaattgatttgattgaatctctaagtttttctttgtgtcaggcatttattcgtgatcaatcatttgatcccaaccacttctatgcagtaggccgtccgtgccctgtaatctgtgacaaacaaacagaaaaaaacatacacggtcaatgtggtagtgtaacagatggtgcaaagactgaagttaggtgtagcaaatgtcatcagtggttgcgtaagaaaacaattgctgatatagttgaagcccttgttggcgcttttatagttgacagtggcttcaaagcagcaattgcatttcttaaatggattggtatctatacagatttcgaagaatcacaagtaaagagtatttgcgctgcaagcaaagtctttatgccacttgctgatgaaattgatattccggccattgaaaatttattaggttatccattcgtccataagggtttgctcattcaggccttcatccatccatcttataacaaccacggaggaggttgctatcaggtatggaatttctctttttcaataacctacttttctgaaactctctggttgttgtgtaggcgatttttagtactttgcagcaacttctttcttagaacatttgatttcctttagcagagactggagtttcttggagatgctgtcctcgattatttgatcacatcctatctatactctgtgtacccaaaactgaagcctggccagttgactgatctgagatcagtttctgtaaacaacaacaccttcgcagttgttgcagtacatcaaagcttccacagccatatactctgtgattatagtagtggcctccgtgagtcaattactagatatgtcaatttcattggaagaccagattctatgaaaaggttggctgaagagccatcttgtccaaaggtattcactatttacatgatgaaatcttcgatattgatggattcatggagtttcttatacagagtttataatagtttgtgtttacacttattgtagtatagagtatttgcccttcatcaaataaacgtaagaatttacattgcaaagttctcccataaaagaattcaggtataagacacttgggtcttcaaatcatttgatattattgctagatagcagattgattatatatttaggcaaaacgacattgtctccacaaataagggtaacgctgtactttgtaaagtgatgtgaaaggtttcaggtgtaaggcgtatgtctctgtcattctccttttctcttatttttttttttttttttcaaattggtaacatttttgtattagatataaacatgagtacactaattcagtgtagtcatccataattacaaaaggaagtacaaagagagacagactaagtctctataaagcttgtgagaaggcacgggagaaaatatgatatattgatattgtgtgtgatgcaatacaagaggtgctatttatagctactctatacaaaggggatactactcttattccaatgtgggacaattacatanaattcagtgtagtcatccataattacaaaaggaagtacaaagagagacagactaagtctctataaagcttgtgagaaggcacgggagaaaatatgatatattgatattgtgtgtgatgcaatacaagaggt

SEQ ID NO: 66 - DNA, 127560 bp, Nicotiana tabacum
  misc_feature (9391)..(9445): n is a, c, g, or t
  misc_feature (20735)..(21018): n is a, c, g, or t
  misc_feature (38289)..(38304): n is a, c, g, or t
  misc_feature (44066)..(44204): n is a, c, g, or t
  misc_feature (56787)..(57063): n is a, c, g, or t
  misc_feature (57340)..(57340): n is a, c, g, or t
  misc_feature (58066)..(59511): n is a, c, g, or t
  misc_feature (70482)..(70482): n is a, c, g, or t
  misc_feature (72737)..(72737): n is a, c, g, or t
  misc_feature (86876)..(87368): n is a, c, g, or t
  misc_feature (99516)..(99516): n is a, c, g, or t
  misc_feature (105463)..(105463): n is a, c, g, or t
  misc_feature (111794)..(111912): n is a, c, g, or t
  misc_feature (121063)..(121063): n is a, c, g, or t
  misc_feature (126389)..(126493): n is a, c, g, or t
cgttaacttatattttgcagaattaatatttacaaatacaggaatattgacgggaataacagtgttctccgaagtactattgacagagatcctcctttgtctgcctgtaaaatcactcgtccaatgcaaatgcatataaagaaaatggcgttctttaatctctagtcctcactttacacgcctcaaaatctcccgtttcagtcctgcaagtggaatcatcttgtaccattcatggtttacatgtaacccgttttacaaatttgtgccttttttaggcctagaaaatccaattccaccaccattccataaattcccttttgcactacatggacttttgctctgttgcaccattcattattctttcacgtttaatcaggactacttcatattcaacccgactactcagcaattcatcacacttcctattatacttatggggatgagttttagcctttgatccttagaaatcgccttgttataaaattatctgcctccgaatttctaaattggaacctgagatcgctcagattgagatttactcatccgaatcaaagaaatggagagtctcaggtccatatatatttgcctatacgccatgatataagttacactactggaatgaagctatttatttctcatataacgaatgcttcaatgtggaacaagaaatatttgaaacaattctcatgcctaattgcaggaatggacaaggccgggatgttgaggatcataagattgtctattttggggaatcttatggccatttgcacttaatacaagtgaatcgccagaaactaactctgtacaatgtttacgagatgaaaaataatggtactaaatcctcttttcgactctcgctctttcaacgtgagtgttgctttgtgcaggaagttcttttagtaggcgtttgcatgattggaattggaaaaaaagaaggaaaaaaaaaaagagaatttgaagttgaataacattattcgaaattagtttggacatgaatttcacttgaaaaaaacaaagttgtatgtgtgtaaaaagcattactcttattccactgtttttactcttttcaaaaaaagtttttaatatttcaccaatattgcaaagaaactcaatatctgcagataatgatgtccaaaccattacttaatagaagtatttgcagattgtgaacttcaatatttacaaataatgaaatatattacgaaactgatcttccttttcaggaaaacaaaaggcaggataatttgatataagcatgaataatgatgcgaggctattttatggtaagtgaagttgtagatgaaaagatcactttctacaatattaacaagatgaaaagtgatggcacatagtgttttttaaaagaagtaactcaaatagtcgtccaccctatcacttaaattaaaaatagccggtgaagatataatatatgcataattcatgtattacatgtgtataattatgtatagtcaatgtatgttctatgtatatagctagaaaacgtaaacaattaatatggttatttatatgtgtaaagatccctttttaaaataaaaaattttacttcctatagcaaagttttacaccttatttattataaataaatacccttttaaaatattattttctatagctaccttttagtttttatagcaaaatatgtatttatggccacttcctaccgttaagccattaaatatactgtctaatatttttctctccttctttcagttttttctctcccaaaatcaccgtatagtatcaaagttctctctccactatctctctctcacaacgccattttcttccccattctatagccacgattctccttttatcatcttcattgtctctctcaattttcaatattttttgctccaaaatattgtggtaagtgttaaagttgttagttttatgctagtactgtgcttcttctttttctctttactaatttttttaaaatattcaccattgttatgctttcaccagaaaacttgaaatttcctattccaatagctgattcaataactccgaagagggttacccgaggtatacccaccaaagcactcaattttgatgggccatctttcgatttgcaaatcaatcaagcggaacatgtatttgcaggttcagcaacaacaactgttgcagaaaggagaataaaaatacgcaatgacacaccgaaacaaggccaatttgagaaatcttcgaaagaatcaatacttcctgatgcggaaaagaaaagaaaacttatggatgatgcttcagggggtaagggaaaagaggtcgatacaagaactagctcgaaaacaccgaacgaaaaggtaattgtagatatatttatatgtatttgagtccgcatacattgattattaccttttacaatagttttctcttcttgtatgttgaagtatttatatgtattcaatagtgcctacactgatttttacttattacggtgacttttgacggttgtatttagatgtattttagttcattcaacaacttttttaaatgcactagttgttctaattttacctatgtattccaactaagtgttttcatctttattttcaagccaactgtattctgttgtatttaaaacaacagtcattgtcacaactcatattttttgtggtaattatcaatacattatgtttgtaattcatgaatgagttatgtattctaaataattgtattcatctatattttcaaccatttattttatctttttaatttttgttgtatgcatgaaataaaactctttgttaaatacccgcctattgtatcatcgcatatcaactcctacactaatacagacatagtctccgacctcaaagaaaagcttactccaaagcaatacaaaatattttgtacttcttattttggaagtttcctcgatatgaagcactgtgaggttcaacatcaattgttcaggtgcttcatggttatggagctggaagaaatccatgatgattcattttcaatatatgtgaatggtacaacattatgctttcaatcagggagtttgtggtagttattggtctcaattgtgttagtgacgctggagatttttagtttaacacaaagatgcctaacaggatgcagatacatactttggcggtgcaaagaatgtcaagaagaaagatttgttgaaatgctttgatgacaagaattggggttatgacaacgatggagatgccattaaaataattatgttgtatttcatacatactttcatattatccaccgagaagaactgcacaaccatcccaagactacattttgacttggtcaagagcgggagatattctgattatccttagggtaaggatgcattccaggcccttattagatctatgagcacgaagatggattcttagaagaagtattacaggatagctgggatgcctctcgctatgcaactatggttctacgagtgttgttcaaaggttgatcccaaaattgcactccgagttgataaccgaattcccagaatactcaattggaaattcacagtcaaccagccaacttgatgaatggcatgttcaatgaccaggaaaacttggtaatttactattatttaattgactggtattttagtcagcatatatttctgttagtaaaagcagtgctgaatttaaaaataaaatatagtttaatacatcatacatcaatgcacatgttttcataattaagtggaatacaacagaatacataatacagtgttaaaattaaaaattaaatacagttgcatacatcatacattaatgcgtacttttcaaaattaactagaatacagtagaatacataatatagtttacaactacacatttaattcaatgatgttaattttgtatacaatacatttatatagataattttccatatacataaagcatattacattagtaatcatacatcaagcgtgatttacattaagtgtatttctattttcagatgaaatacaacaatatttaaccaaacgctagagagcttgttgttattcaggtttctccggatggcgttgatgtagacaaaagtcctattcatatagacaacaatgaatatgattttgatgactttagtcctacaccgcctcttcagcctaagaagaaacgtgatgcaagcgtttgtctatcttcatcaccacctcacaaaaagcgcaaacaacagctaattgatatctcaactgcagagggtccaattccacatgtgaccgcaatacctcaagccaagtttcctcctactgctaatattaaaataaaggaaactgtattgagtgttaagaagccagttgattctaaatctgatgatttgtctgctttgaggcaagacctaaattcattcaaggattatgtatgtattgttaaaacatattcaactttataatttttgtattaactttcatatatgtttctaacccttctctaattatttgagtaattattaggtgatgggcaagttcacttctctacgatcattgatcaataaaaattttaaaaagctttctgaacatgttaaagccaatcagaatacaaaaagggtataccaaagaaaaaaataaactaggagacataatggtggtattcaaatgtcaatcgaagccaacttgcatgataatcccaaaggccgagcacaacatgatataactgttggggagaattcagaggttaataatttttaacctctttagtatatatgtagtataatgatacatgtgcaatcaatgtatttatatgttgctataactatatttatgattttcaaaatatagttacatgtgaatacaatgttatatctgtattgatgattttctaaatacatattgtaaaatagcttttactattctttagtttaatcatgtatacaataacatacatatttcagaatacacttatatacatataaatacattgttatatatgtattcatgattttctaaatacatattataaaataatttttactattcttcagtttaatcatgtatacagcaacataaatgattcagaatacacgcatatacatgtgaagacattgatagaatttaagtttgcacacctactatgaacttatgaagaaatatttttaatctaatctgataaatacaattctttaaaatatatgctatattaatgtattctattaggtgtatgttaaggctacggaaatgaagctgaacacagacattcatgccgatgaacatccatatgctattttaactagatacatgtatttctctggatacatccaaatcaaattatattaaaaatatttattcataaattcatagtaggtatacggacttgtattctgtcaatgtattcacatatatatgagtgtattctgaatcatgtatgttgatgtatacatgattaaaatgaagaatagtaaaagttgttttacaatatgtatttaaaaatcacgaatacatatataacaatgtatttacatgtatataggtgtattctaaaatatgtatgttgttgtatacatgattaaactgaagaatagtaaaagctgatttacaatatatatttagaaaatcatgaatacagatataacactgtattcacatgtatataaatgtattctgaaaatcatgaatatagttgtagcaacatataaatacattgattacacatgtatcactatactacatatatactaaataggttttaaaatattaacctctgaattctccccaacagctatatcatgttgtgcttggactttgggattatcatgcaagttggcctcggttgacatttgaataccaccatcatgtctcccaatatatttctatctttggtataccctttctatattctgattggctttaatatgtttagaaagttttttaaaattttcattgatcaatgatcatagagaagtgaactcgctcattacctaataaatacacaaacaattaaagataggttagaaacatatataaaagttaatacataaactgtaaagttgaatatattttaacaatacttacataatccttgaataaatttaggtcctgcctcaaagcagataaatcatcagatttagaatcagctggcttcttaacactcaatacagtttccttgatttcaatattagaagtaggaggaaacttggcttgaggtattgcagatacatgtggaattgaactctctacagttgagggatcaattagtttttgtttgcgctttttgtgaggtggagatgaaaatggactaacgcttgcatcatgtttcttcttaggctgaagagacggtgtaggactaaaatcatcagaatcatcttcattgttgtctgtatgaataggacttttgtcttcatcaacgccatccagaggaaactgaataacggcaagctctctagcgtttggttgaatgttgttgtattttatctaaaaacagacatacactcaatgcaaatcacgcttgatatatgattactaatgtaatatgctttatgtatatgggaaattatctatataaatgtattcagttgtattgtatataaaattaacaatattgaattaaatatatagctgtaaactgtattatgtattctaatgtattccagtttattttgaaaagtaagcattgatgtatgatgtatgcaactgtatttagttttaaattttaacactgtattatgtattctactgtattccagttaattatgaaaacatgtacattgatgtatgatatattaaactgcatttcatttttaatttcaatactgtttttactaatagaaatatatgttgactaaaataccagtcaattaaataatagtaaattaccaagtttccctagtcgctgaacatgccgttcatcaagtaagtataagttggctggttgactgtgaatttccaattgagtattctggaaattcggttatcaactcggagtgcaattttggagtcaacctttgaataacactcgtagaatcatacttgcatagcgagaggcatcccagctatcctgtaatacttcttctgagaatccatcttcttactaatagatctaataagggcctgaaatgcatccttaccctatggataatcagaatatctctcgctctcgaccaagtcaaaatgtagtcttgggatggttgtgcagttcttctcggtggataatataaaagtatgtatgaaatgcaatacaactattttaatggcatctccaccgttgtcataaccccaattattgtcatcaaagcattccaataaatcttttttcttggcattctttgcaccatcaaagtatgtatccacaatcatgttaggcatctctgtgttaaattgaaaatcttcagcgtcactaacacaattaagaccagtaactaccgcaaactccctaattgaaaagtataatattgttccattcacatatattgaaaatgaatcatcatgactttattccagctgcagaaccatgaagcacctgaacaattgatgttgaacctcatagtgcttcatatcgaggaaacttccaaaacaagtagtaccaaatattttgtactgctctggagtaagcttttctttgaggtcagagactatgtctgtgttagtgtaggaactgatatgcggtgataaaataggcgggtgtttaacaaagagttttattttttccatacaacaaaaattaaaaagataaaataaatggttgaatatatagatgaatacaataatttggaatacataactcagtcatgatttacaaatataatgtattgataattaccacaaaatgcgagttatgacaatgattgttgttttaaatacaacataatacagttggctttagaatacaaatgaaaacacttagttggaatacatagctaaaattagaacaactagtgtattttaaaaagttgttgaatgaactaaaatacatctaaatacaaccgttaaaagtcactgtaataagtaaaaatcagtgtaggcactattgaatacatataatgacaacaacatacaagaagagaaaattattgtagaaagtaataatcaatgtatgcggactcaaatacatataaatacatctacaattaccttttcgttcggtgtttctgagctagttcctgtatcgacctcttttcccttaccccctgaagcatcatccataagttttcttctctttttcgcatcagaaagtgttaattctttcgaagatttctcaaattggccttgtttcggtgtgtcattgcgtatttttgttctcctttctgcaacaattattgttgctaaacctgcaaatatatgttccgcttgagtgatttgcaaatcgaaagatggcccatcgaaattgagtgcattggtgagtatacctcgggtaaccctcttcggagttattgaatcagctattggaataggaaatttcaagttttctggtgaaagcataacaatggtgaatatttaaaaaaaaatagtaaagagaaaaagaagaagcacagtaccgcaaaaaagctaataactttaacacttaccacaactttttggagtaaaaaatattgaaaattgagagagacaatggagaagataaaaggagaatcgtggctatagaatgggaaagaaaatggcattctaagagagatcatggagagagaactttgatactatacggtgattttaggagagaaaaatgttagataacgtatttaatggcttaacggtagggagtgaccaataaatacatattttgctataaaaattaaaacgtagctatagaaaataatattttaaaagatttttatttataataaataaggtgtaaaactttgatgtatgaggtaaaattttcttttatttaacacactttaatccactcaaatttttgacccaacccgtccggttaatctctttgctcttaataagagctataggtagaattttttttaaaaagatacgaggagatcacaaaatataaaggagggcaaatagcatacctcactagttaccatgcaaaggaagtgcatcttgtgttaagaactaaccaaataaatcgctgtaaaaatgttgggcgaatgatatgaatattcaaattatttaatgattcaatgattaaacagtttcttatatttctttttattatcaatttacaccttcacgatgagtttttgaaaaagggaaaacataaatggtttaattttgtcgatgtattattcgaaattgctctattttatcaatcagttaaataattgtaactctagctctacaattcattgagctttgcactaaactctctcacacccatccaatactggctttactagtgtaacgatatcactacatcaaataacaatcaaaataaatattatatttaaaataagaatactattcaaattatcaacaacaaaaaaaaaaaaaaaaaatgtaaagggnnnnnnnnnnnnnnnnnnnnnnnnnnnnnnnnnnnnnnnnnnnnnnnnnnnnnnnaaaaaaaaaaaaaaaaaaatgtaaagggctacacagactagtactattggtcgattagtctttgtcgtagaatagaaagttctttgtccatttgctttctcacagttttaatataataatggagcttttcttagagattaagcttttaaaagtattcctctccacatcaaaaacaaaaaatgtattattattccactagtgagtagtgacttgttcttccttggagaccaaggtttcaacagttcttcctctccacgtcactgaaaaataatattttattacgatttttaaaaaaaataaaaatatattttaaattattaaatatgtagacagacatttaattttcataccccaacatttcttatgtaatatttttaaaatttatgttcaaatttcacacaaaagattaaatgttttaatccccacactttgaattccttcacaaccattttaaaacaatgagtttaaataagacttaaaatattttccttaaatcttatttagccgctttaatcactaattaaacagaatatattgaccttagaataactaaaacccttatccgaatcttatcggttagtagagcgttaggataataaatttcacaaactcataggtactctaatacaccttaaatattaggagatggctatcctttgtcaaaccataaattaattgtattttgttattggtcgatagtgtcctttagtctttgtcgtagtatagaaactaaaaagttctttgtccatttgctttctcttttttttctttcgtttttgtccatttgctttctcagttctaacataataatggagctttcaaaaaataaaaatgaattattccactagtgagtagtgactaatgtttacttggagaccaaggtttcaacagttctccctctctacgtcactgaaaaataatatttctttctatccataataagtgacattttgacttttatattttggttcaaaataattgtttttttacataatcaacaaaaaattaattttatttttccaaaatttttctttatttacatattacaatgtgtcaaggtaataattaattaaaggtaattaagtgaatacatctttttgttcaattttcacacaaaagattaaatattttaatcctcgttctttgaatagaccgtggaagtataatatccactagctataagccctttataatttttttttggtttcacaaacatattgtctgctcgtatcggggctccactaaatttaaatttgtgtctcgaaattctacattgaagagtgaaacatattgtaataaagacaaatttatatctaaagctcgaattcaagaccagatataaaaaagaaaagaaatacttattactccacgacaactttagctgatccatttataagttgttaagatcaacgtcgttttccttgagatcaattcgattgctatttttcctttttgccgttgaacaaatgagaacattttttcttcccgtttctcctcaaatagttgtatgacactgcaatttttccaaaaagtgatgttactctttggttaatatcttaacttattagttttctcaacttaaaagtaatgtaataatatttacatcggcatcttgttagagccaatataataagatcttcaaatttgtaaaattaaaatatcttcaaattgttagaataccgataataatatgtgttaggttagaggtacgtaaatacatttcgcgaaaatactgactggttttcgagagacctactaacctcgactaattcagatacactatctcttaggttccatttaaggggatgcgcgttctaccgataatttggttattacaaagctcaaattttatttttgtcagaaagcaactaattaattttttcaatgtcatattacaagtcgacacaggcgcctgtgtaaacaaagtgctttgtcggtgaactataaatatttcataatccgatattaataatcttttatttaatcaatttctgaccgattcaatagaatatatttgaaattggaatagaaaaaataaacaaaagaacaacattttatttgttttttaaattctttaatctaattaaagggtcatccatccaataaaaaataacttaagctcatttacgaagatttagaataggtcttttaattaaaggaccctagaatctgtttgtacgtcacaacttatactagatcagtaaaaaggggcagcctgatgcactaagcttccgttatgccccggccggaccacaagtatttattgtatgcaaccttagaactttaaaaaaatatatttcatattcaaaatatactactatttcgaatttggatgggtagagcacaccgagtgaaatgttatttcatgcatttttcattgataataaacaaacttaagtcatcaaaattatacacaacatactcattgtgtttgcgtatatttatattactccaacttagcatcacttctttaaatgtaaggtctgagaattactaaattataccgtgatgaaacaattaatggtcattagtaattaattgcaccacttaaattttgtacatgaaatttttaaaaatgattatttttgtttaatcatctgctatgctatcagagactactggctcgattaattcaaagtagcgccgtatagagttcgtttccttcattacaaggttcgaatccaaaactctaattaaaaatagagaaattaattcattatgtcacatagataaaaaaaaataaaaaaaatcagttgtcccactacctatgctaagacaaattaaaggtttcacattccttgcatttgatatcatctttagtaaagtttttttatttttattgcaaactagtgaatcttgtgacaatttttatttatttcaagaaaatcatgtttattattgtcgaaaagtaaataattaatttctcaatgccactttatggaaaaatccttagcgcgtgggtaaaccaggtgctctgccaattacattagaaataatacatgatctggtattatgcatctctttaaatagtattctttagattagtaattagttataagtaatacgatatcagatatttataatcttcaacaagatttagttttatttaaaacgataataacttacttaagcatatataaatttaaaacaacttgacattatattttatttggaagtcattcatgcaaatggccaagaaccaaacaaataaatataattttcctatatttcttttgaaccttttaatatgtttaatttaatttttcttcttttatttcatggtgacgtgcactgcacacggaagcaagccgtagtaggcgtcagttttttccttttcccttgtaacaacggtccatattcttatgaatattgacaaaaatacccttgtctttatcataattccgtctatacaacgaaaggctcacatgtcattaaacattttcttaaggctaaacaaggaatacgtttttttacctcttatgaaatttaaacagaatagtattaaaacttaataggagtactggtttttgtagaagccataaagggctacaatagactagtactattggtcaataatgtcctttagtctttgttacggtatagaaagttctttgtccatttgctttcttagagttgtaatataataatggagctttccttagagattaagatttcaaaagtattcctctccacatcaaaaacaaaaaatgtattattattccactagtgagtattgactagttcttccttggagaccaaggtttcaacagttcttcctctccacatcactgaaaaatggtactttcttcctccctcttacgtggaaatgttattattagggaattaaataatattttattatattttttttaattattaactatgtagacttacagtatttttatataatttttaatttttatatatgtaaaagtttcttagcagagataaggtctgcgtatatattatctaatactattgagtatattattgttgttgttgaattcgcatggcgtagccgtttacagcacaccaaatgatctgatatttcatgcattttttattgatgacaaacaaacttaagtcatcaaaatgtataaaacaacattcacatttaaacagtactcaactactcatccatatatgtttgcctcgtatatatatttattaactcctactttacatcacatatttaaaagggctaatcattactataaattatttactactctcttgccgtgattaaagaattaatggttactagtaattagtgaaattttgtacatgaaattgcaagaggaattatttttagaatgattttttctttcttttaccatcttgttttctagagagctacgagcctcgactaatccatatagacatcgcttagggtctatttaagggagaagcgggtcatacctataatttgtttgttataaggctcgattcgaaatatctagttaaaagtgccacttgtaagactattgatgctaagacaacgaatgtgatgacataaaatgtcatctttaatttaaacattcatttttgtgttctatgacctcgaatagtactattcatcattcctcgacttgcatgtgcggcccgaataatttttcaggaagtttatatatgaaaaattgattaaaatgtgaaatagagctttaaaactcaactgagttgacttcggtcaacattttgagcaaacggactcagattagagttttgacagttccaataggtctgtatcatgatttggaacttgggcgtatgcccggaatcgaattctgaggtccctaattcaagttatggccatttaaaggctaaagattttcaaattttgaccacggatttgactttattgatatcggactcatattgagattttgaaaattggaatagctccgttatggcatttatgacttgtgtgccaaatttgaagtcattctgaattcgtttaatatgtttaggcacgagttttgtaaattgaaaaagtttgaaactcataagttcgaatcgagttgcgaattgtaatcttaaagttgtttgacgtgatttgagaccctgaataagtctgtattgtgttacggaacttgatggtatattcggacgaggacccgagtactccgagtgtgatttagattgaaatcggatcaaaacttggactaagcaaaatctgaaattttggtgcttctagtgtcttcgcttatgcgaagccttggccacagaagcgagaaatccctcacaaaagcggcttgggcaaatgggaaaaaaactgtaggtggggaaccttgaacgcactcgcgcatccccaaaagcgaactctgcgatcgcagaagcggaaggcaacgcaaatgcacattttcctctgcatatgcgacgttttgcctagccaacacttcttcgcataagcgaaatttttagtcgcaaatgcgacaatttcatcgcagaaacgaaagattggtagaaacataagaatcgaaaatcagtcatttcgccatttttgttcaagttttaaaacctcgatttatgggtgatttgaaagagtttttcacaacttcgacttgggtaagtgttatatacccgaaagtaattatatttcataaatccacatctatattcatcgttttatttcggatttagatggaagaaattggttttttttgtaaaactttccaaaaatgaaaatttaggatttgaaggtccatttgacatcagaatttgataatttttgtatggtttgactcgtctcgaaataggtgttcgaatttcgctagtttttccagtattcgagaagtgggtcccactgtcaatttttaaaatgaattccagatttttaatctagaaaatttgtaaattcatatagaattaattcctacgactcgtgtcgagtatattgaattatttgtgactagatttaaaactttcagacatgaattcgcgaggcaaacgtttattgaaatcgtgaatttggttgcgaagcgaggtaagtgtcgtggttaaccttgacttgagggaatagaacccttaaattattgttaagtgaatttcatgtgtaatgatttacaagcgaggtgacgagtgcctatacgtcgtcaaattgattgtttgcataattatctgaatatcataaattatttaaaatcatgaattaattattacattgattatttctctcatattccctgttcaatattatgtcttgaattcatgctataattgctatatgcttaattgaattatgtgtattaattattacttgatatttagcatattaaatattaaattgcctattttctccttgattttcacaattaattgctacttgtcattacttatttttctaaataaatcataattactgtatgctttgttgcctaataatttcttattaatatggtatttattggagtatttttatatttaagagttgttaaattatattgttggatcaggttgcatgccacaacagatttaatttaagtttatattgttggagcaggttgcacgccgcaacagaatttaattgaaagattatattgttggatcaggttgcacgccacaagagattttattttaagtttatattgttggagcggattgcacgccgcaacggaaattaattgagggattatgactactgaattgacttcaattattgatatgctttactgatcttacttctattcctgttattattattattattattattattattattattattattattattattattattattattattattattattattattattattattattattatgtataggttaatgtaagcgacctgccttagcctcgtcactacttcgtcgaggttaggctcgacacttacagagtacacggggtcggttgtactcatactacactctgcacttcttgtgtagatttcggagttggtcccaatgacgtactgtagaattgctcagatttagctatcagaggagacttgaggtatagatgcacgacgttcacaactctgaagtcctcttctactttatcttagctatgtgcttttttttagacaattttattttatttagacccttgtttgtattattctagaagatcatgcacttgtgactccaattttgggatgatatttagacatcatattatttttggtttgcacatttaaattcagatattatttcgattgttgggttttttattatttaattaaaataaattgttaaaaataatttaaattattctaacgttgccttgtctaacaagtgaaatgttaggtttcatcacggttttgaatgtgggaatttcgggccgtgaaaacaaattcacatcattgcaagtgatctcatgaactttatcctaaaacgtttcttgggttgcaaactcgtgaatctagtgacggtttttatttcatttcaagaaattcaatttttatttttgttagaaagcaactaagtaattttgtcaatgtcatattacaagtaaacataggcgcccgtgcaaacaaaatgctctgccattgaactataaatatttcattcataatccaatattaataatctttgatctctttcagaatgctattcttaattgggtttttgcctctgtgtgtttctttttcttttttgtttgagtgaaggccatatgttagaatttggagaagagttagcaggaaaacaaattcctttattttcttctcctaagttgaaatcaagaaaatgacccccattgattgataaggaagttgataaggaacttgtactagatcattgaactttaaaaaaaatatatttcatattcaaaatatactactatttcgaatttggatgggtagagcagacaaaatgaaatgttatttcatcgcatttttgattgataataaacaaacttaagtcatcaaaattatacacaacatactcatttatgtttggctcgtatatttataactcctacattttgagtactaaattataccgtgatgaaacaattaatggtcattagtaactagtaatactagtaccacttaaagtctgcacacgaaatttttttaaaaaattatttttgtttaatcatctactatcagagattattggctcaactaatccaaagtgacatcgtgtaaggtttatttccttcattataagcttcgaatccaaaactctaattaagaatagagaaattaatccattatgtcacatagtaaaaaaaacaatcaattgtcacactacctatgctaagacaaattaaaggtttcacactccttgcatttgatatcatctttagtaaagttttttttgggttgcaaactagtgaatcttgtgacaaattttatttatttcaagaaaatcatgtttattattgtcgaaaagtaaacaattaatttctcaatgccactttatggaaaaaccattagcatgtgggtaaaccaggtgctctgccaattacattagaaataatacatgatctggtatgatgcatctctttaaatggtattctttagattagtaattagttataagtaataagatatccgatatttataatcctcaataggatacccttactttagttttatttaaaataataataacttacttaactcatatataaatttaaatcaacttgacattatattttatttggaagtcattcatgcaaatggccaagaaccaaacaaataaatataattttcctatatttcttttgaaccttttaatatgtttaatttaatttttcttcttttatttcatggtcacgtgcactgcacacggaagcaagcccaagtaggcgtcagtctttttcttttcccttgaattgttatatatattgacgaaaatacccttttctttatcgtaattccgtctatacaacaaaaggctcacatgtcattaaacatattctcaaggctaaataaggaataaaaaacaaatgtacttgctgcgttttttttttcttcttatgaaatttaaacagaatattattaaaatttaataggagtactggtttttgccttcaattggatttcctatataatactataataggccaaaagattttgtcttttcatttcccctctctctctctttcattctgaaaaaagaagtgggattgtattgtattttgattataaattgtactcaaaattgcttgaattactttgtacacaatcttgcaattcaactcttgccagtaagttttcctaactatttttgtgtttcttctaaatgttactcttttttcaggattataaaccgtttaatgccatgctcatccataatatatttgatttgtatatttattactcctacttaatgtcatatcattgattgaaatgcctatcaattgcagtaaattatttactactctctttcacagtgatgaagtaattaattatcattaataattattactcctttcgcctcaatttaggtggcatcatttgaattttaagagtcaaacaaattttgtttcaatgcaattttttcatatggattttaaataatttgattttattagttgtcgtgacttatagtagtactttttatatagtttctaagtgtgtaaattttattttaaaaaagttaataatttttatgttcaaattcacgatccaaattaagaagtttgactctcgaaattcaaactatgccatataaattgggacataagaagtactacttaaatgttttacatgaaaatgcaaaaaaaaaaaaaaaaaaaaaaaaaaaaaaaaaaaaaaaaatagcactcaattatcacactatctatgttgtgacaaagaattcacatcctttgtaagttagcaactgatctgaactttataataaaatattttttttgaatctagtgacaatttttatttaattttaagaaattcaaattaatttttgtcagaaagtaactaattgattttttaatgccactttacaagtaaagtattagcgcgtgtgtaaactaaatgctctgccaatgaaagtataaatattccaatagtaacaactttgatctctttgaatggtattttgagagaagcattaattaatagtacttagtaatttgttatttataattcttgataagatgcacttttatttattcctctttaaaacaataaataattactttaactccgatataaatttgaaatcatcttgacaatttgtctgtagtcaatcatacaaagtactaaattaaggacccgtttaactataaaaaaaattacttttttggggatttttttacttttttcggattcaatgtttgatcatgaaaattttaaatttcacttgaatctgaactttaaaaaatttcagaaatttaaaaaactccaaaaagttatttttcaaaaatttcagttcaaatcactcacaaaaaattataaaatagtttttttgtgtattcatatccaaacataactctaatttttaaatactatttttatttaaaaaaatattttattttttttccggaatttcataattgttatgtccaatcgctcactaaaagccaaacaaataattataattatattttgatagaaaccaactttattttgaacttttaaaatgtttaggtttttccccctcttatttctcggtcacgtgcacggcacacagaggaaagcagcagtaatcgtcagcgtttttcctatttcctcgtaacagtgacgagaaacagaatcccgtatccattttcttatgcaaaatgactaaaatacccttcctattatcaaatttcgttctggatatcagattgtcattaaatagcttctcaagggtaataatgaaataaaaaacaaatgtatatacagagtgcgtttctctctttagagaaattcaaacagaaaagtattaaaggagttttgatttattccttcaatttgggtttcctatatttaataggtcaatatgcttcttgtcttttcatctggggttgtgttgtattgataaaaaattgtactgtcattgctttttccctggggtaggggaaggggtgcggggtatggtggtgttgcagagttactctgaggtgggggagtgtatttttccttgccatagaagctaaagacctctacatatagtccccttgtatctctcttactttttcttcttcactgaacactcaaaactgtttgaaatattctatacaccaatcctgcaattcaactctagccagtgagttttcttaactatttatgtgttttttcccataaatatcactcttttatgcattttattcaagtgggtgtttttcatacagattactcttttatgcatatgtgatttacttgtgatgttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggggggttgattggtattcttttcttgatttttggtttgtccttgtgtgtttttttttaaaaaaaaatggtaaaaagggtatgcaggagttttgtgttttttttttaatgtatgatcacctgattgtcatctcctttaggagtttactgtttcatcattgttactcatagagttttcttggattttttatgaagtgggtgtttacatggtgttaaatctttaattatttgtgccttgtgaaatccttggtttttcttagtgtttccttaaaatggtaaaaagggtatgtaagataaataggcttctattatgtatgatcaactggttgtcattttcttgtagatttttctgtttcatctttgttacttggttggtgtccttatgtttatttggcctgatgttaatgtagataacaatcaatggatccaactgaagttacagtctctgaaaaccaacagctctgcgctgtctctctgccttttgcaagaaggttggcattttacatttattttctcttttaatgtatcatggagttagaaaaaaaaataaaaaaggaaggaacttttgtaatgtgtgttttttatgtatgattttgttatgtagttatcagttagaggcattggagacggcattaaagcagaacactattgtttatttggagactggttcgggaaagacccttatcgctattatgcttctgagaagctatgcttaccttcttcgaaagccgtctccctacatagctgttttcttggtcccgactgttgttttggtggctcaggtactacttctagcttcccaagctagtgcttaaacttcgttatcttaatgtgtatattttcactcctgtctatagtttgtagaactgttttattgagcattggttttaccacattttgcgaattgttatactgtttgaggagtacctctttagtttgtgttttttctaagggctatggttttaggtgcataactctctatttgacttgtcctaatgttgttgatcttgcgattgataattacaaagcaaggagatgctctcattatgcatactgacttgaaagtgggcaagtattggggagaaatgggtgtcgattattgggatgctgctacatggcaacaacaggtggacaatcatgaggtatgcgtgaactatattttatctttagtgataaattattaacacttgcttgagtaggcattagatatggctacgatgttctcaaaaagattccattactgaagttacttaaatactacaattttttgttgggacaaaaatggaattgcatgtctacatgtctcctctatttttcctgcccaaagccaatggctatgaagtataagctaatagtttctctccaagggaattcgcaaacaaagctgcttttttttttgccttgcgatcctttttgttttggcatggctcttggcgaatattttagatttgagtttcagctatacagtcaaacctctctataataacctcgtttgttccaactatttttggatgttatcgcgaattgctgttatagagaacagatattataacataacatgaaaattggttccggaaaaagcttggcttttaatatagggatgctgttatagacaggtcttactgtacatgctaggacttaagttaggaactgtatatatatcacttggatgcccttatcaaaaaatatatacctggatgcttgataagtaattatcgtttctgtctttcaggtgctggtaatgacacccgcaatactacttgctgctttgaggcatagcttcttaaaaattgaaatgatcaaagttctaatatttgatgaatgtcacaatgcacgagccaaacacccttatgcctgcataatgaaggtacgttgtttgtactcatactcatcttctatcggtatcacgctggatctaaacttttgtgcttatcttcgggagagtgccctacttaccatcataaagaagaaagaatggagcaaagttatatcaacaacatttgatttctttgtcaatggatattgattactaatgtgtacttatcgccacttcttcaacagtattttatttcagtacttcctgccgcttgagtgtatgtgttgcatttcacataccacttgtaaaaagttagataaaaaagagatttatgagaatttgggaattagataaaaaagagatttgagagaatacgggaatacatgtcttatggcttaattcttgtataggcatacatttacctgatgtggcactcttatgctcacgtcattgcttttgtacatatgtgacttaagtctaactagtccaaccaatctaactgcacttaagaaaagacccatgataatcttgcttgagcatgcctttattacggtgcttgaacaatttttgtcttaaagggtggattttcttttcaggaattttatcatcgtcaattaaccctcgaaagtgcacaacttccgagaatattgggaatgactgcttcccctataaagactaaaggttactgacatttaattttatagctttgaacatcctttgagatgcttttacctcataattcatactcttagcaaactgtctttctgtggattgtcatattcatacttttatttcaggttctagcgcggaatttacttggaaaatgattcgtgacctcgagaaccttatgaattccaaggttctctccaaatattttccttttatccattcacgtcatgcgatatgttcttaaatgtagttcaaaggatagaagcatgttgtttccttttttaaagaaattgttttggtgatggcatcaacttgatttttgcaaatgtatctttctgtaagccgtgtttattactggactgtgcaagcattaaactttttgcttcagatacttgtcgtcagtacattgcagagcattgcacttgttttttgggtagcatgtctttttaatgtatgcaagtagacagacacaagtagatatatattcagttcttctgattgtgttctgcctttgcaaatgaaggtatacacgtgtgtcagtgaatctgttttggccgagtatatcccattctcaactccaaagctgaagatatatcaacatgtggatattccgtgcacattgtttgttagtttagtttctgacctgataaggttgaaagacaaggtgagcatcaattctttagccagtgggttctgcctttctatttatagaatgttcatacataccttttgtctttctctctggatttactccctttatatcatatgttttttccccttggaacctatatacaacatcatttatcattcagtttatgttggctcttcctttctctatatacagtagtttgtgtgcgagctgaaaacgggtagtgcactgagaaaataatggaacttagaactaaaccttaatatattcttgtaatgagattagaacaatcatgttggcttgttcaacatatggggtctacatttttgctctttcaattctggttgtgttttcttatcattgcagtatgaagattcgatttcaaaatcgagcctatctgatttaagtgctggttctgcaaaaaaaagattgtcaaaactatattcgtccttcgtattttgtttgagtgagctgggtgtttggccggctttcaaggtaaatacgttccttgcttgtgcataaaaaaattaatcagtttggccttgatcattttcctaacatgaattcgttttgaatcaggcagccgagttcttgtcctctgaagaaagcgacttcttttcatggggagaattagatgtttgtgctcaaacgattgttagaaatttcagtttgggtgcatctaaggtcttctcggctcgcataccatctggtatcataattagcaacatccattttcttttcctgagctgtaaatatgtttttttgacttctttcttgtttgttcgtgcgctctctctccctccctggaaacataggttctcattggtcacttggtggtgatattcatgcaaatgttgatgctggatatttgacatccaaaattaatagtcttatagagtctcttcttgagtacaggtgagaggtctgactagtaactctatttgttaaaagcatcaatatcaattcaatctattggatgttctatagaataaatggaaaaaaaagttgtcggatagatttcttaggatttgtggcaacgagagaatgacatgcaattttgtgttgacggctttaaatatttttagaagcacaagatgagtgctaaaaatttcaattctagagtcattgaactcgcaaaccatctaactgtaatggagggagcattatgctgccaagtgactcccttacatgactgtactttcatctatgcttttgaaataacattgtacatggtagtttaccgatggtttatgctattgcttttaatgatcacgttttaatgttaaaactgtaatcctctgcttcttgtctcgtcttatctttgattaaatatgatgctggagtctatcactctatttgctccttgattttgtgagatgtcttaaataatgcaactctcttgttgggaaataggattttctatttggtgattcactggctaacaatctttttttgttgttgacttttgtttccaatccaatagagacctgaaggatttgagatgcatcatatttgttgagagaatcattacagctattgttcttcggtctttgttaaatgagttgcttccggaattaagtggttggaaaactgagtacactgcaggacatagttctatgctgcagtcacagagcagaaatgttcaaaataaaattgttgaagaatttcgcaagggtgtggtaagtccggttttatttttccccatctttgtagataatttctgaagattgaaagtttcatttccatgcaaaacgctgcaggtcaacattattgtagcgacttctattcttgaagagggtttggatgttcaaagttgcaaccttgtcattcgatttgatccctcagcaaccgtttgtagctttatacagtctcgtggtcgagctcgaatgcagaactcacactttattatgatggttgggaggtatgcttttccatccattccctcctcgcaaatacaaatattataacttgagtataattgatatatgaatgttttaaagggttctatcattatatattgttattggtctatttttcacttaaaacattctatttatgctcctgtctgttgtgcaacttttaaaaacattaaattacttcagctcttgattttgtgcaattattattgataaaatttggaagatttaattataacaatggactgattatccttctatttctggcttaattcctagcggggatgcttcaactctcactcgcatgcaaaattatatgaaaagtggggagatcatgcggcaagaatcattacgcaatgcttctattccgtgttcacctcttgatgatgaattgcacaatgaaccttgctataaagttgaaacaactggagcagttgtcaccctcagttctagcgtgtcattgttatacttctattgctcaaggctaccttcggatgggtttgtactgcataatccttgctacgttctaatgatttggtatctttgagtacttcaggtgttaaatgttgcattattgactgcccttcaatgtgattttaggtactataaacccagtcctaggtgtgttattgaggaggaaacagaaacctgcaccctacatcttcccaaaaattgcccactgcaaaaagttattagtgtgaaagggaataccaaaatattgaagcaacttgcttgccttgaagcttgtaaagaacttcatcgtgagggtgcattgaccgacaatcttgttcctgatatcgtggaggaagaagccatcatcaaagaattgggtaattctcgcttttggtaataggttgttttttttagaaaagagccaaatatatccctctactttcatatattgtctacatttaaccttcgttatactattgggccaaatttacccctacaatcagcaaactttgaaaatacccctaaatttaaaagtttgctgattgtaggggtaaatttggcccgatagtataacggagattaaatgctagacaatattatgaaagtagaggggtatatttggtccttcttttttggactaaagttacttgccttctaacccattatattgcgcaggatgtcaaatctatactgatgaagaatcaaagtactttccaccagaattggttagtcattgtgccaatgatacagaagcagtgtactactgctacgtagttgatttgcaacatgactcttacagcaattaccagcttcgtgggataatacttgctgttaggacaaggctcaagttagacgatgaaagattgatttttgacttggatgttgataatggaagcttgctagttcaagtaaattatagtggagttgttaggcttacgtctgaagaggtgactataatttttatgacttctatcctctctttacttgtcacctgtaaaaaagtaggtaatactttttatattctctctccagattttacgatgtcagaggtttcaagtcagtctattcagaattcttcttgaccgcgatctgagtaagctgcaggacgctttggctgcagttcaactgcctgttggttctgcagtttctgattatcttttgcttccatctttgggttcaactcaaaaccctcaaataaattgggaatgtgtgaattctgtgctgtttccttctcaagttcttggggataagcatattgattgttgttctacacaaggtcgcaaacgcagtgtgaacatcaaaactggggtggtatgcagctgcatgttggagaactcattggtctgtacgcctcataatggctatgtgtactgcattaccggttttttggataatttagattgcaactcattgctggaacagagaactggagaatccattacctacaaagaatactacaaaaagcggtttgtacaattaactactaatttcttggtgcagcttttccttgtatgaacttttggtttcaaaatattaactaaggtgcggtttacaggcatgggatcaacttatgctttgaaggagaaccacttcttagggggaaacgccttttcaaagtgcacaattaccttcaaagatgcagaagtcagaaggctaaaggttgaacaaatgtttctctttttattataatgcagaactcaaaacttagtttcatagtttaatggaaaatcacttattcaattttcatttgacctccttggccgactttgcagattcaactgattcatctgttgaattgccccctgaactttgttccatcatcatgtcacctatctctatttctaccctttatacctattcatatgttccttcggtcatgcatcggattgagtcattggttatggcctcaaacttgaaaaggatgcactcggatcagtgcacgcaaaacatttttattccaaccgctaaggtatgtgctgctatgatctcatcacagatttagctccaatttaatcagtcttaaactcttaactttacacatagtctagggcttgactttacatcattggactagctttttctcgacttccttgtgttttagctgatttgtggtccttgtttggttattttaggttctggaggcaattactacaaagaaatgcgttgaaaagtttcatttggaatcacttgagacacttggagactcttttctcaaatatgctgcgagtatacagctgttcaagacttatgaaaatcatcacgagggtctactcactgttaagaaaaacaaaattatttccaatgctgcattgtgcaagcttggatgtgctcgtaaaataccggtaggtattccttggtgatagtttagttgggttttcattatgagtttggatagtatcaactccatgtctatattaatttttcttacttatttgcacgtgcactttaatcaaagatcctttgttgaatgttaattattcctgcaacctaaaagttacgaactatcacgtatgaacattcttttctactgtcccttgcccacacaactaagtctagtgtttgttataatgtaaattagtagttattggccattgctgttatctatatttgtaaaaattgtctactttttgtcagtgcatcaattggagtacgtttgtcgtaggacatgtcaattttgacactgtttttggatccttgaataagtaaatatgcctgtgtatacttaaagatttcctatagttgagtcttaacattttatttgtttaaaacttctggattgcctgatgtgtgctatctcaataaatacctttctttacagggatttattcgtaatgagccattcgatcttaaaggatggctcattcctggtgataattctcaagttcgcaatttcgatgaagaagtgctgatgccttctgtaaaaatgtatagtaggggaaggcagaagattaaaagtaagagagtagctgatgttgttgaagcactgataggtgcctttctcagctcaggtggtgaagtagcggctttgtcatttatgaaatggcttgggatcgacattgattttgtagctgcacctacgctgaggcacttccccatgaatgctgagaagctagttaatattcgatacttggaatcactgctagattacaagttccatgatccttctctgcttgttgaagctctaactcacggatcttacatgctacctgagattccacgatgctatcaggttattctcgtaaataaagatttatccatcttgatgttgattgcatttttgcttcttgtggttgctctatcattaaatcgtcaattagttgggtctatttcttgttaacccgaacatttcattgacctaagcggctcgatagtacttagtgattcttcataaaactgctagtgacaaggaacaaactgcaccgtttaattcaacaacccagtaggatcccacaagtggggtctaaggagggtagagagggtagagtgtacgcataccttacccctacccgtggggggtagagaggctgtcaccgtttaattctttcaagcgattttttcatgattttatttgaatcctgtcttgccgtcttatcttattcgaagttcagatttccttttcctgttctgttgtaaggttgaagttcttacaattgcgtgtgatctgaaccaattgactcagaatatcttaaaaacaaaagcagtaatcggacgttgtttccaattgacttgtagtcttccattagcaatctcgtgaacttaatgttatatgaacaatgaaatatatcaagtttgatgtttcctaaatctaggtaactttcttgattcaactgttgatgatttacctggctatgtagcgcttggaatttcttggagatgcagtgctagattatgctgtcacagcgcatctctatttcaaatatccgggactgtctccaggactcattactgatttgaggtctgcctccgtaaataatgaatgctacgctcaggctgctgttaaggctggcctacacaagcacattcttcatgcttcacaagatctgcagagacagatggttaatactgttctgaattttgagaaattggatcccgtgtccacgttcggatgggagtccgaaactacttttccaaaggtattgcatacttgcctcatagttagaaataacacttcatctgaaaaccataaattttagaaacgaaaggaaaaagttgacggacagctcttcccgcaatgaattaaacatctcctttccatcattctcaacttcatctctattgtcgagagcaacttaagatcaatgtggattaatagtactacctccgtccaattatgtggcaccatttgagtggtcacaaagcttaagaaagaaaggaagacttttgaaatttgtggtctaaaacaagcattagacatttgtgtggcagttaaatcatctcattaagtgtaaaaaggaaaataataagttaaattgtttctaaatatagaaagacattcttttcaggacagactaaaaaggaaagtgtacactgtaccacataaatgggagcagatggagtatccaataatctaatctgattttctattttaggtttattaactcgtattcctttctggttcaatataggtgcttggagatgttattgagtctcttgccggggcaatctttgttgattctggcttcaacaaagatgttgtgtttcagagcatacgaacacttctggaaccgttgattacaccggatacagtaaagctccatccaatcagagagttaagtgaactatgtgaccagaaaggttatataaagaagaagaacgttgtctctcgtgagaatggtgtggcgtatattacagtagaggttgaagcagatggtgttagtcacaagttcacgtgctcggaacgagacaaaaaaatggccgagaaggtggcttgcaaaaatgtactcaaattgctgaaggaatgtgcatctgatgcttagctttagtcgcttttctgcaacttcaggttacaattttcatatgttttggtttctgccctgttccttttgtattctgctattatttcaaagtgcgatgtttgttttgattgtcatttaaaatttattgcgatgtattattggattcattgttatgtgacaatgaagagtctcattttatgaaccaatttgatgttgggggatactcgtgttatttctttgtctcatgttgcccttaattatttgataatctgtttttatccttaaacatgtttttttaactctatcattcagcttgtttggatggttgtatgtatggttttataatgtattgtataatattgtattgttttgatatataccgtgttttgagttgttattttatgatgtcatgcacctacaaatgaacttgtaatattataaagaaaaagtaaggcatggggtataaatattatataaaatggtagggtaaagaataaaatatgattatttaataataaggaaggcaagggcagcccagtgtactaagctttcactatgcgtgagggtccggagaagagccagatcataaaaagttatatcgtacgcagtcttacccagcaaattattttcacggctcgaacctgtgatcttttggttacatagtggcaactttactagttacgccaaggccccttcaataacaaagaaggctgatgagacaaaaaagaaaaggaaacgatgtggccacaccaaattggttgttccataaagtgtcattttttcgttgtttcaatgatagaatttaacgatatgatataataaaatttaaataataattaaaataaatattatattaaaataataatagatgatacaatacaatatataacaaccatgtaaaacaagctgttacctaattgaattgaattcatggctaaaccttttgttagggtattcgagtctctgctgcatatacgtgcttattatttttctgttctataaactcatcattttaacaatacaaaagttaggcttttcttttgggtagcattattatatactcatttcatccttttccaaaccatacattatacatgtaaatattataatttcaaaggatatccttttttggatgctttcattacttccttccatatataatctaataaaagaaaattcacaaattatctgcgcaatggagaaataaacacaaataaatgtacttgaacctaaaatataacaaatcattatttctaaaccttgatactcttaatttatttcatttttttttctataacggaaaaactttcgagggtcaatgacgtatgaatcgaaacttgcgagggtcaatgacgtatggatcgaaactcaatgatgactaatgtactctcccttctgtctttctacacttatatattacttttgtttgtgtcagagtacgaacttgtaacatgagcttaataacatatcacatgttgtgtttttactactccgaagcttaatttgtttcattgaagttcattatagaaaaatgctccttatcagattttttttttatttcacgacgcgaactagactctcttatttagattaaagaaattctatccatcctatcataacacacattaatatttgcttcattttattgaaaaagttgtagaattctttgactataaattggtaaacgacacttgtatttggtgattattcgttgacaatctagaagataatgatagttatctatttatttctcgtggtcttttataggttgcttctgggactttaatttctttgaaatgtgttatgcttctttcgacgtgtctctttttcttcaaaaatctatgctattcaaattctttaaaatccaaaaggatttaaaaagaatgaataaaatttctttttcttcaaaatctatacgattcaaattattttttattttaaaaaagaaaaattaatactattaggggccatttggttggtgacataaggaatttcaatttcgggataaaatgcgggtttattttatcccgcatttggaaatgggtagttattagtttcaataaccattttgtactaaaatggtgggattaactattccaaatataaagtgggatagctaaccccttaaggaatgtagagaaatgttaaatttcttgtcattaatatttaggaaataataagtattctgcaacgtgaattctgattaaactttttgatgaatttcagatatttatttaatggttaaaaacagatataaattcgaacttcataataataggttcacaaatgcaagtcataattcctggagaaaaggcaaaacacttgtcattattatagatatacgaggtgcattctatttatccagaaaatggccccattttcctgttcttttccaccttgtttcttacgtatactccatttttcacgttgtttatatatttcttgccaaagtaaaacttgatataacaataaattttgtcaacaaactttgacaattccctcaatttattagcttttcctttatctggtacacgtgtgactataaatggtctttgtttttattaatgatatgatatatattgctaaaatgagttcgtacaatcacccaagacggtaacggaataaatataatttttttaatccttaagtcttaaatttcgatttaagatatgaaataaagaacacttcctcgtttaatgagcatgttgagaatccaaattaggcgtacatatttacccaccctaagtttttacatctaattaaataattaaccttaaaaattaataaatcaaaatgaaagtacatatatatcacttgacaatgattaggtattaaaaagtgcttcctccatctctgatcatttgtcattatttttataaaaataattatatcaaattatttattattttaaaagttcatgacaaaattaatttccttttattttatcattaatagtaattgttcatgaagacggagataccacataaataaagtaaatattcaatacagagagattatatatcttaggacataaatatagataaaatagttcaaaactcctaatagtatttttttcaagaggcatgtaaaagaaaaatacgacagataatttgagatgaaaagcatatatgtctcactttcattgatttgatgtaaagcatcctaataaatgatttgtctagaatgagaacaattaagggttcgtttgatagaagggataagagataattaatctcgagattaaatttgagatgactttaactcacgttttattagaataaaatcgtgatataaataatctcgggattagttatcctaggatcgtagtattttttttaaaatctccatgggagggtgtgataactaatctcgaaataattaatcacgagataacttgtttcttaaccaaacaaccatatggaaagaagttatcttaggttatcccgagattaattatccccaaattagttattctacctttctatgaaaataaaataacagtacaatcccgaaataacttttaatctcgaaattaattatattttaattttctctcaatcaaacatgatataaacttttcgaattaattattacttatggttcataccaaaccagcctaaatgtgtacgttaaaattcagctgactagaatagtagtaatacaattaaaatggtccccacacccttagattttccattattacacatatctacacacttctttcaatcaaataccagtaggtccaccaaacttttctacaaatatataaaaagcccccctcccctgccacgaaccattcacttcaactaccaccacctccacctaactaaccgtcgttgttcggagctccgactgagcatcaaatgttcaaacaatactgctgttgtctctttccgggtcggagctccgacttcgtcaatgatgttgttgctgatagggaccctgtattgttggtttcgggtcttgccgggtccattctccattcaaagagtaagaaactgggcggttttgaaacccgggtttgagtccgacttctccttgctgaacttgagtttaagaataagttatggtccatttacaatcctaaaacaggtagtaataacatttctttagtattatttggcggaattatcaatgaaaatgcttttattgaatacgcaatgaggtcatttattaggtgactacatgttaatctcttgataagtagttataaattagtaaaaaggcagcccgctatgcgcgccagacacaagggtctgtttccacggctcgaacccgtaacctcctggtcatatgtcagcaacttagttattaattagtatacggtaaaaatgttaaataattgctatcacaggttaaagtttattgatagtgttaaggaatttttacactgctagtgccgtttagtttaaatgggaactagagtttaagttatatgcacttacggagtaagcatttttttttttacataatcaggttacttattaggtaattacatgtagatctattgactaagtaagcttttttcttgtaatattcagggtatacagagtcattggatgacaacacagagatagtagtgcctcaagatgattatgggctatatgcaattgatattttggatccttctatggtatgtttttgcatcatcaagtggtcacaagtgtatgtataggtcctatgtgcattagtatcatgtttcttcaataattttgttgtttgtagttttgctaggtacaaatttttatggggaggtgatgtaatcagtaatcttgaaaagtcatagcacctttatatatatagaagctaataatacctgagtttagaagctaaatagttacttgagggtatcttttttaaaaaataattacttgagtataagtacacttaagtggtaggtaaaatgttttgttccgaaggaaaaaaaaagtgataagtggaacatgaaatatgtgtttcatctatcaaaaagatattaccagttagtttcatctattatactctacatgtcctgcgaaaaataaatgtatgattcgtcttttgtttcttttcctttcctgaagtacttttctctaatgagatcatgatctcttctaattgtgtttgatttccagatggtgaagtgtgtacacttgaccggtgtataccattttcatgatatgattgatatgctggtcaaatgtggatataaaaaaggaaccacattgttcggttttggttacgacttccgccaaagcaataggtattattttaaccggtgaaatttccactgcccttatgccatatcgcattgtattatagcttattatttgaaaagccagttgattgtgatgatagcaaacaggaacagctcagttctatttaggcttatttgattcttctggtgaaactattttcgcacttagggatttggttagcatatggttgcaggattgacaaggcaatgaatgacctgaaagcaaagctagagacagcttataaagcttctgggggaagaaaagtcgatattatttcgcattccatgggtggattgttaatcaaatgtttcatttctctttatagtgatgtaagtccaattgctttagactcattcatttgcaaatcatctatgctatttaatcaagctatattaatcaaaagaagataaagtcgtgtcctaagtggagagaacaagggattggtctgttgttcccgttggtcaactatgtgtctcctatttaagataatgtattcctatttgttctgctcatgtatcttttctgttttgcttttctttctccttaattcctgcctagcttcttgatttagatttgtcactcttccttgagtggcatcgctaaatgcaaacagttgcctttcaggtattttccaagtatgtgaataagtggattaccattgccactccttttcaaggtaactttgctttctctctttgcagctttttgcagatgatgtcttttttgaattgctaggatacgagctaagatatccttatcaatcaggggatgcatagataatacagatagtgagataaatttgattgtcatatttgagcacctccaccttccaaaaaagaaaaaaagaagaagtgtgtaactagagaaggtcatggaagcatcagaaacttagcttttggtgggggaggtaccaattacaaaagtataaggaagaaccaataactaattgaaaaataattgcaaagaatacaaccaaatccatagcttccaaatctttatttatatattcagtaattaaaagaagtgagaaaaaagaagatgcacaacttcttttgaagattactcaaaatgtatgccaataagataatttctgcaaaaaaaatggtaatcagtatattttaagtagcaggtcctgactaaaaggaatacgcatagagcggaatattatatagaattgtataaaatctgagttgaccatttgctattatagtcctgcttagtcgaaagaccaggcgaacagtataaagttctgaaatataggaagaggaggaggaaataggaagagtgttatataaaagttattataaaccagtaatttcctaaagtagaaatactgaatctttaagaatcattgttaaaggaaaaattgtttgcaaatagtaatgcttataattggagggaggggaaggttgtaacttttttcttccagatttctagtgaaacgtattaggattcaccttctatttgtagggtcccaatgtatgagtggcgagggagagtccatggtcctgccagcgctcctgccctaccgcactaaaaagaagaaagaaaaagaacctaattgtgcccccccccnnnnnnnnnnnnnnnncgagggtgggggggtggaggtgggagatggagcttcatgaagaaacaatattcctgtgaaattggtttcgaaagagtctttaccatggctagagttcatgctccttaagaaatgcccctggacctcgaggctgctagttacaagtgtgcagagtactgattgttcatctcttgtaaataggtgcacctggatgcattaacgattctctcttaactggagtgcagttcgttgatggatttgaaagcaactttttcgtgtcaaggtggacaatgcatcaattggtaagctctcttggtcaaggacttttgcaagagccccgttaatagcatatgtaggaaacaaagcttcattttattaatagctccatatctgttacttggctggcaaatatgccttgatgatgaatttaatctacgtgcttccttatttactgaagattagactgtttcaacaaattaagctagtaaatagagattacgctcgagtatgtctaacttcatatgctcaattttcatttttatattatttaatcatatcttcaatggccctaggtagttagtagatgtatcagaagtaagactttccgaaactgtatgtttgaagtgtattgataggctttgccaaaagactttgaaccttactttttcatctcttaaaccatggtatctacaaaattgagttaaactggaaatgtcatttgattgcgggacgactgttcgttcacctggtagttcttgtattttattttttgtgatactcaatggattcactgatacggtctccatcaatactgcaaataattgtttgttcgtcaatcttgtcctttttaatcttttgcaagttggttgagtgcccatcaatctacgagatgttgcccaatcccgacttcaagtgggcaaagcaacctgagattttggtgtggaggaaaaaatctaaggagggggagacagtggttgaactagaatgttatggcgctagtacaagtgtgactctctttcaagaagcattgaagagtaatgaggtatatctccaagctatctttagccactagaatttttaactttatattacaagttcttaagtttcatgcttctgcatctctccaactcgccaattagctattaaaatgagaaggcttgtttgcttgctgagaaagcatttcgatctcatgcatgtctgaactagtcaaagcttaaagtaatgaggctttggtaaaatatgtgtaacatttttccagaggagcctccccagtgatgctgaaaaatgtcacttcgaccaattgtatttttaagtggataaatttaatccaaccaaaagcaagggtgcaactgctgtatgctcactttatttataacaagccagaacatgatgttcatatctgtgctgtttgtataacacatcaaatgaccatgaccttttccttatcttccttctatcaaaaattcactgagctcacttttatgagacaaaggctagcagggcttagaggttagattaaacttgacttacatttgtcttatttgtcatgttagagggcaagatgaaaattgttctattcagttctctgtaaaacatttttttttttttgcaccaatttaaagaatcataacatcgtctatgcagttgaaattttagaacttgtgggtgatgtacctcttcggatgtgtagcaactattgaaggatggccaaaaataaatcgtatgtctcatacataagaatataagatagagggagaatttgttattattattggttttgcatgatattgcttacaacctgcttctcttatgcatctttctatgactagaagccagtgtttgtagcaagtgattttttttatgccccgaattgcagctcaacttaaatgggaaaacagtggctctacctttcaacttatcaattcttgattgggcagctagtactcgcaaaattttaaacactgctcaattaccgcaaggaattcctttttatagcatctacgggacatcttttgatacaccttttgatgtttggtatgtaacttacatcttatcatacaacaagacaaggtagttcataactggttcgtttttgtctgaggttatgcactattcaatgtgtacgtgtttcttttgtagttacggttcaaaggcttcaccaattgaggacctaacgaatgtctgccatacaatggtaactaactttttctttttccttgcaagtacaagtgatattatgacattctaactctttttttaaaaaaaactggtaacttttatgacattctaattcttctgcattttttttctgggtttgtggttttattatcagggaagattcttttgccttgtgttcttattggtgtaggtagaaaccttgtgtctccaaaaaaggttttagagttagtagtgatataacctgtggggttaaggctccaaatagcttggctccccttctggctaaagttcaactcagacttaaggatttcttgggtaatgttatctgagatataaagccgttatgatcaactctttaactaagttattagtatgatatattagactggtttatagtcagcttaactaaggtcgcattcctgactagttattccaccaaatggagctaagaggcgggttaatttctggcaaatatgatatgaatctcgtgtttgttttctataagttttctccaggtatggaatttagtcaattgaccattggttaatcttcttgtaaactttgatccttatcttgtgtctggtttcgacgagtctgctctccaggtatggaattgactatttcccaaaacttggatgagtcttagatagcacttcccttttgtgaatgaccccatacagaatctttccttcaaaggtttcaccaaattttacttcccaaagccattcaaatatcctccaaagatataaatcccacagcttgagggatgatagtacgttttgttccagaagcatcagtaatgcatgtgatttttgttgtggtcacttgggggaatttataaacgaccttttcaatcttatttagggtgctttaatggagtgggtaacgactaaggtgtgaagtgtgcgtggggatgaaattcgttctgtatttagagatttacatgtctttattgttatataatgcttcctgaagcttttctacttactattacctattcctgtttgtgatatagccgcaatactcctacgtggacggtgatggaactgttccagctgaatcagcaaaggtgttattcttcacttctctactttgtttcttactcttcttatatattataaagactaatggtatcttggcacgtataagtaagaatctgattttaatgagtaagaaaaagaaaaacagatagcagtattaattttctgcggtgacatcagttttagttttcctacaggaaaacctggatattaagtaaaatgacagcttgtctatactatggtcgacttcgtgactatgaaacattggataggtgcttgacaaatgctcagtatcttggttgattgttgaccctctgctgataactttgcaggctgataattttgaagcagtggagagagtaggagttcaaggtggtcacagggaactactaagagatgagaaagttttccagcttctcaagaagtggcttggcgtcaccgataaaaagaaggcacattccaggtcctccagggtagtggacctttatccaggtcaaaagttgtaaacatttgttctgctcattcttagagcacgtttctgatctctctctcgctttattagtcgtagttataagtatatacgcgtgtacattcttcgagaatgttgaagttagcttctacatgaatcatcgtatgttagtgtcaatatgtattaggcactccaggatattagaaaatcagtccaaaacttgctgtaagaacaatgtttatcgtgtatatacacgagaatccagaggttttatcttttgttttctttactcctttttagctattcttgatttgtagaattaagaaatgttttcttggttttacctatagttttgtagagttaaaaaatgtaaaatcttgaatgatcttgctttaatcttccttgttgcagttatgtccaggttcaaattcttgttcatttattcatttctttggaggtgctgaaggcatttcgttactttttcttacctctaatagtaatctactcagcaattcatttataccaaacagtttaaagacaagaactgttctttattgaaatcatccttatcaatctcatctaaattgctatatctttggacagactaattcatagccaagtaatatttctacaattttaaataaatacaaaatcaaaatagaatactcaatcatgaataaataccggcaaaataccctctcctatgtatataatgtaaacaaaaaagtctatgtagctaaaatagtagtaaataaataaataaaaagaaaaaaagaaaggagcaatagcatatgtctaacttattatttgttctttttcattgttttgtttcccatactagtgcattacagagggtatctagctatctatatttaccagttagtggaatatacatataacattttgtaaatatattcctatcattcaacaaaccttattaatttaccatggttaagtgagaattgttaacaagatgtgtattgattaattataatttaatgataatagtatacgtgaagacataatattatattttcattgtattggtaccaacactggtgtcacacccttttttactccaaaagatataagttatggatcgtggattgaagagttttttcaattaaagtgacaaacttgagtagggattattttatttacagagtcgtcgcttggaattggttttgggtgttccaagtcaccttttatatgaatacttagtcagaggaagatttgactctattattattggtctgcgaaaataaagtctgggtaaagaattctgttgacaggggaaaaggtgtaaggcattccccgagtcccgtgattctagcacggtcgctttattgacttaaatttggcttgaattaattttggattaactgtgctttatttggttttcatgttttgcctatatctgtttttattgttgatgattttattaatctcaaactaggacgagtattcccgatcctagcttacactaaccaagacgagtgcccccgaccttagtttgtgcgttgtacctaaaattggagcctactcgaagttaattaaatagcaaacttttaattgatcgaaagaattggaacaagacgtattctcatgaccttgccattttatttatatctttatttcgaagcgtgtcttctcgacatcggttaagacaaaattaattatggaagaggttcgtgcatccttattgtgaaaaaaataaacttctttgtagctagaagataatgagttcgcatatggataattaattgatgttggaccaatttataaccttacattaaacaaatgatgaaacaagtataaactaagtatttaagagagttatccatattcgtgcctaaaatttgcctaacgcttaaattaaattattagtgactaagttattaaactgttagttaaactttaagagaaggttattcgactaacttagcgtgaagattttgggacagcttaggatatagtaattattgattatagaagatggacctaaaagcatcctagtaatatttggattaaaacagtaaggcaagaaaaaggcatgtatgtgattatttaaagccacccatggcataattttgaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntatatatatccccacaaaaaactagtcaaaataatttattatacgctacttacaagtacaggtaacttgagtcgaaaagaataaattttttttacatggaggaacaaataggatgactaacttgcaatttgaaatttaaacttgctactatgaaggagaatgaagttctctttgtatttgtaaaaaaaattaaatattcgttgttttgggagatattagcagactaacgaacaagataaaaaattgggaaagatcataaattagggctttaatcaataaaaaggtcttgacttctaaatttgatatatttcagttcatttttaaaacttttgagtaattacaagctgacttttgagaatttgggtattatatgaaggacttattcaacaaattttgttttaatttgaaaaagtctctggggcttacggctcaatacaaaaatgcgcctcaaacacccgaacgtacgccctgaacgcccgtgcgtatgccccgaacggctaggcgtacgcctcttgagactttcaccccagatcatcgcctcagagcgtttttggtggggttcgccccggaaacgtcttttaaaacactgattttgaccaatgaaacgaaatagtagatattttggagcccaattccctttatttttgtatccaacacttatatccctaacatgctactttcttttataccttcgaataactgaaactttgttaccaatcttaacaagaagaaatgctaacatatcagttgtagaaggcatataactgaaccaaactaaagatgctatcatgaatgtatataaatgtttcaatggaaaactacataaggaagtcaaaacagagggtcatgtaacctaaactcaaacatggaacatgaactaacaaagtaactttaattactaaagtaaaagtgtgaatgaaacttttttgatgaaactttacactacccagttcgagatagacttagtagaatagagtttcaaccacatatatgtaaagaagaactgacaatttagttaacttctaacagatttacatgatgataacactaacattgatagacttagtagaatagagtttcaaccacatatatgtaaagaagaactgacaatttagctaacttctaacagatttacatgatgataacactaacattgatagacttagtagaatagagtttcaaccacatatatgtaaagaagaactgacaatttagctaacttctaacagatttacatgatgataacactaatataacattcatctttaaacgaagtcagatctaacatgtttcaacagccaaaccaaggtcaaatcctttttattctcatcaagaatctgcaatataggaatttgaaagttacctagtttgtagaataataaaatacagcagtgcagcaacagaaattcagcagcaaagacagttgaaatagcaataggaacaacagtgaaatcagcagcgttaaagcaacccagaaatcttagtaaaaccagtgatttttcaagagaagaaactcagaatcaaactctaagtcttttctatttttctgaaagattatgcactccaagactcactcacaatttcagcttactaaaggaagcttcaactgaattttttcgcttaaagttgtgttctttttccggtgagtagagatatgtattctaagtgtgatagagtgtgtctgtaaagtgagaaaagagcagctttaagtaggcaaagaaagtcacttttcagacagattttggttcaccaaaagtctgtccaaaaagactcactttatgtgccaaaacactgcccaagggttgtccaaaaggtgaacggacagcctgaaaactagttactagtaccctaaatcattatcaaaccaatctaaccttcagattttatcaacatcaataccaccctatttgtaacaaaccaaaaacaagaaaccagtcactaaaactgattaaattgaactattaatcaacaagagatttcaaaacttaataactaaaaccaaagcaagcagaacacatgttaacaatgaagcaactagtgaagtcgattaagttaaatgacaacggaatcaaaaactagattcaatgttaaactaaaacgaaaactaccagaattgaactaaaacaaaagcactaacaccaaatgcaaaaatataaaattaggggcggggagaaagggaattgattttacccgatgtcgaacaccggggcagtggtggtacggtggaagtcgtgcggcaatggtgagggtttgacagtggttcggctagagagagattctattgtcgatcttcatagaaaccaaaactctgatgaatcggagactagtgttaggccccgtaaatgttacctagatcccggggtttcatgatgccgaggtaggctaatgtgttcaaaatttttagaaaacgagtaatggtacagaatttttgggttgtacaatgcatagtggaatgagtagaagaaattgcagaaaatgatgattctgtggttcattatgttatagcggatctgttctgctaaccacgaaatgaccacataatgaaccagaaataattcaaattttgaaaactatttttgctggcaacataatgattctactggccgtataatgattatgctatcatgaaattgcatcgcggaatggactagactcaacccagaatttaaaaattattttgtggccattatgccagctgcatgatcattctgctatcgcagaattgcatcgccgaattgacatatcattttgtggatattctgctgcccacataacgatttcgctgcccacataacaattctgctaccacataattgcaccacatgattgacttggggggtcatttttggaaattttcttatccggtcctattttgatacaaggcctcattttactagataaaatctgatattttagagagaggagagagttgttccagagagagaaagaagggaaccaacatcttcatgcttcaaattctcgctcaatcattggagattcatgggagaaatccacaagatcttcatctaataggtaaggttctactccctaaccctaaatttcgaaatttggactaaaagtagtcatcagaagtatgattcttaggtgtgggaattgctctttgtacatgaatgtaaccataagggtggtagggaggttattgaactaaaaggagtaaactcttggtattgaattggttatgggtgaaggaaatcttgtagaagaaccttgtagtcaaatttgcacacctagtgtgtgatgacccaaaaggtcatcttatattttagacctcgaatctgcactcttaagtcttaaaaatcttatatttatcctcctcgattttcatgagcagtctgggcatgtttccggaaagctttatgttgaaaattgatgaaaacaagaatttatgccttaaaaagttgattttatttgatttcagtcaatatttttggtaaacgggtccagatccatattttgacagtcccggtgggtccgtatcgaattatgagatttgggcatatacccagaatcaaatttcgaggtccatagcccgaaaaataaatttttgactaaaattgaatttcagaaaattaatgattttaaagtatatgcttaatgcttgattttgatggtatcatgcccgtattttggtttcggattccggtacaggtccgttgtaatatttgaactatgtctttgaaatttggtaagaaacggagtttaaatgacgtaattcggaccttcggttgagaaaatagtaactttaagtgttcttcagaatttcattgattttgatgctaaatctgcagtttaaggtgttattttggcgatttgattgcacgagcaagtccgtatgatgttgttagacttgtgcgcatgtttggtttggagtctcgagggctcgggtgagttttggataggttacggggtgatttgtacttagaaaatctgagatttttgctgcagcagctattcttttgtgcccttcttcgcgttcgcgtaggtagtgtcgcgaacgcgaaaggtaaaatgggggcaggggaattttcttcttcgcgaacgcaaagcattgggggtggtacccttcgcgaacgcgatcagtctcccgcgaacgcgatagtttaaggggcctgggggagggggggtcatgttcttctacgcgaacacgaccattgggtcgcgaacgtgaaggcttggagggagatgccttacacgaacgcatagaaagactcgcgaactcgaaggccttaggctgttgtgtatcgcgatcgcgacaggcctatcgcgaatgcgatgaaggcatgtccagtgttttaaaacagacttcaaagacgggatttgttcaaaatttcacattttcttcacttgaactcaaccttgggcgatttttgaagtggaaattcaacaccaaatcataggttcgtgttcttaaactcattcctttcaatttccatcaacacccactagatttctaggcctaaatcttgttctttaagggtagaaattagggattttaggagaattagggattttgtaaatttgggaatttagaccccgatttggggttggatttcggaactaatgacatattcgggctcgggggtgaataggtaaacggagtttggtccgaacctcgagtttggaccaagcgggcccggggtcagttttttacttttttgagaaaatttggaaaatatgtaattattcattaaagttgattcctttagcgataattgatggtattaagctatttgtgactagatacgagtgagttggaggtgaattctaaggaaaaagcggtacttgagcagtgagtggcctgcgaatcgaggtaagtgtcgtgtctaactttggcttgagggaataggtattatgtgtttatttgctacgtgtttggttgttaaatacgatgtataggtgaggtgacaagcatctatgcgtcgttgtcgagacatagcatgcgagtgaaattctatttaattctattttgaagccttaaattctactatccatgcttagcgggttatttgaaatgttgagtgacttatatctggtttcactgagatttggtaactttcgaatattaattcaaggttgaggttacattgtgctattgattatggataaaatactgatttctttgtgatactcctatctatccgttgttattgattctgtgatttgtgaggaggagtgtaaagcacgaagggtgatgtcgtgcatgcattatttatattgtgaggaagagtgtaaagcacgaagggtaatatcgtgtcgtgatatgtgagtgttaatgcacgaagggtgatatcttgccatgatttgtgagtgataaagtacgaagggtgatgtcgtgcatattatgagaggtttaatgcacgaagggtgatgccatgccgttctatttatttatttggtgaggttgagagtaaaagcgcgaagggtgatgctatgcagttttcctttgctgttttatttactcaattcgtttaaagattttcggtttaattctgtcttttcattattctccctctttatattgtattcccccactgtatgttccctttccattatttctgcgttacttctttatttactgttgttgccactggcatgattatactgtttaggttatatgtgggtgtcttgtcctagcctcgtcactactttgctgaggttaggctcgacacttaccagtacatggggtcggttatactgatgatgcactctgcactttctgtgcagattttggtatcggcttaggttgatcgagattttactattggtccgctgtccggagactcaaggtagatctgtcggtgttcacagatcttgaaatccccgtctatcttttatgtcctactgttttctttcattcagacagttgtatttctttcagactattacttatagtaaattctagaatgctcgtgaattgtgactccagatccgggtggtagtaattaatataattttataatattctgcacttattatatttcatcttagttaattattgttatttactgaatggaaataaggaattggtttaatgattctctaacgttggcttacctagcaagtaaaatgttaggcaccatgacggtcccgtcggtgaaaaatttccggtcgtgacagttggtatcagaacactaggttgcctatatctcacgattcacgagcaagcttagtagagtctggaggattggtacggagacgtctgtgcttatcttccagagactatgaagtttaggaacaagtttcacttctattcttctctgtcgtgcaattttgctttctcaatactgattgaactcttctactcttattctctcgcagatggcgagaacacgtaccgcttcctcagctgatcagcagccagagcctccagtggcacctcctacgcggggcagaggtcgaggctgtgccagaggccgaggcagtggcagggctcagcctaaagcccgagcagcagccccagcaatggagcctcatatagagcttgacgaggaggttccagcccagactgttcctaccggaccagcttaggttccggaggggttcattgccaccccagtaattcaggacgttttggtccgtttggtgggccttatggagagtgtggcccaggttggcgcatttcccatggcaccagcagtttcttaggctggaggaggagcccagactcccaccactcccgttccgaagcagatagctccccagaatcaggctccagtagctcagccagtcggattagttcagccggttattgcggcacaggtcggagatgggccagctatgtcttctgaggctttatggagattggacaggtttatcaacctgttcacttcagtggtgctccttcagagaatccccaggagtatcttgacagctatcatgaggttctacggaacatgagtatagtggagaccaactataccttgacagctgttgcgtctatatacgcgagaatccagaggttttatcttttgttttctttactccttttttgctattcttgatttttagaattaagaaatgttttcttggttttacctatagttttgtagaattaaaaaatgtaaaatcttgaatgatcttgctttaatcttcgttgttgcagttatgtccaggctcacattcttgttcatttattcatttctttggaggtgctgaaggcatttcgttactttttcttacctctaatagtaatctgctcagcaattcatttaataccaaacagtttaaagacgaactgttctttattgaaatcatccttatcaatctcatctaaattgctatatctttggacagactaattcatagccaagtcatatttctataattttaaataaatacaaaattaaaatagaatactcaaccatgaataaatactggcaaataccctctcctttgtatataatgtaaacaaaaatatctatgtagctaaaatagtagtaaataaataaataaaaagaaaaaaagaaaggataatagcatatgtctaacttattatttgttctttttcattgttttgtttcccatactagtgcattacagagggtatctagctatctatatttacaaattagtggtatatacatataacattttgtaaatatattcctatcattcaacaaaccttattaatttaccatggttaagtgagaattgttaacaagatgtgtattgattaattataatttaatgataatagtatacgtgaagacataatattatattttcattgtattggtaccaacactggtgtcacacccttttttactccaaaagatataagttatggatcgtggattgaagagttttttcaattaaagtgacaaacttgagtagggattattttatttacagagtcgtcgcttggaattggttttgggtgttccaagtcaccttttatatgaatacttagtcagaggaagatttgactctattattattggtctgcaaaaataaagtctggataaagaattctgttgataggagagaaggtgtaaggcattccccgagtccagtgattctagcacggtcactttattgacttaaatttggcttgaattaattttggattaactgtgctttatttggttttcatgttttgcctatatctgtttttattgttgatgattttattaatctcaaactaggacgagtattcccgatcctagcttacactaaccaagacgagtgcccccgaccttagttcgtgcgttgtacctaaaattggagcctactcgaagttaattaaatagcaaacttttaattgatcgaaagaattggaacaagacgtgttctcatgaccttgccattttatttatatctttatttcgaatcgtgtcttctcgacatcggttaagacaaaattaattattggagaggttcgtgcatccttattgtgaaaaaaataaacttctttgtagctagaagataatgagttcgcatatggataattaattgatgttggaccaatttataaccttacattaaacaaatgatgaaacaagtataaactaagtatttaagagagttatccatattcgtgcctaaaatttgcctaacgcttaaattaaattattagtgactaagttattaaactgttagttaaactttaagagaaggttattcgactaacttagcgtgaagattttgggacagcttaggatatagtaattattgattatagaagatggacctaaaagcatcctagtaatatttggattaaaacagtaaggcaagaaaaaggcatgtatgtgattatttaaagccacccatggcataattttgaatacataatacacaagattttcatctaataggtaaggttctactctcaaatcctaaatttcggaatttggaccgaacgtagtcatgagaagtatgattcttaggtgtgggagttgctctttgtacacgaatgtaaccataagggtggtggggcggttattgaactaaaaggagtaaactcttggtattgaattggttgtggggtgaaggaaatcttgtagaagaaccttgtagtcaaatttgtacacctagtgtttgatgaaatgcttaaatgaggtgaaaccctcaatatctttctaatttacgtttaattctattatgtctcaaaatagattgagattgctaggattttcggaatattgtagtaacttaaggaaagctcaagcaaggtacgttggctaaactactatgatgacccaaaatatcatctttaaatttaataagtaattctatgttctaagaactcaaaaagtaccatttatcattcctcgacttgcgtgcgcagtccgtaaaatttttcgaaaagttttcatgtgaaaaatggattaaaataggaaatagagttttaaaactcaattaagttaatttaggtcaacattttgagcaaacggatccggatcagtattttgaaaattcggataggtccctatcgtgatttggtacttgggcgtatgcccggaatttaatttggaggtccctaactcaagttatgaccatttgacggaattagcaatttaaaggctaaatatttcaagtttgaccacggagttgactttttgatatcggagccggaatccgattctggaaatttgaacagcttcgttatgccatttatgacttgtgtgccaaatttgaagtcattccggattcatttaatatgttttggcacgagatttgcaaattgaaagtttaaaactcaaagttcgaatcggggtgtgaattgtaatttcagtgttgtttgacgtgatttttgaccccgagtcagtccgtattatgttacgggacttgttgttatattcgaacggggtcccgagtacctcgaaagtgaaacggatcaaaatcggaacaagaattggacttaagcaaaaatctgaaattttgagttctggtgtaatcgcacctgcagaattttgaccgcaggtgcgagctcgcagaagcgagacttccatcacagatgcggccttcgcaggtgcggcccttttgcgcagaagcggacgtcgcaggtgcgatattttgtccgcagatgcggaacttcgcctggcagtcccttttcgcagatgcgagctcgcatgtgcgagcccaggcaccgtaaatgcgaatatactgggcagaatacataaaatcgggtcttagccatttttactcatttttgagttttgagtctcgaatttaggtgatttttgggcgatttttacgggaaaatattggggtaagtgttccttatcctatattgattatatttcatgatttcatactcatttatatcatgaatctctgaatttatggaagaaaaaatagatttttataaaatcttccaaaaacgaaaatttaagatttgaaggtccatttgatatcggaattggacaaatttggtatggttgaactcgtatcggaacgggtgttcaaatttcgcgagttttttcaggatttgagacgtgggtcccactgtcgaatattttaatgaatttcggatttttatccgaaaaaattataaattcatatggaattaattcctatgattcgtattgaatatattgaattgtttgtgaatagatttgaaacttttagaggcaaatgtaaaaggaaaagttgtggttgaatagttgattggaaattacaaagcgaggtaagtgtcggggttaaccttgacttgagggaatagaacccttaaattgttcgttatgtgaattgcatgtaaacgacgtataggtgaggtgacgagtgtctatacgtcgtcaaattaattgtttgcctgcttacttaaaaaattataaaaaatttttaatcataaattaattattataatatttgtttctctcttattctttgcaaatataaatttttgaattcctgcattaattgttacatgctatttgaattatgtgccttaattgttatttgacatttagcataataaatattaaattgcctatttgctccctgatttctctaataatttgctatttgtcattgtttgcttcataattaaaccataattattgtatgcttgttgtcttataattttatattaattgttatatttattgggaaaatttcttctataagaattggtaaatgaaaatgttggaggatcgggttgcacgccgcaacagacttattaaaagtcaatattagaggatcgggttgcacgccgcaacaaacttattaaaagccaatattggagaatcgggttgcacgccgcaacaaacttattaaaaagataatattagaggatcgggttgcacgccgcaacagacttgattaaaatgaataagttggaggatcggattgcacgccgcaacagacttgattaaaatgaatatgttggaggagcgggttgcacgccgcaacagaaattgaatgtgaatatattgtgagagcgggttgcacgctataacagaattggttgacataataatggattatgattgctgggttagtttcaattattataaatgagttacctgatttatttatattatttgttgttattatcaatattgcgtacaggttaatgtaagtaaaccgccttagcctcgtcactatttcatcgaggttaggctcaacacttaccagtatatggggtcgattgtactgatactatactctccacttcttgtgcagattttggaattggtcccagcggcgtaccatagatttgctcggattttagctactcggaggagacttgaggtataactgcatggcgtccgcagttctgaagtccccgtctatctcactttagctgtgtgtttatttccagacagctttattttattcagacctttatttgtatttattctagaagctcgtgcacttgtgacaccaattttgggatggtatttagacaccgttattattatggattatttcaaaattacttccgcatttgcttccttgttattaataaattttaaaaaataattaaaatggacaatattattctaacgtcggcttgcctagcaagtaaaatgttaggcgccatcacggtccgaaggtgagaatttcgggtcgtgacagttggtattagagcactaggttacataggtctcacgagtcacgagcaagcttagtagggtttgaaggatcagtacggagacgtctgtacttatccctcagaggctatgaagtttaggaacaatatcacttctttcttattctgtcgtgcgattctattctatcatcaatgattgaacaattctactcttattctctcgcagatggtgagaacatgtaatacttctaccgatggacagtgaccagaacccccggtggcaactatggccaggtgtagaggtcgaggccgaggtcgtgctagaggtcgaggcagaggccgtggtagagctcagtccagagctcgagtagctgcacctgttgtagaacctcagatagaccttcaggaggaggtcccagttcagaatgtaccagttggactagttcaggtcccggaaggattcatagccactccagtacttcaggacgctctagtccgattggtgagtcttatggagggtgtggcccagaatggtacatttccagtggcaccagccatctcacaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncttattgaggctggataagtttactaagctctttccagttcacttaagtggtacatcttctgaggacccacaggattatcttgaacgctgcctcgaggtgctgctaaacatgggtatagttgagaccaatggagttgattttgctgtattccagatgacgagttccgccaagaggtggtggagagattatacattgacccgaccagtcggatcacctatacttacctgggagcagttctctcagctatttctggagaagttcctccctatcacactngaattctgcaagcaatttgagtgtctacagcatggcagtatgattgttactcagtatgagtcccgttttgtggatttggcccgtcattctctccttttactacctacggaaggagagagagtgatgaggttcattgagggactcaatcaccctatcagacttcagatggccaaggagaccggaaatgagatttctttcaagacgactgctaatgtcgcaaggaggatcgagatggtccttgcacaggaaagggggcagaggtccgataagaggcctcatcagtttggtggttacagtggtgcctcgtctaaaggcaggggtaattttggtaggggtcatcctcccagaccgtttcattcaacactttatgtatctcccggtgcttcagagagtcacaatcctattattccttactctgggcagccagtattcagtgcacattcagctcctatcagtgcaccaccactccagagttactatagtggttatccggcccatctgggtcagcttcagcttcagcagccacgacatcaggatggatgttatgagtgtgggaacattggtcacatcacgaggtattgccctagattggcgagtaacagatctcggcaagattctcgtgccatcataccagcaccggttgcttcaccgccttctcagctagctagaggtaggggtcaggcagttagaggtggaggtcaggccattagaggtggaggtcaggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcacacaattccaggtattctattcatccaggtaccgcaaaaatgtatcaagatttgcggcaacattatgggtggaggagaatgaaaaaggataaagttgcacatgtagctcggtgtctgaattgtcagcaagttaagtaggagcatcagagacctggtggtttgtttcagaaattagaaattcctgagtggaagtgggagcgtatcacaatagattttgttgttaggctcccacggactcagagaaaatttgacgcagtttgggtcattgtggacaggctgaccaagtcagcacatttcattccagtggcagttacctattcttcagaaaggttagctgaaatttatattcgtgagattgtctgccttcacggtgtgcccgtgtctattatttcagatcgaggtacgcagtttacctcacatttctggagggatgtacaacgtgagttaggcacgcacgtggagttgagtacaacatttcatccacagacagacggacagtcagagcgcactattcagatattggaagatatgctccgcgcttgtgttatagactttggaggttcttgggatcaattctttccacttgtggagtttccttataataatagctaccaatcgagtattcagatggcttcatatgaggcattatacggaaggtgatgtcgatcaccagttggttggtttgaaccgggagaggctcggttgttgggtaccgatttggtacaggatgccttggataaggtcaagattattcaggatcgacttcgcacagctcagtctaggcaaaagagttatgccgaccgtaaagttcgtgaaattgcattcatggtcggagaacgaatattgctccgggtttcacctatgaaatgtgtaatgaggttcggaaagaagggcaagttgagccctaagtatatcggaccctttgaaattctggaaagggtgggtgaagtagcctacaggattgcattaccacctagtttatcagcggttcatccggtgttccatgtgtctatgctccggaaatatcatggtaaaccgtcccatgtgttagatttcagttcagtccaattggacaaagatttgacttacgaggaggagtcggtggctattctagcccggcagatccgacagttgaggtcaaagagttatccttcagttcgggtgcaatagagaggtcagccggtagaggcatctacttgggagttcgagtcggacatgcggagtaaatatccacaccttttctccagctcaagtactttttctaactctattcgaggacgaacgtttattttagaggtggagaatgtgatgacccaaaatgtcatctttaaatttaataagtaattctatgtcctaagacctcaaaagcaccatttatcattcctcgacttgcgtgcgcagtccgtaaaatttttcgaaaagttttcatgtgaaaaatggattaaaatgggaaatagagctttaaaactcaactaagttgactttggtcaatattttgagcaaacggatccggatcagtgttttaaaaattttggtaggtccgtatcgtgatttgggacttgggcgtatgcccagaatttaatttggaggtccctaactcaagttatgaccatttaacggaattaacaatttaaaggctaaatattccaagtttgaccacggggttgactttttgatataggagccggaatccgattctgaaaattagaacagctcgttttgccatttatgacttgtttgccaaatttgaagtcattccggattcacttaatatgttttggcacgagatttgcaaattgaaagtttaaaactcaaagtttgaatcgtggtgtgaattataatttcagtgttgtttgacatgattttagaccccgagtcagtccgtattatgttacgggacttgttggtatattcgaacggggtcccgagtaccccaaaagtgaaacggatcgaaatcagaacaagaattcgacttaaacaaaaatctaaaattttgagttctagtgtaatcgcacttgtgaaattttgaccgcaggtgcgagctcgcagaagtgagacttccatcgtagatgcggtcttcgcaggttcggcccttttgcacagaagcggacgtcgcaggagcgacattttgtccgcagatgcggaacctcgcctagcagactcttttcacagatgcgagatatttctcgcagatgcgagctcgcatgtgcgagcccaggcaccatagatgcgaaaatactgggcaaaatacataaaatcgggtcttagccatttttactcattttttagttttgagtctcggatttaggcgatatttgggtgatttttacgggaaaatattggggtaagtgttccttatcctatattgattaaatttcatgatttcatactcatttatatcatgaatcagtgaatttatggaagaaaaattagatttttataaaatcatccaaaaacgaaaatttaagatttgaaggtccatttgatatcggaattggacaaatttggtatggttgaactcgcatcgtaacgggtgttcggatttcgcgagtttttccaggatttgagacgtgggtttcactgccgaatattttaatgaatttcgaatttttatccgaaaaatttgtaaattcatatggaattaatttctatgattagtattgaatatattaaattatttgtgaatagatttgaagctttcagaggcaaatttaaaaggaaaagttgtggttgaatagttgattggaaattacaaagcgaggtaagtgtcgagttaaccttgacttgaaggaatagaacccttaaattgttcgttatgtgaattgcatgtaaacgacgtatgggcgaggtgacgagtgtctatacatcgtcaaattaattgtttgcctgcttacttgaaaaattataaattatttttaatcatacattaattattctaatatttgtttctctcttattctttgcaaatataaattcttgaattcctgcattaattgttacatgctatttgaattatgtgccttaattgttatttgacatttagcataataaatattaaactgtctatttgctccctgatttctctaataatttgctatttgtcattgtttgcttcataattaaatcataattattgtatgcttgttgtcttataattttatattaattgttacatttattgggaaaattgcttctataagaattggtaaatgaaaatgttggaggagcaggttgcacgccgcaacatgattaattataatgaatatattgaaggatcgggttgaacgccgcaacagacttattaaaagtcaatattagaggatcgggttgcacaccgcaacagacttattaaaagtcaatattagaggatcggattgcacgccgcaacagacttattaaaagtcaatattggagaatcgggttgcacgtcgcaacaaacttgttaaaaagttaatattggaggatcgggttgcacgccgcaacagaaattgaacgtgaatatattgtgagagcgggttgcacactgcaacagaattagttgaaataataatggattatgactactgggttagtttcaattattataaatgagttacctgatttatttatattatttattattattatcaatattgcgtacaggttaatgtaagtgaaccgccttagcctcgtcactacttcgtcgaggttaggctcatcacttactagtacatggggtcggttgtactgatactatactctccacttcttgtgcagattttggagttggtcccagcggcgtaccatagacttgctcgaatttcagctactttgaggagacttgaggtataactgcatggcatccgcagttctaaagtccccgtttatctcactttaactgtgtgtttatttccagacagctttattttattcagacctttatttgtatttattctagaagctcgtgcacttgtgacaccaattctgggatggtatttagacaccgttattattatgaattattttaaaatttctttcgcattttcttccttgttattaataaatttaaaaatatttttaaaatggacaatattattctaacgttggcttgcctagcaagtgaaatgttaagcgtcatcacggttcgaaggtgggaatttcgggtcgtgacaactactctcttagaattgaattccatgatgttcctgtatgtttcaagtatggtcggctcaagttccttattcccatgttccgagttgttcccaataaatttgattgtcccgagtaagcaaagtgtcgagaattatgtattcaaaatatattccggatattcctatcacattatattatccttcgggagtgtgttcaagaatgatatgtgtattaaaatgttatggctttgagtcgtgtttcaaatgaaagtccattatgctttacttggaaaaactactacatgcgtctaaaactcctattgctcaaatgtgtatttaaagtcttgagtagaaatgccttgttgttgataatctataaggatgcttgaaagtgaaagaagtgggttggagatataaagtgtggccaacgtgccaagaatgagagttttacttgtggtcaatggggtcgaggaaatgaaatgatgtgagaaaagttatgaaataggccttgatttaattgtcccaaattgacctcgaaaatagatttgcctaaaagcttatgtacccaagttatgcccaaatgtgattgttttaactaatgctatgctttgttagtatttccaatgttttttgagctcttatgtcttcatgagttgaaattgtatattgccctttttggagaaaagtatttcaagaacaaatgatgtgttacacgtttatgattttaatttgtgcttcgattgcgaatccatttactcaatttcttgaaaagaaattcattgtgtttaaagtcttcattgctaataacctaaagttgtgattttcggagaattctatttgttgatatttatgatgatgatccatgaaagaaaaggaaataattaaagtgtggaatgtggaatacgaccattgtgccatgaataaagaattatatatatggccaagagagccaataaaataatgatgttgaaagtggttgaaagtgccaatgaggctataaatggtgtgaaaggttatgaggtaaatgtaattgtatatctgtttcccttgtatgaaaatcaaaaatattttttttttctggagtatcgttagtcagtgaggaagggtaggttgacataacctaaccccggaatacacctccgcccgcatacattgtggtgaggtggcatggttgctttgtgtagagattgtgattgtgaatacacctccacccgcatacattggggtgaggcgacatgaccactttgtgtaggtattttgattgtgaatacacctccacctgcacacattggggtgaggcggcagagccactatgaatagagaatgtggtattgaaattacacctacacccgcacgcattggggtgaggcggcatggccactttgtgtagagaatgtggtattgaaattacacctccacccgcacgcattggggtgaggcggcggggccgctttgtgtagaggttgttacaagggatctcccaaccaaaaatcatattactatcgcgttttgaaaattgttatgttttaacttggcatttggatatcattgatcgttgcttgttgtttccatggtttttttttttttttttaatattggcatactattttgaaagaggacaattagctttacatactaatactattctatatgaactaatgtccctttagttgggggcgctgcatcttcaattgatgcaagtagctcgacaacagtcaactttggatctcattagtagttactccctattcaacaggttttggtgatccctactctattctgggcctcatgtcacttgatgttgtactttgtgttttgaggtatagccggggccttgttgtcggcacttttatactactcttttgttctacttagaggctccgtagataggttgtgggtggtgtttgatattgggtaatgagctagaagtgttggtatttggcaaacatgttttccttgtaattatgaacttgtaatattttgaaaattataaattgaatctccttgagtaatgaaaaagaatgtggagcacttgattcttctcacatatatttctagccactgattcttatattgctgatgggcaagattaggtagaaggcatctaggaggctttcttgatcagggtatctcggttgagcgtcggtcgcgctccccgaggtcgagcgtgacaactagcttcaatgaaatcgacaataaaacctcgcttgaggccggtatgaatgcagcaaaggctaaatctcggagatcttgagtggttcaggggcggcagtagagtggccggcggcagtgggagtggttggacggaggtagtcgggttccggaggtaaaactaaaggaacgtttatgtgaaatttgtgtggtggtttggtcggatttgcaatgaaatcaaagagggaagtaagaggtagctatctagggtttcgatttcatgattttcgggattttgattgtgcaccgccggtagtggcgatttacggcaggcagagccaccgtggggagggtgacagcggcagaagtgggaagagggagaagagagagctaagggtgagaggaaagaaagagtaaaaggaaaaatggggtcgtcaaaatttcggagggtaaaggcgttaaatacctagttgagagattgaatgctagtcactggatcaaggaagatcggatggatggggattgatatctaaccatttatccaaaacgacgtagttttaggttaaactgcgccgttttgaatccgcccctgacaggccctctattagaccgggttaggctgccaatttgcctcatatattggcccaatctcggctcacaaatgcaaaacccaatccttttaaccccgtaacaaactaattaataatctaattctaaaatctataaacacacacagtaaacctaggggaaaaaaatagtgtaggagaagtagaaagagaaaatattagatatttacaactggttgtaagtaagctttttccaccccaacccatatcctttgacacttgaccatcactttttatccgtttataattaggaaatgttactagtatatagtaacttaattgaaatttaaaaataaaaacagtgacatagtggttggaaaaaaataatcgacttctcttattttaaatgtgcaacagatgaaccagagtacaaggactaaagctatcattactgctaagtacgaaacttaaggttgaatgcacttaggacttgggagggaaaaatctttttcctctcattctcttttcctatctttaggtcacatacattcaaagagtgaagcagttggactagtatatgtcacatatatgtctttaaaacagtactataaatataaaataaccacgtgaaatgtatgtaaacaatgtatatagaaaatatacatacacaacaaatggaaaaattcagattatctgcattgttacgttatccaaaattacaaagcatggttgatatttttactttccgataagacctataagaatgaagttcccaaaattatagccaagaaaagagactaaacaaagctctctcatcatgctattcatgaacttcagtcttcagttcctataaacaaaattagcatttgcataaaagcaaccaaaataaaaccaaaaaaaaactcttatctttcttgagttgcaaaaaaaaaaaaaggaagattagagattagttatgtatacatcccttgctaactagatttcctcatatagaccatggccttgtgatgaggagttctatgtgtcattccacacagtgaaagtgggggccccaccatgacaaatagcgacagtattattccttgaagcgagagcaggaacagctgttgacatccaattgttacaattaacagactcctgatgatcataaagaccggccacctttactaagccattagatgttgattgttgagaatgatagtaaccaaatatattctcattaccaagttgttgcttcacttcattctctccatatccattaccttggctcaattgtggcaacgcaaatccaccactgggactacttgcatatccaactccttgatatgtaacattattatgttccattgaagtagaagaagtatccaatcccataagattgtgtaaagctgaagttggttgcaagaaattatgagcaatattcccatgttgaagttgatgaaaatcttgaaaattttgagtactactcaaatgatcagaatcttgaacttcttgtttgcaccatagcctttgttgttgctgttgttgttggctataagggtaatgcatattcaatggctgagcctgagcttgttggaatgcaatgttagaccagatatgattcgcgttcgcgccatagctactaattgttccatttgtgagatgagatccaatgttttcttgactaactctttgataatctagagcaatttgatcagcttgttcagcatctttgagacgtttagctgcacctccaataggtaaggtactgctctctaatatgctttttacgtcgtatcggtttatttcaaagttggttactgcatttagtccacggaatttgattgctgctatgtcataagcctctgcagcttcttcttgtgtgcctataaagaggaataaagttagaaggtaaacatcataatatgattttccaggataatggtttatggaaaaagcagttttgtacattgactatatacctaagaggtcatttggtacgcgggataaggtgggataaggtaagagattaaatttataccatgtttggttgatggtataaatttatcggtataaattttattccagacttaaaactaaaactggatatctcacgctatccccttatcccattaaacttggaattttttatccagagattataatcatgtgattattatccgaggataacttagtccgcttaccaaacgacccctaaggacataatatgacactatacctatttaatagtcttttggagcgaaaaaaagcctaaatagtcatccatccagaagcgaagccacgatttgaagtttataagttctaaatttgtttataaagggtataaacaaaaactgttaggttcgcccaaacctgtagatgatacactagctccgcccctacgtcgacccaaccacttaaactaaatatagctggtggatatacaatatgcatataatccatgtatactttgagtataacgtgtacaatcagtgaataatttatgtatgctagctagaaaaagtaaatagttaatttggacggctatttgtgtaaattccaaaaaaaagagaaaaaagacttactgaaagtgccaagataaagatccttgtttccagcaactcgtccaattcttgcttgccatcttccatgttgatggtgccttttattacaaaaaaaaaattacaaacctaagttatttttctttactattttaggaaagaagattttaagcaagagccaaatgatagcatacactaaaagaaatcacatattaccttgtaacacctcgataaatggatgcaccacgagagaacccactactcttcctatattagggaccagaaaacaaattaaaaaagaaaaatattaatttcattcattttctcgccataatttttcatataaacatgttttaaactaacatcaagtgttcagtaggtcagactaaaataatacgtcttaataaaaatatttgacaagattagaggtcaatctatgtatttcgctttttttcttaaaacttgattaattaatgtatagctttaaaatttaccttcgcaaagatgccacatactcttgtctagtcatatgcttcatttcatccagctctttctcataattactaatctgaaaattttgcatccaaaataaaaaattaaattaaattttctttacggaaataaatatgttttcttaccaactagacaaatttcccaggatatagagaagtcctagaattctatgtacttactgggaaattggtggtagttgtcggaccccaatacttcagtgctgctaaatcataagctcgagctgccttttcttctttatcataccctcctattaaagatttacaagaaaaaatagatcagttagataaaattataaaaaaaaaactaacttataaaaattgacagtataaagaagttaacattctcgtgaattttaaaatttattgtaacaattaacatgtcttattttcttggttgctaattccctttttttaacctatatttactatactatttaggtaacatgatagtgtaaaaaatttgttactgtctaaacgttaaaagcattgataaaatagtgagttcttgcattatagtaacatgttgaaaagtgtaagtgtatatatacaaagattagaaggaattacattataacttacccaaataaactgtaggaagtatacaaatgtcgaatccatcaacatgaaatagaaggagagaaaaatgaagaagatatccaagttagtattttaaaggaaaaaagaaggtatatagtagtttaaaaaaaaaaagtaatggcgagtaaaatgaagaaaaaaaaagtgaaattatatagtgggagacaatgaagggaaacataccttgccttcctttgcgagtttgtccctctcttctgcaactattatcccacaaatgtgcctcatatctccccgtccacctatgcctacatacaccaatatataaaattaattacaaaacgtatatcacaaataaaatacttactttacacttaatatcaaccactaaataaagcaagaggaacaagaaagaaatttggttataaaggttgtttaaaataagagaaaaaaatttcgtttcgagtagtagatcattttagggtagacttgntacatatattggcatcaattacgccttagtcccagacaaataaattacgaaatgtatatcacaaataaaatacttactttacacttaatatcaaccactaaatgaagcaagaggaacaagaaagagatttggttataattaaaggttgtttaaataaaaagaacgaaatttcttttttgagtagtatatcatttggtgccttcttaagtgacgataatccaatagtgaaactaaggattccattatttacgtttttgttttggtactaagcacaaaaaaaaaataaagaataacaagagaaactatatagggagaaatgggaagaatgaggctaaatatatttaaagacgattttgtagtttttttttaactcccttcccacttttgtaaggcaatcttgtactccattttagacttctcttgaggacaaagttcattagaaattttcctatatgtaatatgtctcataaatatagtatattttgatttttaaaaaataagaatgcaagaaatgctacttcgttctttttttcaaagtagcatttatttctctaaaacagccaaaaagggcggaagtggcctcgatcttacctataaaatatagcgacaattcaaaaaaaaggaaatagacaataccttgtaacaccacggtaaatcgaagttctttggccaaaagtatcaatagatttccgaggtactgcttctattgcaccattattttgtgcatcaatactaccatttgtaccatttgcattctcttgttgtttgttattttccgacgagcaactattctctccaccaccgccaccgccaccaccggtgctcatagaaagtgacaaggactgtgcattattaccaccacccaccaccacaccctccccctcctttttatcatcttgtaatgatggggtgcttgtattagggttatttcttaaccaattcttgatcatagaaagccctatagtattattattagtgttacttccaatattgttagtggtactagtaacactagaataaatactataatctccattaatgttgttgttgttgttgttttgtacttcaccaataccaaggaagttttctagttttggtggttcttgattttcaagatgttggctagtattgcatgaaatacccattagcattgacatctctgatgttgttttgtagttaggatcatgagagctccaatctgcatatacaaaacaaacaaaaaaatctcaaaaataggcaaaatagttgattttttttaattaatccatttaatcggactttcacctcctgtggtgatgaggggttttgacataaaccccaaatatgtataattaaaaccatagtattgcatctaacaaaaatcgttcttaatttgtctaattcatccgaggtaaataaaggacaacaacaacaactcagtataatacagagtctgtggaggataatgtgtacgcagaacttacgcctatcttggtggatagaaaggctattttgcatagacccttagatcaagaagatgaaaagagacaatagaacaataacatcaacccaagcaaacaaaggaggaactgcaaaaaatggctataaactttcacaacatatgtgtctgatcttgatattttctccttgttttcatttgaaatattagggtttttagtagtaaaaaaagagactaacaaagaggaaattaaattaaatgtagtaactaaccttgtgattgatgatttctgttgaaagcttcaaaaataccaaaaggggtatgaggaacaacattaagagaaggaagtgtagagtcatgagaagtaagatcaaagcattctcctgaaacatcaactgaacctgatatttcttgagatgtttgtgatgttaattcctcatgtggtgaaagagagaatcctaaccagttgttcatagatgaagccatcaaattaatatcttggttaaaatttttttaaaaaaattacttcactagttatatataactattgaaaaacactagtaacattgattttttcccctctaaaagttcatgcaatatgaagtgctactggaaattaaaagagaagaaaaagataagtccatgagttagccatatagtctataagttgtttgaaggtgaaaaaaagaaagaagaagaagaggaaaggtccaaaaactgaagaggaatcatttctcccaaagcaaggttaagatttatatgaaactcaaaacccaaaaaaagaagaaaaaaatcaagttcgaccccccctcccccnctctaaatgaggggcagtgttttctctgatcccaaaaacatgtataggttcaaaatgaagagcaaaatatagggttgatgaggggtaggattgtaaaataattgaaccaagggtagtagtataacaaggacatttcctctttggttctacttatctatagcctattcttcttcactctatgtttaactttctatacacggataatatgcaaatttttgcattatcaacttaagttagacttataataacagatacgatataatatgtttagctatcccgatttagtaaactaaaaatgcaaaaaataacatgttgtaaccggttaaacgatactttttttttgtttttcgtccggtgtccggtacccgtattggagcccaactatatatatttgtaatcacgccgcataggaccccattcgaggggaaagactctctaccaaatatttttccatacccagagctcgaacccgagaggttacattgtttaatgagttaaatcttttttttgttgctaaacaactatagcttgaactttactgttactacgaaattctatacgcatatcaaatggaagctaagataaaagaataataatcaagagtacacgaggactaaataaaaaattgtgtgaattgtagccttctgtacttactaatagtttattttggcagatatatcctaatattttagttttcatatctaatccttttcttgtgtaatctaaagatcgggcaaagtggtatatagcatgttgagatagttgatcctatctttttcattcttgcatatcttctactccatttttttcattatttgaatgtccgataatcgcattggaattcgattaaatccggattcgtttcaattagcggattataaagcactcctaatataggtgactctatatctcaggctcaaatctgaaatctctaattaaaaaatgaagtagttactactccactataaccgttgttgtaatatcttctactcaattgtttgaagtgtatataatttgaggcagtattattctataaatagttattggcctaacacatggagtcatgcatattctttgggggcatgtggtttcttaattttgaaataaactgcactaatgttctttgaacgttcaaagagtatccaagttgcactcttattttacccattttttgttgcctatattattggctaaactcatttgcaagaactttaagaaaggcattccttgtttctataccaaatctctcttagtttattataggaatagggtacgtaacacaatttatttttgttgcctgaactgttgaacatatatctcttcccatctctcacttagtcaaggttgacaagttcaataattgtaggatattactaattaagaaagatatttaataaatgttttaaatacttacttaacactccaccaattagaataatatgattgatttgggccaaaaaaaaaataatttcggtgcataatgcatcccgcgttcacgtaggatctagggaggggcgcactcataaagagtgtattgtagacaacctatatcctaatgcaagtattagtggttgcttcgacggctcaaacccgtaacctatatagatcacacatagacaaatttatcgttctccaaaactcccttcagtatggttgatttgggaaaatataataaagagtccttttttatacaacattcaatattttgaaacaaacttagttggccaaaacgagtaattaatgttttggactaaaggggatacattaatgaataagataacttgattgcaaatcatgtttaaattcaactcagatcagaaaaacgttgactctataaaaagtgatttctataaatgtagaccaagtatatatataccgaattagtctataatttttctactgatcaggaactcatggaaattgttaattacaacattaatttgtgccttttaaatcgtttagctaagataatactagtccaatatcgtagttgtaatacataaagcctgatcattggtatggaaccaggaattttaatcagtggattcaaataaatacaaggaacaccacatgatttaattaagtttcagacatgcgatctaaacaatatttgagcaaattttaaaccacattcaaatctttctctatatgaagaaaaaattcgacaataatatatatatataactaattacttactaattttctatttcgaattaaaatttattattagtgcaagaaaatatgtatattcttgtatataatttaaatacatctatttattcactgaatgggaacttctgagtaagattaaagttattttcatgtgacctataagtcacgagtttcattcgtaaaaataaccactaatagaccattcgcaccacacttttggagtgcgacccttctcccaacgacagaagcggacctatgtaatagaatgaggggtcaccagcaccgtaatattcggcaaaaatctcatgtatacatgtttttacaaaataatgatatattagtagtgataccttatatataaagtaaattttggctgaacgtaaatgtgcacatattacctctaaattttggatttgcctctgttgaacaacatataaatacggaatgatttgtgtattgaattatctttttttacgtcattttattcagtgaatttccagttggatataagtcatatcaatgtcagaaatcctggtcctcttcttccttttctcttcctctatctgtaatctgtccactgccacaccccttaacctaaccaacccactacttttcatttaattttttttctttttcactcccataactttctcacattcttttgccatcttttaaaaaagtttaatttattattttttgcatgttgctgctgctgtttatcctcagcttgtcataatcatcaaactgctcccccgatagaaacgggggacttttcttttccttctttttaaaaataataataattcatttgcatgactgacacttaataattcttccccttttatatattctccttttttaggcagatcattttagagaaactgagatactcagaatctttttctttttcctttttagaacgggcttggcttagctctttttagtcttttgacacgtagtagcacgtaatgtagtcaatcatatacttctaactaggggtatataagtcgggttgcgccggcgaaactagaactttttttgagagtgttctaatttgaaaaaagtaaaaagaaattcttataaagggtattcaatatatgttatatacctctaaaacttgatattttatttatatacgcagtgtaattttttgacaaagggtgatcaatataaagtgtggcttcgcccctgtcgggctggttcaatttttatcaaaatcaaactaaaacaaacctagttgaatttttaaatcgatttgatttgacttttcgatttggtgattttgctggttcgatttgacacccctacttttaacaaggattggaggtaggggtgtacatggatcgagttggtttggttttgacaaaaatcaaaccaaactaactatatcgatttggattggttcgggtttgtcgggttttttgttacttaaatattatttcaattttactttgttaaattttttataagtaaatatatgtttagtaaaaatataaaaaatgataaatatattatctattaaaatattcttataggagattttttagtaacacatgatagttattttttttagtcgtctggcaataatttctcgttgatatatactttcaaggttaaccgaatttagtaattaaacataaaaattaatatgatacataaataataataaccacttcaaattcgaaaaagatataagaatttaatagatcttgaaatatgaatatggaagaacatagattgacgcatttcagtaacacttgataagaaagtgatcatacaacccattatttaggtgaaagaaaaacttattgatccccaccacacaaaagttacttacaagtgatattttcaatccttaatctataatctatcctatctagctcaatgtcaaaattatatctttattctcttctccttgagaatccctactaggtaggtttgcccggttagaagctagtcctacactattgcaaagccgttgttatttctctatatgtacaattgattgatccactttatttctttcctattcttcatgttggcttctatgaattgtagtctgtatttgctatcctcttttatgttcttccacatctttgttggcaatggtactttctgattccatagtgcttcattccttgctcgccatatcccatgcattactgatgtgatgattgaggttacgaatcctcgacatattctcccttttatacatctacccattttcttccaaatgttttgcatatctagttgtttcaactttattcccagccagttgcattgttcatctatgcacattctggagaattgacacacagagtacaaatgatgaattgtttcatcccgcattctacatagtagacatgttctcttatggcttattcccatcatatgtagtatgtctcgtaagtaatctttgtctcatgatcaaccatcctataaaataatattttggaatatttaatctattctaaatctatcgtgcatatggacatttaggtgttgctcctcttctccattagtatccttcatttactatgtacccctttatttgccccttccatcttttttgttcaaaccctgcagcaaaggctgtttggatatcacatatctttttctaataccatgcactatcttgtggtggtttgtattgccaccagtccttgccctttatgtatatgtgatttacccatttaaaccataataaatcacttttttgctctatatttcatatatattttgccaatgctgcttcattccataccaaacagtattgtattcctaatccactctctatttttgttcggcagactaggtcccatgctacctgtgggaatttgttcgtgacacttttctcatcctataggtagttcctacatatagttacaatgtccttcatgaccctttttggtagtaagaagatggatgcccaatatgagtgtaggtacaacaacattgtatttaccaactgagttctccctgcatacgataggttcctggatccccatccttttttctttaccagtatcttgtctatcaacatttcacactctacaacacttagtttctttgaggatataggcactctaagatatttaaatgaaagtttacctctcttatatcctgtcagttcatatatgtctttcatctctctaccattcatattcacatagaagatgttcgatttatttgcattaacagtcataccagatgcattcgaaaatgatttgagtccccttagcatgagcattactgatggataatctcctttactaaatagtatcatatcgtccgcaaaacaaaggtgtgtcatcttcaatccttttcattttgtatgaaaagcaaattcttcctacttagccacacgcaccatgatgcgcgtgaaatattccatatagatcacaaacagtaaaggagagattggatccctctgtcttaatcctctcttcttatttattttgccataacacccaccatttaagtttatgttgtagctagttgatgtgatacatgccataatccaccttatgaagaactcgggaaagtttagtgcatgcaacatttcctccacaaattttcattaaatggaatcatatgccctctttaaatccaccttgattaagcagcttttagtggtttgtttcctattgtataatttgaccaagtcttgacaaatcaagatgttttatactataattctcccaaccacaaaggcactttgattttgtgatatgatgtctgacagtaccaattttaatatgttacacaacatctttaagataattttgtacaccgcattatatcatgctataggtatataatcttcaactgttacaacatgagaggtttttggtatgagggtgatcattgtactattaagaggtctgagcatttttccagattgaaaaaattccaatacaactgcagtgacatcctcacctacaatggtccagctatccttgaaaaattggctgccatacccattatggcatggtgcctttgtccctcttatatcccattgtgcttctttaacttgcttttgtgaaaaatcttctatcaactcgatcctttgttcttttgaaacaatatgtccttctttgactatgtggcttgccacatgatctctgctctactggttagtgccctgtagccttgtatagaattcactaaaagtatcagctactgaatctgttgattgaattgtttccttgcatgttctgaattgtaaatatcatacttgatgctctctttgccttaatcaccgagtgaaagcagcttgtgttcatgtctccatatttaagccattgtattttactcgtctgttgcaagtagtttatttttgcttccttccagtatatgtattgcttcgtcaactctttctcttctttgaatagtagttcattcctaggttccttttgtattttctcttgacactttaccagctttttcatgcgttcatcttcctgtttctccacttttgcaaacttgtcttaatttaactttatcaacacctttttaagtttgtttaatttttccaccactttaaatatgtttgtcccctttataggttgtctccagctaccctccactcttgtcatgtaggaattatccttgctccacatgtgtgtagtatctgaatatcctcatcttaggtgtgtcagttccctcccatttaatcattacaagactatgatcatatactcctgctggcatataataaacctcagatgctggcaacctattcacccagtcaatattggtgatcaccttgtcaatcttgcttagtactctatcttgtccatcttgtttattgctccatgtataaaaacaccccgatgatttcaattcttttagtccacgttgctcaacatactccttaaactcctttatttctgcataagccaccttacttcccactctatcctcttgattgagtataaagttaaaatctctcataattgtccatgtctctaccatttgattagctatcatccttatgtctctccataagtcagaccttagtgttggattattgaatccatataccattatcatattaatcaccttacctgtactactatgagtaatcatactatgtctaagttgttctgtcaccattttcacttttatagcgaagatctgaggcttccacaatagccaaattctacctgcattatgtttagagtaatttgtagtatacgaacaccttgtgcaaagattaagagtagcttgatttgccttagctctcttaatttttatttccaaaagcccaaacagtccaacctgttgtctttgaaggaacatgttcacttccttctgcttggttgtcctgtttaggcccatattgttccaaaaatcaatactatctattaggggggtgggggtttggtcccccatcattccctgatttattaccattttcttgtgattccatctgcttttcattagattgtttctcaccttcaatcctttcattattcctcttattcggttgtgttactgcagcatgagtgttttgattgtttcctcgacccttccatgctttttcccttgtaccacttgccatgcttcctccacttttttgttattttctgcctgattcttagttccctgtgtctcttgttgcttgtctttggtactaccattttcagttggagttatgttacttgcttcaatgttttgttattgttcatctggttcttccctctctttgtagctatttgtattcctcatattgcctgattccttgttaatttcctttaccactttggtctgccttaattgcatcctatattctctgatatcatgtccaaagttgccacatgatttacaaaatactggtttccattcatatctaacctctttatcaatgatcactccacattcattctcaaacatcaccatatctcgatacatctggtttggttctacttcaaccagaattcttgcatattgcaacctctccttttgtgttgttgttctgtccgctttcagaggattaccaatcattcctgctatttttgttagagtattttgtccccaatatttgatatccaggttcatcaatctaatccatacaggaattttatcaactacctgcttatcaagtctaataacatgtctccatggtttaaccacaattggttttttgtcgaatattctaattccttcctccaccaccttctccttattctccattgagtggaatcttactatgaatatacccctgattacctgagctattttatctattcccagtgctcccccaactcttttgaagtagccctccataactgtttgtggtggatttgaacccagcacgtaacaaattacagcagatctccagtagattatctcatcttttatgtctcccattgtatttttcacattttccctcgattgctccccttcctttctggttaggttatacctttcactccgttctcctttccttacaattctactccaattttccccttctgtctgtgcctttgcatggctttgttttcctactaattcagattcactttcctcttcaacccattatttaaggttaataaatatggagcactttatattctattaaatattatatcccataagagaattccaaatatttctagataatatttttttaaaaattcatataaaatcttaaaagtatatataaaaattatatatttatatgtcggtttggttcggatttttttactcaataccaaaccaaatcaaaccaaacctagtcgagttttttttaatcgatttggcttgacttttcggtttggtacggttttttcggttcagtttatacacccataattggagataatatacgttttgcaaaatgtaagtgcatagttgaaacttcgggataaattgaggagatatttatgtgttatctctaatttttcgaaagcatttctttaggattcgtttgtccataaaaaaaattctttttttcgaaatgtgttaatccataaatttttgaaaaattttctaaaatgagtttttgaaaattcaaaatttagaagaaaaaaacatttttctccatccactaacagaattacaatattttttctagtgaaatacatgtccaagtgtatttcaaattttaaatattatattttttcaaaaattataatttttatgtacaaacgcctacttagaaaaagtgtagccgtttttctctttatctacctagaagaattgaaatttgaggaaatatcacactactagaaattgggcaaaaaccgaccattgtcgaccgatcaattttggtcggtcaaaaaaccggccaaaaaaagatcaaagttggtcggtttttaaaaatattttattttaattttttttttttacgaaatcaaccaactttggtcagttttctttggcgcaaaaatgcgaaaaactatttttgagtcccgcgaaatttatttttcaagaagccgaccaactttggtcgatttttttatttaaaataaattaaatttaatattaaaaaaccgaccgaaatcggtcgattaatttggttggccattttaaaaaaccgaccaactttggttggtaattttattttttaataaaatcgacctactttggtcggttattttcgtgcgaaaatacaattaaagagtataaatgaaataaaaggcagtcttaaaacaaaatgcaccaatggtctagtggtagaatagtatccttccacagtacataccctggttcgattcctaaatggtgtattttttaattatataattaaaataccgatcaatattggtcggttttttcggcaatttatttttgaatttaactgaccgaccactttggtcggtatacccttccgaccttcaaaataccgtccatacgtaaatggtcgcgttttggacggttattggccattaccgaccaactttgtttgatttttttggatgattttgcccggatttctagtagtgtgagtagtacatatattaaacttaattggtcaaacatgaagaaggatgattgtatgtgagtggtgatatatttaataaagagtatgaagttacactagtaaactcagtcatagtttttgggcattcacatgcaataactttttaatagcatatcattgtttgtagggcggagtcacaatgttgtttgcaggttcggatgaatccactagtttttattcaaatcttgtatttatgttaagaaattcaataaatatgtacaaatcattaatttaaaacccagtaacttaaaaggattataatcttgaacccataaggttcaaatcctagttccgcatatgattgtttgttatgcaagaacgattacaacaccttaaaattacacccacaaaaagaaaatattttttttccttcaagcttaggtcttggactgtgtcctttaaatggatgggtagcaatagcaacaggttgcaattatagacgaatttaggatttaaattttataagttcaacttttattgtttatcgtatttagcccatcatatttttaaaattatgagttcatatttactattttcataattttgatggacacaaatttatactctgtatcagaagtgctaggttcagttgaatcaagctctcctatgttgtatccaccctggctgcaaaacaaattaatgaaattgaaaatcaagcacttaaggatgtaacctagcggttaatgaagtatcgagatttcagttcaaatctcaacaaatcatttcaattatttgtggacagagttatttgatatttatgttggaggtgaaaaatagtaggtatatcgtgaaattagttgagagatgtgcaaactgatcggacaccatatttataaaaaaattgaaaggtaattaaaaagaaaaaactaataaataaataaaatgcataaggagagagactgcattaggtgtatccctaagcgaggacactaacccttgtcttacgggtcttggggtaattttattcttaacatttagggacagattggaagaaaaatcttgattcctaaggaattttcactctctctctctccctttgtgcgtatctaataatttcttatttaattctttctctctctctctctcaaatgcatgaacaacagatttgactgaatttattattattattttttaggttttttattcggtgtctggtatccgtattgagattcgactaaattcgaatttatgcttataaatttcatattacgggtaaaacgctctctaataaaggagattataaatatagaattcgaaatcgagacctctagttaaggatgaaagagtaattactcaatcactccaccacaatccttgtcggtaagtattgtttcaagtgagagtgaaaacttcattttaaaaaatcctttttctcaattctttatttttttttctttttttagcagaggaatacaaaaagtatatcattctttcacttgtcttagaatctcagtttcgttctttgtcctttgtatgataatgcttatataaatactatctcgtatcaagctagacaatggctaaatatagactcatcgatcgaatgtaaggtacaaaaatactaatgaagtttttttgattgaatttgaaaactggtggaagaaacaaattttactttctcttgaaaatttcccaaatctatcttcttatttttgtttactagaatatgatttttggaatttattgaccacctttcggaataaagaacaataacaaaacaagtgtaattccaagagtggatctgggaagggtagtgagtacggagactttattcctacgttgagaaaatagagagattgttctcgataaacccttgacacaagaaaagatgcaaaaagaaatggtagcattcgatagtaacatcaacaaaataacaagaaaacaaaagtgaaagaaacaacatgcaataatagaaaatcttgaaaaataaagaaaccaaactaaaaacatttccaagaggggggaaaaaaaggaagcaaaatgaaacacttcagtaccttacaataatattaaattgaagcaaactggaaccttagtttctttatttttgttcaatgtattagatatttgatttatgattcccatttgatttattttttttggtcattccaatttgatcaacttgacatttaaccgcactttggttctaggctaatttcatagatgcctcttccaaagatgattacttacaccttgtttggacggttaccaatcgttttataatgtattgtattgtgttgtattgtattgtattgtattgtattgtattgtattgtattgtattgtattgtatcgttttataattaaatacaatgtttggatagattgtattgtttgcgattgttaaataatattttgctgtttgatttgactgtaccgtattgtactgtaactgataagtttactaaaataccctcaattgtttaaatattaaatttatcaagaattaaaaataattttaaaaaatccctttctactaatttatcaccaaatatgtcttataaatagattaagcaattaaattcatgcaaattctaacaaaattagcaattttacgttggagttggaagcgtaagaagttcttgaaaaaagggacgaagacaaagtatgttataggttggagattttgagttaaaataaaaaaaaggtaaacggtaaaaatagaattatgaaataataagttaggacataactagaaagaaaaatagaaaacaatctcaccacatcaaatcagttgtttcaaaaaataagactttttattgttccggaacaattgatttaacaatacaatacaatcaattttaataaaataatcaaaacaaacattattttgagataataatacaatacgatacaactagtaacaaccatccaaacagcagttagttagatatatatctagtcgaaagtgtactttttcactatgcatgagaattataatgttttcaattgttaaatccattcattaccaaattagctgaattaggtcatgattgtctaattaattagttaattcgatgcacaccaactagatgtggcacgtagtcacatataggtggtccaatgaatgtgactatgcacccattagaaaaatatgaaattaaatccttaaacctcattataaatacaagataagagatatcgatctgtaaactctcattcaattcccaagtatttgaaaaaatgtttttgcacattttcaccttctccttttaatatttcagactttcagttaggtaatatatgttttatgtgattgatatctgcaatttgttttccaattgtatgagcatatgaagctctgcagtagtttaagacaaagaatatttattcatagttggtgtttatatcaaattaagttaatttaacaattaaaattaaagtgagaaacacatcacataacgatagttaaatgaaacaatgtatgtgcaaaatatatctttttttctgggcttggtgttaatatcatgggcaaacaagactactaacacttatgagtgtgatgggataaatagaattttcatattcctaattagaagtctcgagtccgagctttagcatcgctttccctttaataagctatacttgacgtgaatccacatggatacctgatatttcggccagttattttataaaatttacaccacaccgatttttcggttattctattatgtataaccaaagttagacttttcgaaaccgtctcaatcatgtcggtttctcttcggtatcggtacgatttggttaattttcggtattttcttaatgtcatataaaaatcgctaatagaagtagaataaaataacatacgtacttttatagaacttagcaatattctatcactactagaaattcggcaaaaaccgaccaaggtcgaccgaccaactttggtcggtaaaaaaaacgaccaaaaaatcgaccaaagttggtcggtttttcaaaatattttttttttttttcgaaaccgaccaactttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttacataattaaaataccgaccaactttggtcggttttttttgtaatattttttttttgaatttaattgaccgaccaaagttggtcggtaattttcgaccaactattcctttccgaccacaaaaataccgtccacacgtaaatggtcgcattttggtcggtttttggccattaccgaccaatgttggtcggtttttttggtcgatttttaccgaatttctagtagtgtatagatatttttactatttaaaggatgatgaattaagaaaacatgaaagatggctagagtataagtccatcaactattctacaacatcgtaaacgaaactaacaaaagacaaaaaaaatataaatcacacgagtaaaaagatattaatcaagcggggcctcaagaataaagtcaatacaagattaaatattcaaaaagataaatctaaattacacgaaagaaacatattcaatactttctttttcacatgtaaatttattcggcacggtttattattttttcggtttatttacgtaaaataaaaaacctaccttaattatcgttacatttataaatttatataaaacactacaattttattacaagaaacctaaaaatcgatttggtctggtacagttcggttggtttagttgtttttataccgaataaaaaatcaaaaaataaaggggagagtttggggggggaggggggggggggttgatgacccaaaaatgtcatctttaatttaaacgttcatttctatgttccatgaagatgttgagagcttacctgctatgagttatgtatcacctgaatagttgatattaatgacgttccttttctagtaatacattgcttatgatgccactgtcagtattattttcatgttgtgttacattgattggactatgtatgtgatgttaggattggtttctgagattctctggcaggtggataggcaaagttacaaaggaaactctagcgaaaattttgaaaattttgggagttagtaaaaattcgggaaattgaaatgggtaaaagaagagataagttatgttatgtacttgaggacgaactctacttctcatttaaggactattaatattatggataccaattggatatgataacaagtgtacggggcatgttataagtgatgtgaggtctaaggaaagacctaagtctaagtcaagtaggaaaatttcataatagactaaagtttcaaatggatacgcacaagaccaaattttgaacgagcatatatatatctgtatataaagaattatgtgatgatctacctatcaaattaaaggtcttcgagtctagtttctaactcttcaaaccgttcgtcatttggacactcctacaggaagttatgactaaattatcaaagattgaaaatatacgattttgcgacctattctgcgatcgcagaaaagttatgcgatcgcgaaactgcttctgcgatcgcaaactggtggcagaatagaccagaacagtccagttctggatagcaatttttgtaaccattgtgcggcccgcataccgattctgcagtccattatgcgaccgcatacccgatttcggagggttaatttttctattttcataactcgatcccattttgataaataggctttggggcttattttggggcaattatctgatgattttagagagaatttagagtgttttagagagaggaagaagctcaagtgctttgttcatcaaaaatcttgttcaagctttgaaatccaataaggaattatcacaagatcttcacgcaagaggtaaggttctaacccctattcttcaatttcgagtttgggtaaagatgggtgattgtgagtgtgattcttgcatgtgagagtattagttatacatgcttttaccaataaggttatgggaagattgttgagttcaaatgggtaaagattagattggaaatggtagaaatcttcaaacaaaactttaattgaggattttaaagccaatccgatgtcggaattcgataatgtttgtatggttgaactcgtatctgaacgggtgttcagatttcgcgagtttttccgggatttgagatgtgggtaccattgttatttttaaaattaattttggatttttatctgaaaaatttataaattcatatggaattaattcctacgttttgtattgagtgtattgaattctttatgactagatttgaggattttgtacatgaattcgtgaggtaagggtttattagattcttgaatttggttgcaaagcgaggtaagtatcgtggttaaccttgacttgagggagtaggacttatttgtctatttgctacatgatttaatatgcgggtacaatatatatgtgaggtgacgagtacttatgtgttatttactatgaataattgcctatttaattaagatattcttttttaagtttattattgattatttgatcattattcgtgaaattattattcatttaattatagttgaatattttggaatgtgaagtcggtattctggtattgaattgattgataagtgcatatcctcgcatttaaatactcttccaaatttatattattattttcatggtaaggaagagtgtaaaagcacgaagggtgatgccgtacaatttttattatttataatattattgtcatggtaagaaagagtgtaaaagcacgaagggtgttgccgtgccatttgtgagggtaaaagcacgaagggtgttgccgtaccatttgtgagtgtaaaagcacgaagggtgatgacgtgtcatgttcagaaagtgtaaaagcacgaagggtgattccgtgctaaatgagagtaaaagcacgaaggatggtgccatgccattttcatattatcaattgctcattttattgttgattaattaaatggttgctaagtgatacttctcgtgctgaaattattatatcgtttcccttcgcatgtcccctcccaatttataaattattatttattatattattgttactttgtatttgtatatacttgtacgggtggtttacgtatgtgtcttgtcatagtctcgtcactacttcgtcgaggttaggctcgatacttaccagtacatgtggtaggttgtactgatactacactctgcacttttgtgcatattttggagttggtcacagcggcgtacaatagacttgctcggattcaactacccagaggagacttgaggtataactgcacatcgtccgtagttctgaagtccccttctatgttagcttagctgtgtcttatctttcaaacagcttgaattttattcagacctttatttgtattatccgagtagctcgtgcacttgtgataccggattcggggatatattttttaattcggtatttatggtcttttgtactttaatttagtaatttgacttctatttaattaaacttgcttagaaatagtcaagattattttaacgttggcttgcctagcaagtgaaatgttatgcaccatcatgatcctgaaggtgggaattttgggtcgtgacagttggtatcaaagcactaggttacataggtctcacgaggtacaagcaagcttagtagagtctgaaggatcggtacggagatgtctgtacttatctttcagaggctataaagtttaggaacaatatcacttctttcttattctgtcatgtgatcttattctatcatcgataattgaaccattttattcttattctcttgaagatggtgagaacacgtaatacatctaccgaaagacagggaccagagcccacggtggcaactatgaccaggggtagaggtcgaggccgaggtcgtgctagaggccgaggcaaaagcagaggtaaagctcagtctagagctctagcagcaacacttgttgtagaacctcagatagatctttaggaggatgttcccattcagactgtaccggttggaccagttcaggtcccggaaggatttatagctacttagtgcttcaggatgctctagtccgtttggtaagtcttatggagggtgtggcccagaatggtacatttcccgtggcaccagccgtttcacaggctgggggaggagcataaactcccactattcccactccggagcaaatggctccccagaatcaggctccaccattcctgccagttggggtagttcaaccagttgttgcggcacagaccgatgataggcgcgccatgtcttctgaggccttattgagactagataagtttactaagctctttccagttcacttcagtggaacaccttctgaggacccacgggattatcttgaccgttgccatgaggtactgtggaacatgggtatagtttagaccaatggggtcgaattttctgtatttcaaatgactggttccgccaagaggtggtggagagattatacattgaccagaccagttggatcgcctgcattgacctgggagcagttttaacggcgaattctaaagaaattcctccctatcacattgagagaggattatcacaggcaatttgagcgtctacagcagggcagtatgactgttactcattatgagtcccgttttgtggatttagcccgttatgctctccttttactacctactaagggagagagagtgcggaggtttattgagggacttacttaccctattaggcttcagatggctaaggagaccgaaagtgagatttcttttcaggcggctgctaatgttgctttgaggatcgagatagttcttgcacaggagaaagggcaggggtctgataagaggcctcgtcagtttggtgggttcagtggtgcctcgtctgtaggcagggctaattttggtaggggtcatcctcccaggccatttcattcagcactccaggcatcctacagtgcaccaccagctcctatcagtgcacctccgatccagagttttcaaggtggttactcaggtcgacaaggacaatttcagggtcagcagtcataacagccgaggtcttgttatacttgtggtgatccgagacacattgctagattctgccccaggtctcagggcagcatgcaacaacagggctctcgtgttatgattccagcaccggttgcttcaccgcctgctcagccagctagaggtaggggccaggcagctagaggtggaggtcagaccattagaggtggaggtcaggccgttagaggtagaggccgtcccagtgacgcagttcagagtggtggggcccacccccaattttatgcttttccagctaggcctgaggccgagtcatctgatgttgtgatcacaagtattattccagtttgccatagagatacttcagttctatttgatctaggatctacttattcttatgtgtcctcttattttgctttgtatttggttgtgcctcgtgattctctgagtgcttctatgtgtgtatctacaccggtgggagactattgtagtagaccatgtctatcgtttatgtgtggttactattggtagtcttgagactattgtgactcttttacttcttgatatggtagattttgatatcattttaggtatggattgactgccaccttattatgctatattggattatcatgccaagacggtgaccctagccatgccggggttaccttggttagagtggaaaagaactcctggtcattctaccagcaaggttatttcttatatgaaggctcgacgtatggtcgagaaagggtgtctagcctattcggcttatattcgcgatcccagtgcgaatgttccttctatggactcagtacgagtagttcgtgaattttcagaagtatttcctgcagatttttaggggatgccacccgacagagatattgacttcttattgatttagctcccggcactaaacccatttctatcctatcataccgtatggccccgccggagttgaaagaattgaaggagcagttacaggacttgcttgatcagggattcattagacccaatgtctcatcctggggtgcacctgtgttatttgtaaagaagaaagatggttcaacgagaatatgtatagattatcggcagttgaacaaggctactatcaagaacaagtaaacactgccaagaattgatgacttattcgatcagcttcagggtgacaaggtgttttcaaagatcgacttgagatctggttaccatcagttgaagattagggcatctgatgtccctaagacagcttttcagactctgtatgggcattacgaattcctagtgatgtcatttgggttgacaaatgccccagtagcatttatgaattttatgaatcgagtgttcaagccttatttggattcgtttgtggtcgtattcattgatgatatcttaatttactccagcagtcgagaggagcatgagaatcatccccggagtgtgcttcagactttgaagaataattagtcatatgctaaattttcacaatgcgagttttgattaaactcagttgccttttgggggcatgttgtatcggtagaaggcataaagatggatcctaataagattgaggatgttcagaattggcctagacctacttcagttatagagattcggagtttcctaggtttggcaggttattatcgtcgattcgtagagggcttttcatctatagcaagcccattgacgagactgacccagaaaggtatcccattcagatggtcagacgagtgtgagttgaacttttagaagctcaagacttctttgactacggcgccagtgttggtattatccacaggttcaggatcttatacggtgtattatgatgcatctcgcgttgggcttggtgtagtattgatgcaagatggttgggtgattgcatatgcgtcgcgacaattgaaagttcacgagaagaattatcatgttcatgacttagaattggcagtcattgttcacgcgcagaagatttggaggcattacctctacggtgtctcgtgtgaggtatttactgatcatcgtagccttcagtatctgttcaaacaaaaatatctcaatttgaggcagagaagatggttggagttattgaaagactatgatatcactattttgtatcacctcgaaaaagctaatgtgatggccgatgctttgagtagaaaggttgtgagtatgagcatgcttgcttatattccgattggtgagagactgttagttgtagatgtccaaactttggctaatcagttcatgaggttagatgttccagagccaagtcgggttttagcttgcacagtcgctcggtcttctttatatgagcgcatgagagggcggcagtatgatgatcctcatttacttgacctcaaggacatagtgcgacacggtgatgccaaacaggttgttatgggggaagatagagtactgcaaatgcagggtcgtatttgtgtgcctaatgtggatgggcttcgtgaatcaatctaggaagaggcccatagttccaggtattatattcatccaggtgccgccaaaatgtatcaagaattgtggaaacattattggcggaggagaatgaagaaggatatagttgcatatgtagcttggtgtctgaattgtcagaaagttaagtacgagcatcagagacttgatggttttcttcaaaagttagaaattcctgagtggaagtgggagcacatcactgtggactttgttgttaggctcccacggactcagagaaagttctacgcaatatgggtcattgtggacaggttgaccaagtcagcacattttattccagtagcaattacctattcttcagagtggttagcggagatttacatccgcgagattgtacgccttcatggtgtgcccgtgtctatcatttcagatcaaggtacgcagttcaccttgtacttctggatggttgtacaacatgagttaggcacgtgggttgagttgagtataacgtttcatccacagacagacggacattcagaacgcaccattcaaatattagaagatatgcttcgcgcttgtgtcatagactttggaggttcttgggatcagttcttgccacttgcggagtttgcttataataatagctaccagtcgagcattaagatggctccatatgaggcattatacggaaaggcgatattgttcgccagttggatggtttgaaccgggagaggctcggttgttaggtaccgatttggtacaggatgcctttgataaggttaaagttattcaagatcgacttcgcacaattaggcaaaagagttatgccgaccgtaaagttcatgatattgaattcatggttagagaaagagtattgtttcaggtttcacctatgaaaggtgtaatgaggttcggaaagaagggcaagttgagccctaggtatataggacccttcgaaattcttgaaagggtgggagaagtagcctacatgcttgcattaccacctagtttatcaacggttcatccggtgttccatgtgtctatgcaccaaaaatatcatagtgatccgtcccatgtgttagatttcagctcagtccaattggacaaggatttgacttatgaggaggagccggtagctattctagcccggcaggtccaatagttgaggtctaagagttacccttcagttcgggttcaatggagaggtcagctgatggaggcagctacctgggagtctaagtcggacatacagagcaaatatccacatctcttaaccagctcaggtacttttctaatttcgttcgaggacgaacgtttgttttagaggtggagattgggatgacccaaaaatgtcatctttaatttaaacattcatttctgtgttccatgaagatgttgagagcttacctgctatgagttatgtatcacctgaatagttgatgttaatggcgttccttttctagtaatatattgcttatgatgccactgttagtattgttttcatgttgtgttacattgattggactatgtttgtgatgttaggattggtttctgggattctctggcaggtggataggcccagttataaaggaaactctggcgaaattttggaaaattttgggggttagtaaaaattcggaaaattgaaatgggtgaaaaaagagataagttatgttatgtgtttgggggcggactctacttctcatttgaggactattaatattatggataccaattggatatgataacaagcgtacaaagtatattataagtgatgtgaggtctaaggagagcctaagtctaagtcaagtaggaaactttcataatagactaaagtttcaaatgggtacgcacaagaataaattttgaacgagcatatatacttgtatataaagaattatgtgatgatctatctatcaaataaaaggtcttcgagtctagtttctaactctttaaattgttcgtcatttggacacacctacaagaagttatgaccaaattatcaaagactgaaaaaatacgattttgcgaccaattctgcgatcgcagaaaagttatacgatcgcgaaactgcttttgcgaccgcaaactggtcgcagaatggacccgaacaacccagttttagggaccaatttttgtgaccattgtgcggcccgcatacccattctgcggtccattatgcgatcgcagacccgatttcggagggttaattttcctataaaaaagatttcataacccgatcccattttgataaataggctttggggcttattttggggcaattatctgataattttagagagaagttagagtattttagagagaggaagaatctcaagtactttgttcatcaaaaatcttgttcaagccttgaaatccaataaggaattatcacaagatcttcacccaagaggtaaggttctaacccctattcttcaatttcgagtttgggtaaagatgggtgattgtgagtgtgattcttgcatgtgagagtattagttatacatacttttactaataaggttatgggaagattgttgagttcaaatgggtaaagattggattgaaaatggtagaaatcttcaaacaaaactttaattgaggattttaaagccaatccgatgtcggaattcgataatgtttgtatggttgaactcgtatcggaacgagtgttcagattttacggtttttttgggattcgagatgtgggtcccactgttatttttaaaataaattttggatttttatccggaaattttatagattcatatggaattaattcctacagtttatattgaatatattgaattgtttatgactagatctaaggattttgggcacgaattcgcgaggcaaggatttattggattcttgaatttggttgcaaagcgaggtaagtgtcgtggttaaccttgacctgagggagtaggacttatttatctatttgctacgtgatttaatgtgcgggtacaatgtatatgtgaggtgacgagtacttatgcgttgtggttgagtcaaagcatgtaggtggaatttgtttactatgaataattgcctatttaattaagatattcttgtttaagtttattcttgattatttgatcactattcatgaaattattattcatttaattattgttgaatattttggaaggtgaagtcggtattctggtattgaattaattgataagtgcatatcctcgcatttaaatactcttccaaatttatcttattattttcatggtaaggaagagtgtaaaagcacgaagtgtgatgccgtaccatttttattatttaaaatatcattgtcatggtaaggaagaatgtaaaagcacgaagggtgttgccatgctatttgtgagtgtaaaagcacgaagggtgttgccatgctatttgtgagtgtaaaagcacgaagggtgttgtcttgtcatttgtgagtgtaaaagcacaaaggttgatgccgtgtcattttcagaaagtgtaaaagcacgaagggtgatgccgtgccaaatgagagtaaaaacacaaagggtggtgccgtgccattttcatattattaattgctcattttattgttgataaattaaatggatgctaagtgatacttctcctgctgaaattattatatcgttccccattgcatgtcccctcccaatttataaattattatttattgtattattattactttgtatttgtatatacttgtacaagtggtttacgcacgtgtcttgtcatagtctcgtcactacttcgtcgaggttaggctcggcacttaccagtacatggggtcggttgtactgatactacactctgcacttcttgtgcatattttggagttggtcccagtggtgtaccataaacttgctcggattcaactacccaaaggagacttgaggtataactgcacatcgtccgcagttctcaagtccccttctatcttatcttagatgtgtgttatctttcaaacaacttaaattttatttagacttttatttgtattatcctagtagctcgtgcacttatgacaccagattcagggatgtattttgataattcagtatttatggtctttcgcactttatttcagtaatttgacttctatttaatcaaatttgcttagaaatagtcaagattattttaaccttggtttggttagcaagtgaaatgttaggcgccatcacgattctgaaggtggaattttgggtcgtgacaaggggagaggcaggcggatctaggatttttataacatcggtgcaccattaaaaaaaggtgaatagaggaagtattaagtgagatttactccttggtcctctaggtaaataactaaatattcaatcaagtgcaccgtataacctctacaaatccgcccctgggtgggggaggattggaggtgtggagacaagggggtgattattatctcgaaggatgtaaaaactcaacagtaaagtttgaccgtcaagaaagggaatcaaaagacggtaaaaaaacacatggcattgccaaccctattaggttttatttcttgtttattaaatactttaatttaatttttctgtttgttgtgatccattattaaaaggctgcctattgtatccctacagtaaaaaaaaatatattatattaaataaatgcgtctcggtaatataatcataatcatgcctcaaactttgcctttgcctttccctccatgtgtaaatagggttgagaggaaattatgacttttgacccctatatcgtttgactaaccctagtttcgtttgaatagtaatttccnatatatatatatcacgctctttgggatgcaatcctttttcgaatctgtatgaacgtgaaatatcttgtacatttggctgatctttatttcctcatgaccaaatagaatgtcttatataagagaagatccattttatgatttattgcaaatgttattcagttatatataattttcttgaaagagaattggtgaataaaacattgaaaaaccttataaagtaattccattttattcaaaattgtaaattggccatacgaaatcattttcttcgagattagtaactgtttttacgactcaaactcgtaagctatagatcacacaaaaatattttattgttgctcagtttccctttaagaaataatgtcgagtttatattatcttataatttgccctttttacaaccaccttatgaagatgttatttttctcccgtttttacgctgttttaaaggtcggttaattaaattaaaaattacggaatactagtctactaggattagtatatatttatttagggggtgtgcatacgtacaaatgttagtaataaattatttactgtcactgcagaatatattgaaactggattctattattcaaacatctctgactgagtgtatccaagtgtcttttccttccttttcttttgtttattgtttttttttccttagtatattaatccacgtgtacctaattgcaacatgttcgagcatcaaataattataggaaaaaactgacacgctgatatgagaattctttcaacccctttcctcttatattaatgctccgatcatgcgcatttgctattatggcctaaagtcaaaaatattaaatttaaagagattaaatgtctactaaacaggcttaccaaattggtatcattattctatctgccattgaaagagctgtagaattagattcagcaaaagtaaaaatttaaataacaccccatgttccaaccaaataaatgaatgaaaatgttttaaatcaaatataagctgtagattttcatttttcaggaatccaaattttatccctacttagtccaaatcccaggttcattaataatcgtcttatcatatggttcagtaatttatatgttggtctaaacgacctgacctctttaaagtaggactatttgataccagggacggagctagagtgcgagaaacgagttcgaccgaatccaatagctttgtttcgaatcttgcatttgtcttaaaaaattcatttaaaatgtataaattattaatttgaaactcactaacttaaaacgattgaaatgacgaacccattagtttgaaatcctagattcacctttatttgatacataatattgcaactaaggatgcagtctaacaatcaaataaaatggataaatatgtgaaatatgagttcagatccgagtaaaataagaaaaatactatttttttctttcccattttatgtgacaaaacttttattttttgtttgtcctagtaagagtgacatctttttatttaaaaataatttaactttaaacttctcacgttatacttaacgataatttttttttttaataaccacgtaattaaatatctatgacgtatcctcgacacaattctcggtagggtttgttttcttgcttaaactccggtcaaatactgtcacataaattgagacaggaaataggtgatttcttcctattctggtggaggtaacaattatctgatagataattgaggcgcgcacaaactgaacgctaacacttattgaagtctgatgtacctggctatatacagtgcgttcagataggccatatctacccccatgaggaataaaaaccgatgatagtatgaataaatgactttaaggtgtatgtgtgtgtacagtcaattaattctcagctgtgtgatgagtcgtctttttctcttcattctctaggctcaactctgtaaagtagtgtgatatatatatatatacacacacactgtcaatgacccatgtgaaaacattaacccttttcttcccaacttgtttggtgtatatggttataaccttagatcagaaaaatggccccctcttcatatgaccattcaatattatgccatgcaaaatgcagcagcatattcagataataacaggattaaaaaaaaaacatcagcagcatgtcctccacggacattcagctgacgtgggtccggctctatgcagtggccaacgtaagaaaattcaataagagactaataggctatatgaaagagtgttcaaagtcaattttttgatcaataataagtactaatattttaccttataaacaatataattttttggtgaagaatattcagttgaccaccctcgacatgacatagtttcaattcgcccatgcctgcatgcagaggcggagctagcttatagtgtgcaggttcagccgaacacatttactttcgcttaaacgtggtatttatactaggaaattcattcaacatatataaatattttattgcgaacacaataactaagaagagctatatgctcggtggtgggttcggtggtacattcagaaaccataaatgtcaaattctggcgcctttgcggctcttcatgcagcctaggataccactcaatacttgtaaatttaggtgagtcaaatggactggagatggagaaaagcttggccaaagcgagaatattcctatacattaagagcccgtttggacataaaatttgattgaagttttttttcaaaaaaattcattttttttcgaaatcagcgtttgttcataaaatttccaatttttcacttgaagatgcattttgaaatttttaaaaatttgaaaaactccaaaaaactgtttttcataattttcacttaaatcactcacaaaacttaaaaaacaacccaaactgaaatttatgtccaaacacaactctaaatttcaaatatcattttcacttaaaaaaaaaatttcaccattttttcgaattttacaattcttatgtccaaacgcctactaagaatatctttgagttgaccgagcaacacaatcagtgcaattttttattcatattaaaggaaaaatggttaaattttctataaaattttctcaattgattttaaaggatgaaaattgaagtcatggccagagctaaaactggtgagcatatttgaatccattcgcatttaagagctatatattgatttactactaatatttttgaagaacttaaacttattaactgaaagagagtcgaaggtgaagtcttcttctgcgaataaccgaaaagatacaaaggaattaaatcctagactagttatcacatcctagttatagataatgttttatatttttcttcattaacctttcttgcgagatgagactaaacatattgttagatattttatatgcattatttttttattaggtgagcagaataatgtaagcttgcagcttttaacaaaattagttaattattattcctctaaatctttgcacaataatctgtgttattattcgcagcctgatattaatacatgtttttagtttaccgcatggcatgtattatgtacaatcattagttcgtgaaccaacaacctctaagtgaatacgcacgagagaaatttatactctccagtcacaatttacgtggcattattcggattttaagagtaaaataaatttttctttgactgtgatattttgatatacattttaaatatttgaattattaattatttcaacttatagtactcgttttaagtagttttcaaatatataatttatctcaaaaaattataagattttatatctgaatttacggccaaaaaaagtttaactcgcgaaattcgaacaatgccacatcaattggcatagaggtagtatatgctttacaattatatatccaattgtacacatacaaaatttcatcaaagatatgaaactactaatacaacgagagagcgaattcaaaagataaactttatgagatcgagttcaaaattctactgtagttttatctaccggattcaaaatctactgcaaaaatattagaattagctacgaacttcgtcgctaatctgtcactaaaccactcgtaccaagcaaaatatcttgataatcttttgctaatccctttctaaatgagattagcaacagatttttctgtttagctacagaatttatccgtcgctaaaacttgattttttagtagtttaattatttttaacacatacaaaaaatttgagctaaagctagtgagttcaagtataataaccatactactaatacaatgtgagttctctataactgaatccttatataacaacactttactataaaaatcaagtttttttaggaaccaaatttcatgttctattataatatatgttttctataacaccaatttattataacattcaaaaatatctgaaacaaacgaaattgttatatagagatttgagtctagtattatttatgcatgcacacatacacacaccaaaattcaaatctcagaaagagacacatagaagaaggaacaaaaaaaataacctgtaaaatagtagtggggaaatgggaactaggattttgcatacattggagaaataggcggagcattcctagcagcatcacccgtgccttctcggtatcgtatattttctgcaactatccccaatttgcacggtcttttatgcaattcaaccatgaaactaaaccaaagcctctttttttataattgtgttaatcggactaatttatatgtacctcaactaacttcacgagctatcgtgtatgtccattaaggcttggactgataagagaaatcagttaacgtgtttgcgttcgccctgaaatttgaacttaaaacattatggttgacaatttcacatccttgttcaccaaaagcaaagccttcttccaatcagatataatatacatttaatagtataaaacttatctgctatcaatgtattttacataatttgtcgtagcaagttatcatttattttttgaattattaattccacttatgatggcaaaaattatttacactttaatatcatagaaaagctaaactcgacatatgagtttacaagaaccaaatagattttgtccagactttgtttatactttatatattaaaatgttcactaaatatctataattatttggcaatgtaaatccaatattattgtaaatttacttaaaattgatataaaaactcataagttataaattctggattcgtctttgctcccaaccaactaccctccacacatacacacacaaagctcttatcttcttttccaaagaattaaatcaatattaactttgagggtctttgtctttcttcatatttggttcaaaagggttggaatcgaaaagataaaaacgtttcgaaagaaggaaaataaataaaataaagcttacttcatgctacgctttcctcaaggtacccaatgaagtgagggtttaagataaaagcaacctacagatatttatcttttcagttgaaatatgcctatgtaccagagcaaaaacgatctactttgaagtatatcgtttatcgatcaattagtcttaaaaattaaattattctttatgttcttaaacatcttatttaattaacgctccattaatttaaatagtgagtatatatagattaaagaaaaaaaatagcttattctatctctatcaaattaaattcacttttccaaaacgactaatacttgtgagagggagtaaaaataagattgagagttattatctagcaatgtgaaaagtagggttttggatttaaaaaaaaaaagtagggttttgcatggaggggagctagaagagtgggatttggactcatatttacacttccaatggacaaattacttaggaaaatgaaaaaatcagatgagtttaagttatatttatcgtcaatataaaagtttttactattaagtcatctttatatatatatanggagatattattctgatactgtaaataaaaattttaatttgacaatgtatataacttaaactcaaaacggataacatgaaaaacaaaaatacatagaaaaataaagtaatataaagccagccacgcgtatctgtggctgtctgtatatgaacccaaatatcatctcgtaagctaacaaaaatgcctaggaatgctcatttgtttctttctctttctctttttcttggtttttgatgctcttaatattatgttataattacaacttcaagaaaatcaaacactgtttgggattcaaaccctatgttttgacctcaacagtttgacccttcttcctatatatatttcaaccaaatcaagttcttttgcactcaacaagagcaacaacttaaatacatgcaaccaaattaagataacagtagatcagaaagaagttagaactgcagtaaaagggaaaacaacagcacataagaaattgtaccaaaaacttttgactgcatattgttttggaaggaagagtatgatttgtattaaaataccatattccaaaattttaatgattggtggaaagaaagaaaaggcaaaataaagtaaaggaatctataaagattatggcagctaaatttggcacttggagcatgcacctaggccagtctaaaagagtgataaaaaggaaattaattttaaaaaaaaaacaattgtcatctttttccttaatatattgtattgtatggaacccttgataatttaaaaagctgatcaaagattgacctttgagtataattcaccatattgtggaaagacaatttatattttgaccaaaaaggggaaaaagaagttttataacaatgggttcataacttcaacctaggctccctacaaccaacattgggctttggtttggagtaatttggacaaagtgcacaaacaaccatttttgggctgctatttaggggttaaccaatatttttatcctgaaattttgaattacaaatcttattgttaggacattcttgtcccgaaaaattgactgaaaaactaaaattcaagatgcattggctaatttctaaatatcagccctttagagtggctacctagtatcatttctacgagcaatttaattattctctagcccggacatctcttagaccttcatttcccataatgcagtcggtttttggagaaatttgactccaatgctgctgcattttttgttgcaaaaatgggggatgaatagtgtcactccaaatttggagcgacattattcatctcctccattgctattccctataactttattattatttttattactgattgatctctttatatatacctaattatgtttatgtaatatctttataatattaattttacatcttaaatttggtgtataattttgataaattaatttttgtgcatttattatttttatgtaaaattataagttaattttattataagttataattgtacaaaaaatatataatcatctcaaaaaatgaatggtgtaaatataaaatattagatgttgctaaaattgaaaagtaaaaattgaaaatacattaaaattgaaaatatattaaattaaaatacataaaaattaaaactgcagtaaatagcataataacttagtagggacctatgtcgtttccagatacaccaaaattattataatattgagtgaatggggtcgagaagtgctacatgatataataactacatgtattatactgcacaacatgataatcgagaatgcgcgtgatcttaatgcaccaattcaagatgattgggaatgtccacctccaatggtagaaatggtagaagatgaaatcaccgatttgaacaatttttagctcgacacaaaagaattaaggacaaagatactcattttacacttcgtgatgcattaatagatcatttatgggagcatactagaggtgaaagttgaatatttatttagtatttaatctaaattttaccataatgtaatatgtatttaattatcttgtatctcaatgatttcacttttatttgaattattcgtgaatatatatatatatatatatatatatatatatatatatatatataaaatctataatattttcttacaaattatattatttaaaaatttatggtgtaaaataattttatattaaatggttatataagaaatgaatataaattatatgtgatgaatatgtaaaaaagaaaaaaaagatagaatttctttaatagaaataagaaaagaaaatctaaataaaaaataataatatgatattgacgagagagaagaatataaaatgaaatattttttttggagtaaaaaatagaatagtggttggagtaactttactccattttggaggagaaaatagaggcaaaggttggagatggccttaacacccctttatccacaatttaaaaaaaacatgcatttgtaaacttagcttaagaaataggtatccattccttctaatgcattttattagctaagttgtagtggttaataaaggttgcagatattttaagataatttttcttcatatgagtttacttgcaaaagagattttcggtttcgaaattgtcaattacaaaaagatgaattaaggaatgttaattaacagtgtacaatctgcgcaaactactcttttgtgacaaaataggtgcaaaatatacctttctttaaatgcaatattaataatacacgagttttatgttggattatttattgtaatacgtaattacaagctaaaaggcaagctctaaggaccttttatcttttatttactgttactcatgttctatttcatattttatctcgcacaacataatctatattatattaaaagtacgaagacacttagtgaaatgtcgttcgccttttttatcttttaaaaataaaggtcacactggataaaatagtcatttgcttatttttctaatattaaaaaatagacatttaattaattccctaacatttaggagtttgtataataaattttttccttatttgaactgtattaactattatgacatttagaactttaaaatcattaatattttaccttatataaattttgcacttaaattatttataaataataaataaaataaataaaggagagaataagaacactaaagaaagaggaagtgcgtttggctttggataacatggaagtggtccaatttgttaacctttatacgttctgatttatatgaaactcttctatattagaatatttaaaaatatatagaagtctattccactaataatagtattctatattataattttagttttcaaagcttttaaattagcaattacaattatttggaatattgagcagaaatcaataacaaactccaaaattatcaattattccaacaagataatttatgaaatcttttaacttctctagattgtattaaatttagctgaatataagcgagtttcttaactataattatggcttctaaaagggcatttgaattgtagactttatttatgatataaagtgattcttaaattttttagatttacaaagaatttcatttgcttttctttggaacatgtcatgcatgaatgatttttcacctaatcggttttttaatatttgaaaaagaattcttatcgaataaaaatataattgcaattaaatacctaaaatttggtatgatctagctaattaatattatttaaatactaatctcacttaaataatgatatttccaaattaaataaacacttcacattctaacttcggtcattctttgaaatgcattttaagttgaatattgagtatttttgtggagcggtttcaatttttattagttatatagaccaatattcattatttttaagaacttatatttttattttataactcaaagataaattttaaataattttttattgagttaggtacacgcgcaaagcgcgtacactaagactagtaggtttttatatacgtaaatgtgatagtatcagtaacgtaaagtttaaacctaaaaactaaatgatacattagttatgcagaactatataacgaaaataaaaaattatttaaattatgcataattacattaataaggattcattgactagttttacgatggtcatcaacttaaccttaataaatttgagccaaaattattttaccttatatcaccatccaaacaatcttgggaaaataccatctatgtccatttataagtagcttattacaaaaattgatcaattgataaaatattactaatattagccaattagctatttgtagtcaaaaaggtcaattttttgctttcttttgagtaagtgttactagaatagattgcatacatcttaaagagtttgaatcttagttttgagatgatttggcgaagttttggtgtggtttggattgaaaattcgaagtagatgatgaaacatgaaaaaattatatgtgtatcacactatgcatcatttgtgtatcacatatgtatcataattatatcaaatgtgtatcacgtgtatatccatatatacttgtgtgtgtgatacatgtttgatacatgtgtcgcaaaaatattttttgacttgattttaactacgaattttgataccaaatcgcctccaatcttcctcaaaatttgtatattgactcatctatatgttttcaatgaatttcaaccatacccattgaaaaaagtccttttgtgcttagattttttaaattttgtatatatatatatatatatatatatatatatatatatttatcaccttatttgctacttcatccatgaaattttctttccgtcttgcatctaatgttgctgatcaagtttaaaaatatgaaaggagatctttgcgtgtgattcttggagagaaagaaccttacttatttgggttttcaatttttatatgtgcttgactaatttttgtaagtttataattaatgactagatgtttgtaagttgaaacttatttgggacatttacgtaagattcccaacaatcttgcccaattactttgccctgcatttgggcctataccagagaagcgaagtgcacccatagccagttttagcatgctatttaaaatatatgtataatttataatgtatttgatgattaattaatttgtccaaacttcaggactaagtatcttgaattttttaactgctaatttaaaattcatgatgtagtgtcctaaatgtttgagcagctaatttgaaatttaggaccaagtatcctaaatttatgaattgctaatttaaaattcatggcataagatttggatttttggacataattttgaactttaggatacggtgttatgagtttaaactttaaggtttaaacttcaagacaaagtgttaggtttaaacttcaagacaaggtgttctaaagtttgagcaaattggctaatatttaaataaattgtaaactatatatatattttaaaccgcatgcttaaaaggctatacgaaaccggaaccgacccgacccgttgctctctccactcctcgatctctttcaaggaggaggaggaggaggagtagaaagaaaaaggtaaggtgaaatgggtattcagaatccattcattttcttctttttcttgttcacattgcatagaagttcagttgaatctttgaataaagaaagagaggcaatagaattaaaaggaggaccaaaagatgtagcatgggtggttcaattatctgatcttcatttcagtgttcatcatcctgaaagggcccttaacttctcaaatattgtgggtcccactctttccatgatcaatccttctttggtcttcatcactggtgaccttactggttagtatacactctactttcccctaaattttcactaatattactccctgtttaaaaatttcgttcttacattctgttgatttgatattgggttttatggattttctgtaaaatatagtcctttacccaaaaaaaaaatgatctttttgtttcttgaaactgttgggttggaagttgaggtaggtgggttttttttatggggtgggggtgtaagggggtagtagtggatgttaaaacaagttgctcaatttgctggaagaaaagatttgaatttttatcaatgtgaagtagaaaaagaatttatgatgcatctgtgctggtgggaggtatgaggtgatcgctaaaatagtcgatgtgtgcacaagctactccgtacaccaccgtcattataaaaaaggctatggaggcattttcatggaaaataaggaaaaataaaccaaaaatctgttctttttgtttcttaatactgttgggtcggaaactgagggtggtgggggttgaatgcgttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngagataagggggtaggagtgggcgtcgaaacaagttacgcaattaaaagatgaatttttatctgtatgaaaaattgactgaaaagttactgcaggggttcttttgttgttgtcgggcctcacactataacattaatgaagggtatagtgcgagtatgatgctcagttttacaaggcagcaatgtttgcaaaatgttgcaatttaagatatgattgatacaaaattaaagtgtagttgcaaaagtagcttagttttgttttttgtctcctttgtgtcccgtactctgtcaaatattacacgaggtagttctagaattgaaggtcaagttctgctattaaaagctatgttttgataattatagttggttcagattgatttgtaagtcataattgagatcattgaccactggttagagtttctgaactgctgtttaaggtttatctctgatgttctggtgtcttactttgtgatataactgacctgtaaattgttcagatgcttgttagatataaagcatttccaccctaaagtactttccgacttgtcggttgctaatcatgttacagtaaagcttttgtaaaagtactatgagtttcagtgctcattgctcttataccactgataatttgaactgttactagatggaaagagcaaggatttactaacaatgaaacaagatgaagaggagtggctagaataccagaaagtccttgaagatgtcactaaaaggagtggactgaagaagaacatgttttatgatctccgaggaaatcatgatgctttcggtgtacccgcctttgggggaaaatttgatttttactcaaaatatagcattaacgggcagctaaaaagaagcggactggtcaatagtgtcactattcaggtatctgttaccttattttgttggatacacactctttttcttaacctttcaagctgacaataacctttcaagtgaaactaattggaaaggaaaaccaaaggagtagaaaagaggcttgggaattttccagaacagggaacaaggtagagggaaattttttaacattggcaacgaggaagagtgtagaatccatgtaggggataaacatatctctgtattcaatattttatgaattctattttggtacttgtcacaagattatataaaggaaaatcttaacaatcaaagaaattcatgaatgctattataaagtttgacctaatcttcttttataatcttttgattccgtgtgttagaactttctccttgttaatgtaatctttccaaacaaaaccactatcggtgtaattattattatgttattaagacacccgtagccttcagaatgaaggtgaattatcactgaactttcatatagatcagtcagctaagcatcgattgcaaacttcacaaactatatgatgccgtaatttgagttttttcacttaatatctcatactgtctgcttcaacctcttacgatgactatttactcttatgatccagactggtgaacgcaatattctgttcgttggatttgacagcgctatgtctttaggtcttcggggaccaaccaatctatttggtcatcccactgatcaactattagatgatttaagctctgagctctcggtactagattctcaacccgcaaaatctgttatcaagattgcttatgggcatttcccactttcattttctgcagcatcacgttctggaagaaccctgaagcatacatttctgacgcataacttgtcagcttacttatgtgggcatctgcatacaaagtttggtaagaatttaaagcggcgtcatgagtcaaatcaacatcccttttactctcacaatcttttccagttgaatggacatggatcgcatctgggtaattcgaaaacttgttcagatggggcaactgaatttaaagagttctgggagtgggaaatgggtgactggagaaaaagtagagcaatgcgcatattggccatcgatagagggtggacgtcttttgttgatgttgacttaagattgggagctaagaatgttattatattgccaacatttcctctggactctcgctttactttggagagatcgtttcacaaatgcaagatggatcctttatacttcaacagtattagagctcttgtattttcttcttcgccgattgtgtcagtcgtggctaggatctacgactcaagtcctggaagtcttgttatagtgttggagactcctatgaaaaaaagtggggatgcttcatctagggcggacctttatacatgtccctggaactttaaagcattcgaagatccatctcccgatagatttttgttgcaaatagaagcagttgatattagaggcagatcaactttgactgaactaaggccattctctgttgacggtcttcgtgctaggctttcatggaattggaaagagttcatggttatgggttgtcagtgggaggctttatattaccccatattatggtccttttatttgttaactctttccgtccttctcgttccaaaagctatctttttattttctaagaagcagtatacttacaagaatttcattagtaataaaggagtggtgagttgtgttgcgtggattctatcagagctatataatattccactagcttggctctcaatgattgtttacttgttttacctcattttatgtccttggctttctggccaagttttcaccgaaggaggagaaagaggatacatgacctacaggggttgggtgctgagattgaacgagagggaaaagctagactttcacggatatccagatataatggtgatcatttttccccatctttactttgtggtgttgcctacagtaattgtaattggggcgttggtggctgagagagcaatattccaagatcatcttcgagctgtttctgcaaaaaaggaagatgattatttggtgacgaataaggtatctgcatctactaatggtagaaataagacgctgacactcctccataggcgctggattaggaaaatacttttgttaatttcttcagcaatttgttggaagcattttttggtaagttttcaactgttcaatttgatgtccatctttattctgatttaatgtgcctttttcttactcaaaatgtttattttccttaaaacactggagagttgtttcctttccaagttcccaccgttaaggctgcaccatcttttgggaactcatttgaaatcacttatatccttcagataaactagtcagttcttttccaaattatggaaagtactttcacaaagataagaggaatggtcaatatccttcgtattcttcttgtttagttattcatttttatgttaaacaaacatgatcaacagaagaggataaaggttgtttaaaatatcctgtttccagaattttacattctgccaatgtttttttcaacgaaaactctcaccaccatcacatagtttaggactttctgtcagatatcctcttacttttgtttatgtggaatacgcaacctcgtgattgttccaactaagtggcaatagcttcaatttgtttccagtgaacatagataacgatacttttagagcattgcaggataatgtgtttcaaggagatgtttcaattctaagttttccttgctttaccttgctcataattgttcactaattcaacaatcgacttcttgctaattttgttcttttacgatgcagaactgcagagctctgatcaaagcttatgcaatgaacccattaatccatttcccaatatatagcatgtcgattcctctctttttggcctatactatctacaaaaccagcagagctgagtagaatttttggaagtgaactacatgtaaagccataagtttaatgcagaagcctgtgaaacatacaccggcttattagtaaagaaggcgtcaacagattgcagctctctttagtatctaaagcatctcatgaggggcaatatgaaagcttcaaattccagttgatagaatagtggaagcatagaatgttctttgttatagacatggaaatgctggttactagaattcagtctttatgtagttttacagttttgttgaaaatgcagcatcgtgtttgcagcaatgtagaacttcagatccatgtaaaagtattatacatcatatgggctaattgaggaagttaacaagcagcaaaagagaaggttagtgattgttgaatagattgctggctaaagcttcctattttattcattgcacatcttgaaatgcaccaatcagaagaacagagacaatgcaaagtaagatgaaccaatgagaatgactggaagctttgttctcccttcctcttttcttctgtttagtataaccatgtggtatccgaagccaactactccggattcagcgtaagggaggatcgccctcccaattcgaagttttttcctggaagtttcaaccttgttgactatgacagcagtcacgactataaacagactcgtgactatggcaggtccatggttgtatttctgcaggggagccctctgcatttcctgttagtgtgagaaagtcaggtcgagatctagttctatttatcaatcagagttagaaagctagctttaagcaggccagtctaagtttgactttgccgctggcattccagggaccggcatcttttgagcatgtttagcttccaaaattcctggagtcctacgctacatcattatcactatatgactagtaaataattattttattattatagaaaacgatcaattttttttatcttggcttttcttttttcctcttttcttcttccttatagtataaccatccagtatcgaagctaacatggtcgactaatttagattcagatcagataatccactgagacaagaaaaaagcactctttaccattaggttttctattagaggtaccaaaacctcgggaagatcacaacctctgcaccacaagcctaggtgttctccttcctcttttcacttatttccttatataaataaaatctagcctttggaaactttccctaaaatatagaacaatgcaaacaattgagagtgcaatatagtaaatgaaacagatcgattttatggtgcatttgcaaaagttcttttagttatatgattatgttcacatgattggtaaaataaagcacaagaaatctttgtgaggttcactttcgtctagaattataactcgtcataagagattatttcctagagttactagcatttatgtaagtcacgtgcattgggaggaaatcaattattcactccgtctcattgtgtttgtttggacataaaatttaagaaagaaaaacttttaaaactgtgatattagacatgtcgtaagtattattgtgactataaaatcaagtcattatgggtgaacagagaagtttaacgttactgttttaagaaattatggtggatgtctatcttcctccatgatcttcctctcaaatgcttaatgacatattcaatgacatatttcttcatttttcatgcctatatcttggcattgtaatagatataaagaacacacaattgaagaagaaattataatctctcatctctttctctctatatctcttagcttattttttctagttctatattattactttgagctatatttcataacacgttatcagcacgagactctaccgtctcaagaagctctttgagaagactaatgtataatttttctcctcttttaattatgactgatattataaaaaaaaagtctgttgcgcttgaaatttcgggctagaactatatgacatgggtgttggatgctgaaatctatttagatacaatgggtcttggagacaccattaaagatataaataaagcatctacccaagactgtgctaaggcctcgattttcttgtgccatcaccttgatgaaggttgaaaatagaatatctcacagtcaaagatctacttgttttgtggaatggcttaaaggaaagatatgacaacataaaattggtcactcttccacaagcacgatataattgggctcatctaaggctccaagactttaagtctgtttctgaatataattctgcgatgttcagaattacttctaaattaaaactatgtggagatagtatcagtgactatgatatgcttgaaaaaatgttcacaatattttatgcctccaatatggtcttgcaacagcagtaccgagagaaaggtttcaagaagtactctaagttgatttctcttctcctagtggctgaacgaaacaacaacttgcttatgagaaatcacgaaaatcgatccactaggtctacaccattgcctaaagtggatgaggtgtattcccattatgctaagcgtggaaaaggtcgtgaccctggtcgtggccgtggttaaggaagaaaattttcaagtgttaatcattctccaaagaaaaataacttccaaaagtggaaagggaaagatgagaagccaaaggcaaagggttcagaaactaaatgctatcgttgcggtggaaaagggcattgggcaaatattcgtcgcataccaaaatatttggtcaagctttatcaagcatctctaaagaataaaggccctgaagctaattttgtctatgacaatgaatttgacatcacccaattggatgtggcaaatttctttgagcaccctgatggaaaaataaaccacttgatcggtgatggattcgtggttaaagatgattgagtagttagatttttatttttgcctattcgtagagctaatgaataaacattatgtaatttttattgcgcttaataatactgcttatgtagttcttaaaaatatatgtatttccgaagaaattattttttttaaagattcaatgtttaacaaatcttacaaatgaggggtaatatctaattaattagtacctagtctaagtgatcttttaacacttttgattttcctttctgttactttaattctctttaaaaaaaagtttacaagcaccctggaacaacttttgttacttgaccctcaattcccatcttcctttttaaaaaaaaaatctagtacatcgggacctcaattttttcatgtacatggataaaatattaggaataaagaaaagagaagcagctaagtatgttctttttttacacagatcaattatttttcatgcaatgtgtaagaaaatataaataacttatacatgtaaataattttgttgtagttatattagtcatatgtgtaatgtacttataattgtattatttttgctacgtaattatttgtatcataattagaatttactcaaaattgcattaaaaagtcactattgaatcattggtttaacttttttttttttcttttctctaaaaatactaatatttattcatatacttgttttttgtatgtcaacccatgggaaacaaatatggatatctctcaaagcaaacttggatcaaagtttaattgtaaaaatatttgcttaattgattcatgtacgacacatacaatattcaaagagaagaaatatttctctcatttaagtatgtgtaaggcagatgttactacaatttttggtagtagttgtaacgacccgaccggtcattttgaatattataacctcatttctccatttactgctcaatttatgccttgttattgatttatgacttatcaggttagttggttagggtccggaagaaattcggagtgaaatgagacacttagtctcataattaaaaaaaattaagttaaaaaagtggaccggatatggacctatgtgtaaacgacctcagattcaaattctgataattccaatagctccgtatggtgactttggacttgggagcgtgtccgaaaatttttttggaggtccggagagaaattagacttgaaatgccgaaagttaaatttttaggaagtttgaccgaggggttgacttttttatatcggggtcaaaatccgattctgaaaattttaataggtatgttatgtcatttatgacttgtatgcaaaatttgaggtcaatcggacgtgatttgataggtttcagcgttatttgtaaaaattagaaatttcaaagttcaataggcttgaatttgggcgtaattcatggttttagcgttgtttgaggtgatttgaggattcgactaagttcgtatgatgttttaggactcgttggtatatttggttgaggtcccgagggcctcaggtaagtttcagatggttaacagatcaaaaattgaactacaacagctgctgcaatttccttatgttgaaaattttttctgccagattcgagcctagaaatctctcagaatcgagtccagaaatcgatcccagatgtgagcctagatcgagcccagggtcaagggccatgatcgaagccatgatcgagcccagggtcgaggaccacgatcgaagccatgatcgagcctagggtcgagggtcacagtcgaaggcagggtcgaagacatgatcgaaggccagggtcgagggccatgatcgaggatcaggatcgaagccacgatcgaggcctaggatcgagggtcaggatcgagtcccaggatcgaggacctcgatcgaaggccaagatcgaggcagaaccgaggttgtctgggaagaattataaaatagggacttcgtcctattcgccatttttgacaaattggagcttgaggagaggcgatttttgatatattttcaagaaaaactttaggtaagttccttgtgatcatttctactccataatattgaattattatcgaataatccgactagattacctcattttgaggtgtaaatcggagattggaacttagaaatttggaaataagatttgtagatttgagggttgagttgaggtcggattttggtaaaattgatatgggtaaactcgtggttgaatcggctttcggattttgtaacttttgtcgggtaccgagacataggccccacgagcgatttttgagctaaatttcgaatttttatgaaaaattagtattttcttatgaaattaatttcaataaattttattgactgaaacgaattattttttactagattcgaggcattcggaggccgatttgcgaggcaaaggcatagcagttttaaatctggtcctgagggtataaaaccccggattttggtatcatgtgattattttggaggtgacgcacatgctaggtgacaggcgtnaagcagattctagatatagttgagccgacaggatcaactcaggcaccaactgtgcccattgagattgcaggcattcaagagactttggcccagatattggcatcctgcactggtcttgctcaggtggtttcggctcaagccgcacctgccacttctcaggccgggggaggtactcatacccctgttacccgtactccagaccaggtagtacaaggactacagataccgggggcactaccagcccagccggttgcagctgctcaggccccggtagttcctgttatggcaaatgatgagcagaggagagttgagcaatttgagaggcttcgacctccaccatttagcagtactgagtcagaggatgcttaggattttctggataggtgtcaacggatgcttcagacaacgggtattctggagaccagtggggtctcattcactacttttcagttttctgggtctacactcagatggtgggagacttacgagagacgtaggcatgttggctcagcaccccttacttggcatcagttcttcatctttttggagaagttcgtacctcagtcctgcagagaagagctacgcagacagtttgagtagcttcgccagggtgatatgtccgtgacgcggtacgagatgagattttatgagttggatcatcacgcagtctggttggttccctcttataggtagaggattaggaggttcattgatggcctcacatatcagttgcggttacttatgactagggagagagtgtgtggtgctacttttgatgaggtagtggacattgctcggcaaatagagatggttcataaccaggaacgtggagagagagagagaggctaagaggcctcgtggtccgggtgattacagcggtgttccttcaggtggtcagttacaccgtggtaggggtcgttcttacagacacgctcagacgggtcgtctagctcatcgtggtgcatcaactggccatggttcttacagtgctcactcaggccagtcttcattcagtgcactactagcgcagagttctcatcatgcctcgtccgcacaggcttctgcaggttattcctcgggttatcaggagcagtagttccattagaggaggggttgtttcgagtgcggagaatttggtcacttcaagagagagtgtcctaggttgttgagtggggctccacagtagagttctcgaccaacggcaccagcaccagcagttacaccacctgcccagccagctcggggtgtgggtcaggcagctaggggtcgcccaagagggggaggccaatcaggtggcaggcaggctcgattctatgcttttcctgccaggccagatgttgttgcctcagacgcagtgatcacaggtgttgtttcagtgtgccatatggaggcttctatattatttgaccctggttccacttattcatatgtatcatcgtattttgctcattatctggatatgccatgtgagtccttagtttcacctgtttgtgtatctataccggtgagcgatattattacgtgtgtatcggtcgtgtgtggaaactattggggaactagagactagagttgatctcttattactcggtatggttgatttcgatgtaatcctgggtatgtattggttgtctccatgtcatgctattctgtactgtcacgcaaaaatcttgacgttgacgatgtcggggttgccaaaggttgaatggaggggttctctaaatcttgttcctagcagggtaatttcttatttgaaggcccaacgtatggttggaaagggatgcttatcatatttggcctttgtgagagatgttgatgcagatgctcctattattgattcagtaccggtcctgcgagacttttcaaatgtatttcctgcagacctaccgggtatgccacccgacatggatattgatttcggtattgacttggtgcaggacactcagcccatttctattcctccgtatcgtatggcaccagctaagttaaaagaattgaaagagcaacttcaggaactccttgaaaaggggtttattaggcctagtgtgtcaccttggggtgcaccggttctgtttgtgaaaaagaaagatggtactatgcggatgtgcattgattataggcagttgaacaaagttacaatcaagaataaatatccattgccgcgtattgatgatttatttgaccagcttcagggagagagggtgttctccaaaattgatttaaggtctgggtatcaccaattaaaaatttgggattcggatattctaaagacggcattcaagactcgttatggccactatgaatttcttgtgatgtattttgggctaaccaattccccaacaacatttatgcatttgatgaatagtgtatttcagccatatcttgatttatttgtcgtggtattcattgatgatattatggtgtactcacatagcgaggaggagcatgcacaacacttgggtattgtattacataggctaagagaggagaaactttatgccaaattctctaagtttgagttctggcttagttcggtggcattcttgggacatatagtgtccagtgaaggaattaaggtggatccgaagaagatagagacagatcagaattaacccagaccatactcagttattgagattcagagttttctcagcttggccggttattatcgttgtttcgtgggggggtttctcgtccattgcatcgcctatgactaaattgacccagaaaggtgctccgttcaggtggtcagatgaatgtgaagagagatttcagaagctcaagacagctttgactacaggtcgagtatcggtgttgcctacaggttcaaagtcttatactatgtattgtgacgcgtcgcagattggtctcggcgcagtacttatgcaagacggtagggtgattgcctatgcgtccagacagttaaaggtacatgagaagaattatccggtccatgatcttgatttagcatctattgttcatgccttgaaaatttggcggcattacttgtacggtgtccagtgtgaggtatataccgattatcggagtctacaacatttgtgtaaacataaggatcttaacttgcggaagtgaaggtggttggagttgcttaaggattatgatatcaccattctctatcatcccggaaaggccaatctggtggccgatgccttgactcgtaaggcagagagtttgggcagcttagcatatttaccggtagaagagaggcctttagccttggaggttcaggccttggctaatcagtttgttagattggatgtttccgagccgaatcgagttttggcctgtgtggtttctcgaacttttttatatgatcgcatcagagagcgccagtatgatgattcaaatctgtttgttcttaaggacatgtttcatcacggtgatgccaaggaagtcactattggagatgacagtgtattacgaatgcagggcaggctatgtgtacctaatgtagatggtttgcgtaagctgattctccaggaagctcacagtttgtggtactctattcatccaggcgacgcgaagatgtataaggatttgagacagtactattggtggaggcggatgaagaaagatatagttgggcttgtatctcggtgtttaaattgtcaacaggtaaagtacgagcaccagagaccgagcggattgctacagagacttgaagttccggagtggaagtgggagcgtattaccatagacttcacagttggaatcccacggactttgagaaagtttgatgttgtttgggtgatagtagatcggttgaccaaatctgtgcattttattccagtcgttactaattattcttcggagcggttggctgggatttatattcgcgagattgttcacctacacggtgtgccggtgtccattatttcagatcggggtacacactttacctcacagttttggagggcagtgcagcgagaattgggcacacaggttcagttgagtacaacattttaccctcagacggacggacagttcgagcgcactattcagatattggaggatattctacgcgcttgtgtcatcgattttgggggttcttgggatcaatttctgccactcatagagtttgcttacaataatagctactagtcaagcattcatatggctccgtatgaagctctatatgggagacggtgtcggtctccggtgggttggtttgagcctggtgaggttagactattgggtactgacttggttcaggatgctttagagaaggtcaaagtaattcaggaacggcttcgcacgacacaatctagacagaagtgttatgccaacaggaaggttcgtgatgttgttcacatggttggggagaaggttctgctcaacatttcacccatgaagggtgtgttgaggttcgggaaaaagggcaagttgagccctcggtatattgggccttttgagacacttaagaaagttggggaggtgccttatgaacttgctttgccacctagtatatcaggtgttcatccagtgttccatgtatccatgctctgaaagtatgtcggggatccgtctcacattcttgatttcagtacagtacagctggacggtaatttgacttatgatgtggagccggtgactattttagactgacaggttcgaaagctgaggtcaaagaatatagcaccagtgaaggtgcagtggatagactagccagtcggagaaaatacttgggagatggagcaggagatgcggaacaaatatccacacttatttgagactgcaggtattattctaaacttgttcgaggacgaacgtttgtttaagaggggaaaatgtaacgacccggcaagtcgttttgaatattataaccctatttccccatttactgctcaatttatgccctgctattgatttatgacttatcgggttagttggttcgggttcgaaaggaattcggagtgaaatgagacacttagtctcataattaaaaataattaagttagaaaagtggaccggatatggacctatgtgtaaacgacctaggattcgaattctgataattccaatagctctgtatggtgatttcggacttaggagcgtgtccggaagaatttttggaggtctgtagaggaattagacttgaaatgccgaaagttgaatttttgggatgtttgaccgaggggttgactttttgatatcagggtcgaaatccgattctaaaatttttaataggtatgttatgtcatttatgacttgtgtgcaaaatttaaggtcaatcggacgtgatttgataggtttcggtgttatttgtagaaattagaaatttcaaagttcaataggcttgaattggggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaatattgaactacaacagctgctgcaatttccttgtgttggacatttcttctgccagattcgagcctagaaatctctcaaaatcgagcccagatgtgagcccagatcgagcccagggtcgagggccatgatcgaagccataatcgagcccagggtcgagggccacgatcgaagccatgaccgagcccagggtcgaaggtcacggtcgaaggcagggtcgaagacatgatcgaaggctaggctcgagggccatggtcgatgatcaggattgaagccacgatcgaggcccaggatcgaggacctcgatcgaaggccaagatcgaggcagaaccaaggttgtctaggcagaattaaaaaaatagggacttcgtcccattcgccatttttgacaaattggagcttgaggagaggcgatttttgatatattttcaaggaaaacttgaggtatgtcccttgtgatcatttctactccataatattgaattattaccgaataatccgactagattacatgattttgaggtgtaaatcggagattggaacttagaaatttggaaataagatttgtagatttgagggtcgagttgaggtcagattttggtaaaattggtatgggtagactcgtggttgaatgagctttcgtattttgtaacttttgtcggattccaagacgatttttgagctaaatttcggatttttatgaaaaattagtatttttttatggaattaattccaataaattttattgactgaaatgaattatttgtgactagattcgaggcattcggaggtcgatttgcgaggcgaaggcatagcatagtaaatagtttcatgttttgaggtaaataacaattttaaatctggtcccgagggtatgaaaccccggattttggtatcatgtgattattttggaggtgacgcacatgctaggtgacaggcgtgtgggcgtacaccgaggggattgtgacttgttccgtcccgggaaactgtaaagttgaataatatattgttagctatatgctctctatgtgttgataaaatttgactgtaaatcatgttagaaatca

SEQ ID NO: 67 - DNA, 7632 bp, Artificial sequence
  N.tab-RDR1A exemplary contig Ntab-TN90_AYMY- SS286
  misc_feature (4586)..(4586): n is a, c, g, or t
tttgcataacttttggctctttctttaccaagtaatttttagctcttcttttagctagcaactcttttaccttaatcttgttattttatttttattatactagtattcttacttgtattacacatgtatgtgacttcagagggtaatctggtggtgattagtatttgacagttgaactgcacttggaatttggctatatgggaaagacaattcaggtttttggattcccttatcttctgtctgccgaagcggtcaagaaattcttagagaatcatacaggaaatggaactgtctatgctgtggaggttaaacagtccaaaggtggacgtagagcattcgccatagttcaatttgccaacaacaaaagcgcagaatttatcatcgatttggctagtaagcggttgtattatggaacttcttatttgaaggcttgggaaatgaaaactgatattgtccaaccgcggacatatgtgcaacacatggatggcgtaactctgaattttggatgtctgatatcagacaagaagtttgcagtgttgggaagtaaaggtgtttcagttaaatttggcattggattgaagaaaatttacttctttttatctcatgcttcagctgattataaacttcagctttcatatgaaaatatatggcaagttgtccttcatcgtccatatggtcaaaatgctcagtttctcctcctacaggtttttgaattattccaaaccttttttgctatgcttttcatccttctccatgtgctagtctttgtgaatatttgtgactatttgattgttggttgaatagtacacagttctcatgtattgagtgtcttattgtttgttgatccgcttcttttgaggcaaaatcatttctgtatatctgttatgcatgcaaaagttagacgcacaccagctttgcacctgtcaattgtttgtgtaatactggaaaaggatatatagtgtgtcaactttccggccgatgatgataagaacctgttctgagatggctctatattatttgcaatctgtgttttcctatgtggttcaataaactatctctgcagcctcttgatttgctttacaaaaggccctagtcactaaatgtatccttgtagcttcttgacttcctttgctcaaaaggccgtagtcaccaaaagccaaaaatagaatggctttcattggttaatgaataatgatagaatatatgctaaagattggcttcagagacaagatctagactgtaacaaggttctttatcccaaagaaaaaagaactcacaagattttatgatacagcaagaatttgcaactattttgtgagctttgaaattgtgctatttagagtctgaaagtgtaggaaattgatattgttgcaatggggaaaactgggccaaatacacagatagccactcggagttggtatgaaatgtcagttagacactaaaagtttactagtgtccagatagacacttcaactcgataagaatgcgacacgtgagcacttgggtcataagacctagtgggtgagtgcgatcatggtcggcgagtaaatttctccattctctccaggttccccaccagccactaccaactcaatctctctcccttttccagcttccctatattgcaatgtggcaacccatttatatcttgcctattgttagtttcctcggttaatttctttctcatttgttgacaaactagaagaacaacatggaaactgttaggaaatgaacctttggcgttaatccaaccccaaaagcaagcttgtgagttgagattgctcaaagactgtgtaaggaaattaattacctcatcccataacgatgtgcgactctaacacccccgttctcctctcacgcccaagattggatatttggaccgtggacaatattgagaaacaagaaagaagagaagagggcggggaatagttcacttgattattccctcaagccattggggtttaaaatgacctttacaagaattacaaataaggaaagaaaataaaaggcagcaacacctgggagaattagacaacaacaacaacccaacaataatcccactaatggagtctggggaggatagagtgtacgtagacctttcccctatctcgagggtagagaggctgtttccgggaaacctgggagaattagacatacctctaaagctcaattccagtataaaaacaccaatataggactctaaattggtttagaattgctaaatttctgctccaaaatggctcctttgttcattcaataattgatgatttctgtaaacttaacatcaagatagttcttcttttcctcctttatcctctctttgctcgctaatcaggattttatgatgagttaaaatatattgctattttttttgggcaaagtttggcatctttaaaattgtaatttatttatttattttagtgcccttttttcagtttatgagtcaacctttaccgtttcagctatagtgcccttttttcagtttatgagtcaacctttaccgtttcagctatagtgcccttttttcagtttatgagtcaacctttaccgtttcagctatagtgcccttttttcagtttatgagtcaacctttaccgtttcagctattctttttgggatatttgctaggtgagacattctaatagtgttatttacattgtcctttttcatttgtcagtgatttttgctttgactacggctcaaacatctattttgaagtactgcattgcttctaatagtcttttaacaagcatgaatatttatcatgtgaaatagactggaaaattactaggtgcaaaagtgtgtttcatagactagagccatttggatagcccttttagttcaattatatttttgtgcttagtttttattaaagaagaaaactcgaaagatgattagtgaacattgacagtttctttttaccaactaaatttcttcacgagtatagttgcatcagttaatatatcgctttatataattgaaatatggtagatctgtagtctactttactgatttaccatgagatttattatcgatttattcattcgatttctgtgatttagttatttggtgctcctcggatctataagagacttgaaggctcctgttatagcttctttaaggaaactcctgatgatcagtgggtaaggacaacagatttcactacatcttggatagggctatcttctagcttatgtttggagtttcgtaatggtggtcgacttccaaatttcagcgaaagttttttttactataaagaaagtgtgagccaagttattttacagactggttacaccttttcattctctcaaaatttggctctggttcccattgtccaccttcctgaaggtgttgagttgccctacaagatattgtttaaaattagttccttgatacaacatggatgctttcctggactagcattaaattttaactttttccaattagttgaccctcgaaggagaaatattgcatgcattgaacacgccttggagaaactgtactatttaaaggagtgttgttatgatccagtgaggtggctaactgagcagtatgatgagtacctcaaccgcagacaacttccaaaatctccgtccatcactttagatgatggattggtgtatgtaagaagggtcctagtaacaccatgcaaagtttatttttgtggtccagaggttaatgtatcgaatcgggttctgcgcaattattctgaagacatagataactttcttcgagtttcttttgttgatgaggagtgggagaaaatacattctacagatttattaacaagagctagtagtggaaatggtatcaggacagacatctatgagaggatcctatcaactcttcggaatggctttataattggtgataaaagatttgaatttcttgcattttcatcgagccagttgcgggataattcagtgtggatgtttgcatcaagacctggccttactgcaaatgatataagaacatggatgggtgattttcagcaaatcaggaatgtcgcaaaatatgctgccagacttggccaatcttttggttcctccagagaaactttgagtgtcggtaggcatgaggctgaagttattcctgatgtagtttgttcgcttcatggaaccaactacatcttttctgatggaattggaaaaatatctgctgactttgctcgtagagttgccataaaatgtggccttcagtatactccatcttcttttcagattcgttatggtggatataaaggtgttgtggctgttgatccatattcatcaatgaagttatctttgagaaagagcatgttgaaatatgaatcaaacaacataaagttagatgtcctgggatggagcaaatatcagccttgttatcttaatcgtcaactggttactctcttgtctactcttggggtgatagatgatgttttagagcagaagcaaaatgaagcagtagatcagcttgatgctattttgcatgattctttgaaggcacaggaagctttagaattgatgtctcctggagagaatactaatattctcaaggagatgctaaattgtggttatatgcctgatgctgagccgtttctttcaatnacttggggtgaaagatgatgttttcgaacagaagcaaaatgaagcggtagagcagcttgatgctatcttgcatgattctttgaaggcacaggaagctttagaattgatgtctcctggagagaacactaatattctcaaggagatgctaaattgtggttatatgcctgatgctgagccgtttctttcaatgatgctgcaaactttccgggcttcaaagttgctcgatttgcggactcgatcaagaatatttattccaaatggaagagcgatgatgggatgcttggatgaatccagaaccctggaatatggtcaggtgtttgttcagtttactggtactggacgtagacagttttatgaggaatcacatccttttaatgacagtggatccaccaactgtaatttcattctcgagggaaatgtggtcgttgcaaaaaatccatgcttgcatcctggtgatattcgtgttttaagggctgttgatgttccagctttgcaccacatggtagattgtgttgtatttccccagaaaggaaaaaggtaaaacattactttggaaacctattatatctgtttgatttccatgtaatcttatagagggatttaccatttcttgcctagtcttactaactatcaaaacgtgttatttcttttagtgagtgaatttagaaaaactttacagaaattgcaactagtcttgtcgtaccatgttgaattcttgattagaatcataactggaattcaagttctaccgagtctttgaccatgtaatgtttggtcactcttgcatgaaagcgcggttgttggcatagatgtttggccaacaactatgaaaatcaattcctggaagaagcttatattaggaatctatatagtcatgaaatttcattctggaaacaagcatatactcaaaatttgtgtagcttgacatgaccattatgaagtagcttacattgtgtttgcattgaaaagaaagtggaatttctattgatttagagtagtatttacttatggcctcgttcttacatattggcatatctaaggggggttcctcggttcacgtgaacccacgctctcctccttagatcatgtatagtaatgttatttttttcaaaaagacttaaatatatgtgtgtacccaagctcaaagtatcacatgatgtgatagttattgggtgcacctctctaagtgatattggggattcaaatcttgtgtctcttgttattttttcgtttctttttctagaattgggcccacccacgagtatgtgcatgcctggtgtcatttcttgtgttttaattaagtacttttttctttttctttctttctttgtttggtttattcttcaaaatttcgaactcttgacacattgaaatctctactatcctttagttaccataaaattttataattaaaccaaatgtgtatagtgaagctagttgcagtggatgatgtagcatatagttaatgggttcaactgatcccaacatttctgccacaaagtgtgtgtgtgtatatataaatatatatttgtgtgtgttactattaaaaaaatataatttgaacctaataatttcaaaagcgtaatgggtacaacagtaagaacctaaaggcctgaaccatcaaaattaatatcttggatcaacctcttgttcttacactgtatgtttggcatgactgaaaagttctatcaaataaactagcttccatccttataagaactgtttttatcttttttttcatggcattgtttctcagtgtttaaactttgatgacagacctcatccaaatgaatgttctggaagtgatttggatggggatatctattttgtttgttgggatccagacctgattccgccaaggcaagtccagccgatggattatactccagcacccagcacacagttggatcatgatgtcacaattgaggtacccctggtttcaaacaactaagttttacttacataatttagcatgcttctgatgtattcaacctctcttaccttatagatgtgtatatgtaattaaaattgcccactctctgcttactgcaggaagttcaagagtacttcaccaactatattgtgaatgacagtttgggaatcatagcgaatgcgcatgttgtatttgcagacagagaacctgatatggccatgagtgatccgtgcaaacaactcgctcagctcttttcaattgcagttgactttccaaagactggtgttcctgctgaaataccatctcagttgcgccctaaagaataccccgacttcatggaaaagccagacaagccaacctatcactcggaaaaagttattggaaaacttttccagaaagtgaaggacaaaacacctcaggcttgctctatcgcgaccttcacaatggatgttgccaggaaatcatatgatggtgatatgatagtcaatggattcgaagattacattgacgaagctttttactacaaaagtgaatacgacaacaagcttggtaatttaatggactactacggcgtaaaaactgaggctgaaatacttagtggtggcatcatgaaagcatcaaaaacttttgaccgcagaaaagatgcagaggccattggtattgctgtgaggtctttgaggaaagaagcaaggacctggttcaagaagcgtagtgatatagatgacatgttagcaaaggcttcggcttggtaccacgttacatatcatcatacatattggggtctctacaatgaggggttgaaaagagatcatttcattagctttccctggtgtgtttatgaccagctgatccagattaagaaggcgaaagcacgtaaaaagccggttctccatttatcttctctcgggaatcaactgagtcgcaaattagtgttgatatgatagcattaagccaatctatataatgtaaaaagggtgtgatcataagagaactgctacgcgttgttaacaatcttgtctattaaaccgctgacatttatgcagctctctttaataacagttgtatagcttagtagtatttagcatgcgattgaagttgttttatctctcttct

SEQ ID NO: 68 - DNA, 11112 bp, Artificial sequence
  N.tab-RDR1B exemplary contig  Ntab-TN90_AYMY- SS10620
  misc_feature (7599)..(7698): n is a, c, g, or t
ggatgtttgcatcaagacctggccttactgcaaatgatataagaacatggatgggtgattttcggcaaatcaggaatgtcgcaaaatatgctgccagacttggccaatcttttggttcctccagagaaactttgagtgttggtaggcatgaggttgaagttattcctgatgtagtttgttcgcttcatggaaccaactacatcttttcagatggaattggaaaaatatctgctgactttgctcatagagttgccataaaatgtggccttcaatatactccatcttcttttcagattcgttatggtggatataaaggtgttgtggctgttgatccatattcatcaatgaagttatctttgagaaagagcatgttgaaatatgaatcaaacaacataaagttagatgtcctgggatggagcaaatatcagccttgttatcttaatcgtcaactggttactctcttgtctactcttggggtgatagatgatgttttagagcagaagcaaaatgaagcagtagatcagcttgatgctattttgcatgattctttgaaggcacaggaagctttagaattgatgtctcctggagagaatactaatattctcaaggagatgctaaattgtggttatatgcctgatgctgagccgtttctttcaatgatgctgcaaactttccgggcttcgaagttgctcgatttgcggactagaacaagaatatttattccaaatggtagaacgatgatgggatgcttggatgaatccagaaccctggaatatggtcaggtgtttgttcagttttctggtgctggacgtagacagttttttgaggaatcgcatccttttaatgacagtggatccgccaactgtgatttcattctcaagggaaacgtggtcgttgcaaaaaatccatgcttgcatcctggtgatattcgtgttttaagggctgttgatgttccagctttgcaccacatggttgattgtgttgtatttccccagaaaggaaaaaggtaaaacatcacgttggaattctattaactgtttgatttccatgttaatcttatatagggatttaccattgcttgcatggtcttactaacaatcaacgtctttttcttttagtgagtgcagttagaaaaactttaaagaaaatgcaactagtcgtgtcctaccatgttgaacggtttctttcttcttttttttttcatggcgttgttactcggtggttaactttgatgacagacctcatccaaatgaatgttctggaagtgatttggatggggatatctactttgtttgttgggatccagacttgattccgccaaggcaagtccagccgatggattatactccagcacccaccacacagttggatcatgatgtcacaattgaggtacccctagtttcgaacaactaagttttacttccataatttagcatgcttctgatgtattcaacctctcttaccttatagatgtgtatatgcaataaaaattgcccactgtctgcttactgcaggaagttgaagagtacttcaccaactatatcattaatgacagtttgggaatcatagcaaatgcgcacgttgtatttgcagacagagaacctgatatggccatgagcgatccgtgcaaacaacttgctcagctcttttcaattgcagttgactttccaaagactggcgttccagctgaaataccatctcagttgcgccctaaagaatacccagacttcatggaaaagccagacaagccaacctatccctcggaaaaagttattggaaagcttttccagaaagtgaagaacaaaacaccccaggctagctccatcgcgaccttcacaagggatgttgccaggaaatcatatgatagtgatatgatagtcgatggattcgaagattacattgacgaagctttttactacaaaagtgaatacgacaacaagcttggtaatttaatggactactacggcataaaaactgaggctgaaatacttagtggtggcatcatgaaagcatcaaaaacttttgaccgcagaaaagatgcagaggccattggtgttgctgtgaggtgtttgaggaaggaggcaagggcctggttcaagaggcgtagtgatattgatgacatgttagcaaaggcttcggcttggtaccatgttacatatcatcatacatattggggtctctacaatgaggggttgaaaagagatcatttcattagctttccctggtgtgtttatgaccagctgatccagattaagaaggcaaaagctcgtaaaaggccggttccccacttgtcttcactcgggagtcaactgagtcgcaaattagtgatatggtagcattaaggcgatctatataatgtaaaaagggtgtgatcataagagaactgctatgccttgttaacaaccttttgtcttttaaactggtgatatatttatgtagcactctttaaaacagttgtatagctaagtagtaattagtgtgcgatttaagtttgttttatctctcttctctacatgtttcatgttatgatttatgtattacgcttaacttcaagtgatttcttgatatactctcctccctcaaacgaagacagcaattaaaggtaatttttctcccacaattttttccttaaagtatcgctatatattggcatgactaatccagattcactatgaaggtttttagtgctcccaatcgaagagaatattctcaaacttggagttggacatgatctctgaactccaccctgtcacgagggttgaaaatgttcaaaacttctagaaaattaatggtcttaaccatttttaaccttagaagaatagccagcgtaaagatgttgtctttctatttcagaggtctatcacacaaaataacaatttagtaggaggaatgcttggaagtcctaactttgaagatcataaatcctgcgaaattcaatgacttttagggtgtgtttggtaggaaggaaaatgttttcctagaaaatatttttcatagaaaatgagtgattttatcacttatttttccttgcttgtttggttggtgagcgaaaaactttttccaaaaaatattttatagtgtttggttagagagcagaaaatattttttatgctactctcctcacccccttcccccatgtttccttgctcgcccccttctaaatttttaacgttttcaggactttatttttttcaacgaatttaattattcttctaaaaattcacacaaacccaaaggaactaatgtgttgcatttttacgcaaaaataaatttaaaatttgtgttccataactaaacagaaaatattcttttttttggttgaaaaataaagtactatttctacaacatgaaaatgaagtactcattttattgaaataaaagaaaatacttttctacatcacgaaaagaaaatacttttttgttgaaatgaaaaactactttttctatggaaagaaagtacttttttttgttaaaatgaaaaaaaatacttttttatatcatttaaaaaaattactcatttgttgaaatgcaaaaaaaaaaactatctataacatgaaaataaaatattattaataatatttttatttaggacggtactattaacaaagacaagtgaattttagacccaagtgaatttcttttcgtttgttttcaatatgtactttgtttggccgtccgctcaaggagaagttccacattgcaaacaacgtgcgactttagagtttagacataacaaaacctgatgcagagcgggttggtggggatgtggaacagggtggggaaggttaaaaaagagttttggaaaatattttctcattctcttaataggaaaaatattttcctccaattggaggaaaatgaattcatgagaaaaatgttttccaaaacatttaagccaaccaaacatgaaaaaattgaaaaatattttcatttcataccaaatacaccctaagtgtaaactacatataaagcttggactttaatgaataaagaagcaaaagataaatgaatatgtttaatgctggaataataactataaacacagacaacaaactatatggacaacaggacaagagaattgaaataattacaataagtaaaactttaattctaattgtttcgtgatgtcctcatcaagatagagagagctcatggttacatgggacacacttttacatcttgatgcctgcattgaagtgtcgcctatgttgcacgacgtttcagtagaacagtgggtactatttagggttgaggcgtagtgttgtaaaacccatctctttgtcacttaaagctattaaatgatgtgaagcaaggagttatagcttcattggtgaagtcatgcacggcaaagttcaatcatatatagccctataccgttacacttgaacaaaaatgcccatatccgtcaagagatccagacccaacactaaaatagacacgtggggggacaataaacattttaaaacaaccggttcatttgtccaaacccattactcgatccatcctgaaatcctcttttaacccatctccattgttcattttagaggaggtgccatacacaaatagtggaagaaccctgtggctctacctctctcactaagtgttgtatttttctttgtgttcagtttctcctaagaggctagatataataggcttgcaaataacctttcaacctattttctttttagttcatgcaaaaagaattattccttttcatatttgaaaatgatcctaacatagggtacaccactccccagttgatgttattttagacatagggtacaccagtccttagtctcattacaagtataggggacaccaatccctatctttgttttccaatatagggtatacctatctctagttgatctgatcttaaatgcagagttcaccactccctgatatctttgttaatatagggtacatcattctctcatcacatttccctaacataaggtacaccaatccttagttgaggcaccatttagacaatcagggtataccattcccaaagtatcatattttcctagggtacacttatcccaaccttttattgctttcaataatgaagtagtatagagttttgttacaaataactcatgaaattttcctagtgaaaactagagcaaaacaatttcatttgtttgtttggtttggtatttgagtaggttttacctcaaggcatggggttcgaaatgaccaaatgaataagtctcaatccaaaataaagaaaagaaaagaaaaaaagtgaatccaaaatgcagaagcggatgaaaggatatggactgctcaagacatgactgaagtcacgagcattgcatttcctgttttgatcaaaagaagccatagaagaatgaactagcacttgcggctagcaagcattaaggttcagatcaaagtttgcgtgaagaactaaccaagactcaagatcaatcttcaaaatacgtatagataggaatgttttaactcatagttgataggcttgtttagttttttttgattttgattttggtgtaataaggagttcagcaagcagtaacagcatcgacattagtgaaatcatggcatttaagtagtcccagctaccaaaatttccagaactacactaacctaattcctttatagccaaggatgtgtaggcaaccccgaaagcaaggtttggtcaaaacttttcaaaatgcttcccatggagtgtcaaacaggaaataattggtcgtaattgctcactttatctttgcgcggaaactcttcatattctcgagaaaagaggggcagctgtgagcgcataatattttcctcacatatgaaatcactcctaaaaatatcccaaataattttttttactttgtagaattttaaaagttttatttgattgcatctttatttcattggtagatcatttgcatcattaggattattttaaaaccatcagaaatattttattattcattttttgcatttttagatgataatcacattattggaatttagttggcatgtagttgcattagttaagcacacaaaatcacaaatattgcattggtcgtttttctttgaattttagttagtaattcaattaaagcttaaggtgataattagttaattattagacaaattggataattaggttagggtcaaattaaaaattcaattaaattggccaagtttgaattagaagcaaaagaaaagctgttgggccaagccctgaggctcaaacacctttaataatccgcccagacgaccctcatatccggaccatatcccttttattaccaaacgaccccattttgtccttaatcaatcccaaccgtccctcaaaatcaatccaacgactccgaactgaactccaaaccccttaaaactgtctgaatgaataccccaaaccctaaataccaatcccaattcctcatccctctcaccctctcacccttagccgcctaccacaccaaagagtatctccgtcggtggctcgtctccaagtctgccaaaatatcacaaaataacctttgttccctcctctttccaaatctcttagcctcatctctcgactccctctttatcgattcgtatagcagatctaaaaatgaccataaaccctagtttgcctatttttacctgatttttcagtggtttatgaactctttttggccactccgattgcctctgaagccgcttcgtcttctccaaatttggcttgtaatgaatatttttccatgtagcattggatcgcatgtgaatttggcggcttgtgctcatgtgacaatctccattacatgccagaattcttgtatttgccggtgttggtttggctttccggattccagcttcacggatatgttaaaggttaatcgtggtaagatttcactgctccagcctctttagctgttatgttttatttgaccataatgactttccattcatgactagctatgtgacttcgtacaagtcattttaagaccgtttctgctgtgaaattgcattcgatttactgttagttaatatttcttttttgagttgtaattttctaagttcaagtgtttgttgccttgtttaatttgcaaattggtttagttgatttgttaggattaattggagcaaacctgttattaggttgatatgagtatagagagagtaagagctttgagattggttaattttctttttcttcaattaaaaaatgcaaaggttcagttatgattatcggagtaaggcccaatacttgtttcttttaagtcagtatagtttaaaaggacggggagctatgggtaatttcttaatttagggaatattacggaaaggttctagaaaagggagaaaaggactagtatgcaataaaaaaacagaacatggcaagtgtttagggggcttaggggtaaatacagaagtaaaaggggctgaaaaataaggaaagaatatctgaagtggacagctgcccaaatctgttaaaaaaatgcattttaccctatttttttagggcagacggattccctagtcattctctcatcttttttgcatatagcacacacacatattctctaataacaaactatgcacatttcctctcctcacagacctgaccccatcttttcttatatatannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttatcaccaattataaagccattccgaagagttgataggatcctctcatagatgtctgtcctggtaccatttccagtactagctcttggtaataaatctgtagaatgtattttctcccactcctcatcaacaaaagaaactcgaagaaagttatttatgtcttcagaataattgcgcagaacccgattggatacgtttacctctggaccacaaaaataaactttgcatggtgttactacgacccttcttacatacaccaatccatcatctaaagtgatggacggagattttggaagttgtctgcccttgaggtactcatcatactgctcagttagccacctcactggatcataacaacactcctttaaatagtacagtttctccaaggcgtgttcaatgcatgcaatatttctccttcgagggtcaactaattggaaaaagttaaaatttaaagctagcccaggaaagcatccatgttgtatcaaggaactaattttgaacaatatcttgtagggcaactcaattccttcaggaaggtggacaatgggaaccagagccaatttttgagagaatgaaaaggtgaaaccagtctgtaaaataaattggttcatactttctttatagtaaaaaaaactttcgctgaaatttggaagttgaacaccattacgaaactccaaacataagctagaagatagccctatccaagatggagtgaaatctgttgtccttacccactgatcatcaggagtttccttaaagaagctataacaggaatcttcaagtctcttatagatccgaggagcaccaaataactaaatcacagaaatcgaatgaataaatcaataataaatctcatggtaaatcagtaaagtagactacagatctaccatatttcaattatataaagcgattatattaactgatgcaactatactcgtgaagaaatttagttggtaaaaagaaactgtcaatgttctctaatcatctttcgagttttcttctttaataaaaactaagcacaaaatataattgaactaaaagggctatccaaatggctctagtctatgaaacacactttttgcacctagtaattttccagtctatttcacatgataaatatagtacatgcttgttaaaagactattagaagcaatgcaatacttcaaaatagatgtctaagccgtagtcaaagcaaaaattactgacaaatgaaaacagattatgtaaataacactattagaatgtctcaactagcaaatatcccaaaaaaatagctgaaacggtaaaggtcgactcaaactcaaaaaaggtcactcagataaagaaataaattacaattttaaagatgccaaactttgcccaaaaaccatagcaatatatttttacccatcataaacccctgattagggagcaaagagagaaaggataaaggaggaaaagaagaactatcttgatgttaagtttacagaaattcctcaattattgaatgaacaaagaaaatttagcaatcctaaaccaatttagagtcctatattggtgtaattatactggaattgagctttataggtatgtctaattctcccaggtgttgctgccttttattttctttccttatttgtaattcttgtaaacgtcattttaaaccccaatggcttgagggaataatcaagtgaactattccccaccttcttctctcccttcttgtttctcaatattgtccacgctccaaatatccaatcttgggcgtgagtgagaacgggggtgtttagagtcccgcaaaggtatgggatgaggtaattaatttccttatatagtcttgagcaatctcaattcatgagcttgcttttggggttggattaaagccaaaggtccatttcctaacagtttccatattgttcttcctgtttgtcaacgaatgagaaagaaaaaaaccgaggaaactaacaataggcagagatgtaaatgggttgtcacattgcaatattggggagctggaaaggggagagagattgagttggtagtggctggtggggaagttggagagaatggagaaattaacaaccacgatcgtaactcacccactaggtcttatgacccaagtgttcacgtgtagcattcttatcaagttgaagtgtctatctggacactagtaaacatttagtgtctaactgacatttcatgccaactccgagtggctatctgtgtatttggcccagttttccccattgaaacaatatcaatttcctacactttcagactctaaatagcacaatttcaaagctcacgaacaacaacaaattcttgctgcatcataaaatcttgtgagatcttttttctttgggataaagaaccttgttacagtctagatcttgtctctgaagccaatctttagcatatattctatcattattcattaaccaatgaaagtctttctatttttggcttttggtgactacggccttttgagcaaaggaagtcaagaagctacaagaatatgtttagtgactagggccttttgtaaagcaatcaagaggaccacataggaaagcacagattgcaaataatatagagccatactcagaacaggttcttatcatcatcggccagaaagttttgacacactatatatccttttccagtattacacaaacaattgacagctgcaaagctggtatgcgtctaacttttgcatgcataacagatatacagaaatgattttgcctcaaaagaaggggatcaacaaacaataagacactaaatacatgagaactgtgtactattcaaccaacaatcaaatagtcacaaatattcacaaagactagcacatggagaaggatgaaaagcatcgcaaaaaaggtttaaaataattcaaaaacctgtaggaggagaaactgagcattttgaccatatggacgatgaaggacaacttgccatatattttcatatgaaagctggagtttataatcagctgaagcatgagataaaaagaagtaaattttcttcaatccaatgccaaatttaattgaaacatctttacttcccaacactgcaaacttcttgtctgatatctgacatccaaaattcagagttacgccatccatgtgttccacatatgtccgcggttggacaatatcagttttcatttcccaagccttcaaataagaaggtccataatacaaccccttactagccaaatcgatgataaattccgcgcttttgttgttggcaaattgaacttttgcgaatgctctacgtcctcctttggactgtttaacctccaaagcatagacagttccatttcctgtatgattctctaagaatttcttgactgcttctgcagacagaagataagggaatccaaaaacctgaattgtctttcccatatagccaaattccaagtgcagttcaactgtc

SEQ ID NO: 69 - DNA, 7412 bp, Artificial sequence
  N.tab-RDR2A exemplary contig Nitab4.5_0006831
tacgcataacgatacaaatctactagacagacaagacaagacctagggagattaaaagaaaaactctgagcccacatcaaataaattgtttatttttacaagactggaacacagaaagtgaaaataaaccctgtaaaaaccccattcaactttcccattcgccattcccgttcatacaatgggtgtagagaagagaattacagcaactgtgagggtctcaaacattcctcaaacagccatagccaaacaactcttcgacttcttcgagtcttcaattgggaaaggatcagtctttgcttgcgatattttctcagagcacaagaattggaaatcaagaggtcatggtcgagtccaattcgaaacagcgcaatccaagttacaatctttatcactatctgaacaagggaagctcgtctttaaagggcaccaactcattctaacctcgtcttttgatgatattattgccaggccaattgagcccaactacaggtttcaaaagggaatcttgcatactggggtactgttaaagaatgattacatggaagttttggagacttgggaaaatgtgaagacgttgatcatgcctgaaagaaagagtcttgagttctgggttagtcatgcaaagggggaatgttataggttggaggtccagtttggcgacattatagagacttgtgggtgctctttggaagatgaaaaacctgcactccttttaaaggttgatttttctgttacccatcttgaaaattttagttatttttacatccttttaatttatattgattttagggcttgccaagagctaaggtggccactgacaagtgctgggagctcagtgctttttcaatattgtgtatgtttttgtgtatctcagttagtggcagagccgcagcaaatgaagggggttcaattgaatcccctttgctgaaaaattgtactgcataaatagggcataaacaattcttctatatataaaaacttatgaatcccctcgacataagggaagtcttcattgtagtggcaaaggtagtacaaaaattgctatagctcgcatgtttgaatattttgaatccccttattaaaattcctggctccaccattgtgtgtttataactttatattagacttcctataatagtgatacactctcgagggttgaagaaaatatgaattgaagactaagagctatttatagctctgcaaatggatttacaatgctttaagtaagaagaaagaggggatcagaacgagcccaaagcacacaatctcactattcacagggatggagtcttaataaaaaaaattatcaacgggatttatgcataataatagaaactactaatgtaaaagaatccaggagttctatggtgttaactcccaaaacttataaccaactgagttttcttaattaaccaatggttacatgcttctgctagctgaacgtacaacccgtgcaagctagtcaaaacctccctcttccacttccattttaaagctgtatcaatttttccataagcacattttatgatttagtaaggatgctagggctaacctttataaagtggcctataaaggttagcccttgaagttcctcttcttcctcacttgcagttgcagttgcacgttttctttttttgaaaatatccaattcgaaggagaagaatactcttggttgtgtatgcaactaaaagtcagtcataagttaaaataagcttcaaagtctgcagatcgtgagctgcgcgagcattggggaagatatgatttaagagcgtgctgggaaaacatagtactccgtatggttttgcgtcttgcactatataaaagacattttagacttttattatatagcaagtcaaggatataatatagagagtcaggtagaacttagatagaagaatcagtactgtgcaagagcattcttctgctgtattgaattaaatcagatatctttttgatttcttatgaaacttgttttacagaatcatacagtttacacagtctgtatggtttctgaatagttagctgatcctaccaaaagtctgtggcaggcctagtgaacaaggggggttccgaagtacttggatacagtttcagtaaaagtcttaataataattttggggctaataaggctattttcatggcagggtaaattagacgggattgtgcatctgtcgtggatgcttctgtcaccatttgttctaagaacacatttttttggtctaagaaaacatttattatgcattgactagtctctgagtttaatttctttcactattcattgtcttatgtagagaacagcataaagggataacagttcttttattgatgtatttggtacatgcaatggaaaataacaatagataacatcctatccgagagattactttatgaaaaaaatacacctatggcataaccttaaaaagagaaacaaaaatcccgaagcaaaccaaaaagaaaaagtgaaaatcctctgaagggaaatggagaaacattatgcggtaatgaaatcttggcattggtgaattttagattcttcaacctttatttctgaggcaaagccaatttctttgattttcatttcagtgagtttttgctttcagctaaaacatgcaccaaagctataccaaagagtttctggacccggtgttgcttcaaagttcagttctgaccggtaccatgtttgcaaggaggattgtgagttcctttgggttcgcacaactgatttttctgccatgaagtctatcggatgttcatcctctctttgttgggaaatcgaagacggattgttaagttcagatttgctttctagtttgccatattgtaacaacgatgtgatggatcttgttctggatgaagtaggagatatttattctgcttctgaattagttcctcttgcaagtttcccatcagatctgaagctaccatatgagatccttttccagcttaactctctcgtacacacacataaaattagtcttggtgctgttaaaactgatcttattgaagttcttagcaaattagaattggacactgcaatgatgatccttcagaaaatgcataagttgcagtcaagctgtttcgagcctgtgccattcatcaagacacgcctgcatgtattgggtaaaaacagtaagaaccaaccttcatcatcttacagcaggttagtgaatcaaaacatgatgagtgttcacagagtcttggtaacaccatcaaaggtttactgcctgggcccagagcttgaaacctccaattacatcgtgaagaactttgcatcacatgcttctgatttcttgagagtaacttttgtagaagaggattggagtaaactttctcctaatgccatttctataagtgttgaacaaggtatctttgcaaagccttacagaactaaaatctatcatcggattttgtctatccttcgagatggacttgttattggaactaaaagatttcttttcctggcgttctcggctagtcagctacgttcaaattcagtttggatgtttgcttccaatgagtatgtcaaggcagaagatatcagagagtggatgggctacttcaacaagattcgtagtgtctcgaaatgtgctgcaaggatgggtcagttgtttagcacctcttttcaaacaatggaagtccaatcgccgcatgttgagattcttccagatattgaagtaacctctgatggcgttagttattgcttctcagatggcattggaaagatttctcaagcttttgccagtcaagttgctcagaaatgtggattgagttataccccatcagcatttcaaatacgatatggtggttataaaggcgtgatagctgttgaccgcaattcctatcgaaagctatctctacgtggcagtatgctcaaatttgaatctaagaacaagatgcttaatattaccaagtggagtgatgccatgccttgctatctgaaccgggagattgttatcctcttagctacgttgggagtggaggataaagtattagaagacctgctagataatcatctccatcttctgggcaagatgttgaccactaatgaggcggccttagacgttttggagagcataggtgggggtgacgtaaaaagaatcttaatgaggatgttgcaccaaggatatgcgcccaatttggagccttatctctcaatgatgcttcaatcccattttgagaatcagctttcagacctaagaagtaggtgtcgtatattcatacccaaaggtcgagttctagtaggatgcctagatgaaacaggtatattgaattatggccaggtttatgcacgaatcaccatgactaaggccgagctacaaagtggtcagcaaaatttcttccagaaggtggatgaaacaactgcagtagtaagaggcaaggttgtggtaacaaaaaatccttgtcttcatcctggagatgtaagagtacttgaagctgtgtatgaggtggcattagaggaaaaggcctgggtggattgcattatttttcctcagaaaggtgaaaggtatatgttcagtatacgatatttgagtagaaaatgttcagtatacggtatttcattgggactttcatatccaaactattttacttgatttgacttgtcggccatacactgtccttttccacagtctccatgaagaccgctggaaagtcttgcaatgttttggatgtttaatgataggacataactgaaattaagtggctagtgctaatagattcaattaaatatttttgattagtactaatagcctaaccctactaccttggggttgacgcgttgcactacttgttataaattatagtactaattactaatatgttctagtagttgcactgcatatcttatcttcttctgctggagattcatgtattctatgatgttactaacagtgttatttcagaggtgttcaggtagctttgtctgtccatattaacagctctgagatcagatggagctataccacaaacattaacctaatgtgtatatccagttgccaggcttttgtaatgaagaagaaacttttaataccattagggagcttgcttggcgactgaaacattagttttcgtcaccctttcacaaagactgtctttcatgtttttcgtcttaacataattcatcataatgttttagcatgtgtgaagagttgaaaagaggtctgagcagagttggttagtcttggagcagaagtctgctttcgacattttccctttcccaacttattatttttattgtgcttccacatccctcctccttaagggggttaaagggaaattataatagatgaagaaaacatttctgagagaatcaagaactctttaagttttctgcgtactgctcaaaggggattaaatgttcttttgagtactcaggagtattgaattgtcatgtacaggcctcatccaaatgaatgctccggtggtgatcttgatggagatctttatttcataagttgggatgaaaacctcattcctcgtcaaactgtgacacccatggactacactgggcgaaaacctcgtataatggatcacgaagtcactctagaggtagcttgaactatttcttccacgagtgaagaaacattttttttttctgcatacctctctgatttttggtttctttccctgatttttcgcaggaaattcagagattctttgttgattacatgattagcgacactcttggtgcgatctctactgcacatttggttcatgcagatcgtgaaccagataaagctctgaatccgaaatgcctccagttagcgacccttcactctatggctgtggacttcgcaaaaactggagctgcagctgagatgccaaggtttctaaaaccaagagagtttcctgatttcttggagaggtgggacaaaccaatgtatatttctgaaggggtactagggaagctttatcgtgctatagtcaagtcttctgttcgcggtaactcagatgaccttggctctgtcagagctatccaggatgcttatgatcatgaccttttgttcgaaggttacgaggcattcatcgtaacagctaaaaatcacaaagaaatgtacttggatagaatgaacagcttactcaactactatggagcagagaaagaggttgagatactgacgggcaatttgcgccagaaatctgtgtatttgcagcgggacaacagaaggtattttgaactaaaagatcgaatcttggtttctgccaaaagcctacagaaggaggtgaaagggtggttcactggttgctgcaaggaggatgagaagaagaaactggcatcagcatggtatcaagttacttatcatcctagttactgccaaggaagtgctaattgcctgggatttccctgggtggtaggtgacgttttgctggatataaagttgcataataccagaaaaatcacctcctagttttcaaccatgaaagatactttatgcattgcccttattaacgtgtgtaatcatcctgtcttactaaaatagatgtatagccctctgcctaatgtagacttttgacattttcttatggaagtgctattgactcaatctgcatccgatctaacaattagatgtattaggggttgattggttggaaacaagttatcccagggttagttatcctgggattagttattccaccctcccatacggataaaaaaacactacaatcccgggattagttctaccttgattttatttcaaccaaacatgagataaacacgtctcaaatttaatcctgggattaattatcccttatccctcataccaaacgagcccttagtgctttgagagcatagcaatattttcatgcaaaattggtcataattttctcaagaccttcgcctaggtcctaacatatttccgagtaagtaatcctcaaacccccaacattggaacaaacagaaggcagcataacgagatgccttatttctcagttcttcatatatgtcaaacatgtaaaaaatgtaaaaccaaattcaagtttaggttcgataatgttagtactgtgttatatagctgaggagccaccacttacccagatatctaaaatatatcttaccaagcactcattatctttatcttcttccaatgaatttaatttagaaataaaaaactggaaatactgcgtaatacaagtgcttgacagttgacagtttggttgagtagggataaaagcacaaaaagaactcatataaagggtggagagaaaagcaaaggattatctccgcttcattttttttttctcctttatttgataaggaggattatctccactttgtgaaattgacaatttcgtgagcagcactctttatttatctaataaatttttatcgcccaacaaaaagaaatatcgcaataataaagcaatctcttcgttccattctatgtaaggtgttaaaatacaaaaatacttct

SEQ ID NO: 70 - DNA, 6795 bp, Artificial sequence
  N.tab-RDR2B exemplary contig Nitab4.5_0010542
tattaatagtagctgaatattcctaatttgtcgagctaaattgaggaatcaatcaatcccgagattttttcctgatttttcgcgggttattaagggaaaattctgccctattaattcctaataaacgaatggtatagaatttgtaggaaaaatatggactaggcggataatttaaatactatacacatatttgataataaagtttcatatatggtacatgaatgaaaaaatgcccgatataaatctcgtggacaagacaatacctaggaaaagatgaaaagaaaaactctgagcccacatcaaattaattgtttattttcacaagactggaaaacaaaaaatgaaaaataaaaccctctaaaaaccccattaaacgctcccattagccattcccattcaagcaatgggtgtagagaagagagttacagcaactgtgagggtctcaaacattcctcaaacagccatagccaaacaactcttcgacttcttcgagtcttcaattaggaaaggatcagtctttgcctgcgatattttctcagagcacaagaattggaaatcaagaggccatggtcgagtccaattcgaaacatcgcaatccaagttacaagctttatcactatctgaacaagggaagctcatctttaaagggcaccaacttattctaacctcgtcttttgatgatattattgtcaggcccattgagcccaactacaggtttcaaaagggaatcttgcatactggggtactgttaaagaatgattacatggaagttttggagacttgggaaaatgtgaagacgttgatcatgcctgaaaggaagagccttgagttctgggttagttatgcaaagggggagtgttataggttggaggtccagtttggtgacattatagagacttgtgggtgctctttggaagatgaaaaacctgcactccttttaaaggtttgatttttatgttacccttcttgaaatgttttgttatttttacatccttttaatttatgttgattttagggcttgccaagagctaaggtgaccactgacaagtgctgggagctcagtgccttttcaatattgtgtgtatcagttagtggcagagccacagccaatgaagggggttcaattgaaccccctttgttgaataattgtactgcataaatggggcataaacaattcttttgtgtatatataaatttatgaatcctctcgacataagggaagttttcattgtagtggcaaaggtagttcaaaaattgctttagctcgcatgtttggatttttttgaattcctggctccgccactgatatctgtgtttgtttatattagacttccaataatagtgatacactctcaaggtttgaacaaaatataaattgaagagtaagagctatttatagctctgcaaatggatttacaatgctttaagtaagaagaaagagggaatcagcacgaatccaaagcacacaatctcactattcacagggatggagtcctaataaaaaagttatcaactggattagtgtgtaacaatagaaacttctaatgtaaaagaatccgggagttctatggtgttaactcccaaaacatataaccaactgagttttcttaatgaacccaatggttacatgcttctgctagctggacgtacgacccatgcaagctagtcaaaacctccctcttccacttccattttaaagttgtgtcaagttttccataagcacattttatgatttaggaaggatgctagggctaacctttataaagtggcctatatgaagttcctcttcttcctcacttgcacttgcacttgcagttgcacgttttctttttttgaaaatatccaattcgaaggagaagggcattcttggttgtgtatgcaactaaaagtcagtcataagttaaaataagctgcaaagtctgcagatcgtgagcgagctttggggaagatatgatttaagagtgtgctgggaaaacatagtactccgtatgttgttgtgtctttcactatataaaagacattttagacttttattatatagcaagtcaaggatataatatagagagtcagaggtagaacttagatagaagaatcagtactgtgcagagcattcttctgctgtattgaattaaatcagatatcttttgatttcttatgaaacttgttttacagaatcatacagtttacacagtctgtatggtttctgaatagttagctgatcctaccaaaagtctgtggcagctttgactaatgaacaagggggttctgaagtacttggatacagtttcagtaaaagtcttaataataattttggggctaataaggctattttcatggcagggtaaattagacgggattgtgcatctgtcgcggatgcttctgtcaccatttgttctaagaacacatttttttggtctaagaaaacatttattatgcattgactagtctcagagtttaatttcttccactatatattgtcttatgtagagaacagaataaagggataacagttcttttattgatgtatttggtacatgcaatggaaaataacaatagataacatcctatccgagagattactttatgaaaaaaatacacctatggcataaccttaaaaagagaaacaaaaatcccggagcaaaccaaaaagaaaaggtgaaaatcctctgaagggaaatggagaaacattatgcggtaatgaaatcttggcattgctgaattttagattctttaacctttatttctgaggcaaagccaatttctttgattttcatttcagtgagtttttgctttcagctaaaacatgcaccaaagctataccaaagagtttctggacctggtgttgcttcaaagttcagttctgaccggtaccatgtttgcaaggaggattgtgagttcctttgggttcgcacaactgatttttccgccatgaagtctattggatgctcatcctctctttgttgggaaatcgaagacggattgttaaattcagatttgctttctagtttgccatattgtaacaatgatgtgatggatcttgttctggatgaagtaggagatatttattctgcttctgaattagttcctcttgcaagtttcccatcagatctgaagctaccatatgagatccttttccagcttaactctctcgtacactcacataaaattagtcttggtgctgttaaaactgatcttattgaagttcttagcaagttagaattggacactgcaatgatgatccttcagaaaatgcataagttgcagtcaacctgtttcgagcccgtgccattcatcaagacacgcttgcatgtattgggtaaaaccagtaagaaccaaccttcatcatcttacagcaggttagtgaatcaaaacatgatgagtgttcacagagtcttggtaacaccatcaaaggtttactgcctgggcccagagcttgaaacctccaattacatcgtgaagaactttgcatcacatgcttctgatttcttgagagtaacttttgtagaagaggattggagtaaactttctcctaatgccatttctatgagtgttgaacaaggtatctttgcaaagccttacagaactaaaatctatcatcggattttgtctatccttcgagatggacttgatattggaactaaaaaatttcttttcctggcgttctcggctagtcagctacgttcaaattcggtttggatgtttgcttccaatgagtatgtcaaggcagaagatatcagagagtggatgggctgcttcaacaagattcgtagtgtctcgaaatgtgctgcaaggatgggtcagttgtttagcacctcttttcaaaccatggaggtccaatcgccgcatgttgagattcttccagatattgaagtaacctctgatggcgttagttattgcttctcagatggcattggaaagatttctcaagcttttgccagtcaagtagctcagaaatgtggattgagttataccccatcagcatttcaaatacgatatggtggttataaaggcgtgatagctgttgaccgcaattcctttcgaaagctatctctacggggcagtatgctcaaatttgaatcaaagaacaggatgcttaatattaccaagtggagtgatgccatgccttgctatctgaaccgggagattgttatcctcttatctacgttgggagtggaggataaagtattagaagacctgctagataatcatctccatcttctgggcaagatgttgaccactaatgaggcagccttagacgttttggagagcataggtgggggtgacgtaaaaagaatcttaatgaggatgttgcaccaaggatatgcgcccaatttggagccttatctctcgatgatgcttcaatcccattttgagaatcagctctcagacctaagaagtaggtgtcgtatattcatacccaaaggtcgagttctagtaggatgcctagatgaaacaggtatattgaattatggccaggtttatgcacgaatcaccatgactaaggccgagctacaaagtggtcagcaaaatttcttccagcaggtggatgaaacaactgcagtagtaagaggcaaggttgtggtaacaaaaaacccttgtcttcatcctggagatgtaagagtacttgaagctgtgtatgaggtggcattggaggaaaaggcctgggtggattgcattatttttcctcagaaaggtgaaaggtatagtttagatgctcagtatacgatatttgagtagaaaatgttcagtatacggtagttcagtgggactttcatatccaaactattttacttgacttgacttgtctgccgtacactctgtccttttctagtcttcatgaagaccgatggaaagtcttcttgcaaagttttggatgtttaatgataggacataactgaaattaagtggctagtgctaatagattcaatttaatatttttgattagtactaatagcctaaccctactaccttgtggttgaggcattgcactttttgttataaattatactaattactaagatgttccagtagttgcgctgcatatcttatcttctacagctggagattcatgtattctatgatgttactaatagtgttatttcagaggtgttcaggtagctttgtctgtccataataacagctctgagatcagatgaagctataccacaaactttaacctagtgtgtatatgcagttgccagacttctgtaatgaagaagaaacttttaataccattagggagcttgcttggcaactgaaacattagttttcgttaccctttcacaaagactgtctttcatgtttttcgtcttaacatatttcatcataatgttttagcatgtgtgaagagttgaaaagagttctgagcagagttggttagtcttggagcagaagtctgctttcgacattttccctttcccaacttattatttttattgtgcttccacatccctcctccttaaccccttaaagggaaattataatagatgaagaaaacatttctgagagaaaatcaagaacacttttaagttctgtgtactgctcaaaggggattaaatgttcttttgagtactcaggagtattgaattgtcatgtacaggcctcatccaaatgaatgctccggtggtgatcttgatggagatctttatttcataagctgggctgaaaacctcattcctcgtcaaactgtgacacccatggactacactgggcgaagacctcgcataatggatcacgaagtcactctagaggtagcttgaactatttcttccacgagtgaagaaacttgttttttttctgaatacctctctgatttttggtttctttcctgatttttgcaggaaattcagagattctttgttgattacatgattagcgacactcttggtgctatctctactgcacatttggttcatgcagatcgtgaaccagataaagctctgaatccgaaatgcctccagttagcaacccttcactctatggctgtggacttcgcaaaaactggagctgcagctgagatgcccaggtttctaaaaccaagagagtttcctgatttcttggagaggtgggacaaacccatgtatatatctgaaggtgtactagggaagctttatcgtgctatagtcaagtcttctgttcgcggtaactcagatgaccttggctctgtgagagctatccaagatgcttatgatcatgaccttttgttcgaaggttacgaggcattcatcgaaacagctaaaaatcacaaagaaatgtacttggatagaatgaacagcttactcaactactatggagcagaaaaagaggttgagatactgacgggcaatttgcgccagaagtctgtgtatttgcagcgggacaacaggaggtattttgaactaaaagatcgaatcttggtttctgccaaaagcctacagaaggaggtcaaagggtggttcactggttactgcaaggaggatgagcataagaaattggcatcggcgtggtatcatgttacttatcatccttcttactgcgaaggaagtgctaattgcctgggctttccctgggtggtaggtgacattttgctggatataaagttgcataataccagaaaaatcacctcctagttctcaaccatgaaagatactttatgcattgcttgcccctattaacatgtgtaatcctgccttactaaaaaagatgtatagccctgtgcttaatgtagacttttgacatttcctgatggaagtgctcttgacttaatctgcatccgacctaacaattagatgtcttagtgttttcggagcatagcaa

SEQ ID NO: 71 - DNA, 4318 bp, Artificial sequence
  N.tab-RDR6A exemplary contig Ntab-TN90_AYMY- SS110011
tattgtgtgaacaagcaaatccaaaagtacttgtgactcgttaaagaaaagatgggatcagagggctctgaaaaggatctcgtagtgactcaaattagtgttggtgggtttgacaatgatgtcaatgcgaaaatgctttcagaatatctggaagaacaagttggacaagtatggagatgtagattaaagatttcagccacccctcctgactcttacccaacttatgacattgatgcagagagggtgcatagaatgaaagattacgaaaaagtggtacctcatgcattcgttcattttgcatcttcagagtccgcaaagcatgctctggctgctgctgggggtaatgagctcatattaggaaagaagcctttgattgttagtttgggacctgagaatccctatcgtctgaatcagcggagaagaactaccatgccctttagattttcagatgttagtgtagagttgggagtactggttagtaaagatgactttgtggttggttggaggggacctcgcactggtgttgacttttttgtggacccattcaacggggcatgcagaatacttttcacaaaggatactgctttctctttcaggggtgaagccagacatgccattataaaatgcaatttcaagattgagtttatggtaagggaaatcaatgagataaagaaatgtaaagactttacgtctttggtaatactgttgcagttggcttcatctccattggttttctatagaactgctgatgatgatattgaagaatcagttgcatttgacttgttagacgatgatgatcaatggatccgtaccacagacattacaggtagtggagccattggtaggtgtaatacctaccgtatttcaattcgacctcgcaatggtcctagctttgagaaggccatggcatatttcagttttcgtagggttcccatggtagaaatgtgtaaccggaagatgctccgggtgagggacgagcctgattttggggtgtccatgtcagatcctttcttctgcttccaaaatgaaggtataagctttaaggtcatgtttctggtgaatgcggttttgcacaaaggcatagttaatcagcatcaaatgactaatgaattcttttacttacttagaagtcgtcaggaggaggttaatttagctgcactgaagcacatgttttcttacaaatggcctgtcaatgatgctattcagaagttagtaggcatccagaaatggctgttgaaaaaccctaaacttctcgagagaactggagagttggatgatattgtagaggttaggagattggttatcaccccaaccaaagcatactgtcttccaccgactgtggagctttccaacagagtcctcaggaattataaacatgttgcagatcgatttttgcgagttacattcatggacgagggcatgcggaacttgaataggaatgtgctgacatactatgctgctaccattgtgagggaaatcacatcaaattcgaatccccagagaactgcaatcttccaaagggtgaaaagtattctgagtaaaggatttcatttgtgtggtcgaaagtactcctttttggcattctcagctaaccagctgagggatcgttctgcctggttttttgcagaagaccctaaaattagggtacccggcatcataagctggatggggaggttcagtaacaggaatgtcgccaaatgtgcagctaggatgggacagtgcttttcatcaacttatgcaacagtggaagtcccttcaagggaggttaactcagagcttccagatattgaaagaaatgggtatgttttctctgatggaatcggcatgatatcagcagatctagctatagaagttgcagagaagctgcatttaagtgtcaaccctccttctgcttatcagataaggtatgctggttgtaaaggtgttgttgcttgttggccgacaaagaatgatggcatccgcctttctctgaggccaagtatgaagaaattcgactcaaatcacactatccttgaaatatgctcttggacgagatttcaacctggtttcctgaaccggcaaatagtaaccctgctctcttccttggacgttaaagatggaatattctgggaaatgcaaaaagaaatgatatcagggttggataaaatgctcgtggattcggacgtggcttttgatgtcatcacagcttcatgtgctgaggcaggaaatactgcagctctaatgttgagtgcaggttttaaacctcaaagtgagcctcatttaagagggatgctctctagcataagagcttctcagcttggggacctcaggaataaggcaaggatatttgttccttcgggaaggtggttgatgggctgtttggatgaattaggtgagcttgagcaaggccaatgctttattcaagtgtcaagcccttctttggagaattgttttattaagcatggtccgaaattttctgatataaagcaaaatcttcaagtagtaaagggccttgttgtaattgcaaagaacccgtgtcttcatcctggggatgtgaggattctggaggctgtagatgttcctggtttacaccatctctatgattgtctggtctttcctcagaagggggataggccacattcaaatgaagcatcaggaagtgacctggatggtgacctctactttgtgacttgggatgaaaatctcatcccacccagtaagaaaagctggatcccgatgaactatgaacctgcggaagtcaaacagttgggtcgtcaagttaatcatatggtagctacccgacaataattatatttcttgcagctttggttcatatcttcctaattatttggttagctttcagatgcttatacgtttgttttgcaacataatgttcatttggttagaatgttgatgaaatatttataagccttctagaaaagctaccattactttttttagctaatatttatatttaacaactgtttttatttttgttgcacaagtaagatatacttgtttctcacttttcttgtcttatgagccccaacttgacgcttctctgatatgtgtattttcctttttatggtaagctgtttgcatttgatcatatttaagttgtttagtttgtccgtagttgatttagttgtaatagaagagggagactctttttctgttggcacactcgctagaaataaaaagataaagctaaagacacttgcaaagatcttgtgctcttcatggctcaaatatttgttctatcaggctatattcaaaaatttgcgcttgattagaaatttaagtctactttttggaacttgttattggttggatcatttaactggttgcctaatattagtttattttttatttttcacgcgccgtatttattcagttttaaatatcttttgctctgtttacaggacataatagattttttttcaaaaaacatggtccaagagagtctaggagaaatctgcaacgcacatgtggttcatgctgacctcagtgaatttggagctttggatgagaagtgcctaaaattggcagagcttgctgctctagccgttgacttccccaaaactggaaaacttgtgaccatgccgtttgacctcaaaccaaaaatgtatcccgacttcatggggaaagaggaattccaatcatacaagtcaaagaaaattttgggcaaattgtataggcaagtgaaagatgtatgtgatacagagggtgaagtatctgctggacttgagcttgtcccaaacgacatcccatatgactctagtcttgagatcccaggatccacagctttcatagacgatgcatggaacagcaaatgttcttatgatggtcagttgcatggacttttagggcaatacaaagttaacagggaagaagaggtggttactggacatatatggtccatgccgaagtacagtgctaagaagcaaggggaattaaaagagaggctgaagcacgcatataatacgctgaggaaggagttcaggaatgtcttcgagcgtatggatcctgattttgatcttcttcccgatgatgagaagaatgatatgtatgaaagaaaggcatctgcatggtatcaggtcacctacaatcctcattgggttgctagatcactggagttgcaattgccagatgctatctcaagtactgtaatgctaagttttgcatggattgcagcagattaccttgctcgaattaaaattaggcacaggggattgcagtattccgactctaccaagccaatcaattcccttggaaggtatcttgttgacaaaatatgatactcatcaactctttggctgcaaggcctctctgccattat

SEQ ID NO: 72 - DNA, 1500 bp, Artificial sequence
  N.tab-RDR6B exemplary contig Ntab-TN90_AYMY- SS48576
aatatttataagccttctagaaaagctaccattacttttttagctaatatttgtatttaacaaatgtttttatttttgttgcacaagtaagatatacttgttgctcacttttcttgtcttatgagccccaacttgatgcttctctgatatgtgtattttcctttttatggtaagttgtttgcatttgatcatatttaagctgtttggtttgtccgtaattgatttagttgtaatagaagagggagactctttttctgtcggcacactcgctagaaataaaaagataaagcaaaagacacttacaaagatcttgtgctcttcatggctcaaatatttgtgattatttgtgctatcaggctatattcaaaactttgcgcttgattagaaatttaagtctactttttggaacttgttattggttggatcatttaactggttgaataatattaatttatgttttatttttcacatgtggtatttattcagtcttaaatatcttttgctctgtttacaggacataatagattttttttcaaaaaacatggtccaagagagtctaggagaaatctgcaacgcacatgtggttcatgctgacctcagtgaatttggagctttggatgagaagtgcctaaaattggcagagcttgctgctctagccgttgatttccccaaaactggaaaacttgtaaccatgccttttgacctcaaaccaaaaatgtatcccgacttcatggggaaagaggaattccaatcatacaagtcaaagaaaattttgggcaaattgtataggcaagtgaaagatgtatgtgatgcagagggtgaagtatctgctggacttgaggttgtcccaaaagacatcccatatgacactactcttgagatcctaggatccacaactttcatagacgatgcatggaacagcaaatgttcttatgatggtcagttgaatggacttttaggacaatacaaagtgaacagggaagaagaggtggttactggatatatatggtccatgccgaagtacagtgctaagaagcaaggagaattaaaagagaggctgaagcacgcatataatatgctgaggaaggagttcaggaatgtcttcgagcgtatggagcctgattttgatcttcttcctgatgatgagaagaatgatatgtatgaaagaaaggcatctgcatggtatcaggtcacctaccatcctcattgggttgctagatcactggagttgcaattggcagatgctgtctcaaatactgtaatgttaagttttgcatggattgcagcagattaccttgctcgaattaaaattaggcacaggggattgcagtattccgactctaccaagccaatcaattcccttggaaggtatcttgttgacaaaatatgatgctcatcaactctttggctgcaaggcctctctgccattatcaatacctggtttgatgtcctttagaacctcctaatctcctgtttccttatgttgatctgttattatat

SEQ ID NO: 73 - DNA, 1512 bp, Artificial sequence
  N.tab-RDR6C  exemplary contig Ntab-TN90_AYMY- SS1589
aatatttataagccttctagaaaagctaccattacttttttagctaatatttgtatttaacaactgtttttatttttgttgcacaagtaagatatacttgttgctcacttttcttgtcttgtgagccccaacttgatgcttctctgatatgtgtattttcctttttatggtaagttgtttgcatttgatcatatttaagctgtttggtttgtccgtaattgatttagttgtaatagaagagggagactctttttctgtcggcacactcgctagaaataaaaagataaagcaaaagacacttacaaagatcttgtgctcttcatggctcaaatatttgtgattatttgtgctatcaggctatattcaaaactttgcgcttgattagaaatttaagtctactttttggaacttgttattggttggatcatttaactggttgaataatattaatttatgttttatttttcacatgtggtatttattcagtcttaaatatcttttgctctgtttacaggacataatagattttttttcaaaaaacatggtccaagagagtctaggagaaatctgcaacgcacatgtggttcatgctgacctcagtgaatttggagctttggatgagaagtgcctaaaattggcagagcttgctgctctagccgttgatttccccaaaactggaaaacttgtaaccatgccttttgacctcaaaccaaaaatgtatcccgacttcatggggaaagaggaattccaatcatacaagtcaaagaaaattttgggcaaattgtataggcaagtgaaagatgtatgtgatgcagagggtgaagtatctgctggacttgaggttgtcccaaaagacatcccatatgacactactcttgagatcctaggatccacaactttcatagacgatgcatggaacagcaaatgttcttatgatggtcagttgaatggacttttaggacaatacaaagtgaacagggaagaagaggtggttactggatatatatggtccatgccgaagtacaatgctaagaagcaaggagaattaaaagagaggctgaagcacgcatataatacgctgaggaaggagttcaggaatgtcttcgagcgtatggagcctgattttgatcttcttcctgatgatgagaagaatgatatgtatgaaagaaaggcatctgcatggtatcaggtcacctaccatcctcattgggttgctagatcactggagttgcaattggcagatgctgtctcaaatactgtaatgttaagttttgcatggattgcagcagattaccttgctcgaattaaaattaggcacaggagattgcagtattccgactctaccaagccaatcaattcccttggaaggtatcttgttgacaaaatatgatgctcatcaactctttggctgcaaggcctctctgccattatcaatacctggtttgatgtcctttaggacctcctaatctcctctttccttatgttgatctgttattatatgtaatttgttcc

SEQ ID NO: 74 - DNA, 4209 bp, Artificial sequence
  DCL2A Cdna
atggatccaactgaagttacagtctctgaaaaccaacagctctgcgctgtctctctgccttttgcaagaagttatcagttagaggcattggagacggcattaaagcagaacactattgtttatttggagactggttcgggaaagacccttatcgctattatgcttctgagaagctatgcttaccttcttcgaaagccgtctccctacatagctgttttcttggtcccgactgttgttttggtggctcagcaaggagatgctctcattatgcatactgacttgaaagtgggcaagtattggggagaaatgggtgtcgattattgggatgctgctacatggcaacaacaggtggacaatcatgaggtgctggtaatgacacccgcaatactacttgctgctttgaggcatagcttcttaaaaattgaaatgatcaaagttctaatatttgatgaatgtcacaatgcacgagccaaacacccttatgcctgcataatgaaggaattttatcatcgtcaattaaccctcgaaagtgcacaacttccgagaatattgggaatgactgcttcccctataaagactaaaggttctagcgcggaatttacttggaaaatgattcgtgacctcgagaaccttatgaattccaaggtatacacgtgtgtcagtgaatctgttttggccgagtatatcccattctcaactccaaagctgaagatatatcaacatgtggatattccgtgcacattgtttgttagtttagtttctgacctgataaggttgaaagacaagtatgaagattcgatttcaaaatcgagcctatctgatttaagtgctggttctgcaaaaaaaagattgtcaaaactatattcgtccttcgtattttgtttgagtgagctgggtgtttggccggctttcaaggcagccgagttcttgtcctctgaagaaagcgacttcttttcatggggagaattagatgtttgtgctcaaacgattgttagaaatttcagtttgggtgcatctaaggtcttctcggctcgcataccatctggttctcattggtcacttggtggtgatattcatgcaaatgttgatgctggatatttgacatccaaaattaatagtcttatagagtctcttcttgagtacagagacctgaaggatttgagatgcatcatatttgttgagagaatcattacagctattgttcttcggtctttgttaaatgagttgcttccggaattaagtggttggaaaactgagtacactgcaggacatagttctatgctgcagtcacagagcagaaatgttcaaaataaaattgttgaagaatttcgcaagggtgtggtcaacattattgtagcgacttctattcttgaagagggtttggatgttcaaagttgcaaccttgtcattcgatttgatccctcagcaaccgtttgtagctttatacagtctcgtggtcgagctcgaatgcagaactcacactttattatgatggttgggagcggggatgcttcaactctcactcgcatgcaaaattatatgaaaagtggggagatcatgcggcaagaatcattacgcaatgcttctattccgtgttcacctcttgatgatgaattgcacaatgaaccttgctataaagttgaaacaactggagcagttgtcaccctcagttctagcgtgtcattgttatacttctattgctcaaggctaccttcggatgggtactataaacccagtcctaggtgtgttattgaggaggaaacagaaacctgcaccctacatcttcccaaaaattgcccactgcaaaaagttattagtgtgaaagggaataccaaaatattgaagcaacttgcttgccttgaagcttgtaaagaacttcatcgtgagggtgcattgaccgacaatcttgttcctgatatcgtggaggaagaagccatcatcaaagaattgggatgtcaaatctatactgatgaagaatcaaagtactttccaccagaattggttagtcattgtgccaatgatacagaagcagtgtactactgctacgtagttgatttgcaacatgactcttacagcaattaccagcttcgtgggataatacttgctgttaggacaaggctcaagttagacgatgaaagattgatttttgacttggatgttgataatggaagcttgctagttcaagtaaattatagtggagttgttaggcttacgtctgaagagattttacgatgtcagaggtttcaagtcagtctattcagaattcttcttgaccgcgatctgagtaagctgcaggacgctttggctgcagttcaactgcctgttggttctgcagtttctgattatcttttgcttccatctttgggttcaactcaaaaccctcaaataaattgggaatgtgtgaattctgtgctgtttccttctcaagttcttggggataagcatattgattgttgttctacacaaggtcgcaaacgcagtgtgaacatcaaaactggggtggtatgcagctgcatgttggagaactcattggtctgtacgcctcataatggctatgtgtactgcattaccggttttttggataatttagattgcaactcattgctggaacagagaactggagaatccattacctacaaagaatactacaaaaagcggcatgggatcaacttatgctttgaaggagaaccacttcttagggggaaacgccttttcaaagtgcacaattaccttcaaagatgcagaagtcagaaggctaaagattcaactgattcatctgttgaattgccccctgaactttgttccatcatcatgtcacctatctctatttctaccctttatacctattcatatgttccttcggtcatgcatcggattgagtcattggttatggcctcaaacttgaaaaggatgcactcggatcagtgcacgcaaaacatttttattccaaccgctaaggttctggaggcaattactacaaagaaatgcgttgaaaagtttcatttggaatcacttgagacacttggagactcttttctcaaatatgctgcgagtatacagctgttcaagacttatgaaaatcatcacgagggtctactcactgttaagaaaaacaaaattatttccaatgctgcattgtgcaagcttggatgtgctcgtaaaataccgggatttattcgtaatgagccattcgatcttaaaggatggctcattcctggtgataattctcaagttcgcaatttcgatgaagaagtgctgatgccttctgtaaaaatgtatagtaggggaaggcagaagattaaaagtaagagagtagctgatgttgttgaagcactgataggtgcctttctcagctcaggtggtgaagtagcggctttgtcatttatgaaatggcttgggatcgacattgattttgtagctgcacctacgctgaggcacttccccatgaatgctgagaagctagttaatattcgatacttggaatcactgctagattacaagttccatgatccttctctgcttgttgaagctctaactcacggatcttacatgctacctgagattccacgatgctatcagcgcttggaatttcttggagatgcagtgctagattatgctgtcacagcgcatctctatttcaaatatccgggactgtctccaggactcattactgatttgaggtctgcctccgtaaataatgaatgctacgctcaggctgctgttaaggctggcctacacaagcacattcttcatgcttcacaagatctgcagagacagatggttaatactgttctgaattttgagaaattggatcccgtgtccacgttcggatgggagtccgaaactacttttccaaaggtgcttggagatgttattgagtctcttgccggggcaatctttgttgattctggcttcaacaaagatgttgtgtttcagagcatacgaacacttctggaaccgttgattacaccggatacagtaaagctccatccaatcagagagttaagtgaactatgtgaccagaaaggttatataaagaagaagaacgttgtctctcgtgagaatggtgtggcgtatattacagtagaggttgaagcagatggtgttagtcacaagttcacgtgctcggaacgagacaaaaaaatggccgagaaggtggcttgcaaaaatgtactcaaattgctgaaggaatgtgcatctgatgcttag

SEQ ID NO: 75 - DNA, 4209 bp, Artificial sequence
  DCL2B Cdna
atggagccagctgaagttacagtctctgaaaaccaacagctctgccctgactctctgccttttgcaagaagttatcagttagaggcattggagacggcattaaagcggaacactattgtttatttggagactggttctggaaagacccttattgctattatgcttttgagaagctatgcttatcttcttcgaaagccttctccatacatagctgttttcttggtcccgacggttgttctggtggctcagcaaggagatgctctcatcatgcataccgacttgaaagtgggcaagtattggggagaaatgggtgtcgattattgggatgctgctacatggcaaaaacaggtggacgatcatgaggtcctcgtaatgacacccgcaatactacttgctgctttgaggcatagtttcttaaaaattgaaatgatcaaagttctaatatttgatgaatgtcacaatgcaagaggcaaacacccttatgcctgcataatgaaggaattttatcatcgtcaattcacccttgaaagcgcacaacttccaagaatattcggaatgactgcttcccctataaagactaaaggttctagcgcggaatttacttgggaaatgattcgtgacctcgagaatcttatgcattccaaggtatacacatgtgtcagtgaatctgttttggcagagtatatcccattctcaactccgaagctgaagatatatcaacatgtggatattccgtgcacattgtttgttagtttagtttctgacctgataaagttgaaagacaagtatgaagatttgatttcaaagtcaagcctatctgatttaagtgctggttctgcaagaaaaagattgtcaaaactatattcgtccttcatattttgtttgagtgagctgggtgtttggctgactttcaaggcagccgagttcttgtcctctgaagaaaccgacttcttttcatggggagaattagatgtttgtgctcaaacgattgttagaaatttcagtttgggtgcatctaaggttttctcggctcgcataccatctggttcacattggtcacttggtggtgatattcatgcaaatgttgatgctggatatttgacatccaaagttaatagtcttatagagtctcttcttgagtacagagacctgaaggatttgagatgcatcatatttgttgagagaatcattacagctattgttcttcggtctttgttaaatgagttgcttccggaattaagtggttggaaaactgagtacacagcaggacatagttctatgctgcagtcacagagtagaaatgttcaaaataaaattgttgaagaatttcgcaagggtgtggtcaacattattgtagcgacttctattcttgaagagggcttggatgttcaaagttgcaaccttgtcattcgatttgatccctcagcaaccgtttgtagctttatacagtctcgtggtcgagctcgaaagcagaactcacactttattctgatggttgggagcggggatgcatcaactcttactcgcatgcaaaattatatgcaaagtggggagatcatgcggcaagaatcattacgccatgcttctattccgtgttcacctcttgatgatgaattgcaaaatgaattgcactataaagttgaaacaactggagcagttgtcaccctcagttctagcgtgtcattgttatacttctattgctcaaggctatcttcggatgggtactataaacccagtcctaggtttgttattgagaaggaaacagaaacctgcaccctacatcttcccaaaaattgcccactgcaaaaagttattagtgtgaaagggaataccaaaatattgaagcaacttgcttgcctcgaagcttgtaaagaacttcatcgtgagggtgccttgaccgacaatcttgttcctgatatcgtggaggaagaagccatcatcaaagaattgggatgtcaaatctctaccgatgaagaatcaaagtactttccaccagaattggttagtcattgtgccaatgatacggaaacagtgtactactgctacgtagttgatttgcaacatgactcttacagcagttaccagcttcgtgggatagtacttgctgttaggacaaggctcaagtttgacgatgaaagactgacttttgacttggatgttgataagggaagcttgctagttcaagtaaattatagtggagttgttagacttacttctgaagagattttacgatgtcagaggtttcaagtcagtctattcagaattcttcttgaccgcgatctgagtaaactgcaggacgctttggctgcagttcaactgcctgttggttctgcagtttctgattatcttttgcttccatctttgggttcaactcaaaaccctcaaataaattgggaatgtgtgaattctgtgctatttccttctctagttcttggggataagcatattgattgttgttctacgcaaggttgcaaacgcagtgtgaacaccaaaactggggtggtatgcagctgcatgttggagaactcattggtctgtacgcctcataatggctatttgtactgcgttaccggtttcttggataatttggattgcaactcattgctggaacagagaactggagaatccgttacctacaaagaatactacaaaaagcggcatgggatcaacttatgctttgaaggagaaccacttcttagggggaaacgcattttcaaagtacacaattaccttcaaagatgtagaagtcagaaggctaaagattcaactgattcatctgttgaattgcctcctgaactttgttccatcatcatgtcacctgtctctatttctaccctttatacctattcatatcttccttcggtcatgcatcgggttgagtccttgattatggcttcaaacttgaaaaggatgcactcagatcagtgcactcaaaacatttttattccaactgctaaggttctggaagcaattacaacaaagaaatgcgtcgagaagtttcatttggaatcacttgagacacttggagactcttttctcaaatatgctgcgagtatacagctgttcaagacttatgaaaatcatcacgagggtctactcactgtgaagaaaaacaaaattatttccaatgctgcattgtgcaagcttggatgtgctcgtaaaataccgggattcattcgtaatgaaccattcgatcttaaagggtggctcattcctggtgataattcccaagttcacaatttcgatgaagaagtgctgatgccttcagtaaaaatgtatagtaggggaaggcagaagatgaaaagtaagagggtagctgatgttgttgaggcactgataggtgccttcctcagctcaggcggggaagtagcggctttgtcatttatgaaatggcttgggatcggcattgattttgtagatgcaccttcgccgaggcacttccctatgaatgctgagaagctagttaatgtcagatacttggaatcactgctacattacaagttccatgatccttctctgcttgtagaggctctaactcacggatcttacatgctacctgagattccacgatgctatcagcgattggaatttcttggagatgcagtgctagattatgctgttacagcgcatctgtatttcaaatatccgggactgtctccaggactcattactgatttgaggtctgcctccgtaaataatgaatgctacgctcaagctgctgttagggctggcctacacaagcacattcttcatgcttcacaggatctgcagagacagatagttaatactgttctgaattttgagaaactggatccggtgtccactttcggatgggagtccgaaactacttttccaaaggtgcttggagatgttattgagtctcttgccggggcgatctttgttgattctggcttcaacaaagatattgtgtttcagagcgtacgaacacttctggaaccgttgattacaccggatacagtaaagctccatccagtcagagagttgagtgaactatgtgaccagaaaggttatataaagaagaagaatgttgtctcacgtgtgaatggcgtggcgtatattacagtagaggttgaagctgacggtgttagtcacaagttcacgtgctcggaacgagacaaaaaaatggccgagaaggtggcctgcaaaaatgtactcaaattgctgaaggaatgtgcatctgatgcttag

SEQ ID NO: 76 - DNA, 4065 bp, Artificial sequence
  DCL4A Cdna
atggaaggcggcgactttgagaacggcactgagagtccaccaagtgctgcaacttcaccgatcactgagcagctttctgctctgtctctcaatggcgacatagattctcctgtttcagttcagaaaccagagaaagaccccagaaaaattgctagaaagtatcagatggatctttgtaaaaaagcattagaggagaatgttgttgtatatctgggaacgggctgtgggaaaactcatattgccgtcttgcttatctatgagatgggacagctgataaggaaaccccagaagagcatttgcgtgtttcttgctcccactgtggcgttggtgcaacagcaagccaaggtcatagaagactctattgacttcaaagttgggacatactgcgggaaatccaagcatttgaagagccacgaagactgggaaaaagaaatggaacagtatgaggtccttgttatgacccctcaaatactattacataatctttcccactgctatatcaggattgaatttattgctcttctgatctttgatgagtgccactatgcacaagttgagagtgatcatccttatgctgagattatgaagatattttacaaacctgatgtagtaaaactgccccgtatacttggcatgactgcatctccaatatcaggaaaaggtgctactgttgaaggccttgagaccttacttcgctctaaggtgtattctgttgaagacaaggatgaactggaacagtttgttgcatcacctaaggtgaatgtatactactatggtcctggcgccgcgtgtcttactaaagcttactctcagaagcttgaagagataaaacaccagtgtgtgatggtgcttcacaagaaagcagttgatcatagcacactcaggaacacaaaaaagatgctcaagagattgcatggacatctaattttttctctggaaaacctaggtgtctttggggcattacaggctagttgcatcctcttaaagggggatcactatgagcgccatcaaatggtggaagctgatgttaatgctagtgatgactctttgtgtgacagatatctgagtcaggtcgccacaatgtttacttctggttgtgctaaagatggtatgaatcctgatttgacacttgtggaagttctgaaggagccttatttctccaaaaagcttttacgtctaattggaatcctttccaacttcggggtacaaccagatatgaagtgcataattttcgtcaataggatcgtgactgcgagatctctttcttatatgcttcagcatctgaaagttttatcttcttggaaatgcgggtttcttgtaggagtgcactcaggattgaagagtatgtcacgaaaaaatacaaatatcattctcaataagttccgttctggtgagctcaacttattggttgccaccaaagttggcgaggaaggacttgatatccagacatgctgccttgttatacgatttgatcttcctgaaactgttgccagttttatacagtctagagggcgtgcccgcatgccaaagtctgaatatgcatttttggttgatagtgataaccaaagggagcttaatttgattgagcatttttcaagaaatgaagctcggatgaatgatgagatttcttccaagaaatcttgcacgacagttatagattttcaagaaaatatatacaaagtagatatgactggtgcaaccatcagttctgcatcaagcatttcgttgcttcaccattattgttccaagcttccacgtgacgagtttttctgccccaagccgcagttcttctactttgatgacatagatggaactatttgcaaattagttttgccttcaaacgctccaatgcatcaaattgtgagtgctcctcaatcttctatagaagcagctaagaaagatgcctgtttaagagcctgcaagagcttgcatgaacttggagcactcactgattatcttttgccggatcaagcagatgaagacctgattcacgttttttctgactcagaaagctcagatggcgaagacgcacgagaagagctccatgagatgattgttcccgctgcctttaaagaaccatggactgagacagagagtcctgtttgccttaactcgtactacatcaacttttcaccttgtccaattgatagggtttataaaaagtttggcctatttttgaaagcacctcttccccaagaggctgagagaatgaaacttaatctcaacttggctcgaggtagatctgtagagacagagcttattccatcaggagctactaattttgagaacaatgagcaggtacaactagcagagaagttccagagaatgttcctcaagatcattcttgaccgctctgagttcatttcagaattcgtatctttggaaaaacaagattatgttgactcagcttcaaaatcttacctacttcttccactcaatttatgtggacacaataaaatttccgtggactgggagcttgtcagaagatgcttgtcatcaccaatttttgggacttcagtatatgcaggcaatagtgagatatccaaatttgatgagcagttgcaacttgccaatggttccaaaagcgtacatgatgttgccaatagtttggtatatgttccatgcaaggatacatttttcttcatatctgatgttgtcaaagaaagcaatgcatacagcatatataaagattcaaaaaaccatgtcaaacactattatgatacttttggtatccatctttcgtaccctgagcaaccactaataaaagcaaaacaactcttctgtttggacaacttgctacgaaagaaaggctattcagagttgcgtgataaagaggagcactttgttgaactacctgctgaaatttgccagctcaagataattgggttttccaaagatattgggagctcattatcactgttgccatcgatcatgcatcgtcttgaaagcctacttgtcgctattgaactaaagggctgcctatctgcttctttccctgagggaagggaagtaacaatagatcatgttcttgaagcacttactacagagaaatgcaatgaacctttttctcttgaaaggcttgaagtgcttggtgatgcatttctcaagtttgcagttgggaggcatgtctttcttacatacgatgcctttgacgaaggacagttaactagaaggcgctcaaacattgtgaacaattcctatctgtacacgatagcagtcagaaacaatttacaggcatttattcgtgatcaatcatttgatcccaaccacttctatgcagtaggccgtccgtgccctgtaatctgtgacaaacaaacagaaaaaaacatacacggtcaatgtggtagtgtaacagatggtgcaaagactgaagttaggtgtagcaaatgtcatcagtggttgcgtaagaaaacaattgctgatatagttgaagcccttgttggcgcttttatagttgacagtggcttcaaagcagcaattgcatttcttaaatggattggtatctatacagatttcgaagaatcacaagtaaagagtatttgcgctgcaagcaaagtctttatgccacttgctgatgaaattgatattccggccattgaaaatttattaggttatccattcgtccataagggtttgctcattcaggccttcatccatccatcttataacaaccacggaggaggttgctatcagagactggagtttcttggagatgctgtcctcgattatttgatcacatcctatctatactctgtgtacccaaaactgaagcctggccagttgactgatctgagatcagtttctgtaaacaacaacaccttcgcagttgttgcagtacatcaaagcttccacagccatatactctgtgattatagtagtggcctccgtgagtcaattactagatatgtcaatttcattggaagaccagattctatgaaaaggttggctgaagagccatcttgtccaaaggtattcactatttacatgatgaaatcttcgatattgatggattcatggagtttcttatacagagtttataatagtttgtgtttacacttattgtag

SEQ ID NO: 77 - DNA, 4794 bp, Artificial sequence
  DCL4A Cdna
  misc_feature (4705)..(4705): n is a, c, g, or t
atggaaggcggcgattttgagaacgacactgagagtccacgaagtgctgcaacttcaccgatcactgagcagctttctgctctgtctctcaatggcgacacagattcccctgtttcagttcagaaaccagagaaagaccccagaaaaattgctagaaagtatcagatggatctttgtaaaaaagcattagaggagaatgttgttgtatatctgggaacgggctgtgggaaaactcatattgccgtcttgcttatctatgagatgggacagctgataaggaaaccccagaagagcatttgcgtgtttcttgctcccactgtggcgttggtgcaacagcaagccaaggtcatagaagactctgtcgacttcaaagttgggacatactgcgggaaatccaagcatttgaagagccacgaagactgggaaaaagaaatggaacagtatgaggtccttgttatgacccctcaaatactattacataatctttcccactgctatatcaggattgaatttattgctcttctgatctttgatgagtgccactatgcacaagttgagagtgatcatccttatgctgagattatgaagatattttacaaacctgatgtagtaaaactgccccgtatatttggcatgactgcatctccaatatcaggaaaaggtgctactgttgaaggccttgagaccttacttcgctctaaggtgtattctgttgaagacaaggatgaactggaacagtttgttgcatcacctaaggtgaatgtatactactatggtcctggcaccgcgtgcctaactaaagcttactttcagaagcttgaagagattaaaagccagtgtgtgatggtgcttcacaagaaagcagctgatcatagcactctcaggaacacaaaaaagatgctcaagagactgcatggacatctaatttttgctagttgcatcctcttaaagggggatcactatgagcgccatcaaatggtggaagctgatgttaatgctagtgatgactctttgtgtgacagatatctgagtcaggtcgctgcagtgtttacttctggttgtgctaatgatggtatgaatcctgatttggcaattgtggaagttctgaaggagccttatttctccaaaaagcttttacgtctaattggaatcctttccaacttcggggtacaaccagatatgaagtgcataatttttgtcaataggatcgtgactgcgagatctctttcatatatgcttcagcatctgaaagttttatcttcttggaagtgtgggtttcttgtaggagtgcactcgggattgaagagtatgtcacgaaaaaatacagatatcattctcaataagttccgttctggtgagctcaacttattggttgccaccaaagttggcgaggaaggacttgatatccagacatgctgcctcgttatacgatttgatcttcctgaaactgttgccagttttatacagtctagagggcgtgcccgcatgccaaagtctgaatatgcatttttggttgatagtgataaccaaagggagcttaatttgatcgagcatttttcaagaaatgaagctcggatgaatgatgagatttcttccaggaaatcttgcacgacagttatagattttcaggaaaatatatacaaagtagatatgactggtgcaaccatcagttctgcatcaagcatttcgttgcttcaccattattgttccaagcttccacgtgacgagtttttctgccccaagccgcagttcttctactttgatgacatagatggaactatttgcaaattagttttaccttcaaatgctccaatgcatcaaatcgtgagtgctcctcaatcttctatagaagcagctaagaaagatgcctgtttaagagcctgcaagagcttgcatgaacttggagcactcactgactatcttttgccggatcaagcagatgaagatgaagacctgattcatggtttttctgactcagaaagctcagatggcgaagatgcacgagaagagctccatgagatgattgttcccgctgcctttaaagaaccatggactgagacagagagtcctgtttgccttaactcgtactacattaacttttcaccttgtccaattgatagggtttataaaaagtttggcctatttctgaaagcacctcttccccaagaggctgagagaatgaaacttgatctcaacttggctcgaggtagatctgtagagacagagcttattccatcaggagctactaattttgagaacaatgagcaggtacaactagcagagaaattccagagaatgttcctcaagatcattcttgaccgctctgagttcatttcagaatttgtatctttggaaaaacaagattctgttgactcagcttcaaaattttacctacttcttcccgtcaatttatgtggacacaataaaatttccatagactgggagcttgtcagaagatgcttgtcatcaccaatttttgggacttcagtatatgcaggcaatagtgagatatccaaatttgatgagcagttgcaacttgccaatggttccaaaagcgtacatgatgttgccaatagtttggtatatgttccatgcaaggatacatttttcttcatatctgatgttgtcaaagaaagcaatgcatacagcatatataaagattcaaaaaaccatgtcaaacactattatgatacttttggtatccatctttcgtaccctgagcaaccactaataaaagcaaaacaactcttctgtttggacaacttactacgaaagaaaggctattcagagttgcgtgataaagaggagcactttgttgaactacctgctgaaatttgccagctcatgataattggattttccaaagatattgggagctcattatcactgttgccatcgatcatgcatcgtcttgaaagcttacttgtcgctattgaacttaagggctggctatctgcttctttccctgagggaagggaagtaacaatagatcatgttcttgaagcacttactacagagaaatgcaatgaacctttttctcttgaaaggcttgaagtgcttggtgatgcatttctcaagtttgcagttgggaggcatgtctttcttacatacgatgcctttgacgaaggacagttaactagaaggcgctcaaacattgtgaacaattcctatctgtacacgatagcagtcagaaacaatttacaggcatttattcgtgatcaatcatttgatcccaaccacttttatgtagtaggccgtccgtgccctgtaatctgtaacaaacaaacagaaaaaagcatacacggtcaatgtggtagtgtaaccgatggtgcaaagactgaagttaggtgtagcaaatgtcatcagtggttgcgtaagaaaacaattgctgatatagttgaagcccttgttggcgcttttgtagttgacagtggcttcaaagcagcaattgcatttcttaaatgtattggtatctatacagatttcgaagaatcacaagtaaagagtatttgcgctgcaagcaaagtctttatgccacttgctgatgaaatagatattcctgccattgaaaatttattaggttatccattcgtccataagggtttgctcattcaggccttcatccatccatcatataacaaccacggaggaggttgctatcagagactggagtttctcggagatgctgtcctcgattatttgatcacatcctatctatactctgtgtacccagatctgaagcctgggcagttgactgatctgagatcagtttctgtaaacaacaacaccttcgcagttgttgcagtacatcaaagcttccacagccgtatactctgtgattctagtgacctccgtgagtcaattactagatatgtcaatttcattggaagaccagattctatgaaaaggctggctgaagagccatcttgtccaaaggcccttggcgacctggtggagtcttgtatgggtgcaatcctgcttgacacagggtttgacttgaaccgtgcttggcatataatgctctccttcttaaagcccgtcatgagctttaccaggttgcagcataatccaaaaagagaactccatgaactttgccagtttcatggctggcatctaaagttcctggtttcaaaaaaagatagcaaatatctagttgaggccaatgtgagtgggaagaatgtttctgaagcggcttctgctcccaatatcaataaaaaagctgccaaaagaatggctgcccagcaagtactttcaagcttgacggctcaaggctataggcgcaagtctaaatcactggagcaagtggtaaaaacagctaaaaagatggaagctaaactgataggatatgatgaaacaccctgtgttcttactgctaggtgcaatgacgtggagaagaacgaagcatctgaaagtgatcgtgacttgatagtattccctattagtgaagaacttgcaagaaattgcaatttcaagttaaagcctgcgagaaagttggccccagaggcagctgtacaatgcaattctgagcagaccatcatgcctaatggttctaattcagattcaaaagctacaggtggtgcaataaatggatcggcaaagtcaattttgcatgaagtttgtgctgccaactgctggaaacctcctagatttgaatgttgcaaggagacagggccaagccacttgaaagacctatggcacctacctnctcgggcgaaaaagaaagatgcagctgaggatgcagcggaaggggcactttggttcttgaagcaggaaggatatatgttcaataactga

SEQ ID NO: 78 - DNA, 3426 bp, Artificial sequence
  RDR1A Cdna
atgggaaagacaattcaggtttttggattcccttatcttctgtctgccgaagcggtcaagaaattcttagagaatcatacaggaaatggaactgtctatgctgtggaggttaaacagtccaaaggtggacgtagagcattcgccatagttcaatttgccaacaacaaaagcgcagaatttatcatcgatttggctagtaagcggttgtattatggaacttcttatttgaaggcttgggaaatgaaaactgatattgtccaaccgcggacatatgtgcaacacatggatggcgtaactctgaattttggatgtctgatatcagacaagaagtttgcagtgttgggaagtaaaggtgtttcagttaaatttggcattggattgaagaaaatttacttctttttatctcatgcttcagctgattataaacttcagctttcatatgaaaatatatggcaagttgtccttcatcgtccatatggtcaaaatgctcagtttctcctcctacagttatttggtgctcctcggatctataagagacttgaaggctcctgttatagcttctttaaggaaactcctgatgatcagtgggtaaggacaacagatttcactacatcttggatagggctatcttctagcttatgtttggagtttcgtaatggtggtcgacttccaaatttcagcgaaagttttttttactataaagaaagtgtgagccaagttattttacagactggttacaccttttcattctctcaaaatttggctctggttcccattgtccaccttcctgaaggtgttgagttgccctacaagatattgtttaaaattagttccttgatacaacatggatgctttcctggactagcattaaattttaactttttccaattagttgaccctcgaaggagaaatattgcatgcattgaacacgccttggagaaactgtactatttaaaggagtgttgttatgatccagtgaggtggctaactgagcagtatgatgagtacctcaaccgcagacaacttccaaaatctccgtccatcactttagatgatggattggtgtatgtaagaagggtcctagtaacaccatgcaaagtttatttttgtggtccagaggttaatgtatcgaatcgggttctgcgcaattattctgaagacatagataactttcttcgagtttcttttgttgatgaggagtgggagaaaatacattctacagatttattaacaagagctagtagtggaaatggtatcaggacagacatctatgagaggatcctatcaactcttcggaatggctttataattggtgataaaagatttgaatttcttgcattttcatcgagccagttgcgggataattcagtgtggatgtttgcatcaagacctggccttactgcaaatgatataagaacatggatgggtgattttcagcaaatcaggaatgtcgcaaaatatgctgccagacttggccaatcttttggttcctccagagaaactttgagtgtcggtaggcatgaggctgaagttattcctgatgtagtttgttcgcttcatggaaccaactacatcttttctgatggaattggaaaaatatctgctgactttgctcgtagagttgccataaaatgtggccttcagtatactccatcttcttttcagattcgttatggtggatataaaggtgttgtggctgttgatccatattcatcaatgaagttatctttgagaaagagcatgttgaaatatgaatcaaacaacataaagttagatgtcctgggatggagcaaatatcagccttgttatcttaatcgtcaactggttactctcttgtctacacttggggtgaaagatgatgttttcgaacagaagcaaaatgaagcggtagagcagcttgatgctatcttgcatgattctttgaaggcacaggaagctttagaattgatgtctcctggagagaacactaatattctcaaggagatgctaaattgtggttatatgcctgatgctgagccgtttctttcaatgatgctgcaaactttccgggcttcaaagttgctcgatttgcggactcgatcaagaatatttattccaaatggaagagcgatgatgggatgcttggatgaatccagaaccctggaatatggtcaggtgtttgttcagtttactggtactggacgtagacagttttatgaggaatcacatccttttaatgacagtggatccaccaactgtaatttcattctcgagggaaatgtggtcgttgcaaaaaatccatgcttgcatcctggtgatattcgtgttttaagggctgttgatgttccagctttgcaccacatggtagattgtgttgtatttccccagaaaggaaaaagcttccatccttataagaactgtttttatcttttttttcatggcattgtttctcagtgtttaaactttgatgacagacctcatccaaatgaatgttctggaagtgatttggatggggatatctattttgtttgttgggatccagacctgattccgccaaggcaagtccagccgatggattatactccagcacccagcacacagttggatcatgatgtcacaattgaggaagttcaagagtacttcaccaactatattgtgaatgacagtttgggaatcatagcgaatgcgcatgttgtatttgcagacagagaacctgatatggccatgagtgatccgtgcaaacaactcgctcagctcttttcaattgcagttgactttccaaagactggtgttcctgctgaaataccatctcagttgcgccctaaagaataccccgacttcatggaaaagccagacaagccaacctatcactcggaaaaagttattggaaaacttttccagaaagtgaaggacaaaacacctcaggcttgctctatcgcgaccttcacaatggatgttgccaggaaatcatatgatggtgatatgatagtcaatggattcgaagattacattgacgaagctttttactacaaaagtgaatacgacaacaagcttggtaatttaatggactactacggcgtaaaaactgaggctgaaatacttagtggtggcatcatgaaagcatcaaaaacttttgaccgcagaaaagatgcagaggccattggtattgctgtgaggtctttgaggaaagaagcaaggacctggttcaagaagcgtagtgatatagatgacatgttagcaaaggcttcggcttggtaccacgttacatatcatcatacatattggggtctctacaatgaggggttgaaaagagatcatttcattagctttccctggtgtgtttatgaccagctgatccagattaagaaggcgaaagcacgtaaaaagccggttctccatttatcttctctcgggaatcaactgagtcgcaaattagtgttgatatga

SEQ ID NO: 79 - DNA, 3351 bp, Artificial sequence
  RDR1B Cdna
  misc_feature (2774)..(2774): n is a, c, g, or t
atgggaaagacaattcaggtttttggattcccttatcttctgtctgcagaagcagtcaagaaattcttagagaatcatacaggaaatggaactgtctatgctttggaggttaaacagtccaaaggaggacgtagagcattcgcaaaagttcaatttgccaacaacaaaagcgcggaatttatcatcgatttggctagtaaggggttgtattatggaccttcttatttgaaggcttgggaaatgaaaactgatattgtccaaccgcggacatatgtggaacacatggatggcgtaactctgaattttggatgtcagatatcagacaagaagtttgcagtgttgggaagtaaagatgtttcaattaaatttggcattggattgaagaaaatttacttctttttatctcatgcttcagctgattataaactccagctttcatatgaaaatatatggcaagttgtccttcatcgtccatatggtcaaaatgctcagtttctcctcctacagttatttggtgctcctcggatctataagagacttgaagattcctgttatagcttctttaaggaaactcctgatgatcagtgggtaaggacaacagatttcactccatcttggatagggctatcttctagcttatgtttggagtttcgtaatggtgttcaacttccaaatttcagcgaaagttttttttactataaagaaagtatgaaccaatttattttacagactggtttcaccttttcattctctcaaaaattggctctggttcccattgtccaccttcctgaaggaattgagttgccctacaagatattgttcaaaattagttccttgatacaacatggatgctttcctgggctagctttaaattttaactttttccaattagttgaccctcgaaggagaaatattgcatgcattgaacacgccttggagaaactgtactatttaaaggagtgttgttatgatccagtgaggtggctaactgagcagtatgatgagtacctcaagggcagacaacttccaaaatctccgtccatcactttagatgatggattggtgtatgtaagaagggtcgtagtaacaccatgcaaagtttatttttgtggtccagaggtaaacgtatccaatcgggttctgcgcaattattctgaagacataaataactttcttcgagtttcttttgttgatgaggagtgggagaaaatacattctacagatttattaccaagagctagtactggaaatggtaccaggacagacatctatgagaggatcctatcaactcttcggaatggctttataattggtgataaaagatttgaatttcttgcattttcatcgagccagttgcgggataattcagtgtggatgtttgcatcaagacctggccttactgcaaatgatataagaacatggatgggtgattttcggcaaatcaggaatgtcgcaaaatatgctgccagacttggccaatcttttggttcctccagagaaactttgagtgttggtaggcatgaggttgaagttattcctgatgtagtttgttcgcttcatggaaccaactacatcttttcagatggaattggaaaaatatctgctgactttgctcatagagttgccataaaatgtggccttcaatatactccatcttcttttcagattcgttatggtggatataaaggtgttgtggctgttgatccatattcatcaatgaagttatctttgagaaagagcatgttgaaatatgaatcaaacaacataaagttagatgtcctgggatggagcaaatatcagccttgttatcttaatcgtcaactggttactctcttgtctactcttggggtgatagatgatgttttagagcagaagcaaaatgaagcagtagatcagcttgatgctattttgcatgattctttgaaggcacaggaagctttagaattgatgtctcctggagagaatactaatattctcaaggagatgctaaattgtggttatatgcctgatgctgagccgtttctttcaatgatgctgcaaactttccgggcttcgaagttgctcgatttgcggactagaacaagaatatttattccaaatggtagaacgatgatgggatgcttggatgaatccagaaccctggaatatggtcaggtgtttgttcagttttctggtgctggacgtagacagttttttgaggaatcgcatccttttaatgacagtggatccgccaactgtgatttcattctcaagggaaacgtggtcgttgcaaaaaatccatgcttgcatcctggtgatattcgtgttttaagggctgttgatgttccagctttgcaccacatggttgattgtgttgtatttccccagaaaggaaaaagacctcatccaaatgaatgttctggaagtgatttggatggggatatctactttgtttgttgggatccagacttgattccgccaaggcaagtccagccgatggattatactccagcacccaccacacagttggatcatgatgtcacaattgaggaagttgaagagtacttcaccaactatatcattaatgacagtttgggaatcatagcaaatgcgcacgttgtatttgcagacagagaacctgatatggccatgagcgatccgtgcaaacaacttgctcagctcttttcaattgcagttgactttccaaagactggcgttccagctgaaataccatctcagttgcgccctaaagaatacccagacttcatggaaaagccagacaagccaacctatcnctcggaaaaagttattggaaagcttttccagaaagtgaagaacaaaacaccccaggctagctccatcgcgaccttcacaagggatgttgccaggaaatcatatgatagtgatatgatagtcgatggattcgaagattacattgacgaagctttttactacaaaagtgaatacgacaacaagcttggtaatttaatggactactacggcataaaaactgaggctgaaatacttagtggtggcatcatgaaagcatcaaaaacttttgaccgcagaaaagatgcagaggccattggtgttgctgtgaggtgtttgaggaaggaggcaagggcctggttcaagaggcgtagtgatattgatgacatgttagcaaaggcttcggcttggtaccatgttacatatcatcatacatattggggtctctacaatgaggggttgaaaagagatcatttcattagctttccctggtgtgtttatgaccagctgatccagattaagaaggcaaaagctcgtaaaaggccggttccccacttgtcttcactcgggagtcaactgagtcgcaaattagtgatatggtag

SEQ ID NO: 80 - DNA, 3363 bp, Artificial sequence
  RDR1B Cdna
atgggtgtagagaagagaattacagcaactgtgagggtctcaaacattcctcaaacagccatagccaaacaactcttcgacttcttcgagtcttcaattgggaaaggatcagtctttgcttgcgatattttctcagagcacaagaattggaaatcaagaggtcatggtcgagtccaattcgaaacagcgcaatccaagttacaatctttatcactatctgaacaagggaagctcgtctttaaagggcaccaactcattctaacctcgtcttttgatgatattattgccaggccaattgagcccaactacaggtttcaaaagggaatcttgcatactggggtactgttaaagaatgattacatggaagttttggagacttgggaaaatgtgaagacgttgatcatgcctgaaagaaagagtcttgagttctgggttagtcatgcaaagggggaatgttataggttggaggtccagtttggcgacattatagagacttgtgggtgctctttggaagatgaaaaacctgcactccttttaaagctaaaacatgcaccaaagctataccaaagagtttctggacccggtgttgcttcaaagttcagttctgaccggtaccatgtttgcaaggaggattgtgagttcctttgggttcgcacaactgatttttctgccatgaagtctatcggatgttcatcctctctttgttgggaaatcgaagacggattgttaagttcagatttgctttctagtttgccatattgtaacaacgatgtgatggatcttgttctggatgaagtaggagatatttattctgcttctgaattagttcctcttgcaagtttcccatcagatctgaagctaccatatgagatccttttccagcttaactctctcgtacacacacataaaattagtcttggtgctgttaaaactgatcttattgaagttcttagcaaattagaattggacactgcaatgatgatccttcagaaaatgcataagttgcagtcaagctgtttcgagcctgtgccattcatcaagacacgcctgcatgtattgggtaaaaacagtaagaaccaaccttcatcatcttacagcaggttagtgaatcaaaacatgatgagtgttcacagagtcttggtaacaccatcaaaggtttactgcctgggcccagagcttgaaacctccaattacatcgtgaagaactttgcatcacatgcttctgatttcttgagagtaacttttgtagaagaggattggagtaaactttctcctaatgccatttctataagtgttgaacaaggtatctttgcaaagccttacagaactaaaatctatcatcggattttgtctatccttcgagatggacttgttattggaactaaaagatttcttttcctggcgttctcggctagtcagctacgttcaaattcagtttggatgtttgcttccaatgagtatgtcaaggcagaagatatcagagagtggatgggctacttcaacaagattcgtagtgtctcgaaatgtgctgcaaggatgggtcagttgtttagcacctcttttcaaacaatggaagtccaatcgccgcatgttgagattcttccagatattgaagtaacctctgatggcgttagttattgcttctcagatggcattggaaagatttctcaagcttttgccagtcaagttgctcagaaatgtggattgagttataccccatcagcatttcaaatacgatatggtggttataaaggcgtgatagctgttgaccgcaattcctatcgaaagctatctctacgtggcagtatgctcaaatttgaatctaagaacaagatgcttaatattaccaagtggagtgatgccatgccttgctatctgaaccgggagattgttatcctcttagctacgttgggagtggaggataaagtattagaagacctgctagataatcatctccatcttctgggcaagatgttgaccactaatgaggcggccttagacgttttggagagcataggtgggggtgacgtaaaaagaatcttaatgaggatgttgcaccaaggatatgcgcccaatttggagccttatctctcaatgatgcttcaatcccattttgagaatcagctttcagacctaagaagtaggtgtcgtatattcatacccaaaggtcgagttctagtaggatgcctagatgaaacaggtatattgaattatggccaggtttatgcacgaatcaccatgactaaggccgagctacaaagtggtcagcaaaatttcttccagaaggtggatgaaacaactgcagtagtaagaggcaaggttgtggtaacaaaaaatccttgtcttcatcctggagatgtaagagtacttgaagctgtgtatgaggtggcattagaggaaaaggcctgggtggattgcattatttttcctcagaaaggtgaaaggcctcatccaaatgaatgctccggtggtgatcttgatggagatctttatttcataagttgggatgaaaacctcattcctcgtcaaactgtgacacccatggactacactgggcgaaaacctcgtataatggatcacgaagtcactctagaggaaattcagagattctttgttgattacatgattagcgacactcttggtgcgatctctactgcacatttggttcatgcagatcgtgaaccagataaagctctgaatccgaaatgcctccagttagcgacccttcactctatggctgtggacttcgcaaaaactggagctgcagctgagatgccaaggtttctaaaaccaagagagtttcctgatttcttggagaggtgggacaaaccaatgtatatttctgaaggggtactagggaagctttatcgtgctatagtcaagtcttctgttcgcggtaactcagatgaccttggctctgtcagagctatccaggatgcttatgatcatgaccttttgttcgaaggttacgaggcattcatcgtaacagctaaaaatcacaaagaaatgtacttggatagaatgaacagcttactcaactactatggagcagagaaagaggttgagatactgacgggcaatttgcgccagaaatctgtgtatttgcagcgggacaacagaaggtattttgaactaaaagatcgaatcttggtttctgccaaaagcctacagaaggaggtgaaagggtggttcactggttgctgcaaggaggatgagaagaagaaactggcatcagcatggtatcaagttacttatcatcctagttactgccaaggaagtgctaattgcctgggatttccctgggtggtaggtgacgttttgctggatataaagttgcataataccagaaaaatcacctcctag

SEQ ID NO: 81 - DNA, 3363 bp, Artificial sequence
  RDR2B Cdna
atgggtgtagagaagagagttacagcaactgtgagggtctcaaacattcctcaaacagccatagccaaacaactcttcgacttcttcgagtcttcaattaggaaaggatcagtctttgcctgcgatattttctcagagcacaagaattggaaatcaagaggccatggtcgagtccaattcgaaacatcgcaatccaagttacaagctttatcactatctgaacaagggaagctcatctttaaagggcaccaacttattctaacctcgtcttttgatgatattattgtcaggcccattgagcccaactacaggtttcaaaagggaatcttgcatactggggtactgttaaagaatgattacatggaagttttggagacttgggaaaatgtgaagacgttgatcatgcctgaaaggaagagccttgagttctgggttagttatgcaaagggggagtgttataggttggaggtccagtttggtgacattatagagacttgtgggtgctctttggaagatgaaaaacctgcactccttttaaagctaaaacatgcaccaaagctataccaaagagtttctggacctggtgttgcttcaaagttcagttctgaccggtaccatgtttgcaaggaggattgtgagttcctttgggttcgcacaactgatttttccgccatgaagtctattggatgctcatcctctctttgttgggaaatcgaagacggattgttaaattcagatttgctttctagtttgccatattgtaacaatgatgtgatggatcttgttctggatgaagtaggagatatttattctgcttctgaattagttcctcttgcaagtttcccatcagatctgaagctaccatatgagatccttttccagcttaactctctcgtacactcacataaaattagtcttggtgctgttaaaactgatcttattgaagttcttagcaagttagaattggacactgcaatgatgatccttcagaaaatgcataagttgcagtcaacctgtttcgagcccgtgccattcatcaagacacgcttgcatgtattgggtaaaaccagtaagaaccaaccttcatcatcttacagcaggttagtgaatcaaaacatgatgagtgttcacagagtcttggtaacaccatcaaaggtttactgcctgggcccagagcttgaaacctccaattacatcgtgaagaactttgcatcacatgcttctgatttcttgagagtaacttttgtagaagaggattggagtaaactttctcctaatgccatttctatgagtgttgaacaaggtatctttgcaaagccttacagaactaaaatctatcatcggattttgtctatccttcgagatggacttgatattggaactaaaaaatttcttttcctggcgttctcggctagtcagctacgttcaaattcggtttggatgtttgcttccaatgagtatgtcaaggcagaagatatcagagagtggatgggctgcttcaacaagattcgtagtgtctcgaaatgtgctgcaaggatgggtcagttgtttagcacctcttttcaaaccatggaggtccaatcgccgcatgttgagattcttccagatattgaagtaacctctgatggcgttagttattgcttctcagatggcattggaaagatttctcaagcttttgccagtcaagtagctcagaaatgtggattgagttataccccatcagcatttcaaatacgatatggtggttataaaggcgtgatagctgttgaccgcaattcctttcgaaagctatctctacggggcagtatgctcaaatttgaatcaaagaacaggatgcttaatattaccaagtggagtgatgccatgccttgctatctgaaccgggagattgttatcctcttatctacgttgggagtggaggataaagtattagaagacctgctagataatcatctccatcttctgggcaagatgttgaccactaatgaggcagccttagacgttttggagagcataggtgggggtgacgtaaaaagaatcttaatgaggatgttgcaccaaggatatgcgcccaatttggagccttatctctcgatgatgcttcaatcccattttgagaatcagctctcagacctaagaagtaggtgtcgtatattcatacccaaaggtcgagttctagtaggatgcctagatgaaacaggtatattgaattatggccaggtttatgcacgaatcaccatgactaaggccgagctacaaagtggtcagcaaaatttcttccagcaggtggatgaaacaactgcagtagtaagaggcaaggttgtggtaacaaaaaacccttgtcttcatcctggagatgtaagagtacttgaagctgtgtatgaggtggcattggaggaaaaggcctgggtggattgcattatttttcctcagaaaggtgaaaggcctcatccaaatgaatgctccggtggtgatcttgatggagatctttatttcataagctgggctgaaaacctcattcctcgtcaaactgtgacacccatggactacactgggcgaagacctcgcataatggatcacgaagtcactctagaggaaattcagagattctttgttgattacatgattagcgacactcttggtgctatctctactgcacatttggttcatgcagatcgtgaaccagataaagctctgaatccgaaatgcctccagttagcaacccttcactctatggctgtggacttcgcaaaaactggagctgcagctgagatgcccaggtttctaaaaccaagagagtttcctgatttcttggagaggtgggacaaacccatgtatatatctgaaggtgtactagggaagctttatcgtgctatagtcaagtcttctgttcgcggtaactcagatgaccttggctctgtgagagctatccaagatgcttatgatcatgaccttttgttcgaaggttacgaggcattcatcgaaacagctaaaaatcacaaagaaatgtacttggatagaatgaacagcttactcaactactatggagcagaaaaagaggttgagatactgacgggcaatttgcgccagaagtctgtgtatttgcagcgggacaacaggaggtattttgaactaaaagatcgaatcttggtttctgccaaaagcctacagaaggaggtcaaagggtggttcactggttactgcaaggaggatgagcataagaaattggcatcggcgtggtatcatgttacttatcatccttcttactgcgaaggaagtgctaattgcctgggctttccctgggtggtaggtgacattttgctggatataaagttgcataataccagaaaaatcacctcctag

SEQ ID NO: 82 - DNA, 3594 bp, Artificial sequence
  RDR6A Cdna
atgggatcagagggctctgaaaaggatctcgtagtgactcaaattagtgttggtgggtttgacaatgatgtcaatgcgaaaatgctttcagaatatctggaagaacaagttggacaagtatggagatgtagattaaagatttcagccacccctcctgactcttacccaacttatgacattgatgcagagagggtgcatagaatgaaagattacgaaaaagtggtacctcatgcattcgttcattttgcatcttcagagtccgcaaagcatgctctggctgctgctgggggtaatgagctcatattaggaaagaagcctttgattgttagtttgggacctgagaatccctatcgtctgaatcagcggagaagaactaccatgccctttagattttcagatgttagtgtagagttgggagtactggttagtaaagatgactttgtggttggttggaggggacctcgcactggtgttgacttttttgtggacccattcaacggggcatgcagaatacttttcacaaaggatactgctttctctttcaggggtgaagccagacatgccattataaaatgcaatttcaagattgagtttatggtaagggaaatcaatgagataaagaaatgtaaagactttacgtctttggtaatactgttgcagttggcttcatctccattggttttctatagaactgctgatgatgatattgaagaatcagttgcatttgacttgttagacgatgatgatcaatggatccgtaccacagacattacaggtagtggagccattggtaggtgtaatacctaccgtatttcaattcgacctcgcaatggtcctagctttgagaaggccatggcatatttcagttttcgtagggttcccatggtagaaatgtgtaaccggaagatgctccgggtgagggacgagcctgattttggggtgtccatgtcagatcctttcttctgcttccaaaatgaaggtataagctttaaggtcatgtttctggtgaatgcggttttgcacaaaggcatagttaatcagcatcaaatgactaatgaattcttttacttacttagaagtcgtcaggaggaggttaatttagctgcactgaagcacatgttttcttacaaatggcctgtcaatgatgctattcagaagttagtaggcatccagaaatggctgttgaaaaaccctaaacttctcgagagaactggagagttggatgatattgtagaggttaggagattggttatcaccccaaccaaagcatactgtcttccaccgactgtggagctttccaacagagtcctcaggaattataaacatgttgcagatcgatttttgcgagttacattcatggacgagggcatgcggaacttgaataggaatgtgctgacatactatgctgctaccattgtgagggaaatcacatcaaattcgaatccccagagaactgcaatcttccaaagggtgaaaagtattctgagtaaaggatttcatttgtgtggtcgaaagtactcctttttggcattctcagctaaccagctgagggatcgttctgcctggttttttgcagaagaccctaaaattagggtacccggcatcataagctggatggggaggttcagtaacaggaatgtcgccaaatgtgcagctaggatgggacagtgcttttcatcaacttatgcaacagtggaagtcccttcaagggaggttaactcagagcttccagatattgaaagaaatgggtatgttttctctgatggaatcggcatgatatcagcagatctagctatagaagttgcagagaagctgcatttaagtgtcaaccctccttctgcttatcagataaggtatgctggttgtaaaggtgttgttgcttgttggccgacaaagaatgatggcatccgcctttctctgaggccaagtatgaagaaattcgactcaaatcacactatccttgaaatatgctcttggacgagatttcaacctggtttcctgaaccggcaaatagtaaccctgctctcttccttggacgttaaagatggaatattctgggaaatgcaaaaagaaatgatatcagggttggataaaatgctcgtggattcggacgtggcttttgatgtcatcacagcttcatgtgctgaggcaggaaatactgcagctctaatgttgagtgcaggttttaaacctcaaagtgagcctcatttaagagggatgctctctagcataagagcttctcagcttggggacctcaggaataaggcaaggatatttgttccttcgggaaggtggttgatgggctgtttggatgaattaggtgagcttgagcaaggccaatgctttattcaagtgtcaagcccttctttggagaattgttttattaagcatggtccgaaattttctgatataaagcaaaatcttcaagtagtaaagggccttgttgtaattgcaaagaacccgtgtcttcatcctggggatgtgaggattctggaggctgtagatgttcctggtttacaccatctctatgattgtctggtctttcctcagaagggggataggccacattcaaatgaagcatcaggaagtgacctggatggtgacctctactttgtgacttgggatgaaaatctcatcccacccagtaagaaaagctggatcccgatgaactatgaacctgcggaagtcaaacagttgggtcgtcaagttaatcatatggacataatagattttttttcaaaaaacatggtccaagagagtctaggagaaatctgcaacgcacatgtggttcatgctgacctcagtgaatttggagctttggatgagaagtgcctaaaattggcagagcttgctgctctagccgttgacttccccaaaactggaaaacttgtgaccatgccgtttgacctcaaaccaaaaatgtatcccgacttcatggggaaagaggaattccaatcatacaagtcaaagaaaattttgggcaaattgtataggcaagtgaaagatgtatgtgatacagagggtgaagtatctgctggacttgagcttgtcccaaacgacatcccatatgactctagtcttgagatcccaggatccacagctttcatagacgatgcatggaacagcaaatgttcttatgatggtcagttgcatggacttttagggcaatacaaagttaacagggaagaagaggtggttactggacatatatggtccatgccgaagtacagtgctaagaagcaaggggaattaaaagagaggctgaagcacgcatataatacgctgaggaaggagttcaggaatgtcttcgagcgtatggatcctgattttgatcttcttcccgatgatgagaagaatgatatgtatgaaagaaaggcatctgcatggtatcaggtcacctacaatcctcattgggttgctagatcactggagttgcaattgccagatgctatctcaagtactgtaatgctaagttttgcatggattgcagcagattaccttgctcgaattaaaattaggcacaggggattgcagtattccgactctaccaagccaatcaattcccttggaaggtatcttgttgacaaaatatga

SEQ ID NO: 83 - DNA, 876 bp, Artificial sequence
  RDR6B Cdna
gacataatagattttttttcaaaaaacatggtccaagagagtctaggagaaatctgcaacgcacatgtggttcatgctgacctcagtgaatttggagctttggatgagaagtgcctaaaattggcagagcttgctgctctagccgttgatttccccaaaactggaaaacttgtaaccatgccttttgacctcaaaccaaaaatgtatcccgacttcatggggaaagaggaattccaatcatacaagtcaaagaaaattttgggcaaattgtataggcaagtgaaagatgtatgtgatgcagagggtgaagtatctgctggacttgaggttgtcccaaaagacatcccatatgacactactcttgagatcctaggatccacaactttcatagacgatgcatggaacagcaaatgttcttatgatggtcagttgaatggacttttaggacaatacaaagtgaacagggaagaagaggtggttactggatatatatggtccatgccgaagtacagtgctaagaagcaaggagaattaaaagagaggctgaagcacgcatataatatgctgaggaaggagttcaggaatgtcttcgagcgtatggagcctgattttgatcttcttcctgatgatgagaagaatgatatgtatgaaagaaaggcatctgcatggtatcaggtcacctaccatcctcattgggttgctagatcactggagttgcaattggcagatgctgtctcaaatactgtaatgttaagttttgcatggattgcagcagattaccttgctcgaattaaaattaggcacaggggattgcagtattccgactctaccaagccaatcaattcccttggaaggtatcttgttgacaaaatatga

SEQ ID NO: 84 - DNA, 876 bp, Artificial sequence
  RDR6C Cdna
gacataatagattttttttcaaaaaacatggtccaagagagtctaggagaaatctgcaacgcacatgtggttcatgctgacctcagtgaatttggagctttggatgagaagtgcctaaaattggcagagcttgctgctctagccgttgatttccccaaaactggaaaacttgtaaccatgccttttgacctcaaaccaaaaatgtatcccgacttcatggggaaagaggaattccaatcatacaagtcaaagaaaattttgggcaaattgtataggcaagtgaaagatgtatgtgatgcagagggtgaagtatctgctggacttgaggttgtcccaaaagacatcccatatgacactactcttgagatcctaggatccacaactttcatagacgatgcatggaacagcaaatgttcttatgatggtcagttgaatggacttttaggacaatacaaagtgaacagggaagaagaggtggttactggatatatatggtccatgccgaagtacaatgctaagaagcaaggagaattaaaagagaggctgaagcacgcatataatacgctgaggaaggagttcaggaatgtcttcgagcgtatggagcctgattttgatcttcttcctgatgatgagaagaatgatatgtatgaaagaaaggcatctgcatggtatcaggtcacctaccatcctcattgggttgctagatcactggagttgcaattggcagatgctgtctcaaatactgtaatgttaagttttgcatggattgcagcagattaccttgctcgaattaaaattaggcacaggagattgcagtattccgactctaccaagccaatcaattcccttggaaggtatcttgttgacaaaatatga

SEQ ID NO: 85 - DNA, 5 bp, Artificial sequence
  PAM sequence for CRISPR
  misc_feature (1)..(2): n is a, c, g, or t
nngrr

SEQ ID NO: 86 - DNA, 8 bp, Artificial sequence
  PAM sequence for CRISPR
  misc_feature (1)..(4): n is a, c, g, or t
nnnngatt

SEQ ID NO: 87 - DNA, 8 bp, Artificial sequence
  PAM sequence for CRISPR
  misc_feature (1)..(4): n is a, c, g, or t
  misc_feature (6)..(8): n is a, c, g, or t
nnnngnnn

SEQ ID NO: 88 - DNA, 4315 bp, Artificial sequence
  Ntab-XylT-A exemplary contig Ntab-BX_AWOK-SS596
atgaacaagaaaaagctgaaaattcttgtttctctcttcgctctcaactcaatcactctctatctctacttctcttcccaccctgatcacttccgccacaaatcccgccaaaaccacttttccttgtcggaaaaccgccatcataatttccactcttcaatcacttctcaatattccaagccttggcctattttgccctcctacctcccttggtctcaaaaccctaatgttgtttggagatcgtgcgagggttacttcggtaatgggtttactctcaaagttgaccttctcaaaacttcgccggagtttcaccggaaattcggcgaaaacaccgtctccggcgacggcggatggtttaggtgttttttcagtgagactttgcagagttcgatctgcgagggaggcgcaatacgaatgaatccggacgagattttgatgtctcgtggaggtgagaaattggagtcggttattggtaggagtgaagatgatgagctgcccgtgttcaaaaatggagcttttcagattaaagttactgataaactgaaaattgggaaaaaattagtggatgaaaaattcttgaataaatacttaccggaaggtgcaatttcaaggcacactatgcgtgagttaattgactctattcagttggttggcgccgatgattttcactgttctgaggttagattttgaatttttgtttgctctttaaattaaaggtttaaactttgtgaatgttggcagatatggaatacactaatggattttgtttgatctgtttaatgaagattgtctagaacctcaatgttataaatatggtttggttgcttcattaattaaagagaattccttaatatcccgactagatgccagataacaccagttagttgacttttggattattggttgcatttcatttgatcagataaattgttcattcttaaatgtttcactaaagaattactcaagatttcagagtttatatgtaggtgtatgtatttggaattctggatttggatctagtattgaatggattactgaacttgtactccccagtcatctggggaggagcaatagatcaaattcaagggttgaaaagtaatactgagtcagaaattaaccactttaacttggaaaacggtaaatgtatgtgttctaagatggttattcctataacttttgatgtctaatatggagaaagtgagttgatttatgctttttccttttccctttattggtgttggtttttaaattctatcaattcctttgtttgattgctactcaaattgaaccttagacggagtagcaatagcaaaaagtgaaggccattcttttctcctttcatctctttatttccgtttgacatacagaatatggtagcatctgtctgaagtggttaattttattccttaaaatttgcataactaattcgagtaaatgccttttgaagctttagttgaatagttctacaactggttgttgcattttgaggactatcgacttgatttgacacttgacattgtctgatacatggcttgtaagttatgaaaacttttatctaggaagaaatcccaaccagagatagggagctgtcacttggttatgagctactggctcaaagttcaagtttgaccagttaattttagatcttcaccaggataacatttagagtctaatcaaattctgaagcagtattgtgcactaataagaggaacacatgaaggatgtagcactactaggttatgttaccttatttactaatgattgacaaccagcttaaatgatgacaaatagtcttatatttgctttttcacattgctcatgacttgggatatttccgaatcaacatattttagttctttatgtacttaactacttatcaaaaaattatccctgctagatgttagtgttcaagcaaccatgctagcttttaaggaagctccttctttgattcatgccatctttccgaaatcgatgccttacgttactgtcatttttctaattttcatttcagtggattgaggagccgtcacttttgattacacgatttgagtatgcaaaccttttccacacagttaccgattggtatagtgcatacgtggcatccagggttactggcttgcccagtcggccacatttggtttttgtagatggccattgtgaggtatgtttgaaagtattgataacgatggcatgcattgtactgtgttacggatgaaagaaatgaaaccagcaattattttctagcaggcaatgctcttgagatgcttgtgtcaaattggtcagacttaatcctgagtttccatttgtttcagctttctgtttgactgactacaataattgtcccaattaggggtgtcaatggatattcgaaagccgactaaaccgaccgaaccgtaccgtaccgatttttaggtttctttttaagaaaccgtaggtttttatataaatctataaccgcaccgataattagggtaggttttttattttataaaaataagccgaaaaaataccgaaccgtaccgaataaattttacatgtggaaaatatatttatttagtaagtttaaaaataataatgcattaaattttctttgggccatggaattatgaaaactattacaagccaacaagtaattagactcaaaatactaattcctaaaacctattatgttacttctacttaaactaagttatttcaagtatctttattagcaagacacaaagtattctagcgattatgagtagcaaactacaatgtattgaatatgtttcctttcatgtaatttagatttatctttttgaatatttaatcttctatagactctattcttgagtcccagcttggtatatctttcaactcgtgtgatttatattttctttgcctttgtttgatttcttttacgttgttgtagaatagtcgatggatctatactctagccatctttctttttctttttttaattcatcaccttttaaacagtaaaaatgtctaaagaatttttctaagtcctataaaagaacgtatgttattgcattctacttctactggtgaattttacatgatattaaaaaattaaccgaaccttaccgctaccgaagagaaaccgacatgattgggacggtttcgaaaagtctaattttggttatacataatagaataaccgaaaaattggtatggtacaaattttataaaataaccggccgaaccgaaccattgacacccctagtcccaatacctagttgttgcagtttgctcattcttacttctatttacgtcactgtttctctgaatggtccctttgtggtgaaaagagcttttgctatgtagaaaaactagcaatgatttcatagctgaaacaatttatttttaccttacatcatgtcttataaaattgcttctaactgtatactttaattcttggagagatgctttcatgtgaagaaagttctttcactccactactggaagcttgccgtatgaattttacttggccatattgtggccgtgctttgatttatcttcaaattcattttcttcatatagttctttcgagtaattcttttttcctcttttctgtttgaaaaaaattcagacacaattggaggaaacatggaaagcacttttttcaagcctcacttatgctaagaactttagtggcccagtttgtttccgtcatgccgccctctcgcctttgggatatgaaactgccctgtttaagggactgtcagaaactatagattgtaatggagcttctgcccatgatttgtggcaaaatcctgatgataagaaaactgcacggttgtccgagtttggggagatgattagggcagcctttggatttcctgtggatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccttgaaccactcggagtgcaaattaaatgtaattaacggattgtttgcccacatgtccatgaaagagcaagttcgagcaatccaagatgcatctgtcatagttggtgctcatggagcaggtctaactcacatagtttctgcagcaccaaaagctgtaatactagaaattataagcagcgaatataggcgcccccattttgctctgattgcacaatggaaaggattggagtaccatcccatatatttggaggggtcttatgcggatcctccagttgtgatcgacaagctcagcagcattttgaggagtcttgggtgctaa

SEQ ID NO: 89 - DNA, 3568 bp, Artificial sequence
  Ntab-XylT-B exemplary contig Ntab-BX_AWOK- SS12784
atgaacaagaaaaagctgaaatttcttgtttctctcttcgctctcaactcaatcactctctatctctacttctcttcccactctgatcacttccgtcacaaatccccccaaaaccactttcctaatacccaaaaccactattccctgtcggaaaaccaccatgataatttccactcttctgtcacttcccaatataccaagccttggccaattttgccctcctacctcccctggtctcagaatcctaatgtttctttgagatcgtgcgagggttacttcggtaatgggtttactctcaaagttgatcttctcaaaacttcgccggagcttcaccagaaattcggcgaaaacaccgtatccggcgacggcggatggtttaggtgttttttcagtgagactttgcagagttcgatttgcgagggaggtgctatacgaatgaatccggacgagattttgatgtctcgtggaggcgagaaattggagtcggttattggtaggagtgaagatgatgagctgcccgtgttcaaaaatggagcttttcagattaaagttactgataaactgaaaattgggaaaaaattagtggatgaaaaaatcttgaataaatacttaccggaaggtgcaatttcaaggcacactatgcgtgaattaattgactctattcagttagttggcgccgatgaatttcactgttctgaggttagattttgaaattttgcttgatctttaaattaaaggtttgaactttgtgaatgttggcagatatggaatacaataatggattttgtttgatctgtttaatgaagattgtctagaacctcattgttataaatatggtttgtttgcttcattaattaaagagcattccttaaaatctcgactagatgccagataacaccagttagttgacttttggattatggattttttttcatttaatcagataagatagtcattcttaaatgtttcactaaagaatttgtcatgatttcagtgtatatctttaagtgtatttggaattttggatttggatctagtactgaatgggtaactgcacttgtactccccagtcatctggggaggagcaacagattaaattcaagggttgaaaagtaatacagagtcagaaattaaccacaagttggaaaatggtaaatgtatgtggtctaagatgattactcctataacttttgatgtctaacatggagaaagttagttgatttatgctctttacttttccctttattgattttggttttcaaattctatcaattcctttgtttgattgctactcagattgaaccttagacggagtagcaatagaaaagtgaagaaaagccattttttctcctttcatctctttatttctgttttacacacagaatatggtagcatctgtctgaactagttaattttattccttaaaatttgcataactaattcgagtaaatgccttttgaagctttagttgtacaactggttgttgcattttgaggactatcgacttgatttgacagtgtctgatacatggcttgtaagttatgaaaacttatatctaggaagaaatcccaaccagagatagggagctgtcgcttggttatgagctactggctcaaagttcgagtttgaccagttaattttagatcttcggagtctaatcaaattctgaagcagtattgtgcactaataagaggaacacatcaaggatgtagcactgccaggttatgttaccttatttactaatgattgagaaccagcttaaatgatgacaaatggtcttagatttgttttttacattgctcatgactttgggatatttctggatcaacattttccagttctttatgtacttatcaaaaaattatccctgctagagcctagatgttagtgttcaagcaaccatgctagcttttaaggaagctccttctttgattcatgccatctttccgtaatcgatgccttacgttactgtcatttttctaattttcatttcagtggattgaggagccgtcacttttgattacacgatttgagtatgcaaaccttttccacacagttaccgattggtatagtgcatacgtggcatccagggttactggcttgcccagtcggccacatttggtttttgtagatggccattgtgaggtatgtttgaaagtattgataacgatggcatgcattgtactgtgttatggatgaaagaaatgaaatcagcaattattttctagcaggcaatgctcttgagatgcttgtgtcaaattggtcagacttaatcctgagtttccatttgtttcagctttctgtttgactgactacaataattgtcccaatacctagttgttccggttggctcattcttacttctatttacgtgtcactgtttctctgaatggtccctttgtggtgaaaagtgcttttgctatgaagaaaaactagcaaagatttcatttctgggacaatttatttttaccttacatcacgtctcataaaattgcttcctattgcatactttaattcttggagagatgctttaatgtgaagaaagttctttcactccacttttaattcttggagagatgctttaatgtgaagaaagttctttcactccacttttaattcttggagagatgctttaatgtgaagaaagttctttcactccacttttaattcttggagagatgctttaatgtgaagaaagttctttcactccactactgtaagcttgctgcatgaattttacttggccatattgggggcgtgttttgatttatcttcaaattcattttcttcatgtaattcttttgagtaatttttttttctgttttctgtttgggaaaaaattcagacacaattggaggaaacatggaaagcactcttttcaagcctcacttatgctaagaactttagtggcccagtttgtttccgtcacgccgttctctcgcctttgggatatgaaactgccctgtttaagggactgacagaaactatagattgtaatggagcttctgcccatgatttgtggcaaaatcctgatgataagagaactgcacggttgtccgagtttggggagatgatcagggcagcctttggatttcctgtggatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccttgaaccactcggagtgcaaattaaatgtaattaacggattgtttgcccacatgtccatgaaagagcaagttcgagcaatccaagatgcttctgtcatagttggtgctcatggagcaggtctaactcacatagtttctgcagcaccaaaagctgtaatactagaaattataagcagcgaatataggcgcccccattttgctctgattgcacaatggaaaggattggagtaccatcccatatatttggaggggtcttatgcggatcctccagttgtgatcgacaagctcagcagcattttgaggagtcttgggtgctaa

SEQ ID NO: 90 - DNA, 6032 bp, Artificial sequence
  Ntab-FucT-A exemplary contig Ntab-K326_AWOJ- SS19752
  misc_feature (542)..(542): n is a, c, g, or t
atggcaacagttgtgccaattcaaagattaccaagatttgaaggtgttgggtcatcatcacctacaaatgttccccaaaagaaatggtccaattggctacctctagtagttgcacttgtggttatagttgaaattgcatttctgggtcgactagacatggctgaaaaagccaacctagtcaactcttggactgactcattttaccagtttacgacgtcgtcttggtcaacctccaaagtggaaattaatgaggctgggttggctgtgttaaggagtggtgagattgatcggaatttggaaactgggagctgtgaggagtggttggaaagggaggattctgtggagtattctagagattttgacaaagatccaatttttgttcatggcggcgaaaaggtgagattgtttcttgtatatgtttattctttttactaataaatggggtgaatagagcagaatggatatagaggattcatatagttgaacacgaatagtttgggaagttctatgcacttaaggatgtaatagtttttttttttttntttttttttttagtgatgagtgaagggcagccttgacgtaaccggtaaagttgttgccatgtgagtcacggttttgagccgtggaaacaaccttttgcagacatgcagggtaaggcggcgtaaaatagacccttgtggtccggctcttctcggaaccctacatagccggagcttagcgcatcgacctgccattttgatgatgagtattttgatgatgagtataagttctatgcactaacgatgtaaaatagatttacaccattaggtcacttaaaaggtagtagcagctaattctccatggaaacattaattggtaatcgagcatctcttttagactattatatggattttgttgcaatttatgtcttgttatttattacatcagttagctgccagagctccagacatatggctttggtagatggaatgataaaaatttgttgtttcaaatgatgcttgggttttcttctcgttttttttttgttcttatatcatgtttacagtggaatatcttataaagtacagactattgacaatattccaaaacctcttctatatgggtaaaaatactcatgaattttatatactagtagggaataaggggaggagccttgagtatcttctttttttcttacctttttttccttcattacattgaattcttcatagagcacttaagtggaatgagcagaaatcaatcagtaaaactgccatttattgcttgagtttatcatgactagttcttgttcccatgttatccactggtatataggtgagagcaggtaacagttactcggttaccatctttcttcttgatttttttcccttaccatatgcaaaaactgatgttccttcgatcgttatctaggattggaagtcttgtgccgtaggatgtaactttggtgtggattctgataagaagcctgatgcggcatttgggacaccacaacaggctggcacggctagcgtgcttcggtcaatggagtctgctcaatactatcctgagaacaacatcattaccgcacgacggtgggtaagcacatctagaaaaagacttaaaacattctcaccacatttggcagcatttgtcgacaattgaattttcatcttgtgatcattttttaatgaaacatatctcacttggaagtttttgcttgcaattagtttctgactagaccttttttctttggataagtaaggtagcatattattagtaagcagtaccaagaaagtacaaaaattgatacttccaaagtctactcaaaacctgaatccagcgactccaagaactcatttgtactaactacaagatccgttttatgccagcaaaagagatattgtaaacatctatacttcataactataatctcctcattttccccctcaaaatatctcatatttctttcttgccaaaccgtcaaaacaatacacaaagaaataagcttccagatattcttccctcttttctccactgtgcttgccagctgatgagtgcacctttgaaattttgtggcattacccagtattacacccaaaatatattttttatcaggataccctctaaatattaaggaataaagaccagatactccaagaaagattacgatgcatcgggagatgactaacagattcacatagacaatcctgatttgaaaccacaactgatcacagttgggaataaatctgtaagtaaaccttcattacaccatctatcagtccaaagcttgactttcctaccattttcaactttttgttttatgttgtctttgaactgtccctagaaattagctattggtctccccaaagcaacctcataaggattacttactggttttggatcctaatatctctccatgccataaattgacttaataacagccttccatactgcatattttccatcgttacaaaaaaaagcagttgcatttgctcaaatagcctttggaaagggcatctacaaacatgcaatcataaagccctcaacaacaataactacaacaacaacctagtaaaatcccacaagtggggtctggggagggtagtgtgtacccaaaccttacccctaccctgagggagtagagaggttgtttccgatagaccctcggctcaagaagatgaaaagagacaatatatcagtactatcaacagatcatagagataataacagcaatcataaaggcctcatagacacaataaccttaggatcatgttgtggttataatttaatttttagatctcctatagttcttctctcaatctttatatctttctctagggaaatctctaaccaactttattattctttctcatgtttcagaaggggatatgatattgtaatgacaacaagcctctcttcggatgttcctgttgggtacttctcttgggcggagtatgatataatggctccagtgcaacctaaaactgagaatgcattagcagctgcttttatttctaattgtggtgctcgcaacttccggttgcaggctcttgaagtccttgaaagggcaaatatcaagattgattcttttggcagttgtcatcgtaaccgggatggaaatggtcagtgtatctccattatatatgataatatattgatggttcttttcttgaagtagttaccattaaggagctaattgtctaaaatatttcaatataatgtgtttttgaaaagccatgtttactggtaatggaaaccttatatttgtttccttggtaaatgtacacatacacatgtaagttttctaaatagtcagattttctgctagtttgaagagttcattttgtggattggttattttgctgttatgcttgttattttttgaataccttcttagtattttgcaacccattaaattgagttgaaaagccgacagtttttgcaaattcattgcaatgaaattagaccctaatttgttataaaagaaaaatttagagaaaatttagttttagtttatttttctgatgtagaatatgcatgcatgtggttaaactttacattatatgtaaatttattagaatagagttgaaaagcagacatctttttgtaaatttattgtgaagaagctagaccagggtttgttggaaagggaaattaagagaaaaggcagtcttaataaatgtcatattagaatgcagaatatgtttattcatgcctctagtttaattgtacattatatcctgtgaatgctcacttgtcatcttgttctcaatttcatggcagtggacaaagtggaaactctcaagcgctacaaatttagcttcgcttttgagaattctaatgaggaggattatgtcactgaaaaattcttccagtccttagtagctggtaataatttttgcctgttaattttggttctgatttgaacacttagtttccgatgtatctattattttctattagcccccacccctctgcaatgatgcattttgttttactttttctgcaattcataattacacaaaagacataggagatattagctatagagcgccatgaagaacaaagcaaaaagcacaaacttctgagcactttatgttacttcaactgcgttttgtacccgaatttgcattttctggtacggcacacaaatatgctctgctttatattcatgtaaaaggctttaggaaaattttaaatgattttacgttaattatcattgttaatcatattctttgtgctcctagtcgatatactttaggctagagttatgcacagaatcctgtttttatgatttttcacaagttactacttcaaaattatgatttatggtagtcaacgctattgtgctgataaaaggaagttcatgtaaacttgcaggatcagtccccgtggtgattggtgctccaaacatcctagattttgctccttctcctaattcacttttacacattaaagagctgaaagacgctgcatcagttgccaagactatgaagtaccttgcagaaaatcctaatgcatataatgagtcattaaggtatgcatcaattagtcatgcttttcttacgtgctcttcttgattatttgaattttcctgtcctaaattaacttttttttgtttgtcctgaagatttatccactctctctaaaaaaaaaccccttttccaacatctttctgtacttttctgttatcatgttattgagaaagtagcactggcctgtctctatagttacaaaagtttattaccttatcctattttgacacactgatatatagttttggtctaactaaaactcctaaattgggaagattattccgtgtgtgtgagtgtgtgtccccttctgcatgtgcggacttgcatttgaccttttcctttcaaaatttattaaagattcttaaacttcctgggtcttgctaacaaatggtttcttttcattgtttaggtggaaatttgagggtccatctgactctttcaaagccctggttgacatggcagcagttcactcttcttgtcgtttgtgtatcttcttagcaactagtattagggagaaagaagagaagagtccaaaatttacgaaacgtccctgcaaatgtaccagaggttcagaaactgtctatcatgtatatgtacgtgaaagagggaggtttgacatggagtctgttttcctaaggtattctcgatctgccatgactaaatatcatgcatatacaagtgccttttctgtttatgttcctgtgccgctgttcttatgtttaatatgtaccatgatgatcaaattgtttaccaatattggaatgaaaaggatccgaaaagagtggaatgtatatagaggattcgtagagctgaccgcaaataggggtgagacatattgatcaaattatttgagtaactactcactgtgtcttactctcgatgtatgagaattgagaagtatatgcttggtagccatggtctatgggcttataaagtagtttacatgtttgtggttggatattccacaaaatcaatgtcaatctatctaaagtagttcttgatcgatttgatagacttaactagggaagttccggaaaatatttggcaagtggtgtttggttcactagtaaagctagaagatagggctggggggttaaagttggggggatccggccacgaaaaagaaatatggacaaccaatgtcataatgtgaattctctcctgcacttctccttttactttctgagcatatacaaactgtttcatgtctcattggcaagtcttatgttatctttgaatcactgttattgctggagcctttgcaggtcatctaatttgtcactggaggcttttgaatctgcagtactgtcaaagttgaaatctctaaagcatgttcctatttggaaagaagaaagacctcaaatacttcgtggaggggatgaactaaagctctacagagtatatcctcttggcatgacacgacaggcattgtacacctttagattcaaaggagacgctgattttaagaatcacatcgaaagccacccatgcgcaaactttgaagccatatttgtatag

SEQ ID NO: 91 - DNA, 7189 bp, Artificial sequence
  Ntab-FucT-B exemplary contig Ntab-BX_AWOK- SS16887
  misc_feature (2757)..(2757): n is a, c, g, or t
atggcaacagttgtgccaattcaaagattaccaagatttgaaggtgttgggtcatcatcacctacaaatgttccccaaaagaaatggtccaattggctacctctagtagttgcacttgtggttatagttgaaattgcatttctgggtcgactagacatggctgaaaaagccaacctagtcaactcttggactgactcattttaccagtttacgacgtcgtcttggtcaacctccaaagtggaaattaatgaggctgggttggctgtgttaaggagtggtgagattgatcggaatttggaaactgggagctgtgaggagtggttggaaagggaggattctgtggagtattctagagattttgacaaagatccaatttttgttcatggcggcgaaaaggtgagattgtttcttgtatatgtttattctttttactaataaatggggtgaatagagcagaatggatatagaggattcatatagttgaacacgaatagtttgggaagttctatgcacttaaggatgtaatagtttttttttttttagtgatgagtgaagggcagccttgacgtaaccggtaaagttgttgccatgtgagtcacggttttgagccgtggaaacaaccttttgcagacatgcagggtaaggcggcgtaaaatagacccttgtggtccggctcttctcggaaccctacatagccggagcttagcgcatcgacctgccattttgatgatgagtattttgatgatgagtataagttctatgcactaacgatgtaaaatagatttacaccattaggtcacttaaaaggtagtagcagctaattctccatggaaacattaattggtaatcgagcatctcttttagactattatatggattttgttgcaatttatgtcttgttatttattacatcagttagctgccagagctccagacatatggctttggtagatggaatgataaaaatttgttgtttcaaatgatgcttgggttttcttctcgtttttttttgttcttatatcatgtttacagtggaatatcttataaagtacagactattgacaatattccaaaacctcttctatatgggtaaaaatactcatgaattttatatactagtagggaataaggggaggagccttgagtatcttctttttttcttacctttttttccttcattacattgaattcttcatagagcacttaagtggaatgagcagaaatcaatcagtaaaactgccatttattgcttgagtttatcatgactagttcttgttcccatgttatccactggtatataggtgagagcaggtaacagttactcggttaccatctttcttcttgatttttttcccttaccatatgcaaaaactgatgttccttcgatcgttatctaggattgaaagtcttgtgccgtaggatgtaactttggtgtggattctgataagaagcctgatgcggcatttgggacaccacaacaggctggcacggctagcgtgcttcggtcaatggagtctgctcaatactatcctgagaacaacatcattaccgcacgacggtgggtaagcacatctagaaaaagacttaaaacattctcaccacatttggcagcatttgtcgacaattgaattttcatcttgtgatcattttttaatgaaacatatctcacttggaagtttttgcttgcaattagtttctgactagaccttttttctttggataagtaaggtagcatattattagtaagcagtaccaagaaagtacaaaaattgatacttccaaagtctactcaaaacctgaatccagcgactccaagaactcatttgtactaactacaagatccgttttatgccagcaaaagagatattgtaaacatctatacttcataactataatctcctcattttccccctcaaaatatctcatatttctttcttgccaaaccgtcaaaacaatacacaaagaaataagcttccagatattcttccctcttttctccactgtgcttgccagctgatgagtgcacctttgaaattttgtggcattacccagtattacacccaaaatatattttttatcaggataccctctaaatattaaggaataaagaccagatactccaagaaagattacgatgcatcgggagatgactaacagattcacatagacaatcctgatttgaaaccacaactgatcacagttgggaataaatctgtaagtaaaccttcattacaccatctatcagtccaaagcttgactttcctaccattttcaactttttgttttatgttgtctttgaactgtccctagaaattagctattggtctccccaaagcaacctcataaggattacttactggttttggatcctaatatctctccatgccataaattgacttaataacagccttccatactgcatattttccatcgttacaaaaaaaagcagttgcatttgctcaaatagcctttggaaagggcatctacaaacatgcaatcataaagccctcaacaacaataactacaacaacaacctagtaaaatcccacaagtggggtctggggagggtagtgtgtacccaaaccttacccctaccctgagggagtagagaggttgtttccgatagaccctcggctcaagaagatgaaaagagacaatatatcagtactatcaacagatcatagagataataacagcaatcataaaggcctcatagacacaataaccttaggatcatgttgtggttataatttaatncaattatggaaaaaccccatagagaaaccatcagggcccggtgctttgtcagctgcacatatcttcaaggtctcaaacacttcttgttcttcaaattctccttgtagcaattcattatcttcctatagttctcctatagttctaattgtctctcaatctttatatctttaaatttaaattttagatctcctatagttcttctctcaatctttatatctttctctagggaaatctctaaccaactttattattctttctcatgtttcagaaggggatatgatattgtaatgacaacaagcctctcttcggatgttcctgttgggtacttctcttgggcggagtatgatataatggctccagtgctacctaaaactgagaatgcgttagcagctgcttttatttctaattgtggtgctcgcaacttccggttacaggctcttgaagtccttgaaagggcaaatatcaagattgattcttttggcagttgtcatcgtaaccgggatggaaatggtcagtgtatctccatataatatgatggttcatttcttgaagtagttaccattaaagagctaaatgtctagcatatttcaatataatgggtttgattctctcttcctcaagtgtattatatcccttgaaaagccaattttactggcaatagaaaccttatatttgtttccttgctaaatggacacataccaatgcaagttttttaaatagccagatgttctgatagtctaaagatttcattttgtggataggttattttgctgttattcttgttatctttttttcttttggataaggttaagttttattaataggagtaccaagaaggtcacataattatatcaaaagaaagacagcaaacactttttttatttatcttttgaatacctattagtttgcaacccattaaaatagagttgaaagcagacacattttgtaaattcattgcaaagaagctagttcccgattagttagaaaagaaaaatttagagaaaaggtagcctagataatttttttttcccggaatgcagaatatatttatgcatacatgtggtttaactgtacgttagatataaattcacgtagaatagagttgagaagcagacatttttttagtaaatccatcgtgaagaagctagaccttggtttattggaaagagaaatgaagagaaaaggtttttatccaacaaaaaaataaaataagagaaaaggcagtcttgataaatctcatatttgaatgcagaatatttttatgcatgcctctagtttaacagtacactagttcttggacacgtgcgttgaatccaaattatagatttattatatttaactcatcccgaaatttaatattcataaagtaagttccaaaataacataatgagttactttagtttactttgctaaaaattgattaaattgatgcctttaaaaaaatacgataaaacaaatgatggagagaaagaagaggaatagaaaaggattatgcggcttttgagttttaaccaaagcacagaaataattatttagtaaaaaactaaggaaagagagagatcaaggtgtttttagagattaatgaagggatgtggctctttgtgtagtgcgatattataacagtggaagataaagaaacaaacaatcaatgaataattaagataattaaactcaaaaaaacttgtataaaaaaacaatcaatgcctacattcacccggttaaaagtaataaaagtattcatttactggaaggtcatttttttggctcaaggataaaattgtcattcagttttttaaggttatattagtaaattaactttgaggataggatgttcccacttttagtataatatgattatatcctgtgaatgctcacttgtcatcttgttctcaatttcatggcagtggacaaagtggaaactctcaagcgctataaatttagcttcgcttttgagaattctaatgaggaggattatgtcaccgaaaaattcttccagtctctggtagctggtattaacttttgcctgttaattttggttctgctttgtacgcttagtttctgatgtaggatctcttattttctctcagccccacccctctacattgatgcattttgttctactttttctgcaatgccatactttcacaaagactttgaagatattagctatagagcgtgccatgaagaacaaatattgcaaaaacacaaacttatgagcattttatggtacttcaactacattttgaacctggatttgcattttctggtacagcgcacaaatatgctctgctctatattcatttaaaaagctttaggaaaattttaaatgattttacatgaattatcattgtttaatcatattctttgtgctcatagtcaatatattttaggctagatttatgcacagaatcctgtttttatgatgtttcacaagttactacttcaaaattaagatttatggtggtcaactctgaagttcatgtaaacttgcaggatcagtccctgtggtgattggtgctccaaacatcctagactttgctccttctcctaattcacttttacacattaaagagctgaaagacgctgcatcagttgccgagactatgaagtaccttgcagaaaatcctagtgcatataatgagtcattaaggtatgcatcaattagtggtgcttttcttacgtgctcttcttgattatttgaattttcctgtcctaaataacttttcattggttgtcctgaagatttatccactctcaaagaacacctttttccagcatctttctgtactttttcggtatcatgttattgagaaaataacaacagccctgtctgtatagttacaaaagtttattaccttatcctatttacatactgatatattgttttggtctaactaaaactcctgaattgggaagattgttctccttgtgtgtgtgtttcccctctctgcttgtctggagttgcaatttactctttcctttcaaatttttttaaggatttcttaaatttcctgtgtctggctaactaatgctttttttcattgtttaggtggaagcttgacggtccatctgactctttcaaagccctggttgacatggcagcagttcactcttcttgtcgtttgtgtatcttcttagcaactagtattagggagaaagaagagaagagtccagaatttacaaaacgtccctgcaaatgtaccagaggttcagaaactgtctatcatatatatgtacgtgaaagagggaggtttgacatggagtccattttcctaaggtattctctatctgtcatgactaatttctattcatgcatatataagtgcattttctgtatatgttcatgtgccactgttcttgtttttaatatgtaccatggcgatcaaattatttacaagtattggaatgaaaagggtccgaacagagtggaatgtacaacaacaacaacaacgacccgttaaaatcccacaaagtgtgtgtacacaaaccttacccataccccgatggggcagagaggctattaccgatagaccctcgaccgaatagagtggaatgtatatagaggattcatatagctgatcgcaaataggggttgaggcatagtgatgaaattgcttgagtaattattcactgtgtcttactatcaatgtatgagaagtatatggcttggctgagagcccatggtctatgggctaagttgcttggatacaggtgcgggtgtccgacacgggtgcggatctagatatcagattcttcgagatataaattttaagattcggggatatggatcctattactagtagggatatgagtgcggagatccggctaaaaataatacaaaaaattaataatataaaagtactctaaattatgagaaattttgtggaatacttagcttgtaaagtgtggatttctttattctcaagttgtaaaagattgatttcctagataaggtatgctatttttttcaaaattacccttgctagaagatggggttagcgggttaaagttgggggggattcaaccgtgaaaaagaaatatggacagcagatatcataatgtgaattctctcctgcacttctccttttacttgcttagcatatacaaactgtttcatgtcttattgtcaagtcttatgttatctttgaataaccgttattgctggaacctttgcaggtcatctaatttgtcattggaggcttttgaatctgcagtactgtcgaagttcaaatctttaaagcatgttcccatttggaaagaagaaagacctcaaatactacgcggaggggatgaactaaagctctacagggtatatcctctcggcatgacacaacgacaggcattgtacacctccagattcaaaggagacgccgattttaggaatcacatcgaaagccacccatgtgcaaattttgaagccatatttgtatag

SEQ ID NO: 92 - DNA, 9264 bp, Artificial sequence
  Ntab-FucT-C exemplary contig Ntab-K326_AWOJ- SS16744
atggcaacagttattccaattcaaaggttaccaagatttgaaggtgttgggtcatcatcacctacaaacgttccccttaagaaatggtccaattggctacctctagtagttgcacttgtggttatagttgaaattacatttctgggtcgactggacatggctgaaaaagccaacctggtcaactcttggactgactcattttaccagtttacgacgtcgtcttggtcaacctccaaagtggaaattagtgagactgggttgggtgtgttgaggagtagtgaggttgatcggaatttggaaactgggagctgtgaggagtggttggaaaaggaggattctgtggagtattctagagattttgacaaagacccaatttttgttcatggcggcgaaaaggtgatatagttttcttgtaaatgtttattttttttttagagcagaatgaatatagaggattcttatagttgaacccgaacagtttaggaggcatagttgattgctctttttttgatgatgcgtattagttctatacaggggattcttggagtaacggtaaagttgtctccgtaggtcacaggttcgagccgtgggttcgagccgtggaatcagccacgatgcttgcatcagggtaggctgcctacattataaccccttcgtgcaccggactactgccctttagtattagttctaggcactaacgatgcaaaaaagatttacgccataaggtcacttaaaaggtagtagcagctaattctccatagaaacattaattggtgatcgagcatctcttttagacgataatttgaattttgttgcaatttatgtcttgctatttattacatcagttagctgccagagctcgtgtgtgtgtgtgtgtgtgtgtgtgtgttccagacttgtggctttggtaggtggaatgataaaaaaattgttgtttcaaatgatgctttggcattttcttctagcttttttgttcttttatcatgtttacggtcaaacatcttataaggactattgaccatattccaaaaccgattatatgggtaaaaatactcatgaattttatatactagtagggaataagtggaggagccttgaatatcttcttttttcttacctttttttccttcattacattgaattcttcatagagcacttaaatggaatgagcagaaattaatcagtaaaactgccatttattgcttaagtttatcatgagtagttcttgttcccatgttatccactggcatgtagtgagagcaggtaacagttacctggttaccatctttcttcttgatttttttttccttaccatatgcgaaaactgatgttccttcaatcattatctaggattggaagtcttgtgccgtaggatgtaactttggtgtggattctgaaaagaagcctgatgcggcatttgggacaccacaacaggctggcacggctagcgtgcttcggtcaatggagtcagctcaatactatcctgagaacaacatcgttatggcacgacggtgggtaagcacatcttgaaaaagatttaaaacattctcacctacatttggcacctgaaagataatagcatttgccacatttgagttttcatcttgtgatcgtatcttaatcaaacatatctcacttggaagtttttgcttgctattagtttctgactaggcctttcttttaaggagcatattattagtaagcagtaccaagaaggtaccaaaaaagtacaaaaatttagacttccaaagacaactcaaaacccgaatctagtgagttgaagaactcatttgtactaactagaggatcggttttatgccagcaaaagagatactgtaaacatctattctttataactgaattatgctcctttcccccctcaaaatatctcgtgtttctttcttgacaaaatctccaaataatacacaaaggaataagcttgcagatcttcttcagtcttttctccactgtgcttgccagctgatgagtccacctttgaaatttgtggcattacccagtattacccccaaaatatattttttatcagtataccctcaaaatattaaggaataaagaccagctactccaagaaagattacgatgcatcaggagatgactaacagattcacagacaatcctgatttggaaccacactgaacacggctgagaataaatctataagtaaaccttcattacactattcatcactccaaagcttgactttcccatcattttcagcttttagttttatgtttatctttgaactgcctccaaaaattagctattggtctccacaagcaacccataaggattacttactggttttggattctaatatctctccatgccataaatcggctttataacagccttccatactgcatattttctatcattacgaaaaaaaagcagttgcatttgctcaaatagcctttggaaagggcatctacaaatatgcaatcataaagccctcatagacaataatcttaggatcatgtttttttttatgataatggtaacatatatattaataaataaaacagcactagagcagtgccaagccatatttacaagaaaacagaaaccagctatgacaacctaagattacagtgcacctatcaggtctactaaagattctgcctcctctatccagttttctttacaccagaaataaaacaaaagtaaacacttcatcttgatgttctgtaaagaattgcttctgtcttcaacagttcttaaatttctctccttccagactgtccaccatatgcacgcaggaaccaatcgccaccatttcttctgtctcactataccctcattgtaattccaacacttcaacatatcacaagtgttagctggcatgctccatttaaggcccacaatattaagaaatagttgccacagttggtcagtaaaaggacaatgaagaaacaaatggctattagtttctatagctgtaccacataataggcatttagagcatagctgaaaacctcttcttctcaagttctcttgggttagacaagcctttcttgtaactaaccatgaaaagcataccaccttgaaaggtgcctttactttccaaatattcctccagggccattgttctaactgttggtttgaagcagcgagtagagtatacgcggatctgaccgtaaaattcccactccttcaatgattccagcatagtttatcttcagtttctccaagaccttggaattgttccaagatattaaagaaatttacaaccctttctaattcccaatcattcaaggcctttctaaaagtaacattccatccttgctgaccccaggcagtgtctaaagtggatgtaggtgatgttgcaatgctgaaaaatttttgaaaaaattctttgagaggaccatgtcctaaccagttatcactccagaaaagtgttttccttccattaccaacctttatggttgtattatcagccagagtattccagtgaagtctgattgacttccataccccaaccccatgtgaactattaacagagttagagcaccatagtccattttgtccatacttgtcacagatgacctttctccacaaagcattagccttcagattgtacctccaaagccacttcaagagtaaactctgattgtgggctttaaggttcctaatgcccaaaccccctttgtctttgcttgtgataagggaattccagttaactaaatgaatagcttccttctctctgtttccttgccatataaagctccttcttagtgcatcaattcttttcctcaccttgcttggtaagggaaacaaagacattacacatgtaggaagtgcatccaagacactattaaccaataccacccttccacccaaggataaatactggctcttccaggttgatagccttttttcacatctttcaagaaccccattccatatttcttgagctttgttcttggagcctagagataaatccaaatatatagttggtagagaacccacctcatatcccagtatagctgctagggcctggatatcatccacttctttaactgggaacaacatgctcttcctccaatttacatggagccctgagactgcttcaaatataacaagaatgactcttagatattttagttgatctgcctttgcctcacaaaaaactaaagagtcatcagcatatagaagatgggtgatctccacactatctccctccctgtttgaagctttaaaacctttcagccaaccattgttgtttgcatttcttaacatttggttcaatccttccatagcaaagaggaaaagaaaaggtgataagggatccccttgtctcaaacctctctcagaaggaaaaaatccctctggagaaccattcaccaaaatggaaaatctgactgttctgatgcagaaagatatccagttgatccacttcctaccaaaacccatatcctgaagaactttgagtaaaaagttccaattaacatgatcataggccttctcaatatcaagcttacagagaattccgggaacctgcccttttaagtcaggaatccacacattcatttgcaatcaaggaagcatccatgatttgcctgcctcttatgaaggccatttgatgcccattcactagtttgtccaccactttcttgagcctctctgtcaatagtttggaaatgatcttgtaaatgcttcctgtaagactaattggtctgaagtccttgagttccttagcaccattcttttttggaataagggctatgtaagtagcattgaaactcttctcaaaatattcattgttgtggaagtttctgatggtatgcatgatgtcttctttcacaatctcccagcaattatggaaaaaccccatagagaaaccatcagggcccggtgctttgtcagctgcacatatcttcaaggtctcaaacacttcttgttcttcaaattctccttgtagcaattcattatcttcctatagttctcctatagttctaattgtctctcaatctttatatctttaaatttaaattttagatctcctatagttcttctctcaatctttatatctttctctagggaaatctctaaccaactttattattctttctcatgtttcagaaggggatatgatattgtaatgacaacaagcctctcttcggatgttcctgttgggtacttctcttgggcggagtatgatataatggctccagtgcaacctaaaactgagaatgcgttagcagctgcttttatttctaattgtggtgctcgcaacttccggttacaggctcttgaagtccttgaaagggcaaatatcaagattgattcttttggcagttgtcatcgtaaccgggatggaaatggtcagtgtatctccatataatatgatggttcatttcttgaagtagttaccattaaagagctaaatgtctagcatatttcaatataatgggtttgattctctcttcctcaagtgtattatatcccttgaaaagccaattttactggcaatagaaaccttatatttgtttccttgctaaatggacacataccaatgcaagttttttaaatagccagatgttctgatagtctaaagatttcattttgtggataggttattttgctgttattcttgttatctttttttcttttggataaggttaagttttattaataggagtaccaagaaggtcacataattatatcaaaagaaagacagcaaacactttttttatttatcttttgaatacctattagtttgcaacccattaaaatagagttgaaagcagacacattttgtaaattcattgcaaagaagctagttcccgattagttagaaaagaaaaatttagagaaaaggtagcctagataatttttttttcccggaatgcagaatatatttatgcatacatgtggtttaactgtacgttagatataaattcacgtagaatagagttgagaagcagacatttttttagtaaatccatcgtgaagaagctagaccttggtttattggaaagagaaatgaagagaaaaggtttttatccaacaaaaaaataaaataagagaaaaggcagtcttgataaatctcatatttgaatgcagaatatttttatgcatgcctctagtttaacagtacactagttcttggacacgtgcgttgaatccaaattatagatttattatatttaactcatcccgaaatttaatattcataaagtaagttccaaaataacataatgagttactttagtttactttgctaaaaattgattaaattgatgcctttaaaaaaatacgataaaacaaatgatggagagaaagaagaggaatagaaaaggattatgcggcttttgagttttaaccaaagcacagaaataattatttagtaaaaaactaaggaaagagagagatcaaggtgtttttagagattaatgaagggatgtggctctttgtgtagtgcgatattataacagtggaagataaagaaacaaacaatcaatgaataattaagataattaaactcaaaaaaacttgtataaaaaaacaatcaatgcctacattcacccggttaaaagtaataaaagtattcatttactggaaggtcatttttttggctcaaggataaaattgtcattcagttttttaaggttatattagtaaattaactttgaggataggatgttcccacttttagtataatatgattatatcctgtgaatgctcacttgtcatcttgttctcaatttcatggcagtggacaaagtggaaactctcaagcgctataaatttagcttcgcttttgagaattctaatgaggaggattatgtcaccgaaaaattcttccagtctctggtagctggtattaacttttgcctgttaattttggttctgctttgtacgcttagtttctgatgtaggatctcttattttctctcagccccacccctctacattgatgcattttgttctactttttctgcaatgccatactttcacaaagactttgaagatattagctatagagcgtgccatgaagaacaaatattgcaaaaacacaaacttatgagcattttatggtacttcaactacattttgaacctggatttgcattttctggtacagcgcacaaatatgctctgctctatattcatttaaaaagctttaggaaaattttaaatgattttacatgaattatcattgtttaatcatattctttgtgctcatagtcaatatattttaggctagatttatgcacagaatcctgtttttatgatgtttcacaagttactacttcaaaattaagatttatggtggtcaactctgaagttcatgtaaacttgcaggatcagtccctgtggtgattggtgctccaaacatcctagactttgctccttctcctaattcacttttacacattaaagagctgaaagacgctgcatcagttgccgagactatgaagtaccttgcagaaaatcctagtgcatataatgagtcattaaggtatgcatcaattagtggtgcttttcttacgtgctcttcttgattatttgaattttcctgtcctaaataacttttcattggttgtcctgaagatttatccactctcaaagaacacctttttccagcatctttctgtactttttcggtatcatgttattgagaaaataacaacagccctgtctgtatagttacaaaagtttattaccttatcctatttacatactgatatattgttttggtctaactaaaactcctgaattgggaagattgttctccttgtgtgtgtgtttcccctctctgcttgtctggagttgcaatttactctttcctttcaaatttttttaaggatttcttaaatttcctgtgtctggctaactaatgctttttttcattgtttaggtggaagcttgacggtccatctgactctttcaaagccctggttgacatggcagcagttcactcttcttgtcgtttgtgtatcttcttagcaactagtattagggagaaagaagagaagagtccagaatttacaaaacgtccctgcaaatgtaccagaggttcagaaactgtctatcatatatatgtacgtgaaagagggaggtttgacatggagtccattttcctaaggtattctctatctgtcatgactaatttctattcatgcatatataagtgcattttctgtatatgttcatgtgccactgttcttgtttttaatatgtaccatggcgatcaaattatttacaagtattggaatgaaaagggtccgaacagagtggaatgtacaacaacaacaacaacgacccgttaaaatcccacaaagtgtgtgtacacaaaccttacccataccccgatggggcagagaggctattaccgatagaccctcgaccgaatagagtggaatgtatatagaggattcatatagctgatcgcaaataggggttgaggcatagtgatgaaattgcttgagtaattattcactgtgtcttactatcaatgtatgagaagtatatggcttggctgagagcccatggtctatgggctaagttgcttggatacaggtgcgggtgtccgacacgggtgcggatctagatatcagattcttcgagatataaattttaagattcggggatatggatcctattactagtagggatatgagtgcggagatccggctaaaaataatacaaaaaattaataatataaaagtactctaaattatgagaaattttgtggaatacttagcttgtaaagtgtggatttctttattctcaagttgtaaaagattgatttcctagataaggtatgctatttttttcaaaattacccttgctagaagatggggttagcgggttaaagttgggggggattcaaccgtgaaaaagaaatatggacagcagatatcataatgtgaattctctcctgcacttctccttttacttgcttagcatatacaaactgtttcatgtcttattgtcaagtcttatgttatctttgaataaccgttattgctggaacctttgcaggtcatctaatttgtcattggaggcttttgaatctgcagtactgtcgaagttcaaatctttaaagcatgttcccatttggaaagaagaaagacctcaaatactacgcggaggggatgaactaaagctctacagggtatatcctctcggcatgacacaacgacaggcattgtacacctccagattcaaaggagacgccgattttaggaatcacatcgaaagccacccatgtgcaaattttgaagccatatttgtatag

SEQ ID NO: 93 - DNA, 5137 bp, Artificial sequence
  Ntab-FucT-D exemplary contig Ntab-K326_AWOJ- SS19661
atgagatcttcgtcaaattcaaacgcacccaataaacaatggcgcaattggttgcctctgttctttgccctagtggttatagcagagatttcttttctggttcgactcgacgtggctgaaaaagccaactcttgggccgactcgttttatcagttcaccacagcctcttggtccacctctaaactggctgttgaccacggcgacgttgaggaggtccagttgggtattttgagtggtgagtttgatcagggcttcgtacccgggagttgcgaggagtggttggaaagggaagattctgtggcttattcgagggattttgataatgaaccaatttttgttcatgggcctggacaggttataaccacttctatttatgactgaatattgaatattgatataattggacttattagttcgggattgagccatagtcgtatttctttttctttttcttttctttttcttttttcgtccgttgtttgtggcatgcctaaatctcttttaatgtgtttatttatttttgtggattttatcgtaattcctagtgttagataatccttaaatacagggtattgaaatattatggacccgaatagagcaattatgatattgaggattcatacagccgactccagctagtttgggatagaggcgtggtagtagtagtagttgttgttgttgttgcaaaaaaatgtattggcatctcagtatgcgtaaggtgcatggttgatttcagacttttgactattattgtagctgggtcatagcaagagaggtttgcttagttgatggattttagttttttagcttcgtttgcttggagcttttcataaggactagagtttctaatacttttatttaaaaaggggaaagagcagagaagcattgtgaattttcatattgattgccttttgaagcatatagtcgttcagtattcctttacttatttcataaaaataacatgctaatggaatgatcagaaatcaatttatcataatgcgaataccacttcttattgttcttggtctctcatgctattcgcttgtgagtttgaatgtttaactgttaggcattacttatctctggctttatgaatgtcctgccctatacgaaattctgatgtctattcaatgattcaatcactatataggaattgaaaacctgttccgtaggatgtaagtttggaacagattccgataagaagcctgatgcagcatttcggctaccacaacaagctggtacagctagtgtgctacggtcaatggagtcagctcaatactatgcagagaacaacattactttggcacgacggtgggtaagcacattgtgaaagaagtcttatttcattccctgcctttattggcaattttctttttaatatttgttgtcattctctttcatttttatcacattcttatttaagttatgtattgctattagttttcccttccaggggtgggggtaaggctgcgtacatcttacggtcttaccctctccagacctactgggtttgttgttgttgttgttgttgttgttgttgttgttgtattgctattagttttagttaagaacttttgcattataagtatattggcagctataggtccttgaccaaacttttgccatagacaagatatttaaaaactgatgacatgaattctttccctttaggcactaaatatatttcctgtagaaaatagttaagattcaccttgactgaatcatcgctgacactgctttttgttgttttaatctggctgggttcctttctctttttcttcattccgttagggcaaaaaaataggaactctgcttttcaattggggagtttttgggatggagtggatcaccaaccatacttattgaagctaatttagagctaaagatgctaaagtacgtttttgataagtcataatgtgaatgtactagctttggttatttaaccgcacaagtcaaactagaacttagttttgacatggtataagtcaaattcttcctattttacttatatagaagttcttctccttttgtttactttataaagggtataagatgataaatatattgtctactcttgggggtctttgggtatgctaaatgagctaagaggtgtttagaactctagcaaggattttcatgacgtattaaggacatgatcagaacccatgttcagtgtttgcacagggttatcaccaactaatggtcaatgagcacatctaatctagtttaatgtttgagttattggattgacttttcaatatcaataaaccattggccaaatttcatgatattttactgagccatctgtaatatgatgtccaaccatgcctattcaataaaatgaaaattttaaaacttacagaattagttgagcgccaccagatacttaaagctatgccaactgcgtctaacagaagttgaaagacgaagttgagtgagagcactgtttttgatgtgtggattaggtgcatgtcacaagttcgaaccctgtagcagacaaaatcctggtatttacgtggagaaggatagagagctgggcgtattatccattgagtttcgaaccgtgcgtcactagcccttagggatttcggtcaacaacaacaacaacaacaacaacaacaacaacaacatacctagtaatttcccataagtggggtctggcgagggtagaatgtacgcagccttacccctaccctggaagggcagagaggctgtttccgatttcggttatcataaaataaaaaaaaaaggttgaaacacaaagctaatttttctatcacaaaaatctttgaatttcattgtagttgagattttggcatcaccttgcttaaaaaattagcttagcatatagacatagatatttaaagctatgccagttgccatgatagagtttaaaattatcttggttagttggttagtgctcttcgttacattgagtcacacgattaatttatgaagccaaagttcttaaggaccatggcgtggttgagatttaaactttgcataagcttgctaaccaattttatttttttcactcacataccagaaggggatatgatgttgtaatgacaacaagcttctcttcagatgttcctgttggatacttctcttgggctgagtatgatatcatggctccagtacaacctaaaacagagaatgtcttagcagccgctttcatttctaattgtggtgctcgcaatttccgcttgcaagctttagaagcccttgaaagggcaaatatcagaattgattcttatggcagttgtcatcataacagggatggaagaggttagtatatttcaattatccaaacttactgaaggattagaggatagaatacggatggtgcaattttaagcagtgtcactagggagctaattcttgtccatagagtagtattatgggtttgattgactcttcctcgggtatcacaccttcctccagaagacaggattttactaccagtgcaaaccttttttttctctcctggctaatgtgagcacgcatgtcgtcgtttttttagtgatttgaatttatgctagtccaatgattgcttgtcaatggattattttgctctttttcttgtttaaaatttgagtttcaattttgccacctgataagaataaagttggaatacaacattcatttaaatagttcgatttcattctgaggaagttaggctgagatttgttggaaagagacgtatagcgagaaaaaatgttgtggacaaatcatctttctggatgccgtgtattttatacatgcatttggtttagggttgcactaatatccaactgaattgcgttatttgtcaataaaaaatttccaatgaaatctaacttctggtctctgttctcaatctgatggcagttgacaaagtggaagcactgaagcggtacaagtttagcttggcttttgagaattctaatgaggaggactatgtaactgaaaaattctttcagtctctggtagctggtaattacatttgttttttcttattgggtttgtagacttggattttcagagttgagagcatctattattttagctcagtccctcccttaacatgatagatacatttgttcctagttgtatttgatgtggttttggttagatcttctgcgtttactagcagaccttggaattgtagtatctaaagcgtacaattatttatagaagtttcaggaaggacaaacttctgagttctgataaattcttgatacattcaacaatggtttggatctagacttgcatttctgtagaatgcacaatgtgctctacactgagatggctcaaatatttttggaattttgttgagatgattttaggggtatgtcttagttgagcattttctttatgctctaagactaaattctctttttcgaggtctatcccatgtttaagattttgacaattttattagttcagaattgagatttaaggtttcaacttgctgataaaagtaagtctataaaacttgtaggatcaatccctgtggtggttggtgctccaaacatccaagactttgctccttctcctaattcagttttacacattaaagagataaaagatgctgaatcaatcgccaataccatgaagtaccttgctcaaaaccctattgcatacaatgagtcattaaggtatgcgtcaataagaattgttgttgttgtttgtttgtgtttttttttttgtttttttgtttttgttactccagttgtttacttgataatgggatggtactcttcttaattgttcgatttcctgttttttgcaattacacactgtccaaatctctctcttttttaagccatttggtaccttttgaaaatagtattacgaagaaaatattacagacccatttcactaaaatgttttcactactgtatttccagttttgaccaatttatatatagatattgccttttgatgttaggtggataactgaattgaacgagaacacaatggatctctctgtttttctgtaattacaagcaacttcttccctttcaagattttacttaatatttcttaaatttactggacatctaacaaatgatttactttaattgttcagatggaagtttgagggcccatctgatgccttcaaagccctggttgatatggcagcagttcattc

SEQ ID NO: 94 - DNA, 6639 bp, Artificial sequence
  Ntab-FucT-E exemplary contig Ntab-K326_AWOJ- SS19849
ggctatagctattccaattcaaagttttcctagcacccacctcaatgtcaaattcaaacgcacccaataaacaatggcgcaattggttgcctctcttcgttgccctagtggttatagcagagttttcttttctggttcgactcgacgtggctgaaaaagccaactcttgggccgactcgttttatcagttcaccacggcgtcttggtccacctctaaactgtctgctgaccacggcgacgttgaggaggtccagttgggtgttttgagtggtgagttattgagtggtgactttgatcagggcttcgtacctgggagttgcgaggaatggttggaaaaggaagattctgtggcttattcgagggattttgataatgaaccaatttttgttcatgggcctggacaggttataaccacttctatttatgactgaattttatgaagaattaacccaaatagccgtccaccaactgcttaaactaaaattagccggtagaggtataatatatgcataatttatgtattatatgtgtataatgaacgtataatggctaaaaaacgtaaaaaatgaatatgaccagctatttatgtaaagatccctattgaatattgatataattggacttactagttcgggattgagccatagtcgtattttttttttccgtccgttgtttgtggcatgcctaaatctcttttaatgtgtttatttatttttgtggattttatcgttattcctagtgttagataatccttaaatacagggtattgaaatattatggacccaaatagagcatttatgatattgaggattcatacagccgactccagctagtttgggatagaggcgtggtagtagtagtagtagttgttgatgttgcaaaaaaatgtattggcatcttagtatgcgtaaggtgcatggttgatttcaggcttttgactattattgtagctgggtcatagcaagagaggtttgcttattgatggattttagttttttagcctcgtttgccgtgacatattaataagtgctagagtttctaatccctttattcaaaaatggggaaagagtagaggagcattgtgaattttcatattaattgcctttcggagcatatattcgttcagcaatttatttcatcacaaaaataacgcgctaatggaatgatcagaaatcaatttatcataatgcaaatacggcctcttattgttcttggtctcgcatgctattcaattgtgtgtttgaatgtttaattgttaggcattacttatctctgactttatgaatgtcctgccctatacggaattctgatctctattcaatcactatataggaattgaaatcctgttccataggatgtaagtttggaacagattccgataagaagcctgatgcggcatttcggctaccacaacaagctggcacagctagtgtgctacggtcgatggagtcagctcaatactatgcagagaacaacattactttggcacgacggtgggtaagcacattgtgaaagaagtcttaattcattccctgcctttattggcaattttctttttaaaatttgttgtcattctctttcatatttatcacatttaagttatgtattgctattagttttagataagaacttttgcattataagtatattggcagctataggtccttgtccaaaattttgccatagacaagatatttgaaaactgatgacatgaattatttccctttaggcactaaatatatttcctgtagtaaatagctaagattcaccttgactgaatcatcgctgaccctgctttttgtcgttttaatctggctgggttcctttctctatttcgtcattccgttagggcaaaaaataggaactctgtttttcaatcggggagttttggggatggagtggaccaccaaccatacttattgaagctaatttagagctaaagatgttaaagtacgtttttgataagtcataatgtgaatgtactagctttggatatttgaccgcacaaatcaaactagaacttagtttcgacatggtataagtcaaactcttcctattttacttatatagaagttcttctccttttgtttactttataaagggtataagatgataaatatattgtctactcttgggggtctttgggtatgctaaatgagctaagaggtgattagaactctagcaaggattgtcatgatgtattaaggacatgatcagaacccatgttcagtgtttgcacagggttatgcaccaactaatggtcaatgagcacatctaatctagtttaatatttgagttgttattggattgacttttcaatatcaataaaccattggtcaaatttcatgatattttactgagccatctgtaatatgatgtccaaccatgcctattcaacaaaatgaaaattttaaaacttacggaattagttgagcgccaccagatacttaaagctatgccaactgcgtctaccagaagcttaaagacaaagttgagtaagagcacagtttttgatgtgtggattaggtgcatgtcacaagttcgaaccctgtaacagacaaagtcctagtatttaagtggagaagggtagatggttgggcgtattatccaccgagtttcgaaccgtgcgtcactagcccttagggatttcggttatcaacaacaacaaaatacccaataacttcccacaagtggggtctggcgcccacaagtggggtctggcgaggataagatgtacgcagccttacccttaccctggaagggcagagacgctgttttcgattttaggtatcataaaatttttaaaaaaaaaaaaagttgaaacacaaagctaatttttctatcacaaaaatctttgaatttcattgtagttgagattttggcatcaccttgcttaaaaaattagcttagcatatagacctagatatttaaagctatgccagttgccatgatagagtttaaaattaccctggttagttggttagtgctcttcgttacattgagtcacaagattaatttatgaagccaaagttcttaaggaccatggcgtggttgagatttaaactttgcataagcttgctaacctattttattttttccactcacataccagaaggggatatgatgttgtaatgacaacaagcctctcttcagatgttcctgttggatatttctcttgggctgagtatgatatcatggctccagtacaacctaaaacagagaatgccttagcagccgctttcatttctaattgtggtgctcgcaacttccgcttgcaagctttagaagcccttgaaagggcaaatatcagaattgattcttatggcagttgtcatcataacagggatggaagaggttagtatatctcaattatccaaacttactgaaggattagaggatagaatatggatggtgcatttttaagcagtgccactagggagctaattcttgtccatagagtagtattatggatttgattgactcttcctcgggtgtcacaccttcctccagaagacaggattctactaccagtgcaaaccttattttttttctcctggctaacgtgagcatgcatgtcgtttttttagtgattcgaatttatgctagtccgatgattgcttgtcaatggattattttgctctttttcttgtttaaaatttgagtttcaattatgccacctgataagaataaagttggaatacaacattcatttaagtagttcgatttcattctgaggaagtcaggctgaaatttgttggaaagagacgtatagtgagaaaaaatgttgtggacaaatcatctttctggatgccgtgtatcttatacatgcaattggtttaggtttgcactaatatccaattgaattgtgttacttgtcaataaaaaatttcctatcaaatctaacttctggtctctgttctcaatctgatggcagttgacaaagtggaagcactgaagcggtacaagtttagcttggcttttgagaattctaatgaggaggactatgtaactgaaaaattctttcagtctctggtagctggtaatcacatttgttttttcttattgggtttgtagacttggattttcagagttgagagcatctattattttagctcagtccctcccttaacatgatagatacatttgttcctagttgtatttgatgtggttttggttagatattctgtgtttactagcagaccttggaattgtagtatctaaagcgtacaattatttgtagaagtttcaggaaggacaaaattctgagttctgataaattcttgatacattcaacaatgttttggatctagacttgcatttctgtagaatgcacaatgtgctctacactgagatggctcgaatatttttggaattttgttgagatgattttgggggtatcgtcttagttgagcattttctttatgctctaagactaaattctctttttcgaggtctatcccatgtttaagattttgacaattttattagttcaggattgagatttaaggtttcaatttgctgataaaagtaagtctataaaacttgtaggatcaatccctgtggtggttggtgctccaaacatccaagactttgctccttctcctaattcagttttacacattaaagagataaaagatgctgaatcaattgccaataccatgaagtaccttgctcaaaaccctattgcatacaatgagtcattaaggtatgcatcaataaaagttattgttgttgttgttgttgtcattgttgttctccagttgtttacttgataatgggatggtactcttcttaatagttcgaattcctgtcgttgcaattatacactgtccaaatctctctttttttaaagccatttggtgccttttgaaaatagtattacaaagaaaatattacatacccatttcactaaaaggttttcactactgtatttccagtttttgaccaatttatatatagatatttccttttgatgttaggtggataactgaattgaacgagaacacaatggatctctctctgtttttctgtagttacaagacacttcttccttacaagatttacttaatatttcttaaatttactggacatctaacaaatgatttgctttaattttcaggtggaagtttgagggcccatctgatgccttcaaagccctggttgatatggcagcagttcattcatcttgtcgtttgtgcatcttcttggcaagtaggatccgggaaagagaagagcagagtccaaaatttatgaagcgtccctgcaaatgtactagagggactgaaactgtctatcatgtatatgtacgtgaaagagggaggtttgagatggattccattttcttaaggtattttcattctccagttactgaattctgaccttgaatgtctaagcaaattttctctgacttgttaaaaagaatatcaaagcatattttctgaatatgttcgaggcagatatgcatctactttttcctatcagttcaactgcttttatattattattattattattattcctttcagcttcgtttgctgttgttttgcactcaatcactcagtgaatgacaatttttgagatatgttctccagaattctaccagacaaagaataatattttagatttctatatcgtgaaatcttctgtgcttttcctttaattcattggggatggggtagactctctctgtccacatgtctgtggtcttgtccaagacacttgtaaagttatcatctgcttatatctttctctttccctcttaccaagctgcgtgattattctcatgttgaagagctaaaagtagaacccgagagtttgcaccttctgctcgcccttccagtctcctccatctgtacaactgtgaggtcaaatagttatgcttttttatcctacagattccaacaagtaaagctgaaagtggagctctcatttacaatgaatgcatgtgaaatacatgatatctcgcaagagttgatagtctaaggggttgaaaagtgtttcaacatgaaaagaggaaaagagatttgtggttggataacaccatagagacactatcaatctgtgtataatcatttctgattgattcgtaggctgaagcaggaagatcctgaaagttgttgtagtgggtagttgcttctgaatttctcggagtaaagaaactaaagaaaaggaaattgaacccttttcatttgaattttcgaaaattcttccatttttctatgggtacttcctgcaccgccattatatcttttgaattctgtcctggaattctcctcctgttagattgcaaagcatcctctcttttgtttaatgttttcttgtcatagctttccttatctttctgatataaactttgaatcaccctaatttctgtatctgtgcagatcgagtaatttgtctttaaaggcgtttgaatctgctatcctctcgaggttcaagtctgttaaacatgttcctgtttggaaggaggaaagacctcaagtactacgcggtgatgatgaactcaaactttacaaagtatatcctgttggcttgacacagagacaagcagtgttttccttcagattcaacggggatactgagtttaagaattacattcaaagccacccatgtgcaaaatttgaagccatattcgtatag

SEQ ID NO: 95 - protein, 301 aa, Artificial sequence
  rasburicase gene of Aspergillus
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

(19) United States
(12) Patent Application Publication
(10) Pub. No.: US 2023/0374447 A1
(43) Pub. Date: Nov. 23, 2023

(54) DICER-LIKE KNOCK-OUT PLANT CELLS

(71) Applicant: Protalix Ltd., Carmiel (IL)

(72) Inventors: Uri HANANIA, Carmiel (IL); Maor SHEVA, Kfar-Vradim (IL)

(73) Assignee: Protalix Ltd., Carmiel (IL)

(21) Appl. No.: 18/030,103

(22) PCT Filed: Oct. 5, 2021
(86) PCT No.: PCT/IL2021/051194

§ 371 Date: Apr. 4, 2023

Related U.S. Application Data
(60) Provisional application No. 63/186,280, filed on May 10, 2021.
(60) Provisional application No. 63/087,366, filed on Oct. 5, 2020.

(51) Int. Cl. C12N 5/04; C12N 15/82; C12N 9/22

(52) U.S. Cl. CPC C12N 5/04; C12N 15/8257; C12N 9/22; C12N 2310/20

(57) ABSTRACT

DICER-liker knock-out plant cells are provided. Accordingly, there is provided an isolated plant cell in suspension comprising loss of function mutations in all alleles of at least two genes selected from the group consisting of DCL2, DCL4, RDR1, RDR2 and RDR6 in said plant cell. Also provided are methods of abolishing expression and/or activity of at least two genes selected from the group consisting of DCL2, DCL4, RDR1, RDR2 and RDR6 in a plant cell.

### RELATED APPLICATIONS

This application claims the benefit of priority of U.S. Provisional Patent Application No. 63/087,366 filed on 5 Oct. 2020 and U.S. Provisional Patent Application No. 63/186,280 filed on 10 May 2021, the contents of which are incorporated herein by reference in their entirety.

### SEQUENCE LISTING STATEMENT

The ASCII file, entitled 89444SequenceListing.txt, created on 5 Oct. 2021, comprising 558,975 bytes, submitted concurrently with the filing of this application is incorporated herein by reference.

### FIELD AND BACKGROUND OF THE INVENTION

The present invention, in some embodiments thereof, relates to DICER-liker knock-out plant cells.

Production of proteins in plants for human health applications offers some advantages, including ease of scaling and lack of human and animal pathogens (Donini and Marusic, 2019; Loh et al., 2017; Tekoah et al., 2015). The first plant-derived pharmaceutical recombinant protein was approved in 2012 for commercial use in humans (Mor, 2016) and the second enzyme alpha-galactosidase-A for the treatment of the Fabry disease that is currently tested in phase III clinical trials (Kizhner et al., 2015; Ruderfer et al., 2018). Despite the success, plant expression systems are characterized by low yields as a result of gene silencing that is one of the fundamental regulatory and defense mechanism controlling gene expression, developmental regulation and epigenetic modifications (Baulcombe, 2004; Moazed, 2009).

RNA interference occurs either by transcriptionally gene silencing (TGS) related to DNA modification by RNA-directed DNA methylation (RdDM) or post-transcriptional gene silencing (PTGS) related to RNA modification by degradation of mRNA or blocking the translation of RNA transcripts. In both situations, small RNA (siRNA) molecule, 21-24 nt in length, are produced (Ghildiyal and Zamore, 2009). Several proteins are known to be involved in these processes. RNA-dependent RNA polymerases (RDRs) are a family of proteins that produce double-stranded RNA (dsRNA) molecules using aberrant single-stranded RNAs, such as viral RNAs or transgene-derived mRNAs, as templates. The endoribonuclease (RNase III) enzymes known in plants as DICER like (DCL), produce the siRNAs or miRNAs (21-24 nt) from long double-stranded RNA or hairpin-loop-structured RNAs (Ji, 2008). A dsRNA-binding protein recruits a Dicer-siRNA complex to Argonaute (Ago) family of proteins, and the Ago cleaves the anti-guide strand of the siRNA duplex (Hammond et al., 2001). The remaining single-stranded (ss) antisense siRNA along with the Ago associates with other RNA-binding proteins that assemble into RNA-induced silencing complex (RISC) (Martinez et al., 2002). In the cytoplasm, mRNA targets can be cleaved via RISC slicer activity by Ago to silence targeted genes, known as PTGS (Rand et al., 2005). In the nucleus, RISC can take the form of an RNA-induced transcriptional silencing (RITS) complex, which interacts with RNA polymerase II (PoIII) and nascent RNA transcripts and directs chromatin remodeling to achieve epigenetic silencing through RdDM (Pratt and MacRae, 2009; Verdel et al., 2004).

Plant genomes contain at least four distinct classes of DCL family proteins (DCL1-4) that play a central role in gene silencing. Like their animal counterparts, each class of DCL participates in a specific function (Fukudome and Fukuhara, 2016). DCL1 gives rise to 21 nucleotides (nt) long siRNAs from miRNA precursors, which are transcribed from non-coding genes (Choudhary et al., 2019). The main activity of DCL3 is to process long dsRNA derived from heterochromatic regions of the genome into 24-nt siRNAs. These molecules then direct DNA methylation of target sequences (Blevins et al., 2015). DCL2 and DCL4 are involved in virus induced RNA silencing (VIGS) and transgene silencing. DCL2 gives rise to 22 (nt) siRNAs and DCL4 is responsible for the production of 21-nt siRNAs (Chen et al., 2018; Parent et al., 2015).

Plants genomes contain six RDRs, divided into two groups: RDRα consisting of RDR1, RDR2 and RDR6 and RDR7 consisting of RDR3, RDR4 and RDR5. RDR1, RDR2, and RDR6 genes share the C-terminal canonical catalytic DLDGD (SEQ ID NO: 32) motif of eukaryotic RDRs, which are involved in plant antiviral and transgene silencing (Wassenegger and Krczal, 2006).

Induced mutations and RNAi technology were implemented in plants to knock-down DCL or RDR genes involved in silencing (e.g. Daxinger et al., 2008; Parent et al., 2015; Seta et al., 2017; Yoshikawa et al., 2005, Konstantina Katsarou et al.Mol Plant Pathol. 2019 March; 20(3): 432-446; Matsuo and Matsumura, 2017; Qin et al., 2017; Suzuki et al., 2019, Deleris et al. Science. 2006 Jul. 7; 313(5783):68-71; Xie et al. Proc Natl Acad Sci USA. 2005 Sep. 6; 102(36): 12984-12989; Hernan Garcia-Ruiz et al. Plant Cell. 2010 February; 22(2): 481-496; Polydore S and Axtell M J (2018) The Plant Journal 94:1051-1063; Matsuo and Atsumi G (2019) Planta 250:463-473; International Patent Application Publication Nos. WO2015054602 and WO2019222379; and US Patent Application Publication Nos. US20120240288 and US20100050294). Notably, few of these studies show that DCL suppression results in distinct developmental defects and higher susceptibility to certain RNA viruses.

### SUMMARY OF THE INVENTION

According to an aspect of some embodiments of the present invention there is provided an isolated plant cell in suspension comprising loss of function mutations in all alleles of at least two genes selected from the group consisting of DCL2, DCL4, RDR1, RDR2 and RDR6 in the plant cell.

According to some embodiments of the invention, the loss of function mutations in the DCL2 are in a region shared by all alleles of the DCL2 in the plant cell.

According to some embodiments of the invention, the loss of function mutations in the DCL2 are located within nucleic acid residues 288-307 and/or 881-900 corresponding to SEQ ID NO: 74; and/or nucleic acid residues 288-307 and/or 881-900 corresponding to SEQ ID NO: 75.

According to some embodiments of the invention, the region shared by all alleles of the DCL2 gene comprises a sequence selected from the group consisting of SEQ ID NO: 1-2.

According to some embodiments of the invention, the loss of function mutations in the DCL4 are in a region shared by all alleles of the DCL4 in the plant cell.

According to some embodiments of the invention, the loss of function mutations in the DCL4 are located within nucleic acid residues 967-986 and/or 1407-1426 corresponding to SEQ ID NO: 76; and/or nucleic acid residues 931-950 and/or 1371-1390 corresponding to SEQ ID NO: 77.

According to some embodiments of the invention, the region shared by all alleles of the DCL4 gene comprises a sequence a sequence selected from the group consisting of SEQ ID NO: 3-4.

According to some embodiments of the invention, the loss of function mutations in the RDR1 are in a region shared by all alleles of the RDR1 in the plant cell.

According to some embodiments of the invention, the loss of function mutations in the RDR1 are located within nucleic acid residues 916-937 and/or 962-984 corresponding to SEQ ID NO: 78; and/or nucleic acid residues 921-937 and/or 962-984 corresponding to SEQ ID NO: 79.

According to some embodiments of the invention, the region shared by all alleles of the RDR1 gene comprises a sequence a sequence selected from the group consisting of SEQ ID NO: 26-27.

According to some embodiments of the invention, the loss of function mutations in the RDR2 are in a region shared by all alleles of the RDR2 in the plant cell.

According to some embodiments of the invention, the loss of function mutations in the RDR2 are located within nucleic acid residues 575-553 and/or 589-612 corresponding to SEQ ID NO: 80; and/or nucleic acid residues 575-553 and/or 589-612 corresponding to SEQ ID NO: 81.

According to some embodiments of the invention, the region shared by all alleles of the RDR2 gene comprises a sequence a sequence selected from the group consisting of SEQ ID NO: 28-29.

According to some embodiments of the invention, the loss of function mutations in the RDR6 are in a region shared by all alleles of the RDR6 in the plant cell.

According to some embodiments of the invention, the loss of function mutations in the RDR6 are located within nucleic acid residues 2767-2785 and/or 2820-2839 corresponding to SEQ ID NO: 82; nucleic acid residues 49-67 and/or 102-121 corresponding to SEQ ID NO: 83; and/or nucleic acid residues 49-67 and/or 102-121 corresponding to SEQ ID NO: 84.

According to some embodiments of the invention, the region shared by all alleles of the RDR6 gene comprises a sequence a sequence selected from the group consisting of SEQ ID NO: 30-31.

According to some embodiments of the invention, the plant cell has reduced expression and/or activity of a glycosylation enzyme as compared to a control plant cell of the same genetic background not subjected to an agent which downregulates expression and/or activity of the glycosylation enzyme.

According to some embodiments of the invention, the isolated plant cell comprising a heterologous nucleic acid sequence for expressing an expression product of interest.

According to an aspect of some embodiments of the present invention there is provided a method of expressing a recombinant expression product of interest in a plant cell, the method comprising culturing the cell under condition which allow expression of the expression product of interest.

According to an aspect of some embodiments of the present invention there is provided a method of abolishing expression and/or activity of at least two genes selected from the group consisting of DCL2, DCL4, RDR1, RDR2 and RDR6 in a plant cell, the method comprising introducing into an isolated plant cell in suspension an agent capable of introducing loss of function mutations in all alleles of at least two genes selected from the group consisting of DCL2, DCL4, RDR1, RDR2 and RDR6 in the plant cell.

According to some embodiments of the invention, the method comprises introducing into the isolated plant cell an agent capable of downregulating expression and/or activity of a glycosylation enzyme.

According to some embodiments of the invention, the plant cell has reduced expression and/or activity of a glycosylation enzyme as compared to a control plant cell of the same genetic background not subjected to an agent which downregulates expression and/or activity of the glycosylation enzyme.

According to some embodiments of the invention, the glycosylating enzyme comprises xylosyltransferase and/or fucosyltransferase.

According to some embodiments of the invention, the agent is a genome editing agent.

According to some embodiments of the invention, the genome editing agent is selected from the group consisting of CRISPR/Cas system, Zinc finger nuclease (ZFN), transcription-activator like effector nuclease (TALEN) or meganuclease.

According to some embodiments of the invention, the agent comprises a DCL2 DNA targeting sequence comprising a nucleic acid sequence selected from the group consisting of SEQ ID NO: 1-2.

According to some embodiments of the invention, the agent comprises a DCL4 DNA targeting sequence comprising a nucleic acid sequence selected from the group consisting of SEQ ID NO: 3-4.

According to some embodiments of the invention, the agent comprises a RDR1 DNA targeting sequence comprising a nucleic acid sequence selected from the group consisting of SEQ ID NO: 26-27.

According to some embodiments of the invention, the agent comprises a RDR2 DNA targeting sequence comprising a nucleic acid sequence selected from the group consisting of SEQ ID NO: 28-29.

According to some embodiments of the invention, the agent comprises a RDR6 DNA targeting sequence comprising a nucleic acid sequence selected from the group consisting of SEQ ID NO: 30-31.

According to some embodiments of the invention, the method further comprising expressing in the isolated plant cell a recombinant expression product of interest other than the agent.

According to some embodiments of the invention, the loss of function mutations abolish expression of the at least two genes, as determined by RT-PCR.

According to some embodiments of the invention, the loss of function mutations abolish expression and/or activity of the DCL2 and DCL4, as determined by no expression of transgene specific 21-nt and 22-nt siRNAs in the plant cell following expression of the transgene in the plant cell.

According to some embodiments of the invention, the plant is selected from the group consisting of Tobacco, Arabidopsis, Aloe Vera, grape seeds, oil palm, plantain, pine, banana, date, eggplant, jojoba, pineapple, rubber tree, cassava, yam, sweet potato and tomato.

According to some embodiments of the invention, the plant is a Tobacco plant.

According to some embodiments of the invention, the Tobacco is Nicotiana tabacum.

According to some embodiments of the invention, the plant cell is a BY-2 line cell.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C show schematic representations of the CRISPR-Cas9 vectors for the knockout of DCL2 and DCL4 genes. FIG. 1A demonstrates the phCas9-DCL2 vector; FIG. 1B demonstrates the phCas9-DCL4 vector; FIG. 1C demonstrates the phCas9-DCL2-DCL4 vector. LB—left border; IRES—Internal ribosome entry site; hptII—Hygromycin phosphotransferase II; Nter—Nopaline synthase terminator; 35S-35S cauliflower mosaic virus promoter with omega enhancer; hCas9—human-optimized Cas9 with the SV40 nuclear localization signal (Nekrasov et al., 2013); Oter—Octopinesynthase terminator; U6—Arabidopsis U6 promoter; sgRNA1-4—chimeras of the various crRNAs with tracrRNA, represent the four different crRNAs that were used to knock out DCL2 (gRNA1,2) and DCL4 (gRNA3,4) genes.

FIG. 2 shows expression levels of recombinant rasburicase in ΔD2ΔD4, ΔD2 and ΔD4 pooled cells relative to line 40, as determined by protein activity assay. Line 40 ΔD2ΔD4—pool of line 40 cells transformed with phCas9-DCL2-DCL4 vector. This pool contains in part, knockout cells of both DCL2 and DCL4 genes. Line 40 ΔD2—pool of line 40 cells transformed with phCas9-DCL2 vector. This pool contains in part, knockout cells of DCL2 genes. Line 40 ΔD4—pool of line 40 cells transformed with phCas9—DCL4 vector. This pool contains in part, knockout cells of DCL4 genes. Line 40—the original line expressing recombinant rasburicase used for the transformation to knock-out DCL2 and/or DCL4 genes. The experiment was conducted in 3 repeats. Error bars represent standard deviation errors.

FIG. 3 shows expression levels of recombinant rasburicase in the indicated ΔD2ΔD4 lines (isolated from the pooled ΔD2ΔD4 cells) relative to line 40, as determined by protein activity assay.

FIG. 4 shows expression levels of recombinant rasburicase in the indicated ΔD2 lines (isolated from the pooled ΔD2 cells) relative to line 40, as determined by protein activity assay.

FIGS. 5A-B demonstrate the amplification fragment length polymorphism (AFLP) assay for detecting indels in DCL2. FIG. 5A is a schematic presentation of the PCR amplification scheme. FP—forward primer. RP—reverse primer. FIG. 5B a DNA gel image demonstrating the PCR amplicons obtained from wild-type BY2 cells (104 bp) as compared to ΔD2ΔD4 lines 3, 18, 34, 58, 65. M—DNA 100 bp ladder. Sizes are indicated on the left.

FIGS. 6A-B demonstrate the restriction fragment length polymorphism (RFLP) assay for DCL4. FIG. 6A is a schematic representation of the EcoNI site (Italic and bold) included in the DCL4-gRNA3 site (underlined letters, SEQ ID NO: 3) followed by the PAM sequence (GGG). A pair of primers upstream and downstream of the EcoNI site indicated by arrows were used for PCR amplification of the targeted DNA locus. FP—forward primer. RP—reverse primer. FIG. 6B is a DNA gel image demonstrating the pattern of EcoNI digested amplicons obtained from wild-type BY2 cells (189 bp and 89 bp) as compared to ΔD2ΔD4 lines 3, 18, 34, 58, 65. M-DNA marker (sizes are indicated on the left).

FIGS. 7A-B demonstrate mutations in the DCL2 and DCL4 genes in ΔD2ΔD4 line 18. Sequencing of the mutated DCL2 genes (FIG. 7A) and sequencing of the mutated DCL4 genes (FIG. 7B). Sequences of the wild type genes are designated in uppercase and sequences of the mutated alleles are shown below in lowercase. The cas9 target site sequence is underlined. Deletions are indicated by dashes and insertions are indicated by uppercase. The arrow indicates the precise cleavage site. The size of indels is shown on the right in bp.

FIGS. 8A-B demonstrate mutations in the DCL2 and DCL4 genes in ΔD2ΔD4 line 65. Sequencing of the mutated DCL2 genes (FIG. 8A) and sequencing of the mutated DCL4 genes (FIG. 8B). Sequences of the wild type genes are designated in uppercase and sequences of the mutated alleles are shown below in lowercase. The cas9 target site sequence is underlined. Deletions are indicated by dashes and insertions are indicated by uppercase. The arrow indicates the precise cleavage site. The size of indels is shown on the right in bp.

FIGS. 9A-C demonstrate that the increased expression of recombinant rasburicase is attributed to knock-out of DCL2 and DCL4 genes. FIG. 9A shows expression levels of recombinant rasburicase in the indicated ΔD2 and ΔD2ΔD4 lines relative to line 40, as determined by protein activity assay. FIG. 9B shows detection of small interfering RNAs (siRNA). Total siRNA of the original line 40 or the indicated ΔD2 or ΔD2ΔD4 lines was isolated and hybridized with a rasburicase RNA probe. The upper panel shows the hybridization signal. The lower panel shows loading control (mainly tRNA) stained with ethidium bromide. siRNA sizes are indicated on the left using a specific rasburicase ladder. FIG. 9C shows expression levels of rasburicase mRNA in the indicated ΔD2ΔD4 lines relative to line 40, as determined by real-time RT-PCR using glycosyltransferase gene as a housekeeping gene.

FIGS. 10A-B demonstrate the amplification fragment length polymorphism (AFLP) assay for detecting indels in DCL2. FIG. 10A is a schematic presentation of the PCR amplification scheme. FP—forward primer. RP—reverse primer. FIG. 10B a DNA gel image demonstrating the PCR amplicons obtained from wild-type BY2 cells (104 bp) as compared to ΔD2ΔD4 lines 5, 35, 71, 93, 97, 103. M—DNA 100 bp ladder. Sizes are indicated on the left.

FIGS. 11A-B demonstrate the restriction fragment length polymorphism (RFLP) assay for DCL2. FIG. 11A is a schematic representation of the PspFI site (Italic and bold) included in the DCL2-gRNA2 site (underlined letters, SEQ ID NO: 2) followed by the PAM sequence (TGG). A pair of primers upstream and downstream of the PspFI site indicated by arrows were used for PCR amplification of the targeted DNA locus. FP—forward primer. RP—reverse primer. FIG. 11B is a DNA gel image demonstrating the pattern of PspFI digested amplicons obtained from wild-type BY2 cells (189 bp and 529 bp) as compared to ΔD2ΔD4 lines 5, 35, 71, 93, 97 and 103. M-DNA marker (sizes are indicated on the left).

FIGS. 12A-B demonstrate the restriction fragment length polymorphism (RFLP) assay for DCL4. FIG. 12A is a schematic representation of the EcoNI site (Italic and bold) included in the DCL4-gRNA3 site (underlined letters, SEQ ID NO: 3) followed bY the PAM sequence (GGG). A pair of primers upstream and downstream of the EcoNI site indicated by arrows were used for PCR amplification of the targeted DNA locus. FP—forward primer. RP—reverse primer. FIG. 12B is a DNA gel image demonstrating the pattern of EcoNI digested amplicons obtained from wild-type BY2 cells (189 bp and 89 bp) as compared to ΔD2ΔD4 lines 5, 35, 71, 93, 97 and 103. M-DNA marker (sizes are indicated on the left).

FIGS. 13A-B demonstrate mutations in the DCL2 and DCL4 genes in ΔD2ΔD4 line 35. Sequencing of the mutated DCL2 genes (FIG. 13A) and sequencing of the mutated DCL4 genes (FIG. 13B). Sequences of the wild type genes are designated in uppercase and sequences of the mutated alleles are shown below in lowercase. The cas9 target site is underlined. Deletions are indicated by dashes. The arrow indicates the precise cleavage site. The size of indels is shown on the right in bp.

FIGS. 14A-B demonstrate expression levels of recombinant rasburicase in ΔD2ΔD4, ΔD2 and ΔD4 pooled cells relative to line 40. Putative DCL2 and DCL4 knockout lines 5, 35, 71, 93, 97, 103 and wild type BY2 were transformed with rasburicase BeYDV vector and pools of each transformation were analyzed. FIG. 14A shows expression level of rasburicase as determined by activity assay. The experiment was conducted in 3 repeats. Error bars represent standard deviation errors. FIG. 14B shows expression level of rasburicase as determined by coomassie blue stained SDS-PAGE of total proteins. Std—1 μg of recombinant rasvyricase. N.C—non-transformed BY2 used as negative control. BY2—wild type BY2 cells transformed with rasburicase. Lines 5, 35 and 93—DCL2 and DCL4 knockout lines transformed with rasburicase. Molecular size in kilo Daltons is indicated on left.

FIG. 15 demonstrates that the increased expression of recombinant rasburicase is attributed to knock-out of DCL2 and DCL4 genes. Total siRNA of BY2 or the indicated ΔD2ΔD4 lines transformed with rasburicase was isolated and hybridized with a rasburicase RNA probe. N.C—non-transformed BY2 used as negative control. The upper panel shows the hybridization signal. The lower panel shows loading control (mainly tRNA) stained with ethidium bromide. siRNA sizes are indicated on the left using a specific rasburicase ladder.

FIG. 16 is a schematic representation of the design of a crRNA to a specific target sequence used by the present inventors.

### DESCRIPTION OF SPECIFIC EMBODIMENTS OF THE INVENTION

The present invention, in some embodiments thereof, relates to DICER-liker knock-out plant cells.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The production of recombinant proteins in plants has many advantages. However, plant expression systems are characterized by low yields as a result of gene silencing. Plants DICER like (DCL) and RNA-dependent RNA polymerases (RDRs) proteins are key components participating in gene silencing.

While reducing specific embodiments of the present invention to practice, the present inventors have now generated DCL2- and DCL4 knockout plant cells. Furthermore, these cells expressed higher amounts of a recombinant protein as compared to their wild type counterparts.

Specifically, the present inventors used CRISPR/Cas9 technology to develop DCL2- and DCL4 knockout Nicotiana tabacum BY2 cells which are fully mutated in all gene alleles (Examples 1-3 of the Examples section which follows). These knocked-out BY2 cells were viable, and their growth rate was similar to native BY2 cells. Furthermore, the expression level of a recombinant protein (Rasburicase) in the DCL2- and DCL4 knockout cell lines was up to 10 fold higher compared to the expression level in the wild-type cells. Corroborating the correlation between DCL2- and DCL4 knock-out and the increased expression of a recombinant protein, the present inventors show that the knock-out lines do not produce transgene specific 21 bp and 22 bp siRNA.

Consequently, specific embodiments of the present invention propose isolated DCL2-and DCL4 knock-out plant cells and methods of generating same. Following, these novel plant cells may be used for e.g. expression of recombinant proteins.

Thus, according to one aspect of the present invention, there is provided a method of abolishing expression and/or activity of at least two genes selected from the group consisting of DCL2, DCL4, RDR1, RDR2 and RDR6in a plant cell, the method comprising introducing into an isolated plant cell in suspension an agent capable of introducing loss of function mutations in all alleles of at least two genes selected from the group consisting of DCL2, DCL4, RDR1, RDR2 and RDR6in said plant cell.

According to specific embodiments, the at least two genes comprise at least DCL2 and DCL4, DCL2 and RDR1, DCL2 and RDR2, DCL2 and RDR6, DCL4 and RDR1, DCL4 and RDR2, DCL4 and RDR6, RDR1 and RDR2, RDR1 and RDR6, or RDR2 and RDR6, each possibility represents a separate embodiments of the invention.

According to specific embodiments, the at least two genes comprise at least three genes, at least four genes or all of the 5 genes.

According to specific embodiments, the at least three genes comprise at least DCL2+DCL4+RDR1, DCL2+DCL4+RDR2, DCL2+DCL4+RDR6, DCL2+RDR1+RDR2, DCL2+RDR1+RDR6, DCL2+RDR2+RDR6, DCL4+RDR1+RDR2, DCL4+RDR1+RDR6, DCL4+RDR2+RDR6, RDR1+RDR2+RDR6, each possibility represents a separate embodiments of the invention.

The agent may be a single agent targeting the different genes or several distinct agents each targeting at least one gene.

Thus, according to specific embodiments, the agent comprises at least two, at least three, at least four or at least 5 distinct agents.

DCL (DICER like) are endoribonuclease enzymes belonging to the RNase III family. DCL enzymes cleave double-stranded RNA (dsRNA) or hairpin-loop-structured RNAs and pre-microRNA (pre-miRNA) into short single-stranded RNA fragments called small interfering RNA and microRNA, respectively. Plant genomes contain at least four distinct classes of DCL family proteins.

As used herein, the term “DCL2” refers to a gene encoding endoribonuclease Dicer like 2. DCL2 creates 22-nt long siRNA from cis-acting antisense transcripts which aid in viral immunity and defense.

The Nicotiana tabacum, TN90 for example, comprises two DCL2 genes, their sequences can be obtained from known databases such as solgenomics (www(dot)solgenomics(dot)net). Exemplary contigs include:
N.tab-DCL2A NW_015902585 (SEQ ID NO: 63) and
N.tab-DCL2B NW_015936378 (SEQ ID NO: 64).

Exemplary cDNA sequences of Nicotiana tabacum DCL2A and DCL2B are provided in SEQ ID NOs: 74 and 75, respectively.

As used herein, the term “DCL4” refers to a gene encoding endoribonuclease Dicer like 4. DCL4 is involved in trans-acting siRNA metabolism and transcript silencing at the post-transcriptional level and creates 21-nt long siRNA.

The Nicotiana tabacum, TN90 for example, comprises two DCL4 genes, their sequences can be obtained from known databases such as solgenomics (www(dot)solgenomics(dot)net). Exemplary contigs include:
N.tab-DCL4A NW_015930707 (SEQ ID NO: 65) and
N.tab-DCL4B NW_015939689 (SEQ ID NO: 66).

Exemplary cDNA sequences of Nicotiana tabacum DCL2A and DCL2B are provided in SEQ ID NOs: 76 and 77, respectively.

RNA-dependent RNA polymerase (RDR), also known as RNA replicase, EC NO: 2.7.7.48, is an enzyme that catalyzes the replication of RNA from an RNA template. There are three major clades of eukaryotic RDRs-RDRα, RDRβ, and RDRγ, while RDRβwas likely lost in the plant lineage. Plant genomes contain at least 6 RDR family proteins. RDRα genes have duplicated in plants to yield separate RDR1, RDR2, and RDR6 subgroups and RDRγ genes have duplicated to yield RDR3, RDR4, and RDR5 subgroups [Zong et al., (2009), Gene 447: 29-39].

As used herein, the term “RDR1” refers to a gene encoding RNA-dependent RNA polymerase 1. RDR1 is involved in pathogen resistance and stress response. RDR1 contributes to the production of vsRNAs (21-nt, 22-nt) and the antiviral defense conferred through these vsRNAs.

The Nicotiana tabacum, TN90 for example, comprises 2 RDR1 genes, their sequences can be obtained from known databases such as solgenomics (www(dot)solgenomics(dot)net). Exemplary contigs include:
N.tab-RDR1A Ntab-TN90_AYMY-SS286 (SEQ ID NO: 67) and
N.tab-RDR1B Ntab-TN90_AYMY-SS10620 (SEQ ID NO: 68).

Exemplary cDNA sequences of Nicotiana tabacum RDR1A and RDR1B are provided in SEQ ID NOs: 78 and 79, respectively.

As used herein, the term “RDR2” refers to a gene encoding RNA-dependent RNA polymerase 2. RDR2 is involved in transgene silencing and essential for the biogenesis of endogenous siRNA (female gamete formation, genome maintenance etc.). It leads to the formation of 24-nt and cytosine methylation, histone modification and RNA-directed DNA methylation (RdDM).

The Nicotiana tabacum, TN90 for example, comprises 2 RDR2 genes, their sequences can be obtained from known databases such as solgenomics (www(dot)solgenomics(dot)net). Exemplary contigs include:
N.tab-RDR2A Nitab4.5_0006831 (SEQ ID NO: 69) and
N.tab-RDR2B Nitab4.5_0010542 (SEQ ID NO: 70).

Exemplary cDNA sequences of Nicotiana tabacum RDR2A and RDR2B are provided in SEQ ID NOs: 80 and 81, respectively.

As used herein, the term “RDR6” refers to a gene encoding RNA-dependent RNA polymerase 6. RDR6 participates in the production of transacting tasiRNAs and natural antisense siRNAs and was also found to be essential for sense transgene-induced posttranscriptional gene silencing (S-PTGS). RDR6 contributes to the antiviral defense conferred through these vsRNAs (21-nt, 24-nt).

The Nicotiana tabacum, TN90 for example, comprises 3 RDR6 genes, their sequences can be obtained from known databases such as solgenomics (www(dot)solgenomics(dot)net). Exemplary contigs include:
N.tab-RDR6A Ntab-TN90_AYMY-SS110011 (SEQ ID NO: 71);
N.tab-RDR6B Ntab-TN90_AYMY-SS48576 (SEQ ID NO: 72); and
N.tab-RDR6C Ntab-TN90_AYMY-SS1589 (SEQ ID NO: 73).

Exemplary cDNA sequences of Nicotiana tabacum RDR6A, RDR6B and RDR6C are provided in SEQ ID NOs: 82, 83 and 84, respectively.

As used herein, the term “isolated plant cell” refers to a plant cell at least partially separated from the natural plant (or part thereof).

In some embodiments, the isolated cell is a plant cell in a suspension culture.

Suspension culture means that the cells are not part of a tissue, but are rather floating as single cells or clusters of not more than 100 cells in a culture medium.

Suitable devices and methods for culturing plant cells in suspension are known in the art, for example, as described in International Patent Application PCT IL2008/000614.

Non-limiting examples of plants useful in the methods of the invention include all plants which belong to the superfamily Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Cannabaceae, Cannabis indica, Cannabis, Cannabis sativa, Hemp, industrial Hemp, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalypfus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively algae and other non-Viridiplantae can be used for the methods of some embodiments of the invention.

According to some embodiments of the invention, the plant is an edible and/or non-toxic plant, which is amenable to genetic modification so as to bring about expression from the nucleic acid construct.

According to some embodiments of the invention, the plant is a crop plant such as, but not limited to, rice, maize, wheat, barley, peanut, potato, sesame, olive tree, palm oil, banana, soybean, sunflower, canola, sugarcane, alfalfa, millet, leguminosae (bean, pea), flax, lupinus, rapeseed, tobacco, tomato, carrot, cucumber, melon, grapes, while clover, celery, ginger, horseradish, poplar and cotton.

According to a specific embodiment, the plant is a carrot plant.

According to specific embodiments, the plant is selected from the group consisting of Tobacco, Arabidopsis, Aloe Vera, grape seeds, oil palm, plantain, pine, banana, date, eggplant, jojoba, pineapple, rubber tree, cassava, yam, sweet potato and tomato.

According to a specific embodiment, the plant is a Tobacco plant.

According to a specific embodiment, the Tobacco plant is Nicotiana tabacum.

According to a specific embodiment, the Tobacco plant is Nicotiana benthamiana.

In one embodiment the Tobacco cells are from a Tobacco cell line, such as, but not limited to Nicotiana tabacum L. cv Bright Yellow (BY-2) cells.

The isolated plant cell of some embodiments of the present invention is introduced with an agent capable of introducing loss of function mutations.

As used herein, the phrase “loss of function mutations” refers to any mutation in the DNA sequence of a gene (e.g., DCL2, DCL4, RDR1, RDR2, RDR6) which results in downregulation of the expression level and/or activity of the expressed product, i.e., the mRNA transcript and/or the translated protein. Non-limiting examples of such loss of function mutations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss of function mutation of a gene may comprise at least one allele of the gene.

According to specific embodiments loss of function mutation of a gene comprises more than one allele of the gene.

According to specific embodiments loss of function mutation of a gene comprises all alleles of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

As DCL2, DCL4, RDR1 and RDR2 are encoded by two distinct genes in Nicotinana plants e.g. Nicotiana tabacum, according to specific embodiments, the loss of function mutation comprises all four alleles of the gene.

As RDR6 is encoded by three distinct genes in Nicotinana plants e.g. Nicotiana tabacum, according to specific embodiments, the loss of function mutation comprises all six alleles of the gene.

In such instances the agent may be a single agent targeting the different alleles of the gene or several distinct agents each targeting at least one allele of the gene.

Thus, according to specific embodiments, the loss of function mutations are in a region shared by all alleles of the gene.

According to other specific embodiments, the loss of function mutations are in region shared by at least 2 alleles of the gene.

According to other specific embodiments, the loss of function mutations are in regions not shared by all allele of the gene.

A shared region, as used herein, refers to a region in a wild type non-mutated allele of a gene having at least 95%, at least 96%, at least 97%, at least 98%, at least 99% or 100% sequence identity to another distinct wild type non-mutated allele of the gene.

According to specific embodiments, the shared region has 100% sequence identity Sequence identity can be determined using any nucleic acid sequence alignment algorithm such as CLC, Vector NTI, Blast, ClustalW, and MUSCLE.

According to specific embodiments, the shared region is at least 10, at least 15, at least 20, at least 25 nucleic acids long.

According to specific embodiments, the loss of function mutations in the DCL2 are located within nucleic acid residues 288-307 and/or 881-900 corresponding to SEQ ID NO: 74 and/or nucleic acid residues 288-307 and/or 881-900 corresponding to SEQ ID NO: 75.

According to specific embodiments, the region shared by all alleles of the DCL2 gene comprises a sequence selected from the group consisting of SEQ ID NO: 1-2.

As the agent may comprise the sequence of the region shared by all alleles of the DCL2 gene (see FIG. 16), according to specific embodiments, the agent comprises a DCL2 DNA targeting sequence comprising a nucleic acid sequence selected from the group consisting of SEQ ID NO: 1-2.

According to specific embodiments, the loss of function mutations in the DCL4 are located within nucleic acid residues 967-986 and/or 1407-1426 corresponding to SEQ ID NO: 76 and/or nucleic acid residues 931-950 and/or 1371-1390 corresponding to SEQ ID NO: 77.

According to specific embodiments, the region shared by all alleles of the DCL4 gene comprises a sequence selected from the group consisting of SEQ ID NO: 3-4.

According to specific embodiments, the agent comprises a DCL4 DNA targeting sequence comprising a nucleic acid sequence selected from the group consisting of SEQ ID NO: 3-4.

According to specific embodiments, the loss of function mutations in the RDR1 are located within nucleic acid residues 916-937 and/or 962-984 corresponding to SEQ ID NO: 78 and/or nucleic acid residues 921-937 and/or 962-984 corresponding to SEQ ID NO: 79.

According to specific embodiments, the region shared by all alleles of the RDR1 gene comprises a sequence selected from the group consisting of SEQ ID NO: 26-27.

According to specific embodiments, the agent comprises a RDR1 DNA targeting sequence comprising a nucleic acid sequence selected from the group consisting of SEQ ID NO: 26-27.

According to specific embodiments, the loss of function mutations in the RDR2 are located within nucleic acid residues 575-553 and/or 589-612 corresponding to SEQ ID NO: 80; and/or nucleic acid residues 575-553 and/or 589-612 corresponding to SEQ ID NO: 81.

According to specific embodiments, the region shared by all alleles of the RDR2 gene comprises a sequence selected from the group consisting of SEQ ID NO: 28-29.

According to specific embodiments, the agent comprises a RDR2 DNA targeting sequence comprising a nucleic acid sequence selected from the group consisting of SEQ ID NO: 28-29.

According to specific embodiments, the loss of function mutations in the RDR6 are located within nucleic acid residues 2767-2785 and/or 2820-2839 corresponding to SEQ ID NO: 82, nucleic acid residues 49-67 and/or 102-121 corresponding to SEQ ID NO: 83; and/or nucleic acid residues 49-67 and/or 102-121 corresponding to SEQ ID NO: 84.

According to specific embodiments, the region shared by all alleles of the RDR6 gene comprises a sequence selected from the group consisting of SEQ ID NO: 30-31.

According to specific embodiments, the agent comprises a RDR6 DNA targeting sequence comprising a nucleic acid sequence selected from the group consisting of SEQ ID NO: 30-31.

As used herein, the phrase “corresponding to SEQ ID NO:” intends to include the nucleic acid or a homolog thereof as defined by its location in the sequence of the recited SEQ ID NO relative to any other sequence encoding the recited enzyme.

Methods of introducing loss of function mutations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce loss of function mutations to a gene of interest and agents for implementing same that can be used according to specific embodiments of the present invention.

According to specific embodiments, the agent is a genome editing agent.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDS) and non-homologous end-joining (NHEJF). NHEJF directly joins the DNA ends in a double-stranded break, while HDR utilizes a homologous donor sequence as a template for regenerating the missing DNA sequence at the break point. In order to introduce specific nucleotide modifications to the genomic DNA, a donor DNA repair template containing the desired sequence must be present during HDR.

Genome editing cannot be performed using traditional restriction endonucleases since most restriction enzymes recognize a few base pairs on the DNA as their target and these sequences often will be found in many locations across the genome resulting in multiple cuts which are not limited to a desired location. To overcome this challenge and create site-specific single- or double-stranded breaks, several distinct classes of nucleases have been discovered and bioengineered to date. These include for example the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location.

This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence.

Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fok1. Additionally Fok1 has the advantage of requiring dimerization to have nuclease activity and this means the specificity increases dramatically as each nuclease partner recognizes a unique DNA sequence. To enhance this effect, Fok1 nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the non-homologous end-joining (NHEJ) pathway often results in small deletions or small sequence insertions. Since each repair made by NHEJ is unique, the use of a single nuclease pair can produce an allelic series with a range of different deletions at the target site.

The deletions typically range anywhere from a few base pairs to a few hundred base pairs in length, but larger deletions have been successfully generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers are typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, CA).

CRISPR-Cas system (also referred to herein as “CRISPR”)—Many bacteria and archea contain endogenous RNA-based adaptive immune systems that can degrade nucleic acids of invading phages and plasmids. These systems consist of clustered regularly interspaced short palindromic repeat (CRISPR) nucleotide sequences that produce RNA components and CRISPR associated (Cas) genes that encode protein components. The CRISPR RNAs (crRNAs) contain short stretches of homology to the DNA of specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is a combination of the target homologous sequence (crRNA) and the endogenous bacterial RNA that links the crRNA to the Cas e.g. Cas9 nuclease (tracrRNA) in a single chimeric transcript. The gRNA/Cas complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded breaks produced by CRISPR/Cas can undergo homologous recombination or NHEJ and are susceptible to specific sequence modification during DNA repair.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs. This creates a system that can be readily modified to target modifications at different genomic sites and/or to target different modifications at the same site. Additionally, protocols have been established which enable simultaneous targeting of multiple genes. The majority of cells carrying the mutation present biallelic mutations in the targeted genes.

However, apparent flexibility in the base-pairing interactions between the gRNA sequence and the genomic DNA target sequence allows imperfect matches to the target sequence to be cut by the endonuclease e.g. Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

In order to cut DNA at a specific site, Cas9 proteins require the presence of a gRNA and a protospacer adjacent motif (PAM), which immediately follows the gRNA target sequence in the targeted polynucleotide gene sequence. The PAM is located at the 3′ end of the gRNA target sequence but is not part of the gRNA. Different Cas proteins require a different PAM. Accordingly, selection of a specific polynucleotide gRNA target sequence by a gRNA is generally based on the recombinant Cas protein used. Non-limiting examples of PAM sequence include 5-NRG-3′, where R is either A or G, NNGRR (SEQ ID NO: 85), “NGG” sequence, “NAG”, NNNNGATT (SEQ ID NO: 86) and NNNNGNNN (SEQ ID NO: 87) where “N” can be any nucleotide (e.g. T, G, A). According to specific embodiments, the PAM sequence is selected from the group consisting of TGG, GGG and AGG.

The gRNA comprises a “gRNA guide sequence” or “gRNA target sequence” which corresponds to the target sequence on the target polynucleotide gene sequence that is followed by a PAM sequence.

Although a perfect match between the gRNA guide sequence and the DNA sequence on the targeted gene is preferred, a mismatch between a gRNA guide sequence and target sequence on the gene sequence of interest is also permitted as long as it still allows hybridization of the gRNA with the complementary strand of the gRNA target polynucleotide sequence on the targeted gene. A seed sequence of between 8-12 consecutive nucleotides in the gRNA, which perfectly matches a corresponding portion of the gRNA target sequence is preferred for proper recognition of the target sequence. The remainder of the guide sequence may comprise one or more mismatches. In general, gRNA activity is inversely correlated with the number of mismatches. According to specific embodiments, the gRNA of the present invention comprises 7 mismatches, 6 mismatches, 5 mismatches, 4 mismatches, 3 mismatches, 2 mismatches, or less, and even no mismatch, with the corresponding gRNA target gene sequence (less the PAM). According to specific embodiments, the gRNA nucleic acid sequence is at least 90%, at least 91%, at least 92%, at least 93%, at least 94%, at least 95%, at least 96%, at least 97%, at least 98% or at least 99% identical to the gRNA target polynucleotide sequence in the gene of interest. Of course, the smaller the number of nucleotides in the gRNA guide sequence the smaller the number of mismatches tolerated. The binding affinity is thought to depend on the sum of matching gRNA-DNA combinations.

Any gRNA guide sequence can be selected in the target nucleic acid sequence, as long as it allows introducing at the proper location, the patch/donor sequence of the present invention. Accordingly, the gRNA guide sequence or target sequence of the present invention may be in coding or non-coding regions a gene (i.e., introns or exons).

In one embodiment, the gRNA is a sgRNA.

As used herein, the term “sgRNA” refers to single guide RNA used in conjunction with CRISPR associated systems (Cas). sgRNAs are a fusion of crRNA and tracrRNA and contain nucleotides of sequence complementary to the desired target site. Jinek et al., “A programmable dual-RNA-guided DNA endonuclease in adaptive bacterial immunity” Science 337(6096):816-821 (2012) Watson-Crick pairing of the sgRNA with the target site permits R-loop formation, which in conjunction with a functional PAM permits DNA cleavage or in the case of nuclease-deficient Cas9 allows binds to the DNA at that locus.

There are a number of publically available tools to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

Non-limiting examples of a gRNA that can be used in the present disclosure include those described in the Example section which follows.

Thus, for example the gRNA sequence that target DCL2 may comprise a nucleic acid sequence selected from the group consisting of SEQ ID NO: 1-2, the gRNA sequence that target DCL4 may comprise a nucleic acid sequence selected from the group consisting of SEQ ID NO: 3-4, the gRNA sequence that target RDR1 may comprise a nucleic acid sequence selected from the group consisting of SEQ ID NO: 26-27, the gRNA sequence that target RDR2 may comprise a nucleic acid sequence selected from the group consisting of SEQ ID NO: 28-29, the gRNA sequence that target RDR6 may comprise a nucleic acid sequence selected from the group consisting of SEQ ID NO: 30-31.

In order to use the CRISPR system, both gRNA and a CAS endonuclease (e.g. Cas9) should be expressed in a target cell. The insertion vector can contain both cassettes on a single plasmid or the cassettes are expressed from two separate plasmids. In addition the insertion vector may contain nucleic acid sequences encoding selectable markers, internal ribosome entry site (IRES) and the like. CRISPR plasmids are commercially available such as the px330 plasmid from Addgene (75 Sidney St, Suite 550A•Cambridge, MA 02139) or the pBIN19 vector from ATCC (catalog number 37327). Use of clustered regularly interspaced short palindromic repeats (CRISPR)-associated (Cas)-guide RNA technology and a Cas endonuclease for modifying plant genomes are also at least disclosed by Svitashev et al., 2015, Plant Physiology, 169 (2): 931-945; Kumar and Jain, 2015, J Exp Bot 66: 47-57; and in U.S. Patent Application Publication No. 20150082478, which is specifically incorporated herein by reference in its entirety. CAS endonucleases that can be used to effect DNA editing with gRNA include, but are not limited to, Cas9, CasX, Cpf1 (Zetsche et al., 2015, Cell. 163(3):759-71), C2c1, C2c2, and C2c3 (Shmakov et al., Mol Cell. 2015 Nov. 5; 60(3):385-97).

T-GEE system (TargetGene's Genome Editing Engine)—A programmable nucleoprotein molecular complex containing a polypeptide moiety and a specificity conferring nucleic acid (SCNA) which assembles in-vivo, in a target cell, and is capable of interacting with the predetermined target nucleic acid sequence. The programmable nucleoprotein molecular complex is capable of specifically modifying and/or editing a target site within the target nucleic acid sequence and/or modifying the function of the target nucleic acid sequence. Nucleoprotein composition comprises (a) polynucleotide molecule encoding a chimeric polypeptide and comprising (i) a functional domain capable of modifying the target site, and (ii) a linking domain that is capable of interacting with a specificity conferring nucleic acid, and (b) specificity conferring nucleic acid (SCNA) comprising (i) a nucleotide sequence complementary to a region of the target nucleic acid flanking the target site, and (ii) a recognition region capable of specifically attaching to the linking domain of the polypeptide. The composition enables modifying a predetermined nucleic acid sequence target precisely, reliably and cost-effectively with high specificity and binding capabilities of molecular complex to the target nucleic acid through base-pairing of specificity-conferring nucleic acid and a target nucleic acid. The composition is less genotoxic, modular in their assembly, utilize single platform without customization, practical for independent use outside of specialized core-facilities, and has shorter development time frame and reduced costs.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Introducing the agent capable of introducing the loss of function mutations to the plant cell may be effected by any method known in the art.

According to specific embodiments, the cell is introduced with a nucleic acid construct comprising a nucleic acid sequence encoding the agent.

As used herein the term “nucleic acid sequence” or “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

Constructs useful in the methods according to some embodiments of the invention may be constructed using recombinant DNA technology well known to persons skilled in the art. The gene constructs may be inserted into vectors, which may be commercially available, suitable for transforming into plants and suitable for expression of the nucleic acid sequence of interest in the transformed cells. The genetic construct can be an expression vector wherein said nucleic acid sequence is operably linked to one or more regulatory sequences allowing expression in the plant cells in a constitutive or inducible manner.

In a particular embodiment of some embodiments of the invention the regulatory sequence is a plant-expressible promoter.

As used herein the phrase “plant-expressible” refers to a promoter sequence, including any additional regulatory elements added thereto or contained therein, is at least capable of inducing, conferring, activating or enhancing expression in a plant cell, preferably a monocotyledonous or dicotyledonous plant cell.

This includes any promoter of plant origin, but also any promoter of non-plant origin which is capable of directing transcription in a plant cell, i.e., certain promoters of viral or bacterial origin such as the CaMV35S (Harpster et al. (1988) Mol Gen Genet. 212(1):182-90, type III RNA polymerase III promoter (U6), the subterranean clover virus promoter No 4 or No 7 (WO9606932), or T-DNA gene promoters but also tissue-specific or organ-specific promoters including but not limited to seed-specific promoters (e.g., WO89/03887), organ-primordia specific promoters (An et al. (1996) Plant Cell 8(1):15-30), stem-specific promoters (Keller et al., (1988) EMBO J. 7(12): 3625-3633), leaf specific promoters (Hudspeth et al. (1989) Plant Mol Biol. 12: 579-589), mesophyl-specific promoters, root-specific promoters (Keller et al. (1989) Genes Dev. 3: 1639-1646), tuber-specific promoters (Keil et al. (1989) EMBO J. 8(5): 1323-1330), vascular tissue specific promoters (Peleman et al. (1989) Gene 84: 359-369), stamen-selective promoters (WO 89/10396, WO 92/13956), dehiscence zone specific promoters (WO 97/13865) and the like.

According to specific embodiments, the promotor is a constitutive promotor.

According to other specific embodiments, the promotor is an inducible promotor.

Nucleic acid sequences of some embodiments of the invention may be optimized for plant expression. Examples of such sequence modifications include, but are not limited to, an altered G/C content to more closely approach that typically found in the plant species of interest, and the removal of codons atypically found in the plant species commonly referred to as codon optimization.

The phrase “codon optimization” refers to the selection of appropriate DNA nucleotides for use within a structural gene or fragment thereof that approaches codon usage within the plant cell of interest. Therefore, an optimized gene or nucleic acid sequence refers to a gene in which the nucleotide sequence of a native or naturally occurring gene has been modified in order to utilize statistically-preferred or statistically-favored codons within the plant cell. The nucleotide sequence typically is examined at the DNA level and the coding region optimized for expression in the plant species determined using any suitable procedure, for example as described in Sardana et al. (1996, Plant Cell Reports 15:677-681). In this method, the standard deviation of codon usage, a measure of codon usage bias, may be calculated by first finding the squared proportional deviation of usage of each codon of the native gene relative to that of highly expressed plant genes, followed by a calculation of the average squared deviation. The formula used is: 1 SDCU=n=1N [(Xn−Yn)/Yn] 2/N, where Xn refers to the frequency of usage of codon n in highly expressed plant genes, where Yn to the frequency of usage of codon n in the gene of interest and N refers to the total number of codons in the gene of interest. A table of codon usage from highly expressed genes of dicotyledonous plants is compiled using the data of Murray et al. (1989, Nuc Acids Res. 17:477-498).

One method of optimizing the nucleic acid sequence in accordance with the preferred codon usage for a particular plant cell type is based on the direct use, without performing any extra statistical calculations, of codon optimization tables such as those provided on-line at the Codon Usage Database through the NIAS (National Institute of Agrobiological Sciences) DNA bank in Japan (www(dot)kazusa(dot)or(dot)jp/codon/). The Codon Usage Database contains codon usage tables for a number of different species, with each codon usage table having been statistically determined based on the data present in Genbank.

By using the above tables to determine the most preferred or most favored codons for each amino acid in a particular species (for example, rice), a naturally-occurring nucleotide sequence encoding a protein of interest can be codon optimized for that particular plant species. This is effected by replacing codons that may have a low statistical incidence in the particular species genome with corresponding codons, in regard to an amino acid, that are statistically more favored. However, one or more less-favored codons may be selected to delete existing restriction sites, to create new ones at potentially useful junctions (5′ and 3′ ends to add signal peptide or termination cassettes, internal sites that might be used to cut and splice segments together to produce a correct full-length sequence), or to eliminate nucleotide sequences that may negatively effect mRNA stability or expression.

The naturally-occurring encoding nucleotide sequence may already, in advance of any modification, contain a number of codons that correspond to a statistically-favored codon in a particular plant species. Therefore, codon optimization of the native nucleotide sequence may comprise determining which codons, within the native nucleotide sequence, are not statistically-favored with regards to a particular plant, and modifying these codons in accordance with a codon usage table of the particular plant to produce a codon optimized derivative. A modified nucleotide sequence may be fully or partially optimized for plant codon usage provided that the protein encoded by the modified nucleotide sequence is produced at a level higher than the protein encoded by the corresponding naturally occurring or native gene. Construction of synthetic genes by altering the codon usage is described in for example PCT Patent Application 93/07278.

The nucleic acid construct of some embodiments of the invention can further include additional polynucleotide sequences that allow, for example, the translation of several proteins from a single mRNA such as an internal ribosome entry site (IRES) and sequences for genomic integration.

Plant cells may be transformed stably or transiently with the nucleic acid constructs of some embodiments. In stable transformation, the nucleic acid molecule of some embodiments is integrated into the plant genome and as such it represents a stable and inherited trait. In transient transformation, the nucleic acid molecule is expressed by the cell transformed but it is not integrated into the genome and as such it represents a transient trait.

According to specific embodiments, the transformation step comprises a stable transformation.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:
(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.
(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant cell vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Although stable transformation is presently preferred, transient transformation of plant cells is also envisaged by some embodiments of the invention.

Transient transformation can be effected by any of the direct DNA transfer methods described above or by viral infection using modified plant viruses.

Viruses that have been shown to be useful for the transformation of plant hosts include CaMV, TMV and BV. Transformation of plant cells using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant cell host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host cell and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant cell under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host cell and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plant cells. The recombinant plant viral nucleic acid is capable of replication in the host cell, systemic spread in the cell culture, and transcription or expression of foreign gene(s) (isolated nucleic acid) in the host cells to produce the desired protein.

In addition to the above, the nucleic acid molecule of some embodiments of the invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

Following the transformation step of, the plant cells may be cultured for at least 6 hours, at least 12 hours, at least 1 day, at least two days, at least a week, at least two weeks or at least three weeks, at least one month, at least 2 months, at least 3 months, at least 4 months, at least 6 months, each possibility represented a separate embodiments of the present invention.

The cells may be cultured in a positive selection medium in order to identify cells that have been successfully transformed.

As used herein, the phrase “positive selective medium” refers to the medium or growth conditions which select for cells which contain a positive selectable marker gene. Transformed cells survive and/or grow when exposed to agents or conditions which would, normally, be detrimental to the survival of a cell that did not contain the positive selectable marker gene.

Sequencing analysis may also be effected in order to identify cells that have been successfully transformed.

If the construct encodes an agent capable of introducing the loss of function mutations to the plant cell (e.g. silencing agent), a sequencing analyses may be carried out to confirm presence of mutations.

If the construct encodes an agent capable of introducing the loss of function mutations to the plant cell (e.g. silencing agent), additional analyses may be carried out to confirm down-regulation of expression and/or activity of the gene of interest (e.g. DCL2, DCL4, RDR1, RDR1, RDR6). Method of determining down-regulation of expression and/or activity are well known in the art and include RT-PCR, ELISA, Western blot, IP and the like.

According to specific embodiments, the loss of function mutation abolishes expression and/or activity of the expressed product of the gene.

Thus, according to specific embodiments, the loss of function mutations abolish expression of DCL2, DCL4, RDR1, RDR2 and/or RDR6, as determined by RT-PCR or Western blot.

According to specific embodiments, the loss of function mutations abolish expression and/or activity of DCL2, as determined by no expression of transgene specific 21-nt siRNAs in the plant cell following expression of the transgene in the plant cell.

According to specific embodiments, the loss of function mutations abolish expression and/or activity of DCL4, as determined by no expression of transgene specific 22-nt siRNAs in the plant cell following expression of the transgene in the plant cell.

Methods of determining transgene specific 21-nt and/or 22-nt siRNAs are well known in the art and are also described in details in the Examples section which follows.

If the construct encodes an expression product of interest other an agent capable of introducing the loss of function mutations to the plant cell (e.g. a recombinant protein of interest) as further described hereinbelow, additional analyses may be carried out to confirm expression and/or activity of expression product of interest. Method of determining expression and/or activity are well known in the art and include RT-PCR, ELISA, western blot, IP and the like.

Specific embodiments of the present invention are also directed to plant cells prepared by the methods disclosed herein.

Hence, according to an aspect of the present invention, there is provided a plant cell obtainable by the method.

According to an additional or an alternative aspect of the present invention, there is provided as isolated plant cell in suspension comprising loss of function mutations in all alleles of at least two genes selected from the group consisting of DCL2, DCL4, RDR1, RDR2 and RDR6 in said plant cell.

According to specific embodiments, the loss of function mutations in DCL2 comprise a mutation selected from the group consisting of:
(i) a deletion of nucleic acids residues 287-307 corresponding to SEQ ID NO: 74; and/or
(ii) an insertion of at least one nucleic acid between nucleic acids residues 304-305 corresponding to SEQ ID NO: 75.

According to specific embodiments, the loss of function mutations in DCL4 comprise a mutation selected from the group consisting of:
(i) a deletion of nucleic acids residues 977-983 corresponding to SEQ ID NO: 76;
(ii) an insertion of at least one nucleic acid between nucleic acids residues 983-984 corresponding to SEQ ID NO: 76;
(ii) a deletion of nucleic acids residues 942-949 corresponding to SEQ ID NO: 77; and/or
(iii) a deletion of nucleic acids residues 944-974 corresponding to SEQ ID NO: 77.

According to specific embodiments, the loss of function mutations in DCL2 comprise a mutation selected from the group consisting of:
(i) a deletion of nucleic acids residues 294-304 corresponding to SEQ ID NO: 74;
(ii) a deletion of nucleic acids residues 263-304 corresponding to SEQ ID NO: 74; and/or
(iii) a deletion of nucleic acids residues 296-395corresponding to SEQ ID NO: 75.

According to specific embodiments, the loss of function mutations in DCL4 comprise a mutation selected from the group consisting of:
(i) an insertion of at least one nucleic acid between nucleic acids residues 983-984 corresponding to SEQ ID NO: 76;
(ii) an insertion of at least two nucleic acid between nucleic acids residues 983-984 corresponding to SEQ ID NO: 76;
(iii) a deletion of nucleic acids residues 949-1390 corresponding to SEQ ID NO: 77; and/or
(iv) an insertion of at least two nucleic acids between nucleic acids residues 944-948 corresponding to SEQ ID NO: 77.

According to specific embodiments, the loss of function mutations in DCL2 comprise a mutation selected from the group consisting of:
(i) a deletion of nucleic acids residues 304-897 corresponding to SEQ ID NO: 74;
(ii) a deletion of nucleic acids residues 281-314 corresponding to SEQ ID NO: 74; and/or
(iii) a deletion of nucleic acids residues 300-369 corresponding to SEQ ID NO: 75.

According to specific embodiments, the loss of function mutations in DCL4 comprise a mutation selected from the group consisting of:
(i) a deletion of at least one nucleic acid between nucleic acids residues 983-984 corresponding to SEQ ID NO: 76;
(ii) a deletion of least one nucleic acid between nucleic acids residues 948-949 corresponding to SEQ ID NO: 77; and/or
(iii) a deletion of least three nucleic acids between nucleic acids residues 945-947 corresponding to SEQ ID NO: 77.

It will be appreciated that the plant cells described herein may have been further genetically modified to express an expression product of interest.

Thus, according to specific embodiments, the method comprises expressing in the isolated plant cell a recombinant expression product of interest.

According to specific embodiments, the isolated plant cell comprises a heterologous nucleic acid sequence for expressing an expression product of interest.

As used herein, the term “heterologous” refers to a nucleic acid sequence which is not native to the plant cell at least in localization or is completely absent from the native plant cell.

According to an aspect of the present invention, there is provided a method of expressing a recombinant expression product of interest in a plant cell, the method comprising culturing the plant cell disclosed herein which has been transformed with the agent capable of introducing the loss of function mutations disclosed herein and further transformed to express the expression product (e.g. polypeptide) of interest under condition which allow expression of the expression product of interest.

Such conditions may be for example an appropriate temperature (e.g., 37° C.), atmosphere (e.g., air plus 5% CO₂), pH, light, medium (e.g. MS-BY-2 medium), carbon source and supplements.

Following, the produced expression product (e.g. polypeptide) may be purified and formulated in accordance with standard procedures.

According to specific embodiments, the expression product of interest is not the agent capable of introducing the loss of function mutations to the plant cell (e.g. silencing agent) described herein.

According to specific embodiments, the expression product of interest is a polypeptide.

According to specific embodiments, the expression product of interest is a mammalian polypeptide.

According to specific embodiments, the expression product of interest is a human polypeptide.

According to some embodiments, the expression product of interest is a pharmaceutical.

Non-limiting Examples of polypeptides of interest that can be expressed by the plant cells and methods disclosed herein include cytokines, cytokine receptors, growth factors (e.g. EGF, HER-2, FGF-alpha, FGF-beta, TGF-alpha, TGF-beta, PDGF, IGF-I, IGF-2, NGF), growth factor receptors, growth hormones (e.g. human growth hormone, bovine growth hormone); insulin (e.g., insulin A chain and insulin B chain), pro-insulin, erythropoietin (EPO), colony stimulating factors (e.g. G-CSF, GM-CSF, M-CSF); interleukins; vascular endothelial growth factor (VEGF) and its receptor (VEGF-R), interferons, tumor necrosis factor and its receptors, thrombopoietin (TPO), thrombin, brain natriuretic peptide (BNP); clotting factors (e.g. Factor VIII, Factor IX, von Willebrands factor and the like), anti-clotting factors; tissue plasminogen activator (TPA), urokinase, follicle stimulating hormone (FSH), luteinizing hormone (LH), calcitonin, CD proteins (e.g., CD2, CD3, CD4, CD5, CD7, CD8, CDI Ia, CDI Ib, CD18, CD19, CD20, CD25, CD33, CD44, CD45, CD71, etc.), CTLA proteins (e.g.CTLA4); T-cell and B-cell receptor proteins, bone morphogenic proteins (BNPs, e.g. BMP-I, BMP-2, BMP-3, etc.), neurotrophic factors, e.g. bone derived neurotrophic factor (BDNF), neurotrophins, e.g. rennin, rheumatoid factor, RANTES, albumin, relaxin, macrophage inhibitory protein (e.g. MIP-I, MIP-2), viral proteins or antigens, surface membrane proteins, ion channel proteins, enzymes, regulatory proteins, immunomodulatory proteins, (e.g. HLA, MHC, the B7 family), homing receptors, transport proteins, superoxide dismutase (SOD), G-protein coupled receptor proteins (GPCRs), neuromodulatory proteins, Alzheimer's Disease associated proteins and peptides. Fusion proteins, chimeric proteins, as well as fragments or portions, or mutants, variants, or analogs of any of the aforementioned proteins and polypeptides are also included among the suitable polypeptides that can be expressed by the plant cells and methods disclosed herein. The polypeptide of interest can be a glycoprotein. One class of glycoproteins are viral glycoproteins, in particular subunits, that can be used to produce for example a vaccine. Some examples of viral proteins comprise proteins from rhinovirus, poliomyelitis virus, herpes virus, bovine herpes virus, influenza virus, newcastle disease virus, respiratory syncitio virus, measles virus, retrovirus, such as human immunodeficiency virus or a parvovirus or a papovavirus, rotavirus or a coronavirus, such as transmissible gastroenteritisvirus or a flavivirus, such as tick-borne encephalitis virus or yellow fever virus, a togavirus, such as rubella virus or eastern-, western-, or venezuelean equine encephalomyelitis virus, a hepatitis causing virus, such as hepatitis A or hepatitis B virus, a pestivirus, such as hog cholera virus or a rhabdovirus, such as rabies virus.

According to a specific embodiment, the polypeptide of interest is an antibody. The term “antibody” as used herein refers to recombinant antibodies (for example of the classes IgD, IgG, IgA, IgM, IgE) and recombinant antibodies such as single-chain antibodies, chimeric and humanized antibodies and multi-specific antibodies. The term “antibody” also refers to fragments and derivatives of all of the foregoing, and may further comprise any modified or derivatised variants thereof that retain the ability to specifically bind an epitope. Antibody derivatives may comprise a protein or chemical moiety conjugated to an antibody. A monoclonal antibody is capable of selectively binding to a target antigen or epitope. Non-limiting examples of antibodies include, monoclonal antibodies (mAbs), humanized or chimeric antibodies, camelized antibodies, camelid antibodies (Nanobodies^{RTM}), single chain antibodies (scFvs), Fab fragments, F(ab′)₂ fragments, disulfide-linked Fvs (sdFv) fragments, anti-idiotypic (anti-Id) antibodies, intra-bodies, synthetic antibodies, and epitope-binding fragments of any of the above. The term “antibody” also refers to fusion protein that includes a region equivalent to the Fc region of an immunoglobulin. Also envisaged is the production in the plant cells of so called dual-specificity antibodies (Bostrom J et al (2009) Science 323, 1610-1614).

Non-limiting examples of antibodies within the scope of the present invention include those comprising the amino acid sequences of the following antibodies: anti-TNFalpha antibodies such as Adalinunab (Humira™), anti-HER2 antibodies including antibodies comprising the heavy and light chain variable regions (see U.S. Pat. No. 5,725,856) or Trastuzumab such as HERCEPTIN™; anti-CD20 antibodies such as chimeric anti-CD20 as in U.S. Pat. No. 5,736,137, a chimeric or humanized variant of the 2H7 antibody as in U.S. Pat. No. 5,721,108; anti-VEGF antibodies including humanized and/or affinity matured anti-VEGF antibodies such as the humanized anti-VEGF antibody huA4.6.1 AVASTIN™ (WO 96/30046 and WO 98/45331); anti-EGFR (chimerized or humanized antibody as in WO 96/40210); anti-CD3 antibodies such as OKT3 (U.S. Pat. No. 4,515,893); anti-CD25 or anti-tac antibodies such as CHI-621 (SIMULECT) and (ZENAPAX) (U.S. Pat. No. 5,693,762).

According to a specific embodiment, the polypeptide of interest is an enzyme.

According to specific embodiments, the enzyme is rasburicase.

According to a specific embodiment, the enzyme is a lysosomal enzyme. Examples include, but are not limited to, a cermidase e.g., N-acetylgalactosamine-4-sulphatase (arylsulphatase B), α-glucocerebrosidase, α-L-iduronidase, alpha-galactosidase A, beta-galactosidase.

According to a specific embodiment the polypeptide of interest is a chimeric polypeptide e.g., the polypeptide of interest-attached to a heterologous polypeptide which is not native to the polypeptide of interest, also referred to as a fusion protein. Examples include, but are not limited to, Etanercept (Enbrel™), a chimeric polypeptide that fuses the TNF receptor to the constant end of the IgG1 antibody.

It will be appreciated that the plant cells described herein may have been further modified to have reduced expression and/or activity of a glycosylation enzyme.

Thus, according to specific embodiments, the plant cell has reduced expression and/or activity of a glycosylation enzyme as compared to a control plant cell of the same genetic background not subjected to an agent which downregulates expression and/or activity of said glycosylation enzyme.

According to specific embodiments, the method comprises introducing into the isolated plant cell an agent capable of downregulating expression and/or activity of a glycosylation enzyme Examples of glycosylating enzymes comprises xylosyltransferase and/or fucosyltransferase.

As used herein “Xylosyltransferase” abbreviated as “XylT” refers to an enzyme that catalyzes the transfer of xylose from GDP-xylose to the beta-linked bisecting mannose in the core of N-glycans while linking it with a beta-1,2 glycosidic linkages (EC 2.4.2.38).

As used herein “Fucosyltransferase”, abbreviated as “FucT” refers to an enzyme that catalyses the transfer of fucose from GDP-fucose to the core alpha-linked N-acetyl glucosamine (GlcNAc) of protein-bound N-glycans (EC 2.4.1.214).

The N. tabacum comprises two XylT genes and 5 FucT genes. These include:
Ntab-BX_AWOK-SS596 (Ntab-XylT-A, SEQ ID NO: 88);
Ntab-BX_AWOK-SS12784 (Ntab-XylT-B, SEQ ID NO: 89).
Ntab-K326_AWOJ-SS19752 (Ntab-FucT-A, SEQ ID NO: 90)
Ntab-BX_AWOK-SS16887 (Ntab-FucT-B, SEQ ID NO: 91).
Ntab-K326_AWOJ-SS16744 (Ntab-FucT-C, SEQ ID NO: 92).
Ntab-K326_AWOJ-SS19661 (Ntab-FucT-D, SEQ ID NO: 93)
Ntab-K326_AWOJ-SS19849 (Ntab-FucT-E, SEQ ID NO: 94).

As used herein, “reduced expression and/or activity” or “downregulating expression and/or activity” refers to a decrease of at least 10% in the level of expression and/or activity of a glycosylation enzyme in comparison to a control cell of the same genetic background which was not subjected to (or contacted with) an agent which downregulates expression and/or activity of the glycosylation enzyme, as may be determined by e.g. PCR, ELISA, Western blot analysis, immunopercipitation, flow cytometry, immuno-staining or activity assays such as comparison of oligosaccharides obtained from PNGaseA to oligosaccharides obtained from PNGaseF. According to a specific embodiment, the decrease is in at least 20%, 30%, 40% or even higher say, 50%, 60%, 70%, 80%, 90% or even 100%.

Methods and agents for reducing (or downregulating) expression and/or activity of a glycosylation enzyme are well known in the art and may be effected at the genomic (e.g. homologous recombination and site specific endonucleases) and/or the transcript level using a variety of molecules which interfere with transcription and/or translation (e.g., RNA silencing agents e.g. transcriptional gene silencing (TGS), post-transcriptional gene silencing (PTGS), quelling, co-suppression, and translational repression RNA interference (RNAi) e.g. siRNA, miRNA, antisense) or on the protein level (e.g., aptamers, small molecules and inhibitory peptides, antagonists, enzymes that cleave the polypeptide, antibodies and the like).

A detailed description on downregulation at the genomic level is further provided hereinabove. Non-limiting examples of specific methods of reducing expression and/or activity of xylosyltransferase and/or fucosyltransferase at the genomic level are disclosed for example in Hanania U, et al. (2017) Plant Biotechnology Journal 15:1120-1129; Jansing J, Sack M, et al. (2019) Plant biotechnology journal 17:350-361, Mercx Sb, et al. (2017) Frontiers in plant science 8:403-403, the contents of which are fully incorporated herein by reference.

It will be appreciated that the heterologous sequences encoding e.g. the agents disclosed herein and optionally the regulatory sequences and/or selectable markers accompanying them may be removed from the isolated plant cell once they are no longer needed (e.g. following introduction of a loss of function mutation). Various techniques are known in the art for the removal of transgenes and markers while leaving only the required ones in place. Such methods include for example: 1) Transient expression of Cas9 (Chen et al., 2018; Zhang et al., 2016); (2) Transfection of preassembled complexes of purified Cas9 protein and guide RNA (RNP) into plant protoplasts (Woo et al., 2015); (3) Using ‘suicide’ transgenes, such as the BARNASE gene under the control of the rice REG2 promoter, that effectively kill all of the CRISPR-Cas9 containing pollen and embryos, assuring that any viable embryos will be free of foreign DNA (He et al., 2018) or (4) Coupling the CRISPR construct with an RNA interference element, which targets an herbicide resistance enzyme in rice (Lu et al., 2017), resulting in transgene-free mutated plants.

It is expected that during the life of a patent maturing from this application many relevant agents capable of reducing or abolishing expression and/or activity of a target protein and specifically introducing loss of function mutations in a gene will be developed and the scope of the term agent which reduces or abolishes expression and/or activity or agent capable of introducing loss of function mutations is intended to include all such new technologies a priori.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Plant cell suspensions—Nicotiana tabacum cv. BY-2 cells (Nagata, 2004) were cultured as a suspension culture in liquid MS-BY-2 medium (Nagata and Kumagai, 1999) at 25° C. with constant agitation on an orbital shaker (85 rpm). The suspensions were grown at 50 mL of volume in 250 mL Erlenmeyers and were sub-cultured weekly at 2.5% (v/v) concentration.

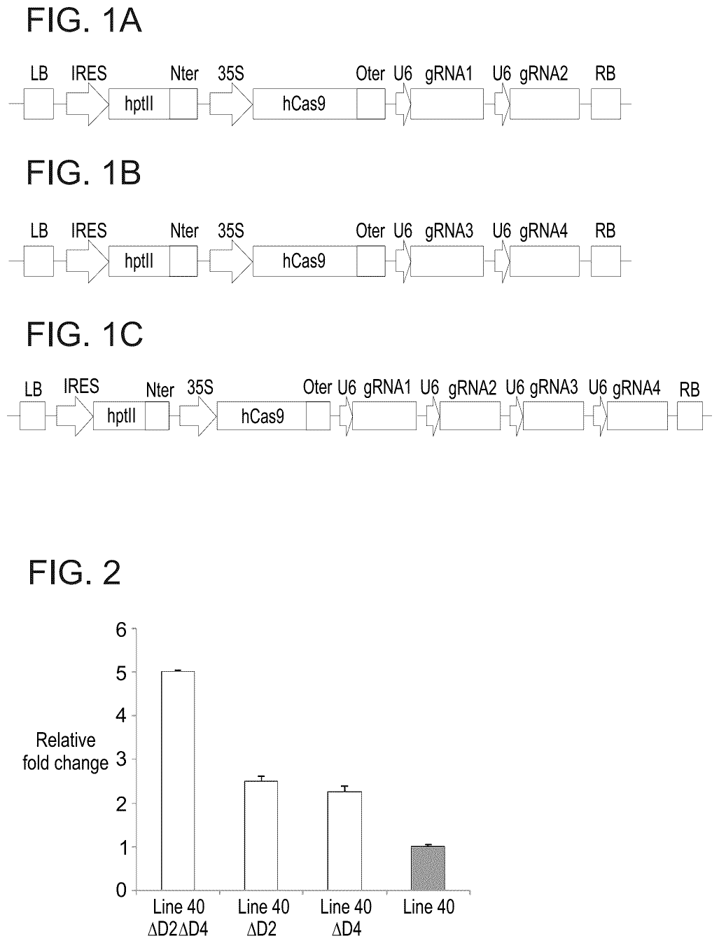

Construction of Cas9/sgRNA vectors to knockout DCL2 and DCL4 genes—Based on alignment analysis two pairs of crRNA were designed: one pair targets both genes of DCL2 (SEQ ID NO: 1-2, Table 1A hereinbelow) and one pair targets both genes of DCL4 (SEQ ID NO: 3-4, Table 1A hereinbelow) in a common homological region. Each of the four crRNAs was fused to the tracrRNA backbone sequence (SEQ ID NO: 5, Table 1A hereinbelow) resulting in the construction of four sgRNAs (designated sgRNA1-sgRNA4, respectively). Three binary vectors, i.e. phCas9-DCL2, phCas9-DCL4 and phCas9-DCL2-DCL4 (FIGS. 1A-C), were constructed using the pBIN19 backbone vector containing the human codon optimized Cas9 cassette and the appropriate cassettes of U6-gRNA directed to the DCL2 and DCL4 genes and the Neomycine phosphotransferase gene placed downstream of IRES sequence and upstream of the nopaline synthase terminator.

TABLE 1A
DNA sequences selected as the CAS9 targets
(crRNAs). Protospacer Adjacent
Motif (PAM) sequence present at
the 3′ end is marked with

| | SEQ | | Targeted cDNA |
|---|---|---|---|
| | ID | Targeted | genes |
| Name | NO: | sequence | (N. Tab) |
| crRNA1 | 1 | 5′-GGGCAAGTATTG | DCL2A (288-307) |
| | | GGGAGAAATGG-3′ | DCL2B (288-307) |
| crRNA2 | 2 | 5′-GTTTGAGTGAGC | DCL2A (881-900) |
| | | TGGGTGTTTGG-3′ | DCL2B (881-900) |
| crRNA3 | 3 | 5′-GCTAGTTGCATC | DCL4A (967-986) |
| | | CTCTTAAAGGG-3′ | DCL4B (931-950) |
| crRNA4 | 4 | 5′-GGTTGCCACCAA | DCL4A (1407-1426) |
| | | AGTTGGCGAGG-3′ | DCL4B (1371-1390) |
| tracrRNA | 5 | 5′-GTTTTAGAGCTA | |
| | | GAAATAGCAAGTTAA | |
| | | AATAAGGCTAGTCCG | |
| | | TTATCAACTTGAAAA | |
| | | AGTGGCACCGAGTCG | |
| | | GTGCTTTTTTT-3′ | |

Construction of Cas9/sgRNA vectors to knockout RDR1, RDR2, RDR6 genes—Based on alignment analysis three pairs of crRNA were designed: one pair targets both genes of RDR1 (SEQ ID NO: 26-27, Table 1B hereinbelow), one pair targets both genes of RDR2 (SEQ ID NO: 28-29, Table 1B hereinbelow) and one pair targets all three genes of RDR6 (SEQ ID NO: 30-31, Table 1B hereinbelow) in a common homological region. Each of the six crRNAs was fused to the tracrRNA backbone sequence (SEQ ID NO: 5, Table 1A hereinabove) resulting in the construction of six sgRNAs (designated sgRNA11-sgRNA16, respectively). Each of these gRNAs was constructed under the U6 promoter and inserted into a binary pBIN19 backbone vector.

TABLE 1B
DNA sequences selected as the CAS9 targets
(crRNAs). Protospacer Adjacent Motif
(PAM) sequence present at the 3′ end
is marked with italic letters.

| | SEQ | | Targeted |
|---|---|---|---|
| | ID | | cDNA genes |
| Name | NO: | Targeted sequence | (N. Tab) |
| crRNA11 | 26 | 5′ GCATGCATTGAA | RDRIA (916-937) |
| | | CACGCCTTGG 3′ | RDRIA (962-984) |
| | | | RDR1B (962-984) |
| CrRNA12 | 27 | 5′ GTTGTTATGATCC | RDRIB (916-937) |
| | | AGTGAGGTGG 3′ | |
| CrRNA13 | 28 | 5′ GAAACTCTTTGGT | RDR2A (575-553) |
| | | ATAGCTTTGG 3′ | RDR2B (575-553) |
| CrRNA14 | 29 | 5′ GCTTCAAAGTTCA | RDR2A (589-612) |
| | | GTTCTGACCGG 3′ | RDR2B (589-612) |
| CrRNA15 | 30 | 5′ GAAATCTGCAACG | RDR6A (2767-2785) |
| | | CACATGTGG 3′ | RDR6C (49-67) |
| | | | RDR6A (2820-2839) |
| | | | RDR6B (102-121) |
| | | | RDR6C (102-121) |
| CrRNA16 | 31 | 5′ GGATGAGAAGTGC | RDR6B (49-67) |
| | | CTAAAATTGG 3′ | |

Construction of a vector encoding resburicase—A Bean yellow dwarf virus (BeYDV) expression vector (Chen et al., 2011; Mor et al., 2002) encoding the rasburicase gene of Aspergillus (DB00049, SEQ ID NO: 95) was constructed. Specifically, the vector encodes the replicon initiator protein (Rep) and deletions of the viral coat and movement genes, contains insertion of an expression cassette for raburicase under the control of a CaMV 35S promoter and Octopine synthase terminator.

Transformation of cells and selection of lines—The final vectors were used to transform the tobacco cells via the Agrobacterium plant transformation procedure (An, 1985). Once a stable transgenic cell suspension was established, it was tested for transgene expression as pools or used for isolating and screening individual cell lines (clones). Establishing of individual cell lines was conducted by using highly diluted aliquots of the transgenic cell suspension and spreading them on solid medium. The cells were allowed to grow until small calli developed. Each callus, representing a single clone, was then re-suspended in liquid medium and sampled.

AFLP assay—Genomic DNA was extracted using the DNeasy plant mini kit (Qiagen). PCR amplification was effected using the appropriate forward and reverse primers (Table 2, SEQ ID 6-7 hereinbelow) in 35 cycles according to the following procedure: 95° C. for 1 minute, 60° C. for 20 seconds and 72° C. for 1 minute. Following, 5 μl of each sample was separated on 1X TBE 15% Polyacrylamide Gel Electrophoresis (PAGE) and then stained for 5 minutes with ethidium bromide.

RFLP assay—Genomic DNA was extracted using the DNeasy plant mini kit (Qiagen). PCR amplification was effected using the appropriate forward and reverse primers (Table 2 SEQ ID 8-11 hereinbelow) in 35 cycles according to the following procedure: 95° C. for 1 minute, 60° C. for 20 seconds and 72° C. for 1 minute. Following, digestion of the PCR products using the 5 restriction enzymes E.conI or PspFI was done by using 10 μl of the PCR product 3 al of restriction buffer 2 μl enzyme and 15 al DDW. The digested products were separated by electrophoresis on an ethidium bromide-stained 2% agarose gel.

DNA Sequencing—Genomic DNA was extracted using DNeasy plant mini kit (Qiagen). 35 cycles of PCR amplification was effected using the appropriate forward and reverse primers (Table 2 SEQ ID 12-19 hereinbelow) according to the following procedure: 95° C. for 1 minute, 60° C. for 20 seconds and 72° C. for 1 minute. Following, the PCR products were sub-cloned into the pGEMT vector. Colonies were sequenced by Sanger method and were aligned with the wild-type target sequences to determine mutations.

TABLE 2
Primers used for identification and characterization of indels by RFLP and
AFLP assays for DCL2 and DCL4 genes and for sequencing of DCL2 and DCL4 gene
mutations.

| SEQ | | | |
|---|---|---|---|
| ID | | | |
| NO | Sequence | Target | Assay |
| 6 | Forward: 5′- | DCL2 | AFLP |
| | TAATTACAAAGCAAGGAGATGCTCTCAT-3′ | gRNA1 | (104 bp) |
| 7 | Reverse: 5′- | | |
| | TTGCCATGTAGCAGCATCCCAATAAT 3′ | | |
| 8 | Forward: 5′- | DCL4 | RFLP |
| | CAGTAGAGTATGCACTCCAGAATCTTG-3′ | gRNA3 | (269 bp) |
| 9 | Reverse: 5′- | | |
| | CTGACTCAGATATCTGTCACACAAAGAG-3′ | | |
| 10 | Forward: 5′- | DCL2 | RFLP |
| | ATATCAACATGTGGATATTCCGTGCAC-3′ | gRNA2 | (718 bp) |
| 11 | Reverse: 5′- | | |
| | TCTAACAATCGTTTGAGCACAAACATC-3′ | | |
| 12 | Forward: 5′- | DCL2 | Sequencing |
| | TACATAGCTGTTTTCTTGGTCCCGAC-3′ | gRNA1 | (expected |
| | | | fragment size |
| 13 | Reverse: 5′- | | 1,130 bp |
| | CTTCATTATGCAGGCATAAGGGTGTTTG-3′ | | DCL2A) |
| | | | (expected |
| | | | fragment size |
| | | | 1,300 bp |
| | | | DCL2B) |
| 14 | Forward: 5′- | DCL2 | Sequencing |
| | TATCAGTTAGAGGCATTGGAGACGG-3′ | gRNA1- | (expected |
| 15 | Reverse: 5′- | gRNA2 | fragment size |
| | TCTAACAATCGTTTGAGCACAAACATC-3′ | | 3189 bp |
| | | | DCL2A) |
| | | | (expected |
| | | | fragment size |
| | | | 3351 bp |
| 16 | Forward: | DCL4 | Sequencing |
| | 5′-CAGTAGAGTATGCACTCCAGAATCTTG-3′ | gRNA3 | (expected |
| 17 | Reverse: 5′- | | fragment size |
| | CTGACTCAGATATCTGTCACACAAAGAG-3′ | | 269 bp |
| | | | DCL4A) |
| | | | (expected |
| | | | fragment size |
| | | | 271 bp |
| | | | DCL4B |
| 18 | Forward: 5′- | DCL4 | Sequencing |
| | CAGTAGAGTATGCACTCCAGAATCTTG-3′ | gRNA3- | (expected |
| 19 | Reverse : 5′ | gRNA4 | fragment size |
| | CGAAATGCTTGATGCAGAACTGATGG-3′ | | 6435 bp |
| | | | DCL4A) |
| | | | (expected |
| | | | fragment size |
| | | | 8954 bp |
| | | | DCL4B) |

siRNA detection—Micro RNA (miRNA) was isolated using the mirPreimer—miRNA isolation kit (Sigma SNC50-1kt) according to the manufacturer instructions. To a 5 μl miRNA sample, 5 μl of 2× loading dye were added and the samples were heated at 90° C. for 5 minutes to denature RNA and then placed on ice. Following, the samples were separated on 15% polyacrylamide MOPS 7M-urea gels for 2.5 hours at 100v in 20 mM MOPS. The miRNA was transferred onto positively charged nylon membrane (REF 11417240001 Roche) using the semi dry transferring cell, bio rad instrument followed by crosslinking the membrane with EDC [1-ethyl-3-(3-dimethylaminopropyl) carbodiimide].

For synthesizing the RNA probe, DIG RNA Labeling kit SP6/T7 (Sigma) was used. DNA template for transcription was inserted into the pGEMT vector and the insert orientation was determined. In order to get sense strand RNA probe, SP6 or T7 polymerase was used. Signal intensity was scanned with high resolution chemiluminescence settings using a ChemiDoc Touch Imaging System (Bio Rad) with Image Lab™ Software ver 5.2.1 (Bio-Rad).

Real-time RT-PCR—Total RNA was extracted from cell cultures using RNeasy Plant Mini Kit (Qiagen) and contaminated genomic DNA in the total RNA was degraded with TURBO DNase (Thermo Fisher Scientific). 500 ng of total RNA was used to generate the first-strand cDNA using the iSCRIPT select cDNA synthesis kit (Bio-Rad) with oligo mix/Random (1:3) primers. The qPCR mixtures were prepared using 15 μl TaqMan master mix (Thermo scientific), 3 μl ready mix of primers and probe, 5 μl template cDNA (0.4 ng to 6.4 ng) and 7 μl ddH₂O. Amplification was performed using the appropriate forward and reverse primers (Table 3 hereinbelow) in a rotor gene Real-Time PCR system (Corbett). The qPCR reaction conditions were as follows: DNA polymerase activation at 95° C. for 10 minutes was followed by 40 cycles of DNA melting at 95° C. for 15 seconds, annealing at 60° C. for 15 seconds and extension at 72° C. for 15 seconds.

TABLE 3
Primers used in real-time RT-PCR.

| SEQ | | |
|---|---|---|
| ID | | |
| NO | Sequence | Target |
| 20 | Forward: | glycosyl |
| | 5′-GAGACGAGTTCCAGAAGTGTATAG-3′ | (AB000623) |
| 21 | Reverse: | |
| | 5′-5′-TTCCTTAGCCAAATCCTTCCA-3′ | |
| 22 | Probe: | |
| | 5′-GGGAGATGCTGAGGAAGGAGAGGA-3′ | |
| 23 | Forward: | Rasburicase |
| | 5′-GCTACATGGCAGTGGAAGAA-3′ | (DB00049). |
| 24 | Reverse: | |
| | 5′-CAGCGAATGTCTTCAAAGTAACC-3′ | |
| 25 | Probe: | |
| | 5′-TGCTACTTGGGCTACAGCTAGGGA-3′ | |

### Example 1

### Construction of Cas9/SgRNA Vectors to Knock-Out all Allels of DCL2 and DCL4 Genes

In the Nicotiana tabacum genome, two genes of DCL2 and two genes of DCL4 are publicly known (Table 4 hereinbelow). In order to increase the possibility of producing a mutation two pairs of CRISPR small guide RNAs (sgRNAs) were designed based on an alignment analysis. One pair targets both genes of DCL2 and one pair targets both genes of DCL4 in a common homological region. Accordingly, the following crRNAs were defined: crRNA1, crRNA2—each 20 bp long shared between the N.tab-DCL2A and the N.tab-DCL2B genes (SEQ ID NO: 1-2, Table 1A hereinabove) and crRNA3, crRNA4—each 20 bp long shared between the N.tab-DCL4A and N.tab-DCL4B genes (SEQ ID NO: 3-4, Table 1A hereinabove). Each of the constructed crRNA was designed to hybridize with the strand complementary to the target gene and hence its sequence is of the selected common homological region of the target (FIG. 16). For the application of the CRISPR/Cas9 technology, the four crRNAs were each fused to the tracrRNA backbone sequence (SEQ ID NO: 5, Table 1A hereinabove) resulting in the construction of four sgRNAs (designated sgRNA1-sgRNA4). Three binary vector namely phCas9-DCL2 (FIG. 1A), phCas9-DCL4 (FIG. 1B) and phCas9-DCL2-DCL4 (FIG. 1C) were then constructed and used in three separate cell transformations aiming at the knockout of either the BY2-DCL2 genes, the BY2-DCL4 genes or both of genes within the same cell.

Following, in order to evaluate if knockout of DCL2 and DCL4 genes induces higher expression amounts of a recombinant protein compared to a wild type cell line, two approaches were selected. In the first approach, the three constructed vectors were used in three separate stable transformations of pre-transformed BY2 cells expressing a recombinant protein (Example 2 hereinbelow). In the second approach, the phCas9-DCL2-DCL4 vector was transformed into BY2 cells, knock-out lines were selected and then re-transformed to express a recombinant protein (Example 3 hereinbelow).

TABLE 4
Details of the N. tabacum DCL2 and DCL4 gene families

| Gene family | Gene name | Accession numbers | Remarks |
|---|---|---|---|
| DCL2 | N.tab- DCL2A | NW_015902585 TN90 | 96.4% identity |
| | N.tab- DCL2B | NW_015936378 TN90 | |
| DCL4 | Ntab- DCL4A | NW_015930707 TN90 | 97.8% identity |
| | Ntab- DCL4B | NW_015939689 TN90 | |

### Example 2

### Knocking-Out all Alleles of DCL2 and DCL4 in Cells Expressing a Recombinant Protein Up-Regulates Expression of the Recombinant Protein

BY2 cells were transformed by Agrobacterium for expression of the Aspergillus rasburicase gene (DB00049) using the Bean yellow dwarf virus (BeYDV) expression vector (Chen et al., 2011; Mor et al., 2002). A total of 100 individual transformed cell lines were isolated and screened for expression of the recombinant rasburicase. One of the lines (line 40), which expressed the higher level of rasburicase, was used for further knock-out of DCL2 and/or DCL4 genes. Thus, in a second transformation, Line 40 was transformed by Agrobacterium with the three binary vectors: phCas9-DCL2, phCas9-DCL4 or phCas9-DCL2-DCL4 (FIGS. 1A-C). The resultant transgenic cell pools of line 40 were named ΔD2, ΔD4, and ΔD2ΔD4, respectively.

Following, the effects of deletion of DCL2 and/or DCL4 on the expression level of the recombinant rasburicase was tested. Specifically, pools of cells were collected eight weeks post transformation and the expression level of rasburicase was tested by activity. The expression level of rasburicase in ΔD2ΔD4 cells was 5 fold higher compared to the expression level in the wild-type line 40, while the expression level of rasburicase in ΔD2 or ΔD4 cells was approximately 2 fold higher (FIG. 2). Importantly, there were no significant morphological or growth rate differences between ΔD2ΔD4, ΔD2 or ΔD4 cells and wild-type line 40 cells.

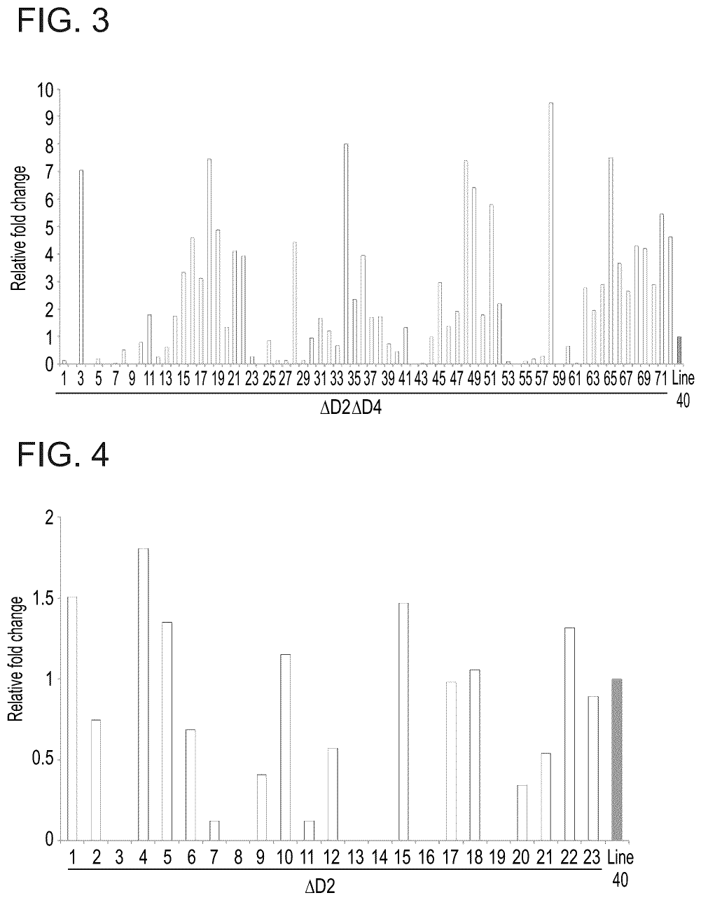

In the next step, a total of 72 individual cell lines were isolated from the transformed ΔD2ΔD4 pooled cells and the expression level of rasburicase was tested. Twenty five of these lines (about 35%) expressed 2-9 fold higher levels of rasburicase compared to the original line 40 (FIG. 3). Lines 3, 18, 34, 58 and 65 were selected for further analysis as further described hereinbelow. From the ΔD2 pooled cells, a total of 23 individual cell lines were isolated and the expression level of rasburicase was tested. Six of these lines (about 26%) expressed higher levels compared to the original line 40 (FIG. 4). Line 4 that showed the highest relative level was selected for further analysis as further described hereinbelow.

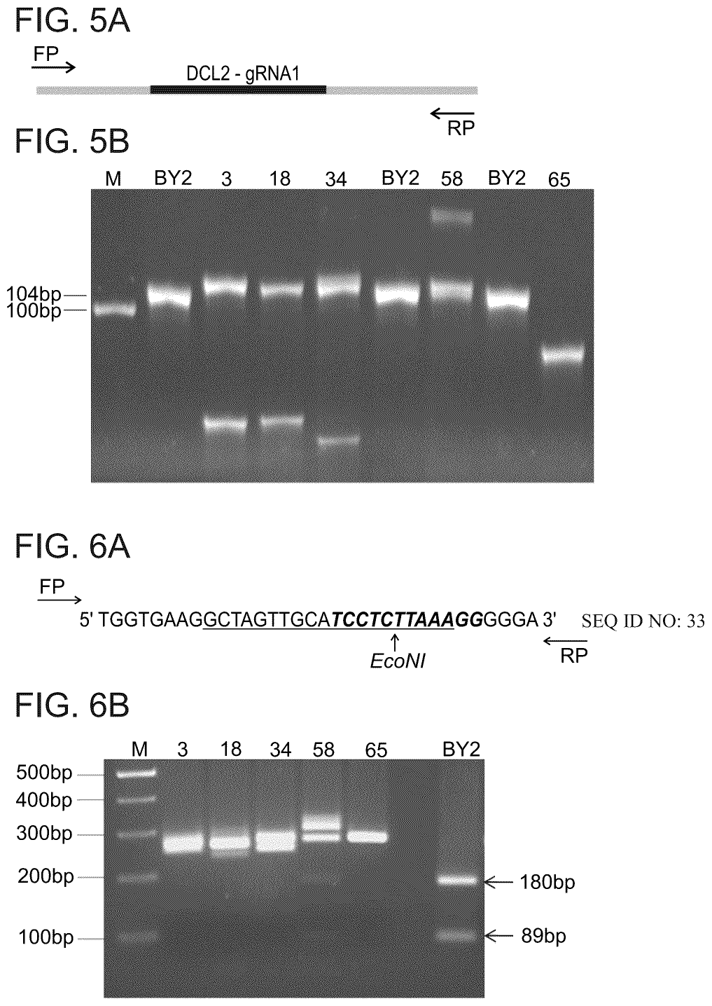

The selected ΔD2ΔD4 lines 3, 18, 34, 58 and 65 were analyzed to detect indels in DCL2 and DCL4 genes. To identify and characterize the targeted mutations in DCL2 genes, an AFLP assay was applied (Liu et al., 2015). PCR primers (SEQ ID NO: 6-7, Table 2 hereinabove) were designed to flank the target site of DCL2-gRNA1, to produce a product of 104 bp of the wild-type genes. Indeed the PCR product obtained from the wild type BY2 cells was 104 bp long. However, the PCR products from ΔD2ΔD4 lines 3, 18, 34, 58 and 65 were shorter and/or longer than the anticipated size (FIGS. 5A-B), indicating indels in all the DCL2 genes in these lines. To identify and characterize the targeted mutations in DCL4 genes, an RFLP assay was applied, which is used to assess the target loci that have restriction sites spanning the last nucleotides adjacent to the NGG (Liu et al., 2015). PCR primers (SEQ ID NO: 8-9, Table 2 hereinabove) were designed to flank the target site of DCL4-gRNA3, to produce a product of 269 bp. The amplicons were then digested with a restriction enzyme EcoNI that recognizes the wild-type target sequences and produces two fragments of 180 bp and 89 bp. Introduced mutations are expected to be resistant to restriction enzyme digestion resulting in un-cleaved bands due to loss of the restriction site. Indeed, while the expected two fragments were obtained from the wild type BY2 cells, un-cleaved bands were obtained from lines 3, 18, 34 and 65 (FIGS. 6A-B), indicating indels in all the DCL2 genes in these lines. Un-cleaved bands were also evident in line 58. This line has also shown faint bands similar to the wild type pattern. However, the presence of these two bands in line 58 does not necessarily indicate absence of mutations because within the restriction enzyme EcoNI recognition site there is a sequence of five random N-nucleotides. In the case where the CAS9 mutation is a substitution of nucleotides (i.e. not insertion or deletion) within this random 5N sequence, the enzyme can still digest the mutated site so even though the mutation occurred, the digestion will appear as a wild-type digestion. In order to analyze if this is the case, sequencing of the target gRNA region needs to be applied.

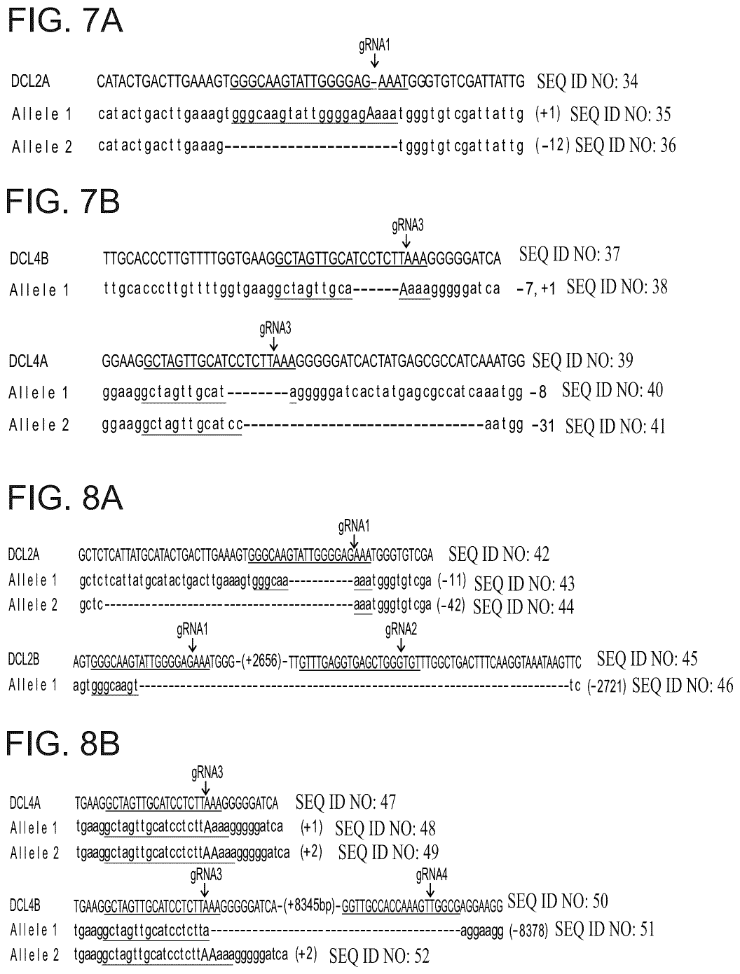

The Cas9 generated mutations in the DCL2 and the DCL4 genes in cell lines 18 and 65 were further characterized by sequencing. Specifically, to sequence the mutations in the DCL2 genes, a PCR was performed using a set of primers (SEQ ID NO: 12-13, Table 2 hereinabove) flanking the gRNA1 Cas9 target site of both DCL2 genes and primers (SEQ ID NO: 14-15, Table 2 hereinabove) flanking the gRNA1-gRNA2 Cas9 target site of both DCL2 genes. To sequence the mutations in the DCL4 genes, a PCR was performed using a set of primers (SEQ ID NO: 16-17, Table 2 hereinabove) flanking the gRNA3 Cas9 target site of both DCL4 genes and primers (SEQ ID NO: 18-19, Table 2 hereinabove) flanking the gRNA3-gRNA4 Cas9 target site of both DCL4 genes. Following, the obtained PCR products were cloned into a pGEMT vector and the sub-clones were sequenced, revealing the presence of assorted insertions and/or deletions:
Line 18: no wild type products were detected among any of the tested genes. Two mutations for the DCL2 genes were identified. A mutation of a 1bp insertion and a mutation of 12 bp deletion were identified in both alleles of DCL2A (FIG. 7A). No PCR product of DCL2B from all sub-clones was obtained, this can happen in case of a long deletion. Three mutations for the DCL4 genes were identified. A mutation of a 1 bp insertion and 7 bp deletion were identified in one allele of DCL4A. No PCR product of the second allele of DCL4A from all sub-clones was obtained, this can happen in case of a long deletion. In addition, a mutation of 8 bp deletion and a mutation of 31 bp deletion were identified in both alleles of DCL4B (FIG. 7B).
Line 65: No wild type products were detected among any of the tested genes. Three mutations for the DCL2 genes were identified, a mutation of 11 bp deletion and a mutation of 42 bp deletion where identified in both alleles of DCL2A; and a mutation of 2,721 bp deletion was identified in one allele of DCL2B (FIG. 8A). No PCR product of the second allele of DCL2B from all sub-clones was obtained, this can happen in case of a long deletion. Four mutations for the DCL4 genes were identified. A mutation of 1 bp insertion and a mutation of 2 bp insertion where identified in both DCL4A alleles; and a mutation of 8,378 bp deletion and a mutation of 2 bp insertion were identified in both DCL4B alleles DCL4B (FIG. 8B).

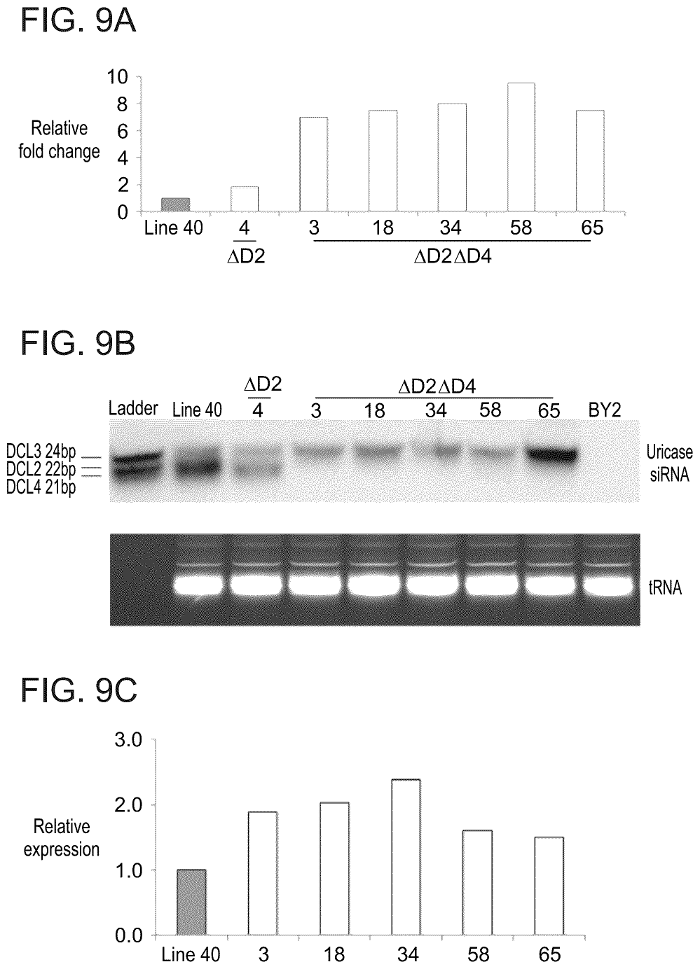

Following, to approve the mutations introduced in the DCL2 and DCL4 genes are indeed loss-of-function mutations leading to the enhanced expression of the recombinant rasburicase, the selected ΔD2ΔD4 lines 3, 18, 34, 58 and 65 which express high levels of rasburicase (7, 7.5, 8, 9.5 and 7.5 fold respectively, compared to line 40, FIG. 9A) were further analyzed for expression of small interfering RNAs (siRNA). Specifically, siRNA were extracted and hybridized with an RNA probe of the rasburicase sense sequence. The original line 40 and the selected ΔD2 line 4 produced 21-nt, 22-nt and 24-nt siRNA, while all the ΔD2ΔD4 lines produced only the 24-nt class obtained by the DCL3 gene and the 22-nt and 21-nt classes obtained by the DCL2 and DCL4 genes were not detected (FIG. 9B). These results suggest that DCL2 and DCL4 genes are active on the recombinant rasburicase transgene in an equilibrium that allows expression at a certain level (in line 40), whereas knock-out of the DCL2 and DCL4 genes abolishes completely the production of 21-nt and 22-nt siRNA and contributes to shifting the equilibrium and to increased expression levels of up to at least 7 fold. Further, the stability of rasburicase mRNA in the ΔD2ΔD4 lines improved through the repression of RNA silencing, as indicated by increased rasburicase mRNA levels in the ΔD2ΔD4 lines compared to line 40, as determined by real-time RT-PCR. Specifically, the ΔD2ΔD4 lines 3, 18 and 34 contained more or less twice; and lines 58 and 65 contained about one and a half times of accumulated rasburicase mRNA compared to the original line 40 (FIG. 9C).

### Example 3

### Expressing a Recombinant Protein in Cells Comprising Loss of Function Mutations in all Alleles of DCL2 and DCL4 Yields Higher Amounts of the Recombinant Protein

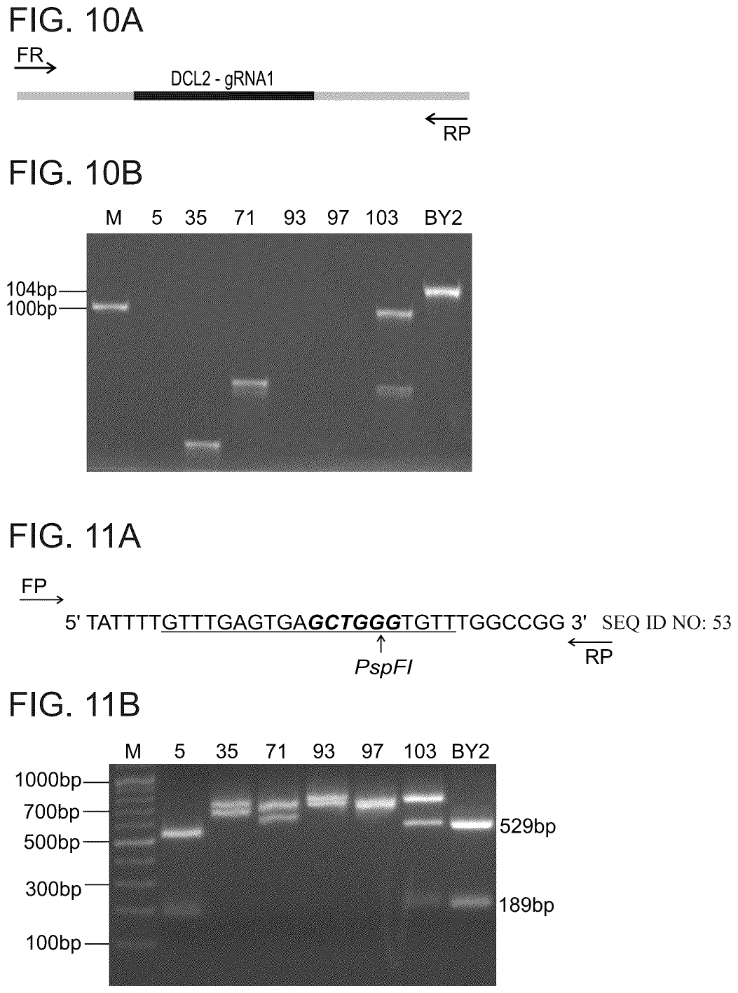

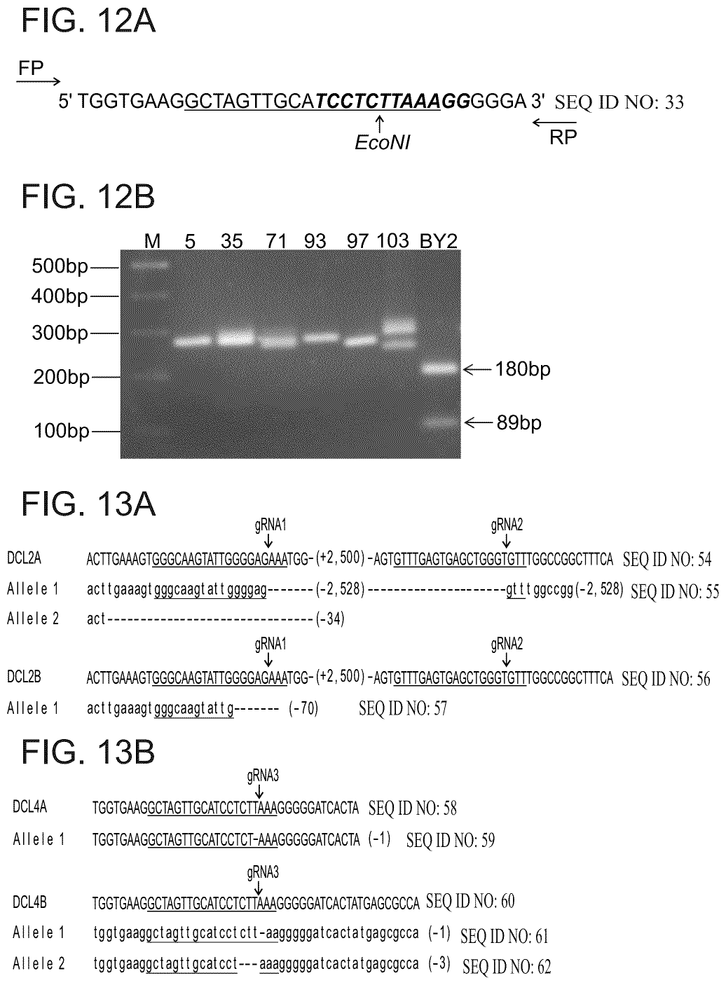

BY2 cells were transformed by Agrobacterium with the binary vector phCas9-DCL2-DCL4 (FIG. 1C) and the resultant transgenic cell pool was named ΔD2ΔD4. Following, a total of 106 individual cell lines were isolated from the transformed ΔD2ΔD4 pool cells and analyzed to detect indels in DCL2 and DCL4 genes using AFLP and RFLP methods (Liu et al., 2015). To detect targeted mutations of the DCL2 genes, a set of primers (SEQ ID NO: 6-7, Table 2 hereinabove) were designed to flank the target site of DCL2-gRNA1, to produce a product of 104 bp product in the wild-type genes for an AFLP assay; and a set of primers (SEQ ID NO: 10-11, Table 2 hereinabove) were designed to flank the target site of DCL2-gRNA2, to produce a product of 718 bp for an RFLP assay. To detect targeted mutations of the DCL4 genes, primers (SEQ ID NO: 8-9, Table 2 hereinabove) were designed to flank the target site of DCL4-gRNA3, to produce a product of 269 bp for an RFLP assay. Out of the 106 screened lines, six lines (lines 5, 35, 71, 93, 97 and 103) demonstrated knock-out of both DCL2 and DCL4 genes by AFLP and RFLP assays (FIGS. 10A-12B). Specifically, in the AFLP assay for DCL2, a 104 bp DNA fragment was produced in wild type BY2 cells while the PCR products produced from lines 35, 71 and 103 showed shorter bands of the anticipated size, and lines 5, 93 and 97 did not produce any band (FIGS. 10A-B). In the RFLP assay for DCL2, 718 bp DNA fragments were digested with a restriction enzyme PspFI that recognizes the wild-type target sequences and expected to produce two fragments 529 bp and 189 bp long. In lines 35, 71, 93, 97 and 103 un-cleaved bands were obtained, while in lines 5, 103 and wild type BY2 the expected two fragments were obtained (FIGS. 11A-B). In the RFLP assay for DCL4, 269 bp DNA fragments were digested with a restriction enzyme EcoNI that recognizes the wild-type target sequences and expected to produce two fragments 180 bp and 89 bp long. While in the wild type BY2 the expected two fragments were obtained, in all six lines (lines 5, 35, 71, 93, 97 and 103) un-cleaved bands were obtained (FIGS. 12A-B). Taken together, the results indicate that all six lines are apparently knocked-out in DCL2 and DCL4 genes. Importantly, there were no significant morphological or growth rate differences between these lines and wild-type BY2 cells.

The Cas9 generated mutations in the DCL2 and the DCL4 genes in cell line 35 were further characterized by sequencing. Specifically, to sequence the mutations in the DCL2 genes, a PCR was performed using a set of primers (SEQ ID NO: 12-13, Table 2 hereinabove) flanking the gRNA1 Cas9 target site of both DCL2 genes and primers (SEQ ID NO: 14-15, Table 2 hereinabove) flanking the gRNA1-gRNA2 Cas9 target site of both DCL2 genes. To sequence the mutations in the DCL4 genes, a PCR was performed using a set of primers (SEQ ID NO: 16-17, Table 2 hereinabove) flanking the gRNA3 Cas9 target site of both DCL4 genes. Following, the obtained PCR products were cloned into a pGEMT vector and the sub-clones were sequenced, revealing the presence of assorted insertions and/or deletions. No wild type products were detected among any of the tested genes. Three mutations for the DCL2 genes were identified: A mutation of 2,528 bp deletion and a mutation of 34 bp deletion were identified in both alleles of DCL2A and a mutation of 70 bp deletion was identified in one allele of DCL2B. No PCR product of the second allele of DCL2B from all sub-clones was obtained, this can happen in case of a long deletion. (FIG. 13A). Three mutations for the DCL4 genes were identified. A mutation of 1 bp deletion was identified in one allele of DCL4A gene. No PCR product of the second allele of DCL4A from all sub-clones was obtained, this can happen in case of a long deletion. In addition, a mutation of 1 bp deletion and a mutation of 3 bp deletion were identified in both alleles of DCL4B gene. (FIG. 13B).

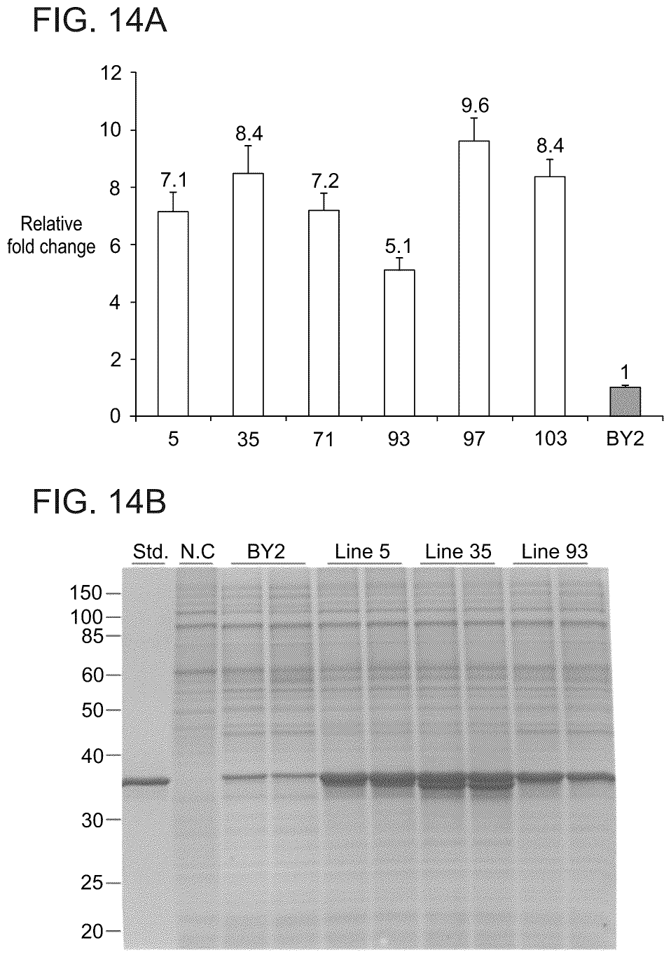

To study the effects of DCL2 and DCL4 knock-out on expression level and gene silencing against transgenes, wild-type BY2 cells and the resultant six transgenic cells lines were re-transformed by Agrobacterium for expression of the Aspergillus rasburicase gene (DB00049) using the Bean yellow dwarf virus (BeYDV) expression vector (Chen et al., 2011; Mor et al., 2002). Following, pools of each re-transformed cell line were collected eight weeks post transformation and the expression level of rasburicase was tested by activity. A significant increase in the rasburicase expression level was obtained in pools of all lines compared to wild type BY2 ranging from 5 fold in the pool of line 97 to 9.6 fold in the pool of line 97 (FIG. 14A). The increased expression level of lines 5, 35 and 93 compared to BY2 was also verified by a Coomassie stained gel (FIG. 14B). Briefly, total proteins were extracted from the appropriate lines and separated on SDS polyacrylamide gel. The gel was stained with Coomassie blue for 2 hours and destained in DDW. The prominent band at ˜38 kDa in ΔD2ΔD4 lines was more intense compared to the same band in wild-type BY2 cells, indicating the reliability of the results obtained in the activity measurements.

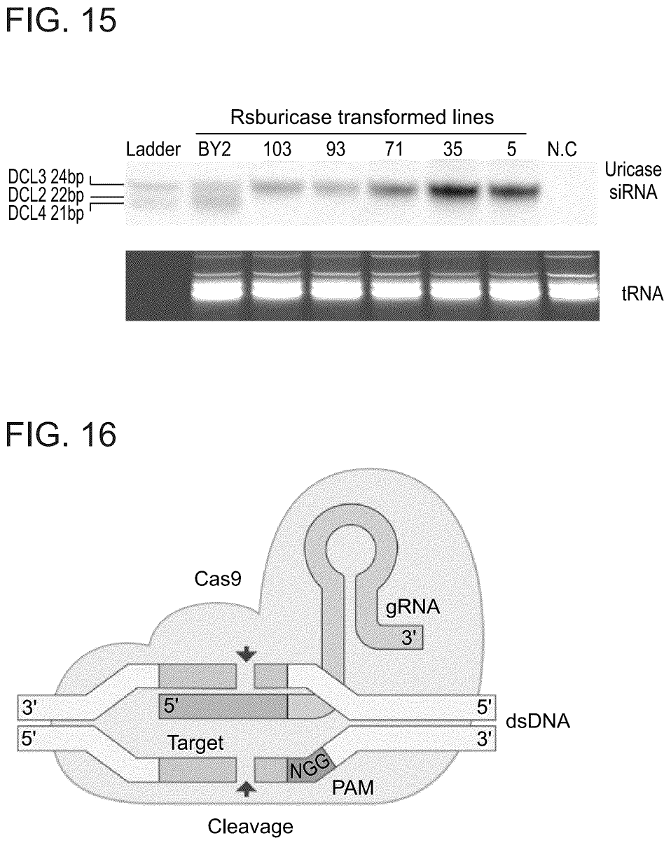

Following, to approve the mutations introduced in the DCL2 and DCL4 genes are indeed loss-of-function mutations leading to the enhanced expression of the recombinant rasburicase, the accumulation of siRNA molecules in the selected ΔD2ΔD4 lines 5, 35, 71, 93 and 103 was examined. Specifically, siRNA were extracted and hybridized with an RNA probe of the rasburicase sense sequence. The rasburicase transformed wild type BY2 cells produced 21-nt, 22-nt and 24-nt siRNAs, while all the tested ΔD2ΔD4 lines produced only the 24-nt class obtained by the DCL3 gene and the 22-nt and 21-nt classes obtained by the DCL2 and DCL4 genes were not detected (FIG. 15). These results suggest that DCL2 and DCL4 genes are active on the recombinant rasburicase transgene in an equilibrium that allows expression at a certain level, whereas knock-out of the DCL2 and DCL4 genes abolishes completely the production of 21-nt and 22-nt siRNA and contributes to shifting the equilibrium and to increased expression levels of up to at least 9 fold.

### Example 4

### Construction of Cas9/SgRNA Vectors to Knock-Out all Allels of RDR1, RDR2 and/or RDR6 Genes

In the Nicotiana tabacum genome, seven genes of the RDRα group are publicly known (Table 5 hereinbelow). Based on alignment analysis, these genes were divided into three homologous groups: Group 1-RDR1-A,B: Group 2-RDR2-A,B; and Group 3-RDR6-A,B,C. In order to increase the possibility of producing a mutation two pairs of CRISPR small guide RNAs (sgRNAs) were designed based on an alignment analysis for each group. Accordingly, the following crRNAs were defined: crRNA11, crRNA12-19 bp and 20 bp long shared between the N.tab-RDR1A and the N.tab-RDR1B genes (SEQ ID NO: 26-27, Table 1B hereinabove), crRNA13, crRNA14-20 bp and 21 bp long shared between the N.tab-RDR2A and the N.tab-RDR2B genes (SEQ ID NO: 28-29, Table 1B hereinabove), and crRNA15, crRNA16-19 bp and 20 bp long shared between the N.tab-RDR6A, N.tab-RDR6B and the N.tab-RDR6C genes (SEQ ID NO: 30-31, Table 1B hereinabove). For the application of the CRISPR/Cas9 technology, the four crRNAs were each fused to the tracrRNA backbone sequence (SEQ ID NO: 5, Table 1A hereinabove) resulting in the construction of four sgRNAs (designated sgRNA11-sgRNA16, respectively). Each of these gRNA is constructed into a binary vector namely.

Following, in order to evaluate if knockout of RDR1, RDR2 and/or RDR6 and possibly in combination with DCL2 and/or DCL4 genes induces higher expression amounts of a recombinant protein compared to a wild type cell line, two approaches were selected as described in Examples 2-3 hereinabove.

TABLE 5
Details of the N. tabacum RDR1, RDR 2 and RDR6 gene families

| Gene family | Gene name | Accession numbers |
|---|---|---|
| RDR1 | N.tab- RDR1-A | Ntab-TN90_AYMY-SS286 |
| | N.tab- RDR1-B | Ntab-TN90_AYMY-SS10620 |
| RDR2 | N.tab- RDR2-A | Nitab4.5_0006831 |
| | N.tab- RDR2-B | Nitab4.5_0010542 |
| RDR6 | N.tab- RDR6-A | Ntab-TN90_AYMY-SS110011 |
| | N.tab- RDR6-B | Ntab-TN90_AYMY-SS48576 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

An G (1985) High efficiency transformation of cultured tobacco cells. Plant physiology 79:568-570.
Baulcombe D (2004) RNA silencing in plants. Nature 431:356-363.
Blevins T, Podicheti R, Mishra V, Marasco M, Wang J, Rusch D, Tang H and Pikaard C S (2015) Identification of Pol I V and RDR2-dependent precursors of 24 nt siRNAs guiding de novo DNA methylation in Arabidopsis. eLife 4:e09591-e09591.
Canto T (2016) Transient Expression Systems in Plants: Potentialities and Constraints, in Advanced Technologies for Protein Complex Production and Characterization pp 287-301, Cham: Springer International Publishing.
Chen Q, He J, Phoolcharoen W and Mason H S (2011) Geminiviral vectors based on bean yellow dwarf virus for production of vaccine antigens and monoclonal antibodies in plants. Human Vaccines 7:331-338.
Chen W, Zhang X, Fan Y, Li B, Ryabov E, Shi N, Zhao M, Yu Z, Qin C, Zheng Q, Zhang P, Wang H, Jackson S, Cheng Q, Liu Y, Gallusci P and Hong Y (2018) A Genetic Network for Systemic RNA Silencing in Plants. Plant Physiology 176:2700.
Choudhary S, Thakur S and Bhardwaj P (2019) Molecular basis of transitivity in plant RNA silencing.
Cong L, Ran F A, Cox D, Lin S, Barretto R, Habib N, Hsu P D, Wu X, Jiang W, Marraffini L A and Zhang F (2013) Multiplex genome engineering using CRISPR/Cas systems. Science (New York, NY) 339:819-823.
Cornehssen M and Vandewiele M (1989) Both RNA level and translation efficiency are reduced by anti-sense RNA in transgenic tobacco. Nucleic Acids Research 17:833-843.
Curtin S J, Michno J-M, Campbell B W, Gil-Humanes J, Mathioni S M, Hammond R, Gutierrez-Gonzalez J J, Donohue R C, Kantar M B, Eamens A L, Meyers B C, Voytas D F and Stupar R M (2015) MicroRNA Maturation and MicroRNA Target Gene Expression Regulation Are Severely Disrupted in Soybean dicer-like1 Double Mutants. G3 (Bethesda, Md) 6:423-433.
Dadami E, Boutla A, Vrettos N, Tzortzakaki S, Karakasilioti I and Kalantidis K (2013) DICER-LIKE 4 but not DICER-LIKE 2 may have a positive effect on potato spindle tuber viroid accumulation in Nicotiana benthamiana.
Daxinger L, Hunter B, Sheikh M, Jauvion V, Gasciolli V, Vaucheret H, Matzke M and Furner I (2008) Unexpected silencing effects from T-DNA tags in Arabidopsis.
Daxinger L, Kanno T, Bucher E, van der Winden J, Naumann U, Matzke A J M and Matzke M (2009) A stepwise pathway for biogenesis of 24nt secondary siRNAs and spreading of DNA methylation. The EMBO Journal 28:48.
Donini M and Marusic C (2019) Current state-of-the-art in plant-based antibody production systems. Biotechnology letters 41:335-346.
Fire A, Xu S, Montgomery M K, Kostas S A, Driver S E and Mello C C (1998) Potent and specific genetic interference by double-stranded RNA in Caenorhabditis elegans. Nature 391:806-811.
Fukudome A and Fukuhara T (2016) Plant dicer-like proteins: double-stranded RNA-cleaving enzymes for small RNA biogenesis. Journal of Plant Research 130:33-44.
Fusaro A F, Matthew L, Smith N A, Curtin S J, Dedic-Hagan J, Ellacott G A, Watson J M, Wang M-B, Brosnan C, Carroll B J and Waterhouse P M (2006) RNA interference-inducing hairpin RNAs in plants act through the viral defence pathway. EMBO reports 7:1168-1175.
Garcia-Ruiz H, Takeda A, Chapman E J, Sullivan C M, Fahlgren N, Brempelis K J and Carrington J C (2010) Arabidopsis RNA-Dependent RNA Polymerases and Dicer-Like Proteins in Antiviral Defense and Small Interfering RNA Biogenesis during Turnip Mosaic Virus Infection. The Plant Cell 22:481.
Ghildiyal M and Zamore P D (2009) Small silencing RNAs: an expanding universe. Nature Reviews Genetics 10:94.
Hamilton A J and Baulcombe D C (1999) A Species of Small Antisense RNA in Posttranscriptional Gene Silencing in Plants. Science 286:950-952.
Hammond S M, Boettcher S, Caudy A A, Kobayashi R and Hannon G J (2001) Argonaute2, a Link Between Genetic and Biochemical Analyses of RNAi. Science 293:1146.
Ji X (2008) The Mechanism of RNase III Action: How Dicer Dices, in RNA Interference pp 99-116, Berlin, Heidelberg: Springer Berlin Heidelberg.
Jinek M, Chylinski K, Fonfara I, Hauer M, Doudna J A and Charpentier E (2012) A programmable dual-RNA-guided DNA endonuclease in adaptive bacterial immunity. Science (New York, NY) 337:816-821.
Kamthan A, Chaudhuri A, Kamthan M and Datta A (2015) Small RNAs in plants: recent development and application for crop improvement. Frontiers in plant science 6:208-208.
Katsarou K, Mitta E, Bardani E, Oulas A, Dadami E and Kalantidis K (2019) DCL-suppressed Nicotiana benthamiana plants: valuable tools in research and biotechnology. Molecular Plant Pathology 20:432-446.
Kizhner T, Azulay Y, Hainrichson M, Tekoah Y, Arvatz G, Shulman A, Ruderfer I, Aviezer D and Shaaltiel Y (2015) Characterization of a chemically modified plant cell culture expressed human-±-Galactosidase-A enzyme for treatment of Fabry disease. Molecular Genetics and Metabolism 114:259-267.
Laubinger S, Zeller G, Henz S R, Buechel S, Sachsenberg T, Wang J-W, [symbol] G and Weigel D (2010) Global effects of the small RNA biogenesis machinery on the &lt;em&gt;Arabidopsis thaliana&lt;/em&gt; transcriptome. Proceedings of the National Academy of Sciences 107:17466.
Liu W, Zhu X, Lei M, Xia Q, Botella J R, Zhu J-K and Mao Y (2015) A detailed procedure for CRISPR/Cas9-mediated gene editing in Arabidopsis thaliana. Science Bulletin 60:1332-1347.
Liu Q, Feng Y and Zhu Z (2009) Dicer-like (DCL) proteins in plants. Functional & Integrative Genomics 9:277-286.
Loh H-S, Green B J and Yusibov V (2017) Using transgenic plants and modified plant viruses for the development of treatments for human diseases. Current Opinion in Virology 26:81-89.
Martinez J, Patkaniowska A, Urlaub H, Ly¼hrmann R and Tuschl T (2002) Single-Stranded Antisense siRNAs Guide Target RNA Cleavage in RNAi. Cell 110:563-574.
Matsuo K and Matsumura T (2017) Repression of the DCL2 and DCL4 genes in Nicotiana benthamiana plants for the transient expression of recombinant proteins. Journal of Bioscience and Bioengineering 124:215-220.
Matsuo K and Atsumi G (2019) CRISPR Cas9-mediated knockout of the RDR6 gene in Nicotiana benthamiana for efficient transient expression of recombinant proteins. Planta 250:463-473
Moazed D (2009) Small RNAs in transcriptional gene silencing and genome defence. Nature 457:413.
Mor T, Moon Y-S, Palmer K and Mason H (2002) Geminivirus vectors for high-level expression of foreign proteins in plant cells.
Mor T S (2016) Molecular pharming's foot in the FDA's door: Protalix's trailblazing story. Biotechnology letters 37:2147-2150.
Mukherjee K, Campos H and Kolaczkowski B (2013) Evolution of animal and plant dicers: early parallel duplications and recurrent adaptation of antiviral RNA binding in plants. Molecular biology and evolution 30:627-641.
Napoli C, Lemieux C and Jorgensen R (1990) Introduction of a Chimeric Chalcone Synthase Gene into Petunia Results in Reversible Co-Suppression of Homologous Genes in trans. The Plant Cell 2:279.
Nekrasov V, Staskawicz B, Weigel D, Jones J D G and Kamoun S (2013) Targeted mutagenesis in the model plant Nicotiana benthamiana using Cas9 RNA-guided endonuclease. Nature Biotechnology 31:691.
Nagata T (2004) When I Encountered Tobacco BY-2 Cells!, in Tobacco BY-2 Cells pp 1-6, Berlin, Heidelberg: Springer Berlin Heidelberg.
Nagata T and Kumagai F (1999) Plant cell biology through the window of highly sychronized tobacco BY-2 cell line. Methods Cell Science 21:123-7.
Nicholson A W (2014) Ribonuclease III mechanisms of double-stranded RNA cleavage. Wiley interdisciplinary reviews RNA 5:31-48.
Obbard D J, Gordon K H J, Buck A H and Jiggins F M (2009) The evolution of RNAi as a defence against viruses and transposable elements. Philosophical transactions of the Royal Society of London Series B, Biological sciences 364:99-115.
Parent J-Sb, Bouteiller N, Elmayan T and Vaucheret H (2015) Respective contributions of Arabidopsis DCL2 and DCL4 to RNA silencing. The Plant Journal 81:223-232.
Polydore S and Axtell M J (2018) Analysis of RDR1/RDR2/RDR6-independent small RNAs in Arabidopsis thaliana improves MIRNA annotations and reveals unexplained types of short interfering RNA loci. The Plant Journal 94:1051-1063
Pratt A J and MacRae IJ (2009) The RNA-induced silencing complex: a versatile gene-silencing machine. The Journal of biological chemistry 284:17897-17901.
Qin C, Li B, Fan Y, Zhang X, Yu Z, Ryabov E, Zhao M, Wang H, Shi N, Zhang P, Jackson S, [symbol] M, Cheng Q, Liu Y, Gallusci P and Hong Y (2017) Roles of Dicer-Like Proteins 2 and 4 in Intra- and Intercellular Antiviral Silencing. Plant Physiology 174:1067.
Qu F, Ye X, Hou G, Sato S, Clemente T E and Morris T J (2005) RDR6 Has a Broad-Spectrum but Temperature-Dependent Antiviral Defense Role in &lt;em&gt;Nicotiana benthamiana&lt;/em&gt. Journal of Virology 79:15209.
Qu F, Ye X and Morris T J (2008) Arabidopsis DRB4, AGO1, AGO7, and RDR6 participate in a DCL4-initiated antiviral RNA silencing pathway negatively regulated by DCL1. Proceedings of the National Academy of Sciences 105:14732.
Rand T A, Petersen S, Du F and Wang X (2005) Argonaute2 Cleaves the Anti-Guide Strand of siRNA during RISC Activation. Cell 123:621-629.
Ruderfer I, Shulman A, Kizhner T, Azulay Y, Nataf Y, Tekoah Y and Shaaltiel Y (2018) Development and Analytical Characterization of Pegunigalsidase Alfa, a Chemically Cross-Linked Plant Recombinant Human-±-Galactosidase-A for Treatment of Fabry Disease. Bioconjugate Chemistry 29:1630-1639.
Seta A, Tabara M, Nishibori Y, Hiraguri A, Ohkama-Ohtsu N, Yokoyama T, Hara S, Yoshida K, Hisabori T, Fukudome A, Koiwa H, Moriyama H, Takahashi N and Fukuhara T (2017) Post-Translational Regulation of the Dicing Activities of Arabidopsis DICER-LIKE 3 and 4 by Inorganic Phosphate and the Redox State. Plant and Cell Physiology 58:485-495.
Song M-S and Rossi J J (2017) Molecular mechanisms of Dicer: endonuclease and enzymatic activity. Biochemical Journal 474:1603.
Suzuki T, Ikeda S, Kasai A, Taneda A, Fujibayashi M, Sugawara K, Okuta M, Maeda H and Sano T (2019) RNAi-Mediated Down-Regulation of Dicer-Like 2 and 4 Changes the Response of ‘Moneymaker’ Tomato to Potato Spindle Tuber Viroid Infection from Tolerance to Lethal Systemic Necrosis, Accompanied by Up-Regulation of miR398, 398a-3p and Production of Excessive Amount of Reactive Oxygen Species. Viruses 11:344.
Tekoah Y, Shulman A, Kizhner T, Ruderfer I, Fux L, Nataf Y, Bartfeld D, Ariel T, [symbol] “Velitski S, Hanania U and Shaaltiel Y (2015) Large-scale production of pharmaceutical proteins in plant cell culture the protalix experience. Plant Biotechnology Journal 13:1199-1208
van der Krol A R, Mur L A, Beld M, Mol J N and Stuitje A R (1990) Flavonoid genes in petunia: addition of a limited number of gene copies may lead to a suppression of gene expression. The Plant Cell 2:291.
Verdel A, Jia S, Gerber S, Sugiyama T, Gygi S, Grewal SIS and Moazed D (2004) RNAi-mediated targeting of heterochromatin by the RITS complex. Science (New York, NY) 303:672-676.
Voinnet O (2005) Non-cell autonomous RNA silencing. FEBS Letters 579:5858-5871.
Voinnet O (2008) Use, tolerance and avoidance of amplified RNA silencing by plants.
Voinnet O (2009) Origin, Biogenesis, and Activity of Plant MicroRNAs. Cell 136:669-687.
Xie M and Yu B (2015) siRNA-directed DNA Methylation in Plants. Current genomics 16:23-31.
Yoshikawa M, Peragine A, Park M Y and Poethig R S (2005) A pathway for the biogenesis of trans-acting siRNAs in Arabidopsis. Genes & development 19:2164-2175.
Zhang X, Zhu Y, Liu X, Hong X, Xu Y, Zhu P, Shen Y, Wu H, Ji Y, Wen X, Zhang C, Zhao Q, Wang Y, Lu J and Guo H (2015) Suppression of endogenous gene silencing by bidirectional cytoplasmic RNA decay in &lt;em&gt;Arabidopsis&lt;/em&gt. Science 348:120.
Zhang X, Zhu Y, Wu H and Guo H (2016) Post-transcriptional gene silencing in plants: a double-edged sword. Science China Life Sciences 59:271-276.

1. An isolated plant cell in suspension comprising loss of function mutations in all alleles of at least two genes selected from the group consisting of DCL2, DCL4, RDR1, RDR2 and RDR6 in said plant cell.

2. The isolated plant cell of claim 1, wherein:
said loss of function mutations in said DCL2 are in a region shared by all alleles of said DCL2 in said plant cell;
said loss of function mutations in said DCL4 are in a region shared by all alleles of said DCL4 in said plant cell;
said loss of function mutations in said RDR1 are in a region shared by all alleles of said RDR1 in said plant cell;
said loss of function mutations in said RDR2 are in a region shared by all alleles of said RDR2 in said plant cell; and/or
said loss of function mutations in said RDR6 are in a region shared by all alleles of said RDR6 in said plant cell.

3. The isolated plant cell of claim 1, wherein:
said loss of function mutations in said DCL2 are located within nucleic acid residues 288-307 and/or 881-900 corresponding to SEQ ID NO: 74; and/or nucleic acid residues 288-307 and/or 881-900 corresponding to SEQ ID NO: 75;
said loss of function mutations in said DCL4 are located within nucleic acid residues 967-986 and/or 1407-1426 corresponding to SEQ ID NO: 76; and/or nucleic acid residues 931-950 and/or 1371-1390 corresponding to SEQ ID NO: 77;
said loss of function mutations in said RDR1 are located within nucleic acid residues 916-937 and/or 962-984 corresponding to SEQ ID NO: 78; and/or nucleic acid residues 921-937 and/or 962-984 corresponding to SEQ ID NO: 79;
said loss of function mutations in said RDR2 are located within nucleic acid residues 575-553 and/or 589-612 corresponding to SEQ ID NO: 80; and/or nucleic acid residues 575-553 and/or 589-612 corresponding to SEQ ID NO: 81; and/or
said loss of function mutations in said RDR6 are located within nucleic acid residues 2767-2785 and/or 2820-2839 corresponding to SEO ID NO: 82; nucleic acid residues 49-67 and/or 102-121 corresponding to SEQ ID NO: 83; and/or nucleic acid residues 49-67 and/or 102-121 corresponding to SEQ ID NO: 84.

4. The isolated plant cell of claim 2, wherein:
said region shared by all alleles of said DCL2 gene comprises a sequence selected from the group consisting of SEQ ID NO: 1-2;
said region shared by all alleles of said DCL4 gene comprises a sequence a sequence selected from the group consisting of SEQ ID NO: 3-4;
said region shared by all alleles of said RDR1 gene comprises a sequence a sequence selected from the group consisting of SEQ ID NO: 26-27;
said region shared by all alleles of said RDR2 gene comprises a sequence a sequence selected from the group consisting of SEQ ID NO: 28-29; and/or
said region shared by all alleles of said RDR6 gene comprises a sequence a sequence selected from the group consisting of SEQ ID NO: 30-31.

5-16. (canceled)

17. The isolated plant cell of claim 1, wherein said plant cell has reduced expression and/or activity of a glycosylation enzyme as compared to a control plant cell of the same genetic background not subjected to an agent which downregulates expression and/or activity of said glycosylation enzyme.

18. The isolated plant cell of claim 1, comprising a heterologous nucleic acid sequence for expressing an expression product of interest.

19. A method of expressing a recombinant expression product of interest in a plant cell, the method comprising culturing the cell of claim 18 under condition which allow expression of the expression product of interest.

20. A method of abolishing expression and/or activity of at least two genes selected from the group consisting of DCL2, DCL4, RDR1, RDR2 and RDR6 in a plant cell, the method comprising introducing into an isolated plant cell in suspension an agent capable of introducing loss of function mutations in all alleles of at least two genes selected from the group consisting of DCL2, DCL4, RDR1, RDR2 and RDR6 in said plant cell.

21. The method of claim 20, wherein said method comprises introducing into said isolated plant cell an agent capable of downregulating expression and/or activity of a glycosylation enzyme.

22. The method of claim 20, wherein said plant cell has reduced expression and/or activity of a glycosylation enzyme as compared to a control plant cell of the same genetic background not subjected to an agent which downregulates expression and/or activity of said glycosylation enzyme.

23. (canceled)

24. The method of claim 20, wherein said agent is a genome editing agent.

25. The method of claim 24, wherein said genome editing agent is selected from the group consisting of CRISPR/Cas system, Zinc finger nuclease (ZFN), transcription-activator like effector nuclease (TALEN) or meganuclease.

26. The method of claim 20, wherein said agent comprises:
a DCL2 DNA targeting sequence comprising a nucleic acid sequence selected from the group consisting of SEQ ID NO: 1-2;
a DCL4 DNA targeting sequence comprising a nucleic acid sequence selected from the group consisting of SEQ ID NO: 3-4;
a RDR1 DNA targeting sequence comprising a nucleic acid sequence selected from the group consisting of SEO ID NO: 26-27;
a RDR2 DNA targeting sequence comprising a nucleic acid sequence selected from the group consisting of SEQ ID NO: 28-29; and/or
a RDR6 DNA targeting sequence comprising a nucleic acid sequence selected from the group consisting of SEQ ID NO: 30-31.

27-30. (canceled)

31. The method of claim 20, further comprising expressing in said isolated plant cell a recombinant expression product of interest other than said agent.

32. The isolated plant cell of claim 1, wherein said loss of function mutations abolish expression of said at least two genes, as determined by RT-PCR.

33. The isolated plant cell of claim 1, wherein said loss of function mutations abolish expression and/or activity of said DCL2 and DCL4, as determined by no expression of transgene specific 21-nt and 22-nt siRNAs in said plant cell following expression of said transgene in said plant cell.

34. The isolated plant cell of claim 1, wherein said plant is selected from the group consisting of Tobacco, Arabidopsis, Aloe Vera, grape seeds, oil palm, plantain, pine, banana, date, eggplant, jojoba, pineapple, rubber tree, cassava, yam, sweet potato and tomato.

35. The isolated plant cell of claim 1, wherein said plant is a Tobacco plant.

36. The isolated plant cell of claim 35, wherein said Tobacco is Nicotiana tabacum.

37. The isolated plant cell of claim 1, wherein said plant cell is a BY-2 line cell.
